(19) United States
(12) Patent Application Publication
(10) Pub. No.: US 2025/0270586 A1
(43) Pub. Date: Aug. 28, 2025

(54) Conditional male sterility in wheat

(71) Applicants: Donald Danforth Plant Science Center, St. Louis, MO (US); THE JOHN INNES CENTRE, NORWICH RESEARCH PARK, Norwich (GB); The Curators of the University of Missouri, Columbia, MO (US)

(72) Inventors: Blake Meyers, Davis, CA (US); Sebastien Belanger, Angus (GB); Graham Moore, Bury St Edmunds (GB); Azahara Martin, Cordoba (ES)

(21) Appl. No.: 18/858,966

(22) PCT Filed: Apr. 24, 2023
(86) PCT No.: PCT/US2023/066137

§ 371 Date: Oct. 22, 2024

Related U.S. Application Data
(60) Provisional application No. 63/333,988, filed on Apr. 22, 2022.
(60) Provisional application No. 63/334,177, filed on Apr. 24, 2022.

(51) Int. Cl. C12N 15/82; C07K 14/415; C12N 9/22; C12N 15/11

(52) U.S. Cl. CPC C12N 15/8289; C07K 14/415; C12N 9/22; C12N 15/111; C12N 2310/20

(57) ABSTRACT

Disclosed are genetically modified plants in the Pooideae or Bambusoideae subfamilies of plants which exhibit a conditional male-sterile phenotype. Methods of using the plants to produce hybrid seed of a Pooideae or Bambusoideae plant are also disclosed.

### CROSS REFERENCE TO RELATED APPLICATIONS

This application claims priority from Provisional Application Nos. 63/333,988, filed Apr. 22, 2022, and 63/3334, 177, filed Apr. 24, 2022, the entire contents of which are hereby incorporated by reference.

### GOVERNMENTAL RIGHTS

This invention was made with government support under 2019-67013-29010 awarded by the United States Department of Agriculture-National Institute of Food and Agriculture. The government has certain rights in the invention.

### FIELD OF THE INVENTION

The present disclosure relates generally to genetically modified plants in the Pooideae or Bambusoideae subfamilies of plants comprising an environmentally-sensitive conditional male-sterile phenotype and methods of using the plants to produce hybrid seed.

### BACKGROUND OF THE INVENTION

The improvement of crop plants through the production of hybrid varieties is a major goal of plant breeding. Crosses between inbred plant lines often result in progeny with higher yield, increased resistance to disease, and enhanced performance in different environments compared with the parental lines. Hybrid vigor boosts yield by 55% in rice, 47% in common bean (Proteus vulgaris), 68% in foxtail millet (Setaria italica), and 200% in Brassica oilseed crops.

However, the production of hybrid seed on a large scale is challenging because many crops have both male and female reproductive organs (stamen and pistil) on the same plant, either within a single flower (for example grasses, oilseed rape, tomato) or in separate flowers (for example corn). This arrangement results in a high level of self-pollination and makes large-scale directed crosses between inbred lines difficult to accomplish. To guarantee that outcrossing will occur to produce hybrid seed, breeders have either manually or mechanically removed stamens from one parental line, used natural self-incompatibility systems that prevent self-pollination, or exploited male sterility mutations that disrupt pollen development. Each of these strategies presents its own set of problems. Many crop plants do not have self-incompatibility and/or male sterility genes and use of male sterility requires a fertility restorer system. Manual emasculation is labor intensive and impractical for plants with small bisexual flowers.

Bread wheat (Triticum aestivum) and barley (Hordeum vulgare ssp. vulgare) are two self-fertilized species that respectively rank first and fourth among economically important cereal crops. Even though a deployment of hybrid seed in these grasses would have important benefits on food security in a changing world, manual emasculation is essentially impossible as a means to produce hybrid seeds on a large scale in these economically critical plants.

Accordingly, there is a need for effective hybrid seed production, and methods for controlled male sterility in grasses for effective production of hybrid seed in these economically essential plants.

### SUMMARY OF THE INVENTION

One aspect of the instant disclosure encompasses a plant or plant cell selected from the Pooideae subfamily or the Bambusoideae subfamily of plants. The plant comprises a genetic modification of at least one target site that confers a conditional male-sterile phenotype to the plant. The modification of the at least one target site comprises a modification of a reproductive 24-nt phased, secondary small interfering RNA in male reproductive tissues (reproductive 24-nt phasiRNA), expression of the reproductive 24-nt phasiRNA, expression of a polynucleotide in a biogenesis pathway of the reproductive 24-nt phasiRNA, or any combination thereof, thereby resulting in conditional male sterility.

The male-sterile phenotype can be conditional on environmental conditions selected from temperature, photoperiod, light quality, light intensity, or any combination thereof. In some aspects, the conditional male-sterile phenotype is conditional on temperature. In some aspects, the plant comprises a male-sterile phenotype when exposed to a temperature of about 18° C. to about 20° C. or below before flowering, during flowering, or both. In some aspects, the plant comprises a male-fertile phenotype when exposed to a temperature ranging from about 22° C. to about 26° C. or above before flowering, during flowering, or both.

The genetic modification can comprise defective biogenesis of pre-meiotic and mid-meiotic 24-nt phasiRNAs in male reproductive tissues, thereby resulting in conditional male sterility. In some aspects, the genetic modification comprises a modification of the expression of a polynucleotide in a biogenesis pathway of the reproductive 24-nt phasiRNA. In some aspects, the genetic modification comprises a modification of a miR2275 miRNA trigger or a modification of a biogenesis pathway of the miR2275 miRNA trigger.

The genetic modification can comprise a modification of a target nucleic acid sequence motif of miR2275 of a PHAS transcript. In some aspects, the target nucleic acid sequence motif of miR2275 comprises at least about 75% or more, at least about 85% or more, at least about 95% or more, or 100% sequence identity with a nucleic acid sequence of SEQ ID NO: 30. In one aspect, the target nucleic acid sequence motif of miR2275 comprises a nucleic acid sequence of SEQ ID NO: 30.

In some aspects, the genetic modification comprises a modification of a nucleic acid sequence encoding a PHAS precursor transcript comprising a target nucleic acid sequence motif of an sRNA trigger of pre-meiotic reproductive 24-nt phasiRNAs synthesis or a modification of a biogenesis pathway of the PHAS precursor transcript. The nucleic acid sequence of the target nucleic acid sequence motif of an sRNA trigger of pre-meiotic reproductive 24-nt phasiRNA synthesis can comprise at least about 75% or more, at least about 85% or more, at least about 95% or more, or 100% sequence identity with a nucleic acid sequence of SEQ ID NO: 31.

In some aspects, the genetic modification comprises a modification of an sRNA trigger of pre-meiotic reproductive 24-nt phasiRNAs synthesis or a modification of a biogenesis pathway of the sRNA trigger. The sRNA trigger of pre-meiotic reproductive 24-nt phasiRNAs synthesis can comprise at least about 75% or more, at least about 85% or more, at least about 95% or more, or 100% sequence identity with a nucleic acid sequence of SEQ ID NO: 48 or SEQ ID NO: 50. In some aspects, the sRNA trigger of pre-meiotic reproductive 24-nt phasiRNAs synthesis comprises a nucleic acid sequence of SEQ ID NO: 48 or SEQ ID NO: 50.

The genetic modification can comprise a modification of a target nucleic acid sequence motif of an sRNA trigger of pre-meiotic reproductive 24-nt phasiRNAs synthesis of a PHAS transcript. In some aspects, the target nucleic acid sequence motif of the sRNA trigger comprises at least about 75% or more, at least about 85% or more, at least about 95% or more, or 100% sequence identity with a nucleic acid sequence of SEQ ID NO: 31 or SEQ ID NO: 49. In one aspect, the target nucleic acid sequence motif of the sRNA trigger comprises a nucleic acid sequence of SEQ ID NO: 31 or SEQ ID NO: 49.

In some aspects, the genetic modification comprises a modification of a polynucleotide encoding a polypeptide in the biogenesis pathway of reproductive 24-nt phasiRNAs. The polypeptide in the biogenesis pathway of reproductive 24-nt phasiRNAs can be a dicer-like protein (DCL protein), a miRNA partner argonaute protein, an RNA-dependent RNA polymerase (RDR), a phasiRNA partner argonaute protein, Suppressor of gene silencing 3 (SGS3) protein, Doubled-stranded RNA binding protein (DRB), or any combination thereof. In some aspects, the miRNA partner argonaute protein comprises an AGO1 protein capable of triggering the biogenesis of 24-nt phasiRNAs. In some aspects, the phasiRNA partner argonaute protein is an AGO4 or AGO6 protein. In some aspects, the RDR protein is an RDR6 protein.

In some aspects, the DCL protein is a DCL5 protein. When the DCL protein is a DCL5 protein, the genetic modification can comprise a modification of a polynucleotide encoding a DCL5 protein. In some aspects, the genetic modification reduces the expression of the DCL5 protein.

The plant can be selected from Avena sativa (oats), Hordeum vulgare (barley), Secale cereale (rye), Triticum durum (Triticum turgidum subsp. durum), Triticum aestivum (bread wheat), a Brachypodium sp (e.g., Brachypodium distachyon), Aegilops tauschii, Triticum monococcum (Einkorn wheat), Triticum urartu (red wild einkorn wheat), ×Triticale, and Olyra latifolia.

In some aspects, the plant is barley (Hordeum vulgare). When the plant is barley, the DCL5 protein can comprise an amino acid sequence comprising at least about 75% or more, at least about 85% or more, at least about 95% or more, or 100% sequence identity with the amino acid sequence of SEQ ID NO: 1. In some aspects, the polynucleotide encoding the DCL5 protein comprises a nucleic acid sequence comprising at least about 75% or more, at least about 85% or more, at least about 95% or more, or 100% sequence identity with a nucleic acid sequence selected from SEQ ID NO: 2, SEQ ID NO: 32, and SEQ ID NO: 33. In some aspects, the genetic modification in the polynucleotide encoding the DCL5 protein comprises a deletion of a nucleic acid sequence comprising about 75% or more, at least about 85% or more, at least about 95% or more, or 100% sequence identity with the nucleic acid sequence of SEQ ID NO: 3 or SEQ ID NO: 51, a deletion of a nucleic acid sequence comprising about 75% or more, at least about 85% or more, at least about 95% or more, or 100% sequence identity with the nucleic acid sequence of SEQ ID NO: 19, or both.

In some aspects, the plant is bread wheat (Triticum aestivum). When the plant is bread wheat, the DCL5 protein can comprise an amino acid sequence comprising at least about 75% or more, at least about 85% or more, at least about 95% or more, or 100% sequence identity with the amino acid sequence of SEQ ID NO: 4, SEQ ID NO: 6, or SEQ ID NO: 8. In some aspects, the polynucleotide encoding the DCL5 protein comprises a nucleic acid sequence comprising at least about 75% or more, at least about 85% or more, at least about 95% or more, or 100% sequence identity with a nucleic acid sequence selected from SEQ ID NO: 5, SEQ ID NO: 34, SEQ ID NO: 35, SEQ ID NO: 7, SEQ ID NO: 36, SEQ ID NO: 37, SEQ ID NO: 9, SEQ ID NO: 38, or SEQ ID NO: 39.

In some aspects, the plant is durum wheat (T. turgidum). When the plant is durum wheat, the DCL5 protein can comprise an amino acid sequence comprising at least about 75% or more, at least about 85% or more, at least about 95% or more, or 100% sequence identity with an amino acid sequence of SEQ ID NO: 10 or SEQ ID NO: 12. In some aspects, the polynucleotide encoding the DCL5 protein comprises a nucleic acid sequence comprising at least about 75% or more, at least about 85% or more, at least about 95% or more, or 100% sequence identity with a nucleic acid sequence of SEQ ID NO: 11, SEQ ID NO: 40, SEQ ID NO: 41, SEQ ID NO: 13, SEQ ID NO: 42, or SEQ ID NO: 43. In other aspects, the plant comprises a polynucleotide encoding the DCL5 protein comprising a genetic modification encodes a transcript comprising a nucleic acid sequence comprising about 75% or more, at least about 85% or more, at least about 95% or more, or 100% sequence identity with nucleic acid sequence of SEQ ID NO: 44, a polynucleotide encoding the DCL5 protein comprising a genetic modification encodes a transcript comprising a nucleic acid sequence comprising about 75% or more, at least about 85% or more, at least about 95% or more, or 100% sequence identity with nucleic acid sequence of SEQ ID NO: 46, or both. In some aspects, the transcript encodes a DCL5 protein fragment comprising an amino acid sequence comprising about 75% or more, at least about 85% or more, at least about 95% or more, or 100% sequence identity with nucleic acid sequence of SEQ ID NO: 45 or a DCL5 protein fragment comprising an amino acid sequence comprising about 75% or more, at least about 85% or more, at least about 95% or more, or 100% sequence identity with nucleic acid sequence of SEQ ID NO: 47.

Another aspect of the instant disclosure encompasses one or more expression constructs for introducing a genetic modification of at least one target site that confers a conditional male-sterile phenotype to a plant or plant cell selected from the Pooideae subfamily or the Bambusoideae subfamily of plants. The one or more expression constructs comprise a promoter operably linked to a nucleic acid sequence encoding a programmable nucleic acid modification system targeted to a nucleotide sequence encoding a reproductive 24-nt phasiRNA; or a promoter operably linked to a nucleic acid sequence encoding a programmable nucleic acid modification system targeted to a polynucleotide in a biogenesis pathway responsible for biogenesis of the reproductive 24-nt phasiRNA. Expression of the nucleic acid modification system in the plant or plant cell introduces a genetic modification in the nucleotide sequence encoding the reproductive 24-nt phasiRNA, or a genetic modification of a polynucleotide in a biogenesis pathway of the reproductive 24-nt phasiRNA, or any combination thereof.

In some aspects, the programmable nucleic acid modification system comprises a Cas9 nuclease and a guide RNA (gRNA) comprising a sequence complementary to a target nucleic acid sequence within the polynucleotide encoding the polypeptide. The Cas9 nuclease can comprise a Cas9 nuclease comprising an amino acid sequence comprising at least about 75% or more, at least about 85% or more, at least about 95% or more, or 100% sequence identity with an amino acid sequence of SEQ ID NO: 14.

In some aspects, the genetic modification comprises a modification of a nucleic acid sequence in a polynucleotide encoding a DCL5 protein. The genetic modification can reduce the expression of the DCL5 protein.

In some aspects, the plant is H. vulgare. When the plant is H. vulgare, the polypeptide in the phasiRNA biogenesis pathway can be a DCL5 protein encoded by a nucleic acid sequence comprising at least about 75% or more, at least about 85% or more, at least about 95% or more, or 100% sequence identity with a nucleic acid sequence of SEQ ID NO: 2, SEQ ID NO: 32, or SEQ ID NO: 33. In some aspects, the gRNA comprises a nucleic acid sequence selected from SEQ ID NO: 15 (gRNA1), SEQ ID NO: 16 (gRNA2), SEQ ID NO: 17 (gRNA3), SEQ ID NO: 18 (gRNA4), and any combination thereof. In some aspects, the one or more expression constructs comprise an expression construct comprising a nucleic acid sequence comprising about 75% or more, at least about 85% or more, at least about 95% or more, or 100% sequence identity with the nucleic acid sequence of SEQ ID NO: 52 (HvuDCL-Binary-vector-pcoCAS9-HvDCL5).

The plant can be T. aestivum. When the plant is T. aestivum, the polypeptide in the phasiRNA biogenesis pathway can be a DCL5 protein comprising an amino acid sequence comprising at least about 75% or more, at least about 85% or more, at least about 95% or more, or 100% sequence identity with an amino acid sequence of SEQ ID NO: 4, SEQ ID NO: 6, or SEQ ID NO: 8. In some aspects, the gRNA comprises a nucleic acid sequence selected from SEQ ID NO: 20 (gRNA1), SEQ ID NO: 21 (gRNA2), SEQ ID NO: 22 (gRNA3), SEQ ID NO: 23 (gRNA4), SEQ ID NO: 24 (gRNA5), SEQ ID NO: 25 (gRNA6), and any combination thereof. The gRNA can comprise a nucleic acid sequence complementary to a target sequence within a nucleotide sequence comprising at least about 75% or more, at least about 85% or more, at least about 95% or more, or 100% sequence identity with a nucleic acid sequence of SEQ ID NO: 29.

In some aspects, the one or more expression constructs comprise an expression construct comprising a nucleic acid sequence comprising about 75% or more, at least about 85% or more, at least about 95% or more, or 100% sequence identity with the nucleic acid sequence of SEQ ID NO: 53 (pggg-tadcl-guides135). In other aspects, the one or more expression constructs comprise an expression construct comprising a nucleic acid sequence comprising about 75% or more, at least about 85% or more, at least about 95% or more, or 100% sequence identity with the nucleic acid sequence of SEQ ID NO: 54 (pggg-tadcl-guides246). In some aspects, the one or more expression constructs comprise an expression construct comprising a nucleic acid sequence comprising about 75% or more, at least about 85% or more, at least about 95% or more, or 100% sequence identity with the nucleic acid sequence of SEQ ID NO: 53 (pggg-tadcl-guides135) and an expression construct comprising a nucleic acid sequence comprising about 75% or more, at least about 85% or more, at least about 95% or more, or 100% sequence identity with the nucleic acid sequence of SEQ ID NO: 54 (pggg-tadcl-guides246).

Yet another aspect of the instant disclosure encompasses one or more plants or plant cells comprising one or more expression constructs described herein above.

An additional aspect of the instant disclosure encompasses a method of generating a genetically modified Pooideae or Bambusoideae plant comprising a conditional male-sterile phenotype. The method comprises introducing one or more expression constructs for introducing a genetic modification of at least one target site that confers a conditional male-sterile phenotype to a plant or plant cell selected from the Pooideae subfamily or the Bambusoideae subfamily of plants; and growing the plant or plant cell for a time and under conditions sufficient for the one or more nucleic acid expression constructs to express the engineered nucleic acid modification system in the plant or plant cell. Expressing the programmable nucleic acid modification system introduces a nucleic acid modification in the nucleic acid sequence encoding a reproductive 24-nt phasiRNA or in a polynucleotide in the phasiRNA biogenesis pathway, thereby modifying the expression of the reproductive 24-nt phasiRNA, modifying the expression of the reproductive 24-nt phasiRNA, modifying the expression of a polynucleotide in a biogenesis pathway of the reproductive 24-nt phasiRNA, or any combination thereof, thereby generating a genetically modified plant comprising a conditional male-sterile phenotype.

One aspect of the instant disclosure encompasses a method of producing hybrid seed of a Pooideae or Bambusoideae plant. The method comprises planting seeds of a first genetically modified parent Pooideae or Bambusoideae plant comprising a conditional male-sterile phenotype and a second parent plant; allowing the seeds to germinate and grow into plants; submitting the first parent plants before flowering, during flowering, or both for a time and under conditions sufficient for the plants to develop the conditional male-sterile phenotype; and allowing the second parent plants to pollinate the first parent plants to thereby produce the hybrid seed on the first parent plant. The genetically modified Pooideae or Bambusoideae plant can be as described herein above.

Another aspect of the instant disclosure encompasses a hybrid seed of a plant of a Pooideae or Bambusoideae plant comprising a conditional male-sterile phenotype. The plant is produced using a method described herein above.

Yet another aspect of the instant disclosure encompasses a kit for generating a plant of a Pooideae or Bambusoideae plant comprising a conditional male-sterile phenotype or for producing hybrid seed of the Pooideae or Bambusoideae plant. The kit comprises one or more genetically modified plants or plant cells in the Pooideae or Bambusoideae subfamily of plants comprising a conditional male-sterile phenotype; one or more expression constructs described herein above; one or more plants or plant cells described herein above; or any combination thereof.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 is a diagram depicting biogenesis of reproductive phasiRNAs in rice and maize.

FIG. 2 is a diagram depicting biogenesis of reproductive phasiRNAs in Pooideae and Bambusoideae plants.

FIG. 3A is a sequence logo of the putative nucleic acid target sequence motif of an unknown miRNA (or other sRNA type) present in the nucleic acid sequences encoding PHAS precursor transcripts of pre-meiotic 24-nt phasiRNAs. The motifs of FIGS. 3A and 3B are present in the nucleic acid sequences encoding over 75% PHAS precursor transcripts of pre-meiotic and mid-/post-meiotic 24-nt phasiRNAs. Shown are all species merged; Pre-meiotic motif; no miRNA matching with the motif; (n=5293/7024); Length: 22; E-value: 9.5e-183

FIG. 3B is a sequence logo of the putative nucleic acid target sequence motif of miR2275 present in the nucleic acid sequences encoding PHAS precursor transcripts of mid-/post-meiotic 24-nt phasiRNAs. The motifs of FIGS. 3A and 3B are present in the nucleic acid sequences encoding over 75% PHAS precursor transcripts of pre-meiotic and mid-/post-meiotic 24-nt phasiRNAs. Shown are all species merged; Mid-/Post-meiotic motif; matching with miR2275; (n=4089/5352); Length: 22; E-value: 4.2e-247.

FIG. 4 is an evolutionary tree showing the emergence of pre-meiotic 24-nt reproductive phasiRNAs before the split between Pooideae and Bambusoideae plants while absent in maize and rice.

FIG. 5 is a diagram showing conservation of miRNA target motifs across the Pooideae and Bambusoideae plants found in pre-meiotic and mid-/post-meiotic 24-nt phasiRNA groups.

FIG. 6 are heatmaps showing distribution of 24-nt reproductive phasiRNAs in anthers of seven sampled Pooideae and Bambusoideae species at three development stages.

FIG. 7 are heat maps showing distribution of 21-nt reproductive phasiRNAs in anthers of seven sampled species of Pooideae and Bambusoideae species at three stages of development of pollen.

FIG. 8A are the nucleic frequency biases observed between class of 21-nt and 24-nt reproductive phasiRNAs expressed at pre-meiotic and mid-/post-meiotic developmental stages. The frequency of nucleotides was calculated at each position of the most abundant sRNA found in all PHAS loci merged from all six Pooideae and one Bambusoideae species.

FIG. 8B are the nucleic frequency biases observed between class of 21-nt and 24-nt reproductive phasiRNAs expressed at pre-meiotic and mid-/post-meiotic developmental stages. The frequency of nucleotides was calculated at each position of all sRNA found in all PHAS loci merged from all six Pooideae and one Bambusoideae

FIG. 9 is a diagrammatic representation of DCL5 genes of H. vulgare, T. turgidum, and T. aestivum. The diagrams show the locations of mutations generating a premature stop codon in T. turgidum DCL5 genes and the target sites for each gRNA used to generate H. vulgare and T. aestivum CRISPR mutants. HvuDCL5: Barley; TtuDCL5: Tetraploid wheat; TaeDCL5: Hexaploid wheat; g1-g6: guide RNA; Kro4585; Kro2086. Kronos lines have mutation generating STOP codons in DCL5 of A and B sub-genomes

FIG. 10 is a photograph of the whole plant and a representative inflorescence in wildtype T. turgidum and all allelic combinations dcl5 loss-of-function mutants. Photographs show that a single allele is enough to maintain the male fertility while a homozygous dcl5 double mutant is male sterile. The genotype of each plant is depicted.

FIG. 11A shows the temperature-sensitive male sterile phenotype in dcl5 loss-of-function mutant in T. turgidum. Photographs of inflorescences from the homozygous dc/5 loss-of-function T. turgidum mutant grown at various temperatures compared to the wildtype plant growth at normal growth condition.

FIG. 11B are box plots showing the number of seeds produced by homozygous loss-of-function dcl5 T. turgidum mutants illustrating the gradation in the conditional male sterile phenotype while plants are sterile at low temperature (18° C.) and recover the fertility with rising temperatures (maximum recovery at 26° C.)

FIG. 12 are photomicrographs showing cross sections of anthers from the homozygous loss-of-function dcl5 (aabb) T. turgidum mutant grown under sterile (18° C.) and fertile (26° C.) temperatures compared to the wildtype plant in T. turgidum. Pre-meiotic, mid-meiotic, early post-meiotic, and pollen developmental stages. Anthers were fixed with a 2% paraformaldehyde:glutaraldehyde solution and embedded using the Quetol epoxy resin, sectioned to 0.5 μm and stained using the toluidine blue for epoxy resin. Scale bars=20 μm.

FIG. 13 are photomicrographs showing a time-series cross sections of anthers from the homozygous loss-of-function dcl5 (aabb) T. turgidum mutant grown at 18° C. (sterile development) at 13 developmental stages of the anther. Anthers were fixed with a 2% paraformaldehyde:glutaraldehyde solution and embedded using the Quetol epoxy resin, sectioned to 0.5 μm and stained using the toluidine blue for epoxy resin. Scale bars=20 μm.

FIG. 14 are photomicrographs showing a time-series cross sections of anthers from the homozygous loss-of-function dcl5 (aabb) T. turgidum mutant grown at 26° C. (fertile development) at 13 developmental stages of the anther. Anthers were fixed with a 2% paraformaldehyde:glutaraldehyde solution and embedded using the Quetol epoxy resin, sectioned to 0.5 μm and stained using the toluidine blue for epoxy resin. Scale bars=20 μm.

FIG. 15 are photomicrographs showing a time-series cross sections of anthers from the wildtype (AABB) T. turgidum anthers grown at 20° C. at 13 developmental stages of the anther. Anthers were fixed with a 2% paraformaldehyde:glutaraldehyde solution and embedded using the Quetol epoxy resin, sectioned to 0.5 μm and stained using the toluidine blue for epoxy resin. Scale bars=20 μm.

FIG. 16 are scanning electron microscopy (SEM) micrographs of anther dehiscence zones and mature pollen grains of homozygous loss-of-function dc/5 (aabb) T. turgidum grown at 18° C. (Sterile) and 26° C. (Fertile) and wild type homozygous (AABB) T. turgidum grown at 20° C. The magnification is 500×.

FIG. 17 are SEM micrographs of anther dehiscence zones and mature pollen grains of homozygous null dcl5 (aabb) T. turgidum grown at 18° C. (Sterile). The magnification are 500×, 2000× and 5000×.

FIG. 18 are SEM micrographs of anther dehiscence zones and mature pollen grains of homozygous null dcl5 (aabb) T. turgidum grown at 26° C. (Fertile). The magnification are 500×, 2000× and 5000×.

FIG. 19 are SEM micrographs of anther dehiscence zones and mature pollen grains of wild type homozygous (AABB) T. turgidum grown at 20° C. (Fertile). The magnifications are 500×, 2000× and 5000×.

FIG. 20 is a MDS plot of phasiRNAs accumulating in four DCL5 durum wheat genotypes. Green highlights developmental stages unique to the aabb genotype grown at three temperatures regulating the sterile/fertile developmental switch, and other colors highlight developmental stages common to AABB, aAbb and aabB genotypes.

FIG. 21 are heatmaps showing 21-nt reproductive phasiRNAs in pre-, mid-, and post-meiotic reproductive tissues from wild type and various mutant dcl5 genotypes grown at various temperatures.

FIG. 22 are heatmaps showing 24-nt reproductive phasiRNAs in pre-, mid-, and post-meiotic reproductive tissues from wild type and various mutant dcl5 genotypes grown at various temperatures.

FIG. 23A are box plots showing the distribution of phasiRNA abundance of 21-nt reproductive phasiRNAs at pre-, mid-, and post-meiotic developmental stages of anthers in various genotypes of wheat. The distribution of abundance describes the absolute count of phasiRNAs in Reads Per Million Mapped (RPMM) or the abundance transformed using the logarithm in base 10 (Log10RPMM) and the square root (sqrt RPMM) functions.

FIG. 23B are box plots showing the distribution of phasiRNA abundance of 24-nt (B) reproductive phasiRNAs at pre-, mid-, and post-meiotic developmental stages of anthers in various genotypes of wheat. The distribution of abundance describes the absolute count of phasiRNAs in Reads Per Million Mapped (RPMM) or the abundance transformed using the logarithm in base 10 (Log10RPMM) and the square root (sqrt RPMM) functions.

### DETAILED DESCRIPTION

The present disclosure is based in part on the surprising demonstration of conditional male-sterility in grasses where no other methods of producing hybrid seed exists. More specifically, the inventors surprisingly and unexpectedly discovered that unlike crop grasses such as maize and rice, plants in the Pooideae or Bambusoideae subfamilies of plants such as wheat, barley, oats (Avena sativa), and rye (Secale cereale) comprise a distinctive 24-nt phased small interfering RNAs (phasiRNAs) at the pre-meiotic stage of development of male reproductive tissue not found in maize and rice. Importantly, the inventors also discovered that altering the biogenesis of the 24 nt reproductive phasiRNAs results in male sterility in durum wheat (Triticum turgidum) and barley (Hordeum vulgare), two Pooideae species and potentially reproducible in other Pooideae and Bambusoideae species as the distinctive evolution of pre-meiotic 24-nt reproductive phasiRNAs is found exclusively in these sub-families. The male sterility phenotype can be conditional on environmental growth conditions. Surprisingly, there is a near complete reversal of the environmental conditions that induce male sterility in plants of durum wheat and barley when compared to other plants outside the Pooideae and Bambusoideae subfamilies such as maize and rice. The availability of these genetically engineered male-sterile plants can facilitate the development of new breeding and production systems for hybrid crops where such methods did not previously exist for the economically important plants of the Pooideae or Bambusoideae subfamilies.

### I. Genetically Modified Plants

One aspect of the present disclosure encompasses a plant in the Pooideae or Bambusoideae subfamilies of plants comprising a genetic modification of at least one target site. The genetic modification modifies a reproductive 24-nt phasiRNA, a secondary small interfering RNA in male reproductive tissues (reproductive 24-nt phasiRNA), expression of the reproductive 24-nt phasiRNA, expression of a polynucleotide in a biogenesis pathway of the reproductive 24-nt phasiRNA, or any combination thereof. The at least one modification of the at least one target site confers a conditional male-sterile phenotype to the plant.

(a) Reproductive phasiRNAs

PhasiRNAs constitute a major category of small 21 or 24 nucleotide-long RNAs in plants, but most of their functions are still poorly defined. One subclass of phasiRNAs is involved in reproductive development (reproductive phasiRNAs) and represent over 90% of all sRNAs expressing in barley and wheat anthers.

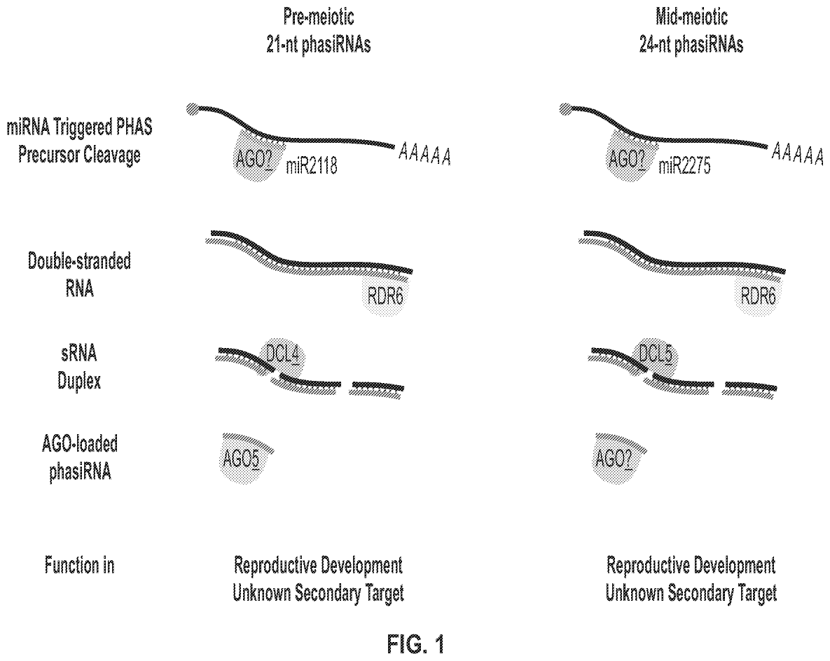

The 21-nt and 24-nt reproductive phasiRNAs exhibit a strict temporal accumulation in reproductive tissues. In rice and maize (schematized in FIG. 1), the 21-nucleotide reproductive phasiRNAs are enriched in early-stage anthers and are thus known as pre-meiotic reproductive phasiRNAs. A different phasiRNA accumulation pattern for 24-nt phasiRNAs is observed. The 24-nt phasiRNAs are almost undetectable until the anthers enter the early meiotic stage and are thus known as mid-meiotic phasiRNAs.

The inventors discovered that biogenesis and temporal distribution of 24-nucleotide phasiRNAs in the Pooideae or Bambusoideae subfamilies of plants is distinct from biogenesis and temporal distribution in other grasses. More specifically, the inventors discovered that at their peak in quantity and diversity (in the 0.2 to 0.8 mm anthers), 21-nt phasiRNAs represented more than 90% of all 21-nt sRNAs detected in anthers of Pooideae and Bambusoideae plants; significantly higher than the 60% peak proportion of 21-nt reproductive phasiRNAs observed in maize. In addition, a different phasiRNA accumulation pattern for 24-nt phasiRNAs is observed at the same developmental stage as 21-nt phasiRNAs; which contrast to reproductive phasiRNA described in maize and rice. Another group of mid-meiotic 24-nt phasiRNAs, at their peak, reached 93% of all 24-nt sRNAs detected in anthers. This was again substantially greater than the 64% peak proportion observed in maize.

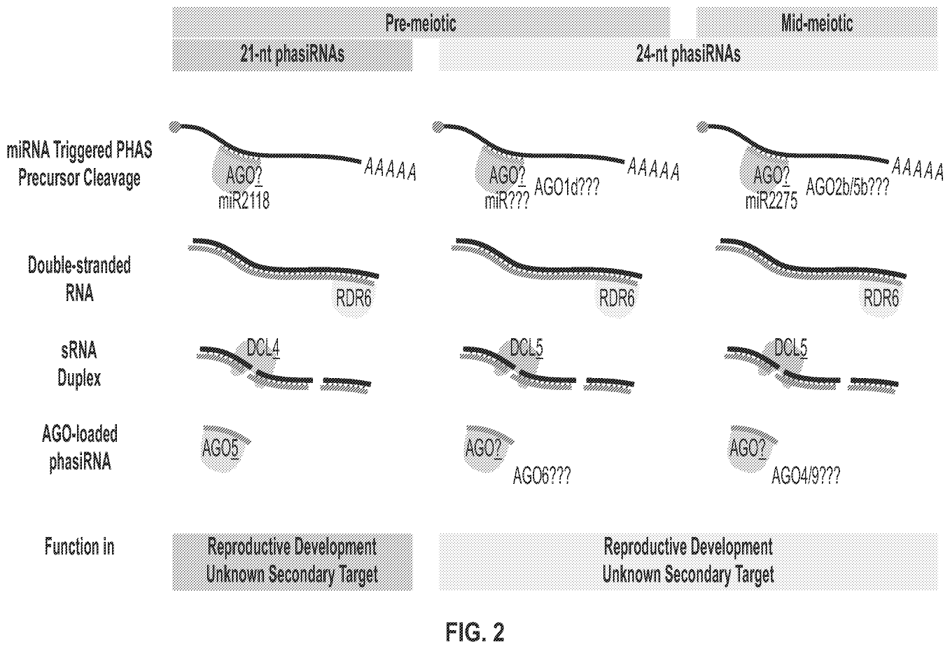

Importantly, the inventors also discovered that, unlike the single pattern of accumulation of 24-nt reproductive phasiRNAs in maize and rice, 24-nt phasiRNAs in Pooideae and Bambusoideae plants comprise two distinct groups of reproductive 24-nt phasiRNAs exhibiting two distinct patterns of accumulation (FIG. 2). A first group of 24-nt reproductive phasiRNAs accumulate more like the previously characterized 24-nt phasiRNAs in maize and rice, at the mid-meiotic stage. As with the previously characterized 24-nt phasiRNAs in maize and rice, biogenesis of the mid-meiotic group of 24-nt phasiRNAs is mediated by the miR2275 miRNA trigger. Accordingly, a genetically modified plant of the instant disclosure can comprise a genetic modification in a miR2275 miRNA trigger or in a biogenesis pathway of the miR2275 miRNA trigger or one of the Argonaute (AGO) protein initiating the biogenesis or the effector of produced phasiRNAs.

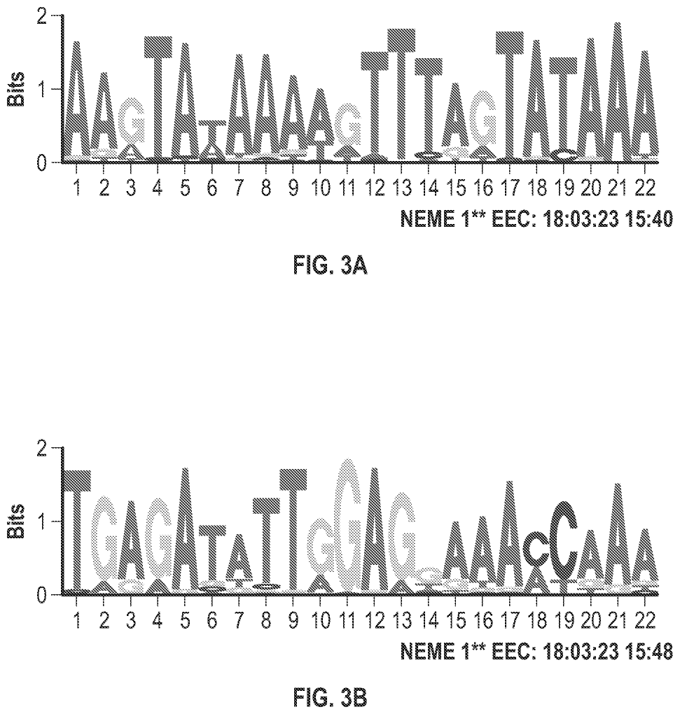

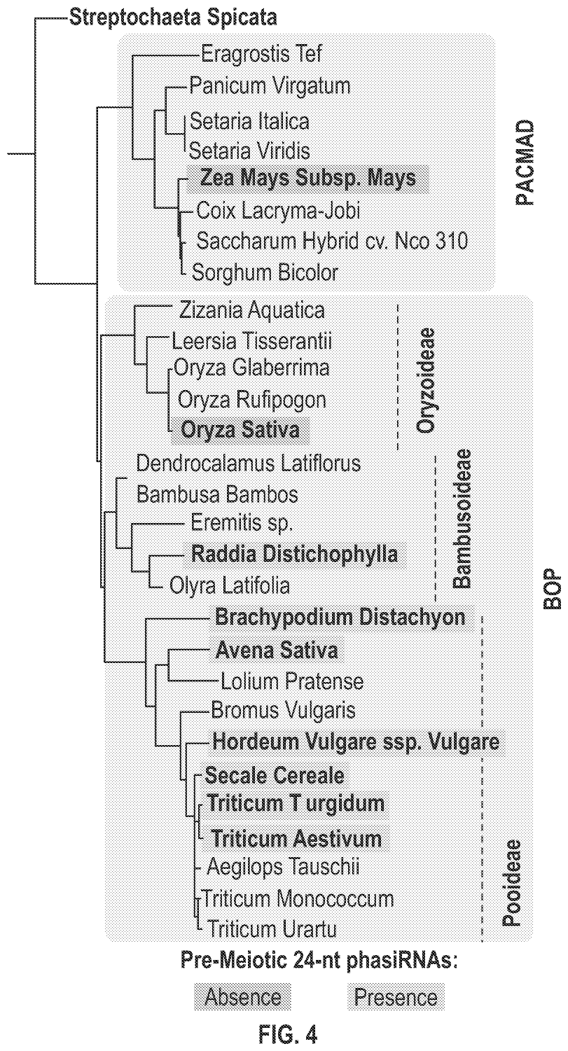

Conversely, the accumulation pattern for a second group of 24-nt phasiRNAs discovered by the inventors is drastically different from the accumulation pattern of the first group of phasiRNAs. 24-nt phasiRNAs of the second group accumulate at the pre-meiotic stage, more like the previously characterized 21-nt phasiRNAs of plants other than plants in the Pooideae or Bambusoideae subfamilies of plants such as maize and rice. For these pre-meiotic 24-nt phasiRNAs, although the miRNA trigger(s) (or another type of unknown sRNA) for biogenesis of the pre-meiotic 24-nt phasiRNAs is yet to be identified, the inventors discovered a putative nucleic acid sequence motif of a cleavage site in target PHAS transcripts, different from the nucleic acid sequence motif of the target sequence of miR2275 in the PHAS RNAs for group a (FIG. 3B). Accordingly, when the phasiRNAs are pre-meiotic phasiRNAs, a genetic modification of the instant disclosure can be in a nucleic acid sequence encoding a PHAS precursor transcript comprising a target nucleic acid sequence motif of a miRNA/sRNA trigger of pre-meiotic reproductive 24-nt phasiRNAs synthesis or one of the AGO proteins initiating the biogenesis or the effector of produced phasiRNAs.

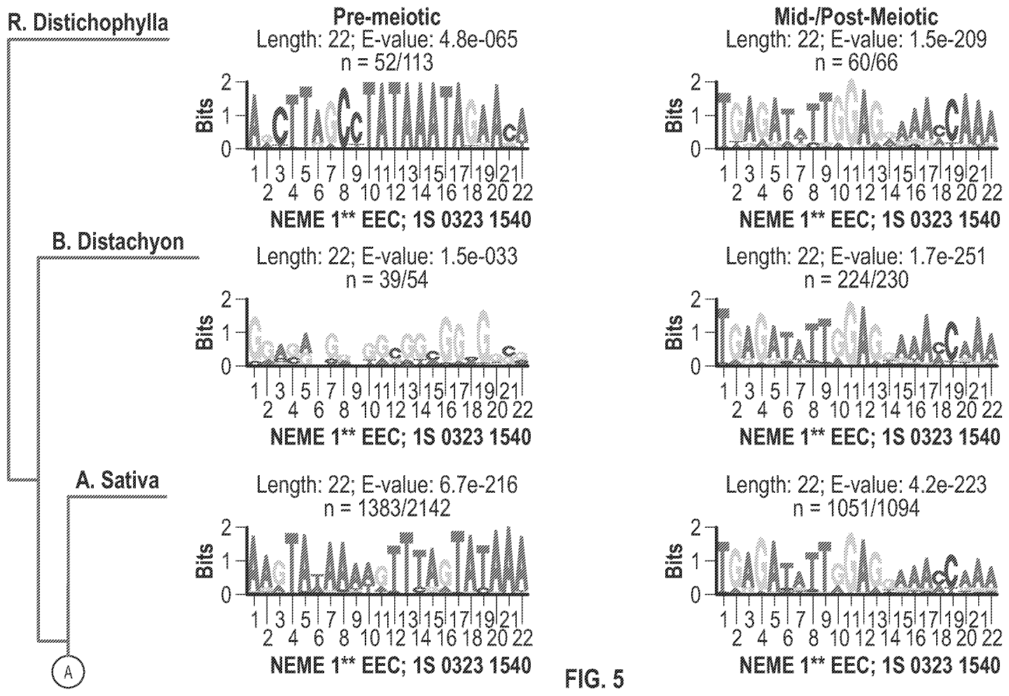

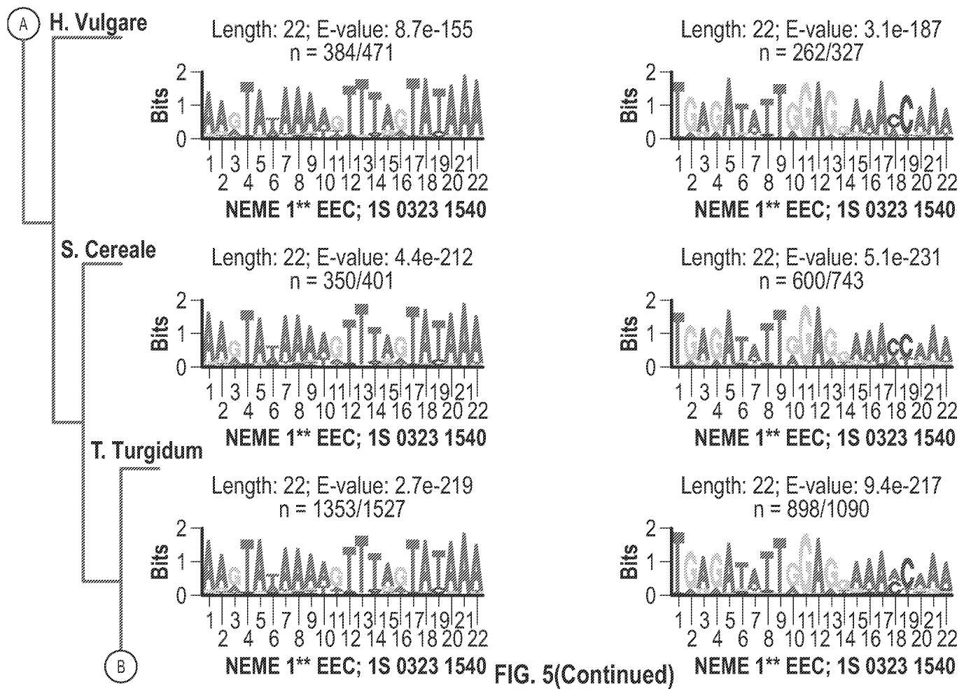

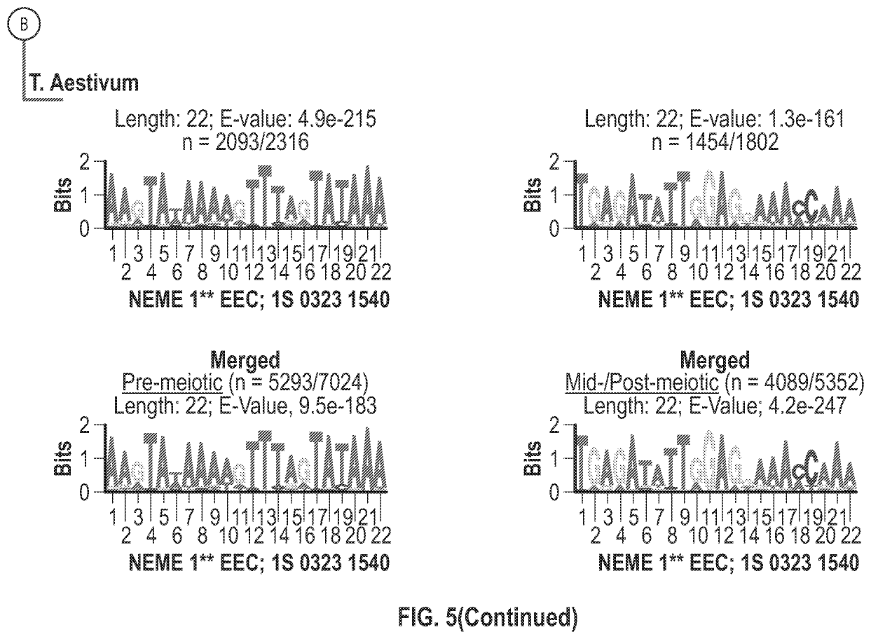

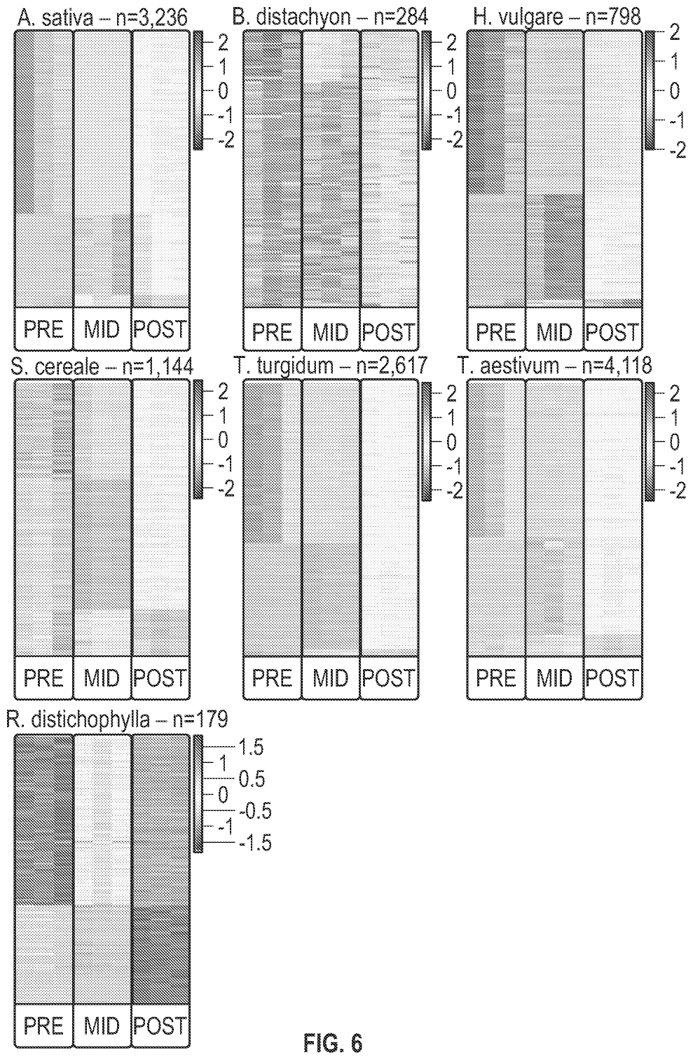

These previously uncharacterized pre-meiotic 24-nt phasiRNAs have not been reported and are not present in either maize or rice or any other species. Considering the evolutionary relationship of the Pooideae and Bambusoideae plants when compared to rice and maize, this absence of pre-meiotic 24-nt phasiRNAs in maize and rice suggests a divergence in grass species of the Pooideae and Bambusoideae subfamilies of plants (FIG. 4, FIG. 5, FIG. 6, and FIG. 7) and that pre-meiotic phasiRNA emerged in a common ancestor to Bambusoideae and Pooideae

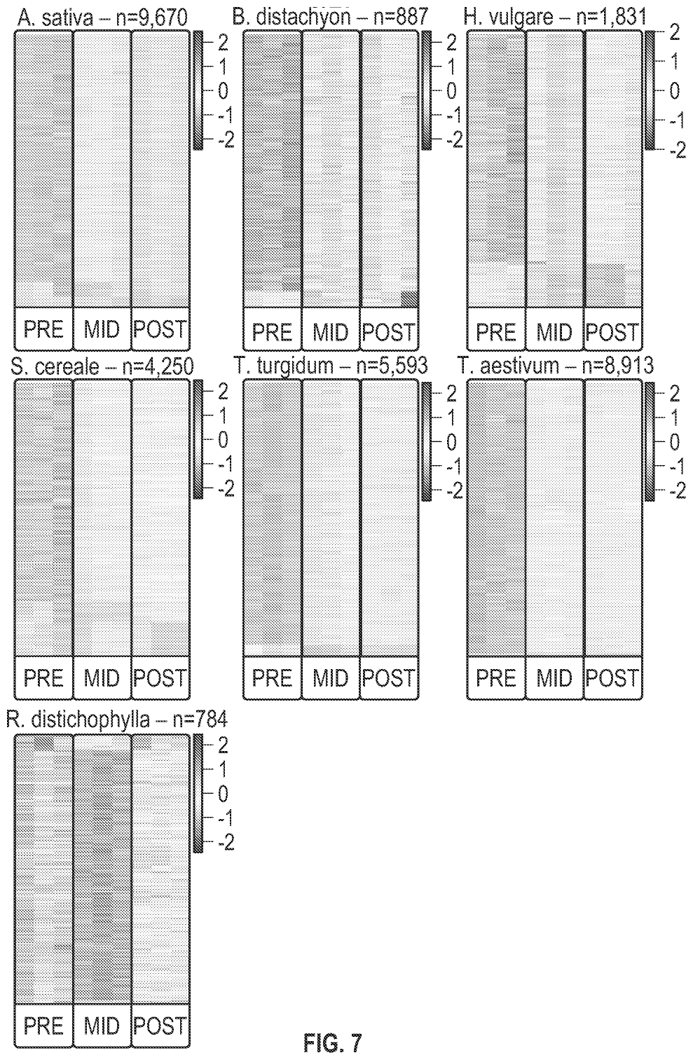

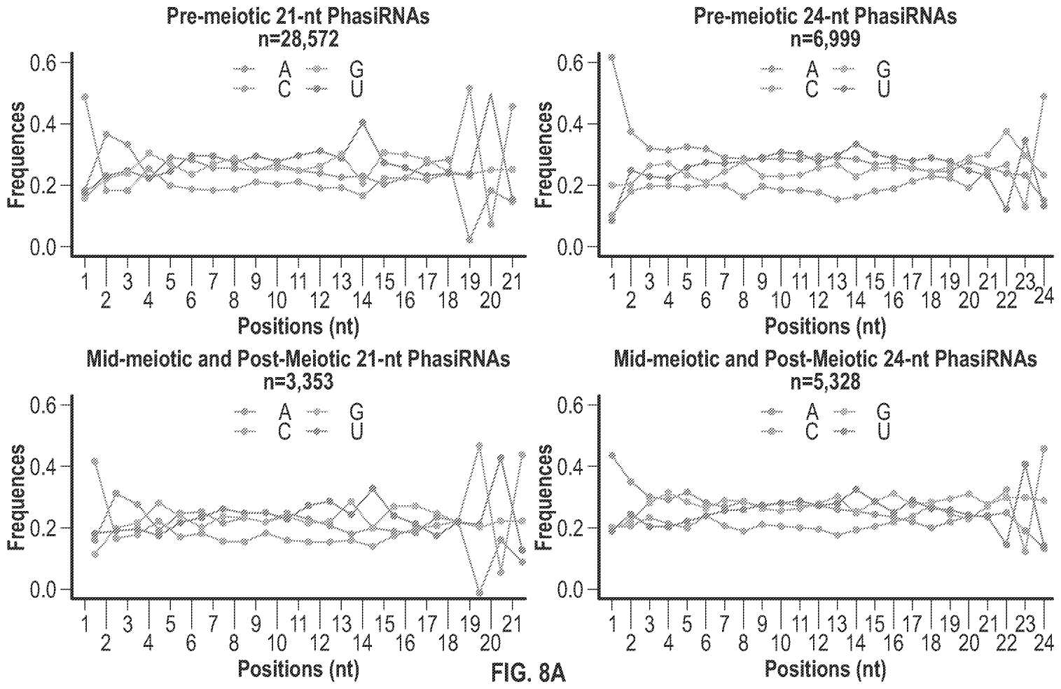

Additional differences between the 21-nt phasiRNAs and 24-nt phasiRNAs include a nucleotide bias observed at 5′ and 3′ ends of sRNA triggers of each group. Within categories of 21-nt and 24-nt phasiRNA, there is no difference between group of pre-meiotic and mid-post-meiotic phasiRNAs (FIGS. 8A and 8B). However, the nucleotides conserved at 5′ ends differ between 21-nt and 24-nt phasiRNAs.

The peak abundance of a third group (FIG. 6) was observed in the post-meiotic stage of anthers. This cluster, accumulating in post-meiotic stages, can have a biological function in gametogenesis.

The distinct temporal accumulation of 21- and 24-nt phasiRNAs requires precise regulation of PHAS precursor transcription and of the biogenesis components of phasiRNA pathways. The biogenesis and regulation of phasiRNAs requires polynucleotides and polypeptides comprising, without limitation, a miRNA trigger that target nucleic acid sequence of an RNA transcript, RNA polymerases (Pol), Dicer-like (DCL) proteins, double stranded RNA (dsRNA)-binding (DRB) proteins, RNA-directed RNA polymerases (RDRs), SKI2 helicases, exoribonucleases, and Argonaute (AGO) proteins. Loci that generate phasiRNAs are known as PHAS loci. The PHAS precursor RNAs can be protein-coding mRNAs or long, noncoding RNA (lncRNAs); lncRNAs are generally recognized as RNAs lacking an open reading frame encoding a protein of at least 100 amino acids. During miRNA-mediated secondary siRNA biogenesis, RDR6, recruited by AGO (with the assistance of SGS3), converts the RNA substrate into dsRNA, followed by processing into 21- or 24-nt RNA duplexes by a DCL protein, respectively DCL4 or DCL5. After cleavage, the 5′ fragment of the target mRNA is rapidly degraded by a 3′→5′ exonucleolytic complex to produce phasiRNAs, which are then loaded onto AGO protein partners to produce AGO-loaded phasiRNAs.

Biogenesis of 21-nt phasiRNAs as it was recognized by individuals of skill in the art before the invention was made (FIG. 1), is dependent on miR2118, RDR6, DCL4, MEIOSIS ARRESTED AT LEPTOTENE 1 (MEL1, also called AGO5c), and presumably a copy of AGO1, the AGO protein partner of miR2118, whereas biogenesis of mid-meiotic 24-nt phasiRNAs (FIG. 2) is dependent on miR2275, RDR6, DCL5, a copy of an AGO1 miRNA partner to load miR2275, and an unknown AGO protein partner of phasiRNAs to load the 24-nt phasiRNAs.

The inventors discovered that genetically modified plants in the Pooideae or Bambusoideae subfamilies comprising a nucleic acid modification that modifies pre-meiotic and mid-meiotic reproductive 24-nt phasiRNA, modifies the expression of the pre-meiotic and mid-meiotic reproductive 24-nt phasiRNA, modifies the expression of a polynucleotide in a biogenesis pathway of the pre-meiotic and mid-meiotic reproductive 24-nt phasiRNAs, or any combination thereof, are male-sterile. In some aspects, the genetically modified plants have disrupted biogenesis resulting in a depletion of pre-meiotic and/or mid-meiotic phasiRNAs in male reproductive tissues. Accordingly, the nucleic acid modification can be in any miRNA trigger(s), Pol, AGO, DCL, RDR, DRB, SGS3, any polynucleotide encoding the miRNA, Pol, AGO, DCL, RDR, DRB, SGS3, or any combination thereof in the biogenesis pathway.

In some aspects, a genetically modified plant of the instant disclosure comprises a genetic modification in a polynucleotide encoding a polypeptide in the biogenesis pathway of reproductive 24-nt phasiRNAs. In some aspects, the polypeptide in the biogenesis pathway of reproductive 24-nt phasiRNAs is a dicer-like protein (DCL protein), a miRNA partner argonaute protein, an RNA-dependent RNA polymerase (RDR), a phasiRNA partner argonaute protein, a suppressor of gene silencing 3 (SGS3) protein, a double-stranded RNA binding protein (DRB), or any combination thereof.

In some aspects, the polypeptide in the biogenesis pathway of reproductive 24-nt phasiRNAs is a miRNA partner argonaute protein, a phasiRNA partner argonaute protein, or both. Non-limiting examples of suitable argonaute proteins can be AGO1b/d, AGO4a/b/c (AGO9), AGO5a/b/c/d/e, AGO6, AGO7, and AGO10a/b. In some aspects, the miRNA partner argonaute protein for the 24-nt pre-meiotic phasiRNAs is an AGO1b/d protein. In some aspects, the phasiRNA partner argonaute protein for the 24-nt pre-meiotic phasiRNAs is an AGO4/9 protein. In yet other aspects, the phasiRNA partner argonaute protein for the 24-nt pre-meiotic phasiRNAs is an AGO7 protein. In additional aspects, the phasiRNA partner argonaute protein for the 24-nt pre-meiotic phasiRNAs is an AGO6 protein. In some aspects, the phasiRNA partner argonaute protein for the 24-nt pre-meiotic phasiRNAs is an AGO10 protein.

In some aspects, the polypeptide in the biogenesis pathway of reproductive 24-nt phasiRNAs is a DRB protein. Non-limiting examples of suitable DRB proteins include DRB1, DRB2, DRB3, DRB4, DRB5, and DRB6. In some aspects, the polypeptide in the biogenesis pathway of reproductive 24-nt phasiRNAs is a DRB1 protein. In other aspects, the polypeptide in the biogenesis pathway of reproductive 24-nt phasiRNAs is a DRB2 protein. In other aspects, the polypeptide in the biogenesis pathway of reproductive 24-nt phasiRNAs is a DRB5 protein. In other aspects, the polypeptide in the biogenesis pathway of reproductive 24-nt phasiRNAs is a DRB6 protein.

In other aspects, a genetically modified plant of the instant disclosure comprises a genetic modification in a nucleic acid sequence encoding a miRNA partner argonaute protein. In yet other aspects, a plant of the instant disclosure comprises a genetic modification in a nucleic acid sequence encoding a miRNA partner argonaute protein. In additional aspects, a plant of the instant disclosure comprises a genetic modification in a nucleic acid sequence encoding a phasiRNA partner AGO protein. In some aspects, a plant of the instant disclosure comprises a genetic modification in a nucleic acid sequence encoding an RDR protein. In other aspects, a plant of the instant disclosure comprises a genetic modification in a nucleic acid sequence encoding a DRB protein.

In part due to extensive experimentation, the inventors discovered that biogenesis of the pre-meiotic 24-nt phasiRNAs discovered by the inventors in Pooideae or Bambusoideae plant, the mid-meiotic 24-nt phasiRNAs, or both, is dependent on DCL5. Accordingly, in some aspects, the polypeptide in the biogenesis pathway of reproductive 24-nt phasiRNAs is a DCL5 protein. In some aspects, a genetic modification in a genetically modified plant of the instant disclosure reduces the expression of the DCL5 protein. Nucleic acid sequences encoding DCL proteins and DCL5 proteins can be as described in Section I (b) herein below.

In some aspects, a genetically modified plant of the instant disclosure comprises a genetic modification in one or more miRNA triggers of reproductive 24-nt phasiRNAs or in a polynucleotide encoding a factor in a biogenesis pathway of the miRNA trigger of reproductive 24-nt phasiRNAs. The reproductive 24-nt phasiRNA can be a mid-meiotic reproductive 24-nt phasiRNAs, a pre-meiotic reproductive 24-nt phasiRNAs, or a combination thereof.

When the phasiRNAs are mid-meiotic phasiRNAs, the genetic modification can be in a nucleic acid sequence encoding a PHAS transcript comprising a target nucleic acid sequence motif of a miRNA trigger of pre-meiotic reproductive 24-nt phasiRNAs synthesis, in a PHAS transcript comprising a target nucleic acid sequence motif of a miRNA trigger of pre-meiotic reproductive 24-nt phasiRNAs synthesis, in a miRNA trigger of pre-meiotic reproductive 24-nt phasiRNAs synthesis, in a biogenesis pathway of the miRNA trigger of pre-meiotic reproductive 24-nt phasiRNAs synthesis, or any combination thereof.

In some aspects, a genetically modified plant of the instant disclosure comprises a genetic modification in one or more miRNA triggers of mid-meiotic 24-nt phasiRNAs, in a polynucleotide encoding a factor in a biogenesis pathway of the miRNA trigger of mid-meiotic reproductive 24-nt phasiRNAs, or a combination thereof. In some aspects, a genetically modified plant of the instant disclosure comprises a genetic modification in a miR2275 miRNA trigger, in a polynucleotide encoding a factor in a biogenesis pathway of miR2275, or both. In some aspects, the genetic modification is in a nucleic acid sequence encoding a PHAS transcript comprising a target nucleic acid sequence motif of miR2275 (FIG. 3A). In some aspects, the genetic modification is in a PHAS transcript comprising a target nucleic acid sequence motif of miR2275 (FIG. 3A). In some aspects, the target nucleic acid sequence motif of miR2275 comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 30. In some aspects, the target nucleic acid sequence motif of miR2275 comprises at least about 75% or more, at least about 85% or more, at least about 95% or more, or 100% sequence identity with a nucleic acid sequence of SEQ ID NO: 30. In some aspects, the target nucleic acid sequence motif of miR2275 comprises a nucleic acid sequence of SEQ ID NO: 30.

When the phasiRNAs are pre-meiotic phasiRNAs, the genetic modification can be in a nucleic acid sequence encoding a PHAS transcript comprising a target nucleic acid sequence motif of a miRNA trigger of pre-meiotic reproductive 24-nt phasiRNAs synthesis, in a PHAS transcript comprising a target nucleic acid sequence motif of a miRNA trigger of pre-meiotic reproductive 24-nt phasiRNAs synthesis, in a miRNA trigger of pre-meiotic reproductive 24-nt phasiRNAs synthesis, in a biogenesis pathway of the miRNA trigger of pre-meiotic reproductive 24-nt phasiRNAs synthesis, or any combination thereof.

In some aspects, the genetic modification can be in a nucleic acid sequence encoding a PHAS transcript comprising a target nucleic acid sequence motif of a miRNA trigger of pre-meiotic reproductive 24-nt phasiRNAs synthesis. In some aspects, a nucleic acid sequence encoding a PHAS transcript comprising a target nucleic acid sequence motif of a miRNA trigger of pre-meiotic reproductive 24-nt phasiRNAs synthesis comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 31. In some aspects, a nucleic acid sequence encoding a PHAS transcript comprising a target nucleic acid sequence motif of a miRNA trigger of pre-meiotic reproductive 24-nt phasiRNAs synthesis comprises at least about 75% or more, at least about 85% or more, at least about 95% or more, or 100% sequence identity with a nucleic acid sequence of SEQ ID NO: 31.

In some aspects, the genetic modification can be in a PHAS transcript comprising a target nucleic acid sequence motif of a miRNA trigger of pre-meiotic reproductive 24-nt phasiRNAs synthesis. In other aspects, the PHAS precursor transcript comprising a target nucleic acid sequence motif of a miRNA trigger of pre-meiotic reproductive 24-nt phasiRNAs synthesis comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 49. In other aspects, the PHAS precursor transcript comprising a target nucleic acid sequence motif of a miRNA trigger of pre-meiotic reproductive 24-nt phasiRNAs synthesis comprises at least about 75% or more, at least about 85% or more, at least about 95% or more, or 100% sequence identity with a nucleic acid sequence of SEQ ID NO: 49.

When the phasiRNAs are pre-meiotic phasiRNAs, the genetic modification can be in a miRNA trigger of pre-meiotic reproductive 24-nt phasiRNAs synthesis or in a biogenesis pathway of the miRNA trigger of pre-meiotic reproductive 24-nt phasiRNAs synthesis. In some aspects, the miRNA trigger of pre-meiotic reproductive 24-nt phasiRNAs synthesis comprises a nucleic acid sequence comprising at least about 75% or more, at least about 85% or more, at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 48 or SEQ ID NO: 50. In some aspects, the miRNA trigger of pre-meiotic reproductive 24-nt phasiRNAs synthesis comprises a nucleic acid sequence comprising at least about 75% or more, at least about 85% or more, at least about 95% or more, or 100% sequence identity with a nucleic acid sequence of SEQ ID NO: 48 or SEQ ID NO: 50. In other aspects, the miRNA trigger of pre-meiotic reproductive 24-nt phasiRNAs synthesis comprises a nucleic acid sequence comprising nucleic acid sequence of SEQ ID NO: 48 or SEQ ID NO: 50. In other aspects, the miRNA trigger of pre-meiotic reproductive 24-nt phasiRNAs synthesis comprises a nucleic acid sequence comprising nucleic acid sequence of SEQ ID NO: 48 or SEQ ID NO: 50.

### (b) Genetically Modified Plants

In some aspects, a genetically modified plant of the instant disclosure is a plant selected from the Pooideae subfamily or the Bambusoideae subfamily of plants. Plants in Pooideae subfamily or the Bambusoideae subfamily of plants, including wheat and barley, have perfect flowers having male and female reproductive organs in the flower. Glumes remain closed until pollen release resulting to self-fertilisation. There is no natural outcrossing in domesticated species Pooideae and Bambusoideae plants. These characteristics make it difficult to deploy a robust system for large-scale, cost-effective, and sustainable hybrid seed programs.

A plant of the instant disclosure comprises a genetic modification that modifies a reproductive 24-nt phased, secondary small interfering RNA in male reproductive tissues (reproductive 24-nt phasiRNA), modifies the expression of the reproductive 24-nt phasiRNAs, modifies the expression in a polynucleotide in a phasiRNA biogenesis pathway responsible for biogenesis of phasiRNAs in male reproductive tissues, or any combination thereof,

In some aspects, plant of the instant disclosure comprises a genetic modification in a polynucleotide in a phasiRNA biogenesis pathway responsible for biogenesis of phasiRNAs in male reproductive tissues. The genetic modification can be any nucleic acid modification in the plant that can reduce the biogenesis of pre-meiotic phasiRNAs. The genetic modification can comprise a modification of a polynucleotide in the phasiRNA biogenesis pathway, or a modification of a polynucleotide having a sequence encoding a polypeptide in the phasiRNA biogenesis pathway.

As described above in Section I (a) herein above, the biogenesis and regulation of phasiRNAs requires a miRNA trigger, RNA polymerases (Pol), DCL proteins, DRB proteins, RDRs, and AGO proteins among other factors. PhasiRNA biogenesis initiates via miRNA-directed, AGO-catalyzed cleavage of a single-stranded RNA precursor, which is then converted to dsRNA by an RDR protein before being processed into 21- or 24-nt RNA duplexes by a DCL protein. PhasiRNAs are then loaded onto AGO protein partners to produce AGO-loaded phasiRNAs. In some aspects, a genetically modified plant of the instant disclosure comprises a genetic modification in a polynucleotide encoding a DCL5 protein. In some aspects, a genetically modified plant of the instant disclosure comprises a genetic modification in a polynucleotide encoding a DCL5 protein.

As described above, reproductive 24-nt phasiRNAs in Pooideae and Bambusoideae plants differ significantly from reproductive 24-nt phasiRNAs maize and rice. An evolutionary tree showing the evolutionary relationship of the Pooideae and Bambusoideae plants with maize and rice plants is shown in FIG. 4. FIG. 4 shows that all plants that comprise the pre-meiotic 24-nt phasiRNAs discovered by the inventors are in the Pooideae and Bambusoideae subfamilies of plants. Maize and rice are classified in ancestor and distinct subfamilies to Pooideae and Bambusoideae. This absence of pre-meiotic 24-nt phasiRNAs in maize and rice suggests a molecular innovation in Pooideae and Bambusoideae subfamilies. Accordingly, a plant of the instant disclosure can be any plant the Pooideae and Bambusoideae subfamilies of plants. Non-limiting examples of these plants can be Avena sativa (oats), Hordeum vulgare subsp. (barley), Secale cereale (rye), Triticum turgidum subsp. durum (durum wheat), Triticum aestivum (bread wheat), Brachypodium subsp. (e.g., Brachypodium distachyon), Aegilops tauschii, Triticum monococcum (Einkorn wheat), Triticum urartu (red wild einkorn wheat), ×Triticale (hybrid of wheat (Triticum) and rye (Secale)) or Olyra latifolia.

In some aspects, the genetically modified plant of the instant disclosure is Triticum turgidum. When the plant is Triticum turgidum, a genetically modified plant of the instant disclosure can comprise a genetic modification in a polynucleotide encoding a DCL5 protein. In some aspects, the genetic modification in the polynucleotide encoding a DCL5 protein reduces the expression or generates a loss-of-function of the DCL5 protein. In some aspects, the DCL5 protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 10 or SEQ ID NO: 12. In some aspects, the DCL5 protein comprises an amino acid sequence comprising about 75% or more, at least about 85% or more, at least about 95% or more, or 100% sequence identity with the nucleic acid sequence of SEQ ID NO: 10 or SEQ ID NO: 12. In some aspects, the DCL5 protein is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 11, SEQ ID NO: 40, SEQ ID NO: 41, SEQ ID NO: 13, SEQ ID NO: 42, or SEQ ID NO: 43. In some aspects, the DCL5 protein is encoded by a nucleic acid sequence comprising at least about 75% or more, at least about 85% or more, at least about 95% or more, or 100% sequence identity with the nucleic acid sequence of SEQ ID NO: 11, SEQ ID NO: 40, SEQ ID NO: 41, SEQ ID NO: 13, SEQ ID NO: 42, or SEQ ID NO: 43.

In some aspects, the genetically modified plant of the instant disclosure is a TILLING mutant of Triticum turgidum. In some aspects, the TILLING mutant of the Triticum turgidum plant comprises a nucleic acid modification in the nucleic acid sequence encoding the DCL5 protein. In some aspects, the genetically modified plant of the instant disclosure is a TILLING mutant of Triticum turgidum comprising a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 44, a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 46, or both. In some aspects, the genetically modified plant of the instant disclosure is a TILLING mutant of Triticum turgidum comprises a nucleic acid sequence comprising about 75% or more, at least about 85% or more, at least about 95% or more, or 100% sequence identity with the nucleic acid sequence of SEQ ID NO: 44, a nucleic acid sequence comprising about 75% or more, at least about 85% or more, at least about 95% or more, or 100% sequence identity with the nucleic acid sequence of SEQ ID NO: 46, or both.

In some aspects, the genetically modified plant of the instant disclosure is a TILLING mutant of Triticum turgidum comprising a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 45, a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 47, or both. In some aspects, the genetically modified plant of the instant disclosure is a TILLING mutant of Triticum turgidum comprises a nucleic acid sequence comprising about 75% or more, at least about 85% or more, at least about 95% or more, or 100% sequence identity with the nucleic acid sequence of SEQ ID NO: 45, a nucleic acid sequence comprising about 75% or more, at least about 85% or more, at least about 95% or more, or 100% sequence identity with the nucleic acid sequence of SEQ ID NO: 47, or both.

In some aspects, the genetically modified plant of the instant disclosure is a Triticum turgidum plant comprising a nucleic acid sequence comprising about 75% or more, at least about 85% or more, at least about 95% or more, or 100% sequence identity with the nucleic acid sequence of SEQ ID NO: 44, a nucleic acid sequence comprising about 75% or more, at least about 85% or more, at least about 95% or more, or 100% sequence identity with the nucleic acid sequence of SEQ ID NO: 46, a nucleic acid sequence comprising about 75% or more, at least about 85% or more, at least about 95% or more, or 100% sequence identity with the nucleic acid sequence of SEQ ID NO: 45, a nucleic acid sequence comprising about 75% or more, at least about 85% or more, at least about 95% or more, or 100% sequence identity with the nucleic acid sequence of SEQ ID NO: 47, or any combination thereof.

In some aspects, the genetically modified plant of the instant disclosure is a Triticum turgidum plant comprising a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 44, a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 46, a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 45, a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 47, or any combination thereof.

In some aspects, the genetically modified plant of the instant disclosure is barley (Hordeum vulgare). When the plant is barley, the polypeptide in the phasiRNA biogenesis pathway can be a DCL5 protein. In some aspects, the DCL5 protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 1. In some aspects, the DCL5 protein comprises an amino acid sequence comprising about 75% or more, at least about 85% or more, at least about 95% or more, or 100% sequence identity with the amino acid sequence of SEQ ID NO: 1. In some aspects, the DCL5 protein is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 2, SEQ ID NO: 32, or SEQ ID NO: 33. In some aspects, the DCL5 protein is encoded by a nucleic acid sequence comprising at least about 75% or more, at least about 85% or more, at least about 95% or more, or 100% sequence identity with the amino acid sequence of SEQ ID NO: 2, SEQ ID NO: 32, or SEQ ID NO: 33.

In some aspects, the genetically modified H. vulgare plant of the instant disclosure comprises a nucleic acid deletion in a nucleic acid sequence encoding the DCL5 protein. In some aspects, the genetically modified H. vulgare plant of the instant disclosure comprises a nucleic acid modification in the nucleic acid sequence encoding the DCL5 protein, wherein the nucleic acid modification comprises a deletion of a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 3, or SEQ ID NO: 51, SEQ ID NO: 19, or any combination thereof. In some aspects, the genetically modified H. vulgare plant of the instant disclosure comprises a nucleic acid modification in the nucleic acid sequence encoding the DCL5 protein, wherein the nucleic acid modification comprises a deletion of a nucleic acid sequence comprising about 75% or more, at least about 85% or more, at least about 95% or more, or 100% sequence identity with the nucleic acid sequence of SEQ ID NO: 3, or SEQ ID NO: 51, SEQ ID NO: 19, or any combination thereof.

In some aspects, the deletion in the genetically modified H. vulgare plant is generated using a CRISPR/Cas system with a gRNA comprising a nucleic acid sequence of SEQ ID NO: 15 (gRNA1) and SEQ ID NO: 16 (gRNA2), and the genetically modified H. vulgare plant comprises a deletion of a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 3. In some aspects, the deletion in the genetically modified H. vulgare plant is generated using a CRISPR/Cas system with a gRNA comprising a nucleic acid sequence of SEQ ID NO: 15 (gRNA1) and SEQ ID NO: 16 (gRNA2), and the genetically modified H. vulgare plant comprises a deletion of a nucleic acid sequence comprising about 75% or more, at least about 85% or more, at least about 95% or more, or 100% sequence identity with the nucleic acid sequence of SEQ ID NO: 3 or SEQ ID NO: 51. In some aspects, the deletion in the genetically modified H. vulgare plant comprises a deletion of a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 3 or SEQ ID NO: 51. In some aspects, the deletion in the genetically modified H. vulgare plant comprises a deletion of a nucleic acid sequence comprising about 75% or more, at least about 85% or more, at least about 95% or more, or 100% sequence identity with the nucleic acid sequence of SEQ ID NO: 3 or SEQ ID NO: 51.

In some aspects, the deletion in the genetically modified H. vulgare plant is generated using a CRISPR/Cas system with a gRNA comprising a nucleic acid sequence of SEQ ID NO: 17 (gRNA3) and SEQ ID NO: 18 (gRNA4), and the genetically modified H. vulgare plant comprises a deletion of a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 19. In some aspects, the deletion the genetically modified H. vulgare plant is generated using a CRISPR/Cas system with a gRNA comprising a nucleic acid sequence of SEQ ID NO: 17 (gRNA3) and SEQ ID NO: 18 (gRNA4), and the genetically modified H. vulgare plant comprises a deletion of a nucleic acid sequence comprising about 75% or more, at least about 85% or more, at least about 95% or more, or 100% sequence identity with the nucleic acid sequence of SEQ ID NO: 19. In some aspects, the genetically modified H. vulgare plant comprises a deletion of a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 19. In some aspects, the genetically modified H. vulgare plant comprises a deletion of a nucleic acid sequence comprising about 75% or more, at least about 85% or more, at least about 95% or more, or 100% sequence identity with the nucleic acid sequence of SEQ ID NO: 19.

In some aspects, the deletion in the genetically modified H. vulgare plant is generated using a CRISPR/Cas system with a gRNA comprising a nucleic acid sequence of SEQ ID NO: 15 (gRNA1), SEQ ID NO: 16 (gRNA2), SEQ ID NO: 17 (gRNA3) and SEQ ID NO: 18 (gRNA4), and the genetically modified H. vulgare plant comprises a deletion of a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 3 or SEQ ID NO: 51 and a deletion of a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 19. In some aspects, the deletion in the genetically modified H. vulgare plant is generated using a CRISPR/Cas system with a gRNA comprising a nucleic acid sequence of SEQ ID NO: 15 (gRNA1), SEQ ID NO: 16 (gRNA2), SEQ ID NO: 17 (gRNA3) and SEQ ID NO: 18 (gRNA4), and the genetically modified H. vulgare plant comprises a deletion of a nucleic acid sequence comprising about 75% or more, at least about 85% or more, at least about 95% or more, or 100% sequence identity with the nucleic acid sequence of SEQ ID NO: 3 or SEQ ID NO: 51 and a deletion of a nucleic acid sequence comprising about 75% or more, at least about 85% or more, at least about 95% or more, or 100% sequence identity with the nucleic acid sequence of SEQ ID NO: 19.

In some aspects, the deletion in the genetically modified H. vulgare plant comprises a deletion of a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 3 or SEQ ID NO: 51 and a deletion of a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 19. In some aspects, the deletion in the genetically modified H. vulgare plant comprises a deletion of a nucleic acid sequence comprising about 75% or more, at least about 85% or more, at least about 95% or more, or 100% sequence identity with the nucleic acid sequence of SEQ ID NO: 3 or SEQ ID NO: 51 and a deletion of a nucleic acid sequence comprising about 75% or more, at least about 85% or more, at least about 95% or more, or 100% sequence identity with the nucleic acid sequence of SEQ ID NO: 19.

In some aspects, the genetically modified plant of the instant disclosure is Triticum aestivum. When the plant is T. aestivum, the polypeptide in the phasiRNA biogenesis pathway can be a DCL5 protein. In some aspects, the DCL5 protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 4, SEQ ID NO: 6, SEQ ID NO: 8, or any combination thereof. In some aspects, the DCL5 protein comprises an amino acid sequence comprising about 75% or more, at least about 85% or more, at least about 95% or more, or 100% sequence identity with the amino acid sequence of SEQ ID NO: 4, SEQ ID NO: 6, SEQ ID NO: 8, or any combination thereof. In some aspects, the DCL5 protein is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 5, SEQ ID NO: 34, SEQ ID NO: 35, or any combination thereof. In some aspects, the DCL5 protein is encoded by a nucleic acid sequence comprising at least about 75% or more, at least about 85% or more, at least about 95% or more, or 100% sequence identity with the nucleic acid sequence of SEQ ID NO: 5, SEQ ID NO: 34, SEQ ID NO: 35, or any combination thereof.

In some aspects, the deletion in the genetically modified T. aestivum plant is generated using a CRISPR/Cas system with a gRNA comprising a nucleic acid sequence of SEQ ID NO: 20 (gRNA1), SEQ ID NO: 21 (gRNA2), SEQ ID NO: 22 (gRNA3), SEQ ID NO: 23 (gRNA4), SEQ ID NO: 24 (gRNA4), SEQ ID NO: 23 (gRNA5), or any combination thereof.

One aspect of the present disclosure also encompasses one or more plants comprising one or more nucleic acid constructs described in Section III.

### (c) Conditional Male-Sterility

The genetically modified Pooideae or Bambusoideae plants of the instant disclosure comprise a conditional male-sterile phenotype. Plants comprising a conditional male-sterile phenotype are male-sterile when grown under a first set of growth conditions (male-sterile growth conditions), but fertile when grown under a second growth conditions (fertile growth conditions). As explained herein above in Section I (a), plants of the instant disclosure comprise a depletion of pre-meiotic and mid-meiotic 24-nt phasiRNAs in male reproductive tissues, which results in a conditional male sterile phenotype. In some aspects, the pre-meiotic and mid-meiotic 24-nt phasiRNAs are depleted in male reproductive tissues even when the plants are grown under growth fertile growth conditions.

In some aspects, the conditional male-sterility is conditional on environmental growth conditions. Non-limiting examples of growth conditions under which the plant can exhibit the male-sterile phenotype include temperature, photoperiod, light quality, light intensity, or any combination thereof. In some aspects, the conditional male-sterile phenotype is conditional on temperature (temperature sensitive). Surprisingly, when the conditional male-sterile phenotype is conditional on temperature, there is a complete reversal of the environmental conditions that induce male sterility in plants of the Pooideae and Bambusoideae subfamilies when compared to other plants outside the Pooideae and Bambusoideae subfamilies such maize and rice. For instance, whereas the Pooideae and Bambusoideae plants of the instant disclosure can comprise a male-sterile phenotype when exposed to a temperature lower than a threshold temperature or threshold light conditions before flowering, during flowering, or both, a male-sterile phenotype is induced in maize and rice at temperatures above a threshold temperature or threshold light conditions.

In some aspects, the plant comprises a male-sterile phenotype when exposed to a temperature equal to or below about 24, 23, 22, 21, 20, 19, 18, 17, 16, or a temperature equal to or below about 15° C. before flowering, during flowering, or both. In some aspects, the plant comprises a male-sterile phenotype when exposed to a temperature equal to or below about 20° C. before flowering, during flowering, or both. In some aspects, the plant comprises a male-sterile phenotype when exposed to a temperature equal to or below about 19° C. before flowering, during flowering, or both. In some aspects, the plant comprises a male-sterile phenotype when exposed to a temperature equal to or below about 18° C. before flowering, during flowering, or both. In some aspects, the plant comprises a male-sterile phenotype when exposed to a temperature equal to or below about 17° C. before flowering, during flowering, or both. In some aspects, the plant comprises a male-sterile phenotype when exposed to a temperature equal to or below about 16° C. before flowering, during flowering, or both. In some aspects, the plant comprises a male-sterile phenotype when exposed to a temperature equal to or below about 15° C. before flowering, during flowering, or both.

In some aspects, the plant comprises a fertile phenotype when exposed to a temperature equal to or above about 15, 16, 17, 18, 19, 20, 21, 22, 23, 24, 25, or a temperature equal to or above about 26° C. before flowering, during flowering, or both. In some aspects, the plant comprises a fertile phenotype when exposed to a temperature equal to or above about 20° C. before flowering, during flowering, or both. In some aspects, the plant comprises a fertile phenotype when exposed to a temperature equal to or above about 21° C. before flowering, during flowering, or both. In some aspects, the plant comprises a fertile phenotype when exposed to a temperature equal to or above about 22° C. before flowering, during flowering, or both. In some aspects, the plant comprises a fertile phenotype when exposed to a temperature equal to or above about 23° C. before flowering, during flowering, or both. In some aspects, the plant comprises a fertile phenotype when exposed to a temperature equal to or above about 24° C. before flowering, during flowering, or both. In some aspects, the plant comprises a fertile phenotype when exposed to a temperature equal to or above about 25° C. before flowering, during flowering, or both. In some aspects, the plant comprises a fertile phenotype when exposed to a temperature equal to or above about 26° C. before flowering, during flowering, or both.

### II. Engineered Nucleic Acid Modification System

One aspect of the present disclosure encompasses an engineered nucleic acid modification system for introducing a genetic modification of a reproductive 24-nt phasiRNA, modifying the expression of the reproductive 24-nt phasiRNA, modifying the expression of a polynucleotide in a biogenesis pathway of the reproductive 24-nt phasiRNA, or any combination thereof, in a plant or plant cell selected from the Pooideae subfamily or the Bambusoideae subfamily of plants. Non-limiting examples of suitable protein expression modification systems include programmable nucleic acid modification systems, an expression construct encoding a protein or variants thereof, and any combination thereof.

In some aspects, the nucleic acid modification system is an expression construct comprising a nucleotide sequence encoding the polypeptide or polynucleotide operably linked to a promoter. In other aspects, the nucleic acid modification system is a programmable nucleic acid modification system targeted to a nucleic acid sequence in a nucleotide sequence encoding the polypeptide or polynucleotide in the 24-nt pre-meiotic phasiRNA biogenesis pathway. As used herein, a “programmable nucleic acid modification system” is a system capable of targeting and modifying the nucleic acid or modifying the expression or stability of a nucleic acid to alter a polynucleotide sequence or a protein or the expression of a polynucleotide sequence or protein encoded by the nucleic acid. The programmable nucleic acid modification system can comprise an interfering nucleic acid molecule or a nucleic acid editing system. The programmable protein expression modification system is specifically targeted to a sequence within a nucleic acid sequence encoding a polypeptide or a polynucleotide responsible for biogenesis of phasiRNAs in male reproductive tissues in a plant in the Pooideae or Bambusoideae subfamilies of plants.

In some aspects, the programmable expression modification system comprises an interfering nucleic acid (RNAi) molecule having a nucleotide sequence complementary to a target sequence within a gene encoding the polypeptide or polynucleotide used to inhibit expression of the polypeptide or polynucleotide. RNAi molecules generally act by forming a heteroduplex with a target RNA molecule, which is selectively degraded or “knocked down,” hence inactivating the target RNA. Under some conditions, an interfering RNA molecule can also inactivate a target transcript by repressing transcript translation and/or inhibiting transcription. An interfering RNA is more generally said to be “targeted against” a biologically relevant target, such as a protein, when it is targeted against the nucleic acid encoding the target. For example, an interfering RNA molecule has a nucleotide (nt) sequence which is complementary to an endogenous mRNA of a target gene sequence. Thus, given a target gene sequence, an interfering RNA molecule can be prepared which has a nucleotide sequence at least a portion of which is complementary to a target gene sequence. When introduced into cells, the interfering RNA binds to the target mRNA, thereby functionally inactivating the target mRNA and/or leading to degradation of the target mRNA.

Interfering RNA molecules include, inter alia, small interfering RNA (siRNA), microRNA (miRNA), piwi-interacting RNA (piRNA), long non-coding RNAs (long ncRNAs or lncRNAs), and small hairpin RNAs (shRNA). lncRNAs are widely expressed and have key roles in gene regulation. Depending on their localization and their specific interactions with DNA, RNA and proteins, lncRNAs can modulate chromatin function, regulate the assembly and function of membraneless nuclear bodies, alter the stability and translation of cytoplasmic mRNAs, and interfere with signaling pathways. Piwi-interacting RNA (piRNA) is the largest class of small non-coding RNA molecules expressed in animal cells. piRNAs regulate gene expression through interactions with piwi-subfamily Argonaute proteins. SiRNA are double-stranded RNA molecules, preferably about 19-25 nucleotides in length. When transfected into cells, siRNA inhibit the target mRNA transiently until they are also degraded within the cell. MiRNA and siRNA are biochemically and functionally indistinguishable. Both are about the same in nucleotide length with 5′-phosphate and 3′-hydroxyl ends, and assemble into an RNA-induced silencing complex (RISC) to silence specific gene expression. siRNA and miRNA are distinguished based on origin. siRNA is obtained from long double-stranded RNA (dsRNA), while miRNA is derived from the double-stranded region of a 60-70 nt RNA hairpin precursor. Small hairpin RNAs (shRNA) are sequences of RNA, typically about 50-80 base pairs, or about 50, 55, 60, 65, 70, 75, or about 80 base pairs in length, that include a region of internal hybridization forming a stem loop structure consisting of a base-pair region of about 19-29 base pairs of double-strand RNA (the stem) bridged by a region of single-strand RNA (the loop) and a short 3′ overhang. shRNA molecules are processed within the cell to form siRNA which in turn knock down target gene expression. shRNA can be incorporated into plasmid vectors and integrated into genomic DNA for longer-term or stable expression, and thus longer knockdown of the target mRNA.

Interfering nucleic acid molecules can contain RNA bases, non-RNA bases, or a mixture of RNA bases and non-RNA bases. For example, interfering nucleic acid molecules provided herein can be primarily composed of RNA bases but also contain DNA bases or non-naturally occurring nucleotides. The interfering nucleic acids can employ a variety of oligonucleotide chemistries. Examples of oligonucleotide chemistries include, without limitation, peptide nucleic acid (PNA), linked nucleic acid (LNA), phosphorothioate, 2′O-Me-modified oligonucleotides, and morpholino chemistries, including combinations of any of the foregoing. In general, PNA and LNA chemistries can utilize shorter targeting sequences because of their relatively high target binding strength relative to 2′O-Me oligonucleotides. Phosphorothioate and 2′O-Me-modified chemistries are often combined to generate 2′O-Me-modified oligonucleotides having a phosphorothioate backbone.

In some aspects, the programmable nucleic acid modification system is a nucleic acid editing system. Such modification system can be used to edit DNA or RNA sequences to repress transcription or translation of an mRNA encoded by the gene, and/or produce mutant proteins with reduced activity or stability. Non-limiting examples of programmable nucleic acid editing systems include, without limit, an RNA-guided clustered regularly interspersed short palindromic repeats (CRISPR)/CRISPR-associated (Cas) (CRISPR/Cas) nuclease system, a CRISPR/Cpf1 nuclease system, a zinc finger nuclease (ZFN), a transcription activator-like effector nuclease (TALEN), a meganuclease, a ribozyme, or a programmable DNA binding domain linked to a nuclease domain. Other suitable programmable nucleic acid modification systems will be recognized by individuals skilled in the art.

Such systems rely for specificity on the delivery of exogenous protein(s), and/or a guide RNA (gRNA) or single guide RNA (sgRNA) having a sequence which binds specifically to a gene sequence of interest. When the programmable nucleic acid modification system comprises more than one component, such as a protein and a guide nucleic acid, the multi-component modification system can be modular, in that the different components can optionally be distributed among two or more nucleic acid constructs as described herein. The system components can be delivered by a plasmid or viral vector or as a synthetic oligonucleotide. More detailed descriptions of programmable nucleic acid editing systems can be as described further below.

In some aspects, the programmable nucleic acid modification system is a CRISPR/Cas tool modified for transcriptional regulation of a locus. In some aspects, the programmable nucleic acid modification system is CRISPR/Cas system comprising a Cas9 nuclease and a guide RNA (gRNA) comprising a sequence complementary to a target sequence within the nucleotide sequence encoding the polypeptide or polynucleotide in the phasiRNA biogenesis pathway.

In some aspects, the Cas9 nuclease comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 14. In some aspects, the Cas9 nuclease comprises an amino acid sequence comprising about 75% or more, at least about 85% or more, at least about 95% or more, or 100% sequence identity with the nucleic acid sequence of SEQ ID NO: 14.

In some aspects, the genetically modified plant is H. vulgare. In some aspects, the polypeptide in the phasiRNA biogenesis pathway is a DCL5 protein comprising an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 2. In some aspects, the polypeptide in the phasiRNA biogenesis pathway is a DCL5 protein comprising an amino acid sequence comprising about 75% or more, at least about 85% or more, at least about 95% or more, or 100% sequence identity with the amino acid sequence of SEQ ID NO: 2. When the programmable nucleic acid modification system is a CRISPR/Cas system and the polypeptide is a DCL5 protein, the gRNA can comprise a nucleic acid sequence of SEQ ID NO: 15 (gRNA1), SEQ ID NO: 16 (gRNA2), SEQ ID NO: 17 (gRNA3), SEQ ID NO: 18 (gRNA4), or any combination thereof.

In some aspects, the genetically modified plant is T. aestivum. In some aspects, the polypeptide in the phasiRNA biogenesis pathway is a DCL5 protein comprising an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 4, SEQ ID NO: 6, or SEQ ID NO: 8. In some aspects, the polypeptide in the phasiRNA biogenesis pathway is a DCL5 protein comprising an amino acid sequence comprising about 75% or more, at least about 85% or more, at least about 95% or more, or 100% sequence identity with the amino acid sequence of SEQ ID NO: 4, SEQ ID NO: 6, or SEQ ID NO: 8. When the programmable nucleic acid modification system is a CRISPR/Cas system and the polypeptide is a DCL5 protein, the gRNA can comprise a nucleic acid sequence of SEQ ID NO: 20 (gRNA1), SEQ ID NO: 21 (gRNA2), SEQ ID NO: 22 (gRNA3), SEQ ID NO: 23 (gRNA4), SEQ ID NO: 24 (gRNA5), SEQ ID NO: 25 (gRNA6), or any combination thereof. In some aspects, the gRNA comprises a nucleic acid sequence complementary to a target sequence within the nucleotide sequence encoding the DCL5 protein comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 29. In some aspects, the gRNA comprises a nucleic acid sequence complementary to a target sequence within the nucleotide sequence encoding the DCL protein comprising at least about 75% or more, at least about 85% or more, at least about 95% or more, or 100% sequence identity with a nucleic acid sequence of SEQ ID NO: 29.

i. CRISPR Nuclease Systems.

The programmable targeting nuclease can be an RNA-guided CRISPR endonuclease system. The CRISPR system comprises a guide RNA or sgRNA to a target sequence at which a protein of the system introduces a double-stranded break in a target nucleic acid sequence, and a CRISPR-associated endonuclease. The gRNA is a short synthetic RNA comprising a sequence necessary for endonuclease binding, and a preselected ˜20 nucleotide spacer sequence targeting the sequence of interest in a genomic target. Non-limiting examples of endonucleases include Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, or a homolog thereof, a recombination of the naturally occurring molecule thereof, a codon-optimized version thereof, or a modified version thereof, or any combination thereof.

The CRISPR nuclease system can be derived from any type of CRISPR system, including a type I (i.e., IA, IB, IC, ID, IE, or IF), type II (i.e., IIA, IIB, or IIC), type III (i.e., IIIA or IIIB), or type V CRISPR system. The CRISPR/Cas system can be from Streptococcus sp. (e.g., Streptococcus pyogenes), Campylobacter sp. (e.g., Campylobacter jejuni), Francisella sp. (e.g., Francisella novicida), Acaryochloris sp., Acetohalobium sp., Acidaminococcus sp., Acidithiobacillus sp., Alicyclobacillus sp., Allochromatium sp., Ammonifex sp., Anabaena sp., Arthrospira sp., Bacillus sp., Burkholderiales sp., Caldicelulosiruptor sp., Candidatus sp., Clostridium sp., Crocosphaera sp., Cyanothece sp., Exiguobacterium sp., Finegoldia sp., Ktedonobacter sp., Lactobacillus sp., Lyngbya sp., Marinobacter sp., Methanohalobium sp., Microscilla sp., Microcoleus sp., Microcystis sp., Natranaerobius sp., Neisseria sp., Nitrosococcus sp., Nocardiopsis sp., Nodularia sp., Nostoc sp., Oscillatoria sp., Polaromonas sp., Pelotomaculum sp., Pseudoalteromonas sp., Petrotoga sp., Prevotella sp., Staphylococcus sp., Streptomyces sp., Streptosporangium sp., Synechococcus sp., or Thermosipho sp.

Non-limiting examples of suitable CRISPR systems include CRISPR/Cas systems, CRISPR/Cpf systems, CRISPR/Cmr systems, CRISPR/Csa systems, CRISPR/Csb systems, CRISPR/Csc systems, CRISPR/Cse systems, CRISPR/Csf systems, CRISPR/Csm systems, CRISPR/Csn systems, CRISPR/Csx systems, CRISPR/Csy systems, CRISPR/Csz systems, and derivatives or variants thereof. Preferably, the CRISPR system can be a type II Cas9 protein, a type V Cpf1 protein, or a derivative thereof. In some aspects, the CRISPR/Cas nuclease is Streptococcus pyogenes Cas9 (SpCas9), Streptococcus thermophilus Cas9 (StCas9), Campylobacter jejuni Cas9 (CjCas9), Francisella novicida Cas9 (FnCas9), or Francisella novicida Cpf1 (FnCpf1).

In general, a protein of the CRISPR system comprises an RNA recognition and/or RNA binding domain, which interacts with the guide RNA. A protein of the CRISPR system also comprises at least one nuclease domain having endonuclease activity. For example, a Cas9 protein can comprise a RuvC-like nuclease domain and an HNH-like nuclease domain, and a Cpf1 protein can comprise a RuvC-like domain. A protein of the CRISPR system can also comprise DNA binding domains, helicase domains, RNase domains, protein-protein interaction domains, dimerization domains, as well as other domains.

A protein of the CRISPR system can be associated with guide RNAs (gRNA). The guide RNA can be a single guide RNA (i.e., sgRNA), or can comprise two RNA molecules (i.e., crRNA and tracrRNA). The guide RNA interacts with a protein of the CRISPR system to guide it to a target site in the DNA. The target site has no sequence limitation except that the sequence is bordered by a protospacer adjacent motif (PAM). For example, PAM sequences for Cas9 include 3′-NGG, 3′-NGGNG, 3′-NNAGAAW, and 3′-ACAY, and PAM sequences for Cpf1 include 5′-TTN (wherein N is defined as any nucleotide, W is defined as either A or T, and Y is defined as either C or T). Each gRNA comprises a sequence that is complementary to the target sequence (e.g., a Cas9 gRNA can comprise GN17-20GG). The gRNA can also comprise a scaffold sequence that forms a stem loop structure and a single-stranded region. The scaffold region can be the same in every gRNA. In some aspects, the gRNA can be a single molecule (i.e., sgRNA). In other aspects, the gRNA can be two separate molecules. Those skilled in the art are familiar with gRNA design and construction, e.g., gRNA design tools are available on the internet or from commercial sources.

A CRISPR system can comprise one or more nucleic acid binding domains associated with one or more, or two or more selected guide RNAs used to direct the CRISPR system to one or more, or two or more selected target nucleic acid loci. For instance, a nucleic acid binding domain can be associated with one or more, or two or more selected guide RNAs, each selected guide RNA, when complexed with a nucleic acid binding domain, causing the CRISPR system to localize to the target of the guide RNA.

ii. CRISPR Nickase Systems.

The programmable targeting nuclease can also be a CRISPR nickase system. CRISPR nickase systems are similar to the CRISPR nuclease systems described above except that a CRISPR nuclease of the system is modified to cleave only one strand of a double-stranded nucleic acid sequence. Thus, a CRISPR nickase, in combination with a guide RNA of the system, can create a single-stranded break or nick in the target nucleic acid sequence. Alternatively, a CRISPR nickase in combination with a pair of offset gRNAs can create a double-stranded break in the nucleic acid sequence.

A CRISPR nuclease of the system can be converted to a nickase by one or more mutations and/or deletions. For example, a Cas9 nickase can comprise one or more mutations in one of the nuclease domains, wherein the one or more mutations can be D10A, E762A, and/or D986A in the RuvC-like domain, or the one or more mutations can be H840A (or H839A), N854A and/or N863A in the HNH-like domain.

iii. ssDNA-Guided Argonaute Systems.

Alternatively, the programmable targeting nuclease can comprise a single-stranded DNA-guided Argonaute endonuclease. Argonaute (AGO) proteins are a family of endonucleases that use 5′-phosphorylated short single-stranded nucleic acids as guides to cleave nucleic acid targets. Some prokaryotic AGO proteins use single-stranded guide DNAs and create double-stranded breaks in nucleic acid sequences. The ssDNA-guided AGO endonuclease can be associated with a single-stranded guide DNA.

The AGO endonuclease can be derived from Alistipes sp., Aquifex sp., Archaeoglobus sp., Bacteroides sp., Bradyrhizobium sp., Burkholderia sp., Cellvibrio sp., Chlorobium sp., Geobacter sp., Mariprofundus sp., Natronobacterium sp., Parabacteriodes sp., Parvularcula sp., Planctomyces sp., Pseudomonas sp., Pyrococcus sp., Thermus sp., or Xanthomonas sp. For instance, the AGO endonuclease can be Natronobacterium gregoryi AGO (NgAGO). Alternatively, the AGO endonuclease can be Thermus thermophilus AGO (TtAGO). The AGO endonuclease can also be Pyrococcus furiosus (PfAGO).

The single-stranded guide DNA (gDNA) of an ssDNA-guided Argonaute system is complementary to the target site in the nucleic acid sequence. The target site has no sequence limitations and does not require a PAM. The gDNA generally ranges in length from about 15-30 nucleotides. The gDNA can comprise a 5′ phosphate group. Those skilled in the art are familiar with ssDNA oligonucleotide design and construction.

iv. Zinc Finger Nucleases.

The programmable targeting nuclease can be a zinc finger nuclease (ZFN). A ZFN comprises a DNA-binding zinc finger region and a nuclease domain. The zinc finger region can comprise from about two to seven zinc fingers, for example, about four to six zinc fingers, wherein each zinc finger binds three nucleotides. The zinc finger region can be engineered to recognize and bind to any DNA sequence. Zinc finger design tools or algorithms are available on the internet or from commercial sources. The zinc fingers can be linked together using suitable linker sequences.

A ZFN also comprises a nuclease domain, which can be obtained from any endonuclease or exonuclease. Non-limiting examples of endonucleases from which a nuclease domain can be derived include, but are not limited to, restriction endonucleases and homing endonucleases. The nuclease domain can be derived from a type II-S restriction endonuclease. Type II-S endonucleases cleave DNA at sites that are typically several base pairs away from the recognition/binding site and, as such, have separable binding and cleavage domains. These enzymes generally are monomers that transiently associate to form dimers to cleave each strand of DNA at staggered locations. Non-limiting examples of suitable type II-S endonucleases include BfiI, BpmI, BsaI, BsgI, BsmBI, BsmI, BspMI, FokI, MbolI, and SapI. The type II-S nuclease domain can be modified to facilitate dimerization of two different nuclease domains. For example, the cleavage domain of FokI can be modified by mutating certain amino acid residues. By way of non-limiting example, amino acid residues at positions 446, 447, 479, 483, 484, 486, 487, 490, 491, 496, 498, 499, 500, 531, 534, 537, and 538 of FokI nuclease domains are targets for modification. For example, one modified FokI domain can comprise Q486E, 1499L, and/or N496D mutations, and the other modified FokI domain can comprise E490K, 1538K, and/or H537R mutations.

v. Transcription Activator-Like Effector Nuclease Systems.

The programmable targeting nuclease can also be a transcription activator-like effector nuclease (TALEN) or the like. TALENs comprise a DNA-binding domain composed of highly conserved repeats derived from transcription activator-like effectors (TALEs) that are linked to a nuclease domain. TALEs are proteins secreted by plant pathogen Xanthomonas to alter transcription of genes in host plant cells. TALE repeat arrays can be engineered via modular protein design to target any DNA sequence of interest. Other transcription activator-like effector nuclease systems can comprise, but are not limited to, the repetitive sequence, transcription activator like effector (RipTAL) system from the bacterial plant pathogenic Ralstonia solanacearum species complex (Rssc). The nuclease domain of TALEs can be any nuclease domain as described above in Section II (i).

vi. Meganucleases or Rare-Cutting Endonuclease Systems.

The programmable targeting nuclease can also be a meganuclease or derivative thereof. Meganucleases are endodeoxyribonucleases characterized by long recognition sequences, i.e., the recognition sequence generally ranges from about 12 base pairs to about 45 base pairs. As a consequence of this requirement, the recognition sequence generally occurs only once in any given genome. Among meganucleases, the family of homing endonucleases named LAGLIDADG has become a valuable tool for the study of genomes and genome engineering. Non-limiting examples of meganucleases that can be suitable for the instant disclosure include I-Scel, I-Crel, I-Dmol, or variants and combinations thereof. A meganuclease can be targeted to a specific nucleic acid sequence by modifying its recognition sequence using techniques well known to those skilled in the art.

The programmable targeting nuclease can be a rare-cutting endonuclease or derivative thereof. Rare-cutting endonucleases are site-specific endonucleases whose recognition sequence occurs rarely in a genome, such as only once in a genome. The rare-cutting endonuclease can recognize a 7-nucleotide sequence, an 8-nucleotide sequence, or longer recognition sequence. Non-limiting examples of rare-cutting endonucleases include NotI, AscI, PacI, AsiSI, SbfI, and FseI.

vii. Optional Additional Domains.

The programmable targeting nuclease can further comprise at least one nuclear localization signal (NLS), at least one cell-penetrating domain, at least one reporter domain, and/or at least one linker.

In general, an NLS comprises a stretch of basic amino acids. Nuclear localization signals are known in the art (see, e.g., Lange et al., J. Biol. Chem., 2007, 282:5101-5105). The NLS can be located at the N-terminus, the C-terminal, or in an internal location of the fusion protein.

A cell-penetrating domain can be a cell-penetrating peptide sequence derived from the HIV-1 TAT protein. The cell-penetrating domain can be located at the N-terminus, the C-terminal, or in an internal location of the fusion protein.

A programmable targeting nuclease can further comprise at least one linker. For example, the programmable targeting nuclease, the nuclease domain of the targeting nuclease, and other optional domains can be linked via one or more linkers. The linker can be flexible (e.g., comprising small, non-polar (e.g., Gly) or polar (e.g., Ser, Thr) amino acids). Examples of suitable linkers are well known in the art, and programs to design linkers are readily available (Crasto et al., Protein Eng., 2000, 13 (5): 3096-312). In alternate aspects, the programmable targeting nuclease, the cell cycle regulated protein, and other optional domains can be linked directly.

A programmable targeting nuclease can further comprise an organelle localization or targeting signal that directs a molecule to a specific organelle. A signal can be a polynucleotide or polypeptide signal, or can be an organic or inorganic compound sufficient to direct an attached molecule to a desired organelle. Organelle localization signals can be as described in U.S. Patent Publication No. 20070196334, the disclosure of which is incorporated herein in its entirety.

### III. Nucleic Acid Constructs

A further aspect of the present disclosure provides a system of one or more nucleic acid constructs encoding the components of the engineered nucleic acid modification system described in Section II herein above.

Any of the multi-component systems described herein are to be considered modular, in that the different components can optionally be distributed among two or more nucleic acid constructs as described herein. The nucleic acid constructs can be DNA or RNA, linear or circular, single-stranded or double-stranded, or any combination thereof. The nucleic acid constructs can be codon-optimized for efficient translation into protein, and possibly for transcription into an RNA donor polynucleotide transcript in the cell of interest. Codon optimization programs are available as freeware or from commercial sources.

The nucleic acid constructs can be used to express one or more components of the system for later introduction into a cell to be genetically modified. Alternatively, the nucleic acid constructs can be introduced into the cell to be genetically modified for expression of the components of the system in the cell. In some aspects, the nucleic acid constructs transiently express the various components of the system. Transiently expressing the system in a plant overcomes the cumbersome regulatory hurdles required for traditionally genetically modified crops. In some aspects, the engineered nucleic acid modification system is expressed in male reproductive tissues, modifies expression of various factors described herein above in male reproductive tissues, or both.

Expression constructs generally comprise DNA coding sequences operably linked to at least one promoter control sequence for expression in a cell of interest. Promoter control sequences can control expression of the transposase, the programmable targeting nuclease, the donor polynucleotide, or combinations thereof in bacterial (e.g., E. coli) cells or eukaryotic (e.g., yeast, insect, mammalian, or plant) cells. Suitable bacterial promoters include, without limit, T7 promoters, lac operon promoters, trp promoters, tac promoters (which are hybrids of trp and lac promoters), variations of any of the foregoing, and combinations of any of the foregoing. Non-limiting examples of suitable eukaryotic promoters include constitutive, regulated, or cell- or tissue-specific promoters. As explained above, methylation of the MeSWEET10a gene can be targeted in leaves by specifically expressing the system in leaves using a leaf-specific promoter, allowing for fine-tuning pathogen resistance and normal plant growth and development.

Suitable eukaryotic constitutive promoter control sequences include, but are not limited to, cytomegalovirus immediate early promoter (CMV), simian virus (SV40) promoter, adenovirus major late promoter, Rous sarcoma virus (RSV) promoter, mouse mammary tumor virus (MMTV) promoter, phosphoglycerate kinase (PGK) promoter, elongation factor (ED1)-alpha promoter, ubiquitin promoters, actin promoters, tubulin promoters, immunoglobulin promoters, fragments thereof, or combinations of any of the foregoing. Examples of suitable eukaryotic regulated promoter control sequences include, without limit, those regulated by heat shock, metals, steroids, antibiotics, or alcohol. Non-limiting examples of tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, NphsI promoter, OG-2 promoter, SP-B promoter, SYN1 promoter, and WASP promoter.

Promoters can also be plant-specific promoters, or promoters that can be used in plants. A wide variety of plant promoters are known to those of ordinary skill in the art, as are other regulatory elements that can be used alone or in combination with promoters. Preferably, promoter control sequences control expression in a Pooideae or Bambusoideae plant, such as promoters disclosed in Wilson et al., 2017, The New Phytologist, 213 (4): 1632-1641 and Coussens et al., 212, J. Exp. Bot., 63 (11): 4263-73, the disclosure of both of which is incorporated herein in its entirety.

Promoters can be divided into two types, namely, constitutive promoters and non-constitutive promoters. Constitutive promoters are classified as providing for a range of constitutive expression. Thus, some are weak constitutive promoters, and others are strong constitutive promoters. Non-constitutive promoters include tissue-preferred promoters, tissue-specific promoters, cell-type specific promoters, and inducible promoters. Suitable plant-specific constitutive promoter control sequences include, but are not limited to, a CaMV35S promoter, CaMV 19S, GOS2, Arabidopsis At6669 promoter, Rice cyclophilin, Maize H3 histone, Synthetic Super MAS, an opine promoter, a plant ubiquitin (Ubi) promoter, an actin 1 (Act-1) promoter, pEMU, Cestrum yellow leaf curling virus promoter (CYMLV promoter), and an alcohol dehydrogenase 1 (Adh-1) promoter. Other constitutive promoters include those in U.S. Pat. Nos. 5,659,026; 5,608,149; 5,608,144; 5,604, 121; 5,569,597; 5,466,785; 5,399,680; 5,268,463; and 5,608,142.

Regulated plant promoters respond to various forms of environmental stresses, or other stimuli, including, for example, mechanical shock, heat, cold, flooding, drought, salt, anoxia, pathogens such as bacteria, fungi, and viruses, and nutritional deprivation, including deprivation during times of flowering and/or fruiting, and other forms of plant stress. For example, the promoter can be a promoter which is induced by one or more, but not limited to one of the following: abiotic stresses such as wounding, cold, desiccation, ultraviolet-B, heat shock or other heat stress, drought stress or water stress. The promoter can further be one induced by biotic stresses including pathogen stress, such as stress induced by a virus or fungi, stresses induced as part of the plant defense pathway or by other environmental signals, such as light, carbon dioxide, hormones or other signaling molecules such as auxin, hydrogen peroxide and salicylic acid, sugars and gibberellin or abscisic acid and ethylene. Suitable regulated plant promoter control sequences include, but are not limited to, salt-inducible promoters such as RD29A; drought-inducible promoters such as maize rab17 gene promoter, maize rab28 gene promoter, and maize lvr2 gene promoter; heat-inducible promoters such as heat tomato hsp80-promoter from tomato.

Tissue-specific promoters can include, but are not limited to, fiber-specific, green tissue-specific, root-specific, stem-specific, flower-specific, callus-specific, pollen-specific, egg-specific, promoters specific to male or female reproductive tissues, and seed coat-specific. Suitable tissue-specific plant promoter control sequences include, but are not limited to, leaf-specific promoters [such as described, for example, by Yamamoto et al., Plant J. 12:255-265, 1997; Kwon et al., Plant Physiol. 105:357-67, 1994; Yamamoto et al., Plant Cell Physiol. 35:773-778, 1994; Gotor et al., Plant J. 3:509-18, 1993; Orozco et al., Plant Mol. Biol. 23:1129-1138, 1993; and Matsuoka et al., Proc. Natl. Acad. Sci. USA 90:9586-9590, 1993], seed-preferred promoters [e.g., from seed-specific genes (Simon et al., Plant Mol. Biol. 5. 191, 1985; Scofield et al., J. Biol. Chem. 262:12202, 1987; Baszczynski et al., Plant Mol. Biol. 14:633, 1990), Brazil Nut albumin (Pearson et al., Plant Mol. Biol. 18:235-245, 1992), legumin (Ellis et al., Plant Mol. Biol. 10:203-214, 1988), Glutelin (rice) (Takaiwa et al., Mol. Gen. Genet. 208:15-22, 1986; Takaiwa et al., FEBS Letts. 221:43-47, 1987), Zein (Matzke et al., Plant Mol Biol, 143:323-32, 1990), napA (Stalberg et al., Planta 199:515-519, 1996), Wheat SPA (Albani et al, Plant Cell, 9:171-184, 1997), sunflower oleosin (Cummins et al., Plant Mol. Biol. 19:873-876, 1992)], endosperm specific promoters [e.g., wheat LMW and HMW, glutenin-1 (Mol Gen Genet 216:81-90, 1989; NAR 17:461-2), wheat a, b, and g gliadins (EMBO3: 1409-15, 1984), Barley Itrl promoter, barley B1, C, D hordein (Theor Appl Gen 98:1253-62, 1999; Plant J 4:343-55, 1993; Mol Gen Genet 250:750-60, 1996), Barley DOF (Mena et al., The Plant Journal, 116 (1): 53-62, 1998), Biz2 (EP99106056.7), Synthetic promoter (Vicente-Carbajosa et al., Plant J. 13:629-640, 1998), rice prolamin NRP33, rice-globulin Glb-1 (Wu et al., Plant Cell Physiology 39 (8) 885-889, 1998), rice alpha-globulin REB/OHP-1 (Nakase et al., Plant Mol. Biol. 33:513-S22, 1997), rice ADP-glucose PP (Trans Res 6:157-68, 1997), maize ESR gene family (Plant J 12:235-46, 1997), sorgum gamma-kafirin (PMB 32:1029-35, 1996)], embryo-specific promoters [e.g., rice OSH1 (Sato et al., Proc. Natl. Acad. Sci. USA, 93:8117-8122), KNOX (Postma-Haarsma et al., Plant Mol. Biol. 39:257-71, 1999), rice oleosin (Wu et al., J. Biochem., 123:386, 1998)], and flower-specific promoters [e.g., AtPRP4, chalene synthase (chsA) (Van der Meer et al., Plant Mol. Biol. 15, 95-109, 1990), LAT52 (Twell et al., Mol. Gen Genet. 217:240-245; 1989), apetala-3], TaGH9 from wheat Liqing Luo et al., (Int J Mol Sci. 2022 June; 23 (11): 6324), truncated Ms2 promoter containing a TRIM element or a rice promoter OsLTP (Szabala Plant Cell Rep. 2023), and promoters of selected RKD-induced genes were shown to be predominantly active in the egg cell (Koszegi et al., Plant J. 2011; 67 (2): 280-91), the disclosures of all of which are incorporated herein by reference in their entirety.

Any of the promoter sequences can be wild type or can be modified for more efficient or efficacious expression. The DNA coding sequence also can be linked to a polyadenylation signal (e.g., SV40 polyA signal, bovine growth hormone (BGH) polyA signal, etc.) and/or at least one transcriptional termination sequence. In some situations, the complex or fusion protein can be purified from the bacterial or eukaryotic cells.

Nucleic acids encoding one or more components of an engineered DNA methylation system and/or transcription activation system can be present in a construct. Suitable constructs include plasmid constructs, viral constructs, and self-replicating RNA (Yoshioka et al., Cell Stem Cell, 2013, 13:246-254). For instance, the nucleic acid encoding one or more components of an engineered DNA methylation system and/or transcription activation system can be present in a plasmid construct.

Non-limiting examples of suitable plasmid constructs include pUC, pBR322, pET, pBluescript, and variants thereof. Alternatively, the nucleic acid encoding one or more components of an engineered DNA methylation system and/or transcription activation system can be part of a viral vector (e.g., lentiviral vectors, adeno-associated viral vectors, adenoviral vectors, and so forth).

The plasmid or viral vector can comprise additional expression control sequences (e.g., enhancer sequences, Kozak sequences, polyadenylation sequences, transcriptional termination sequences, etc.), selectable reporter sequences (e.g., antibiotic resistance genes), origins of replication, T-DNA border sequences, and the like. The plasmid or viral vector can further comprise RNA processing elements such as glycine tRNAs, or Csy4 recognition sites. Such RNA processing elements can, for instance, intersperse polynucleotide sequences encoding multiple gRNAs under the control of a single promoter to produce the multiple gRNAs from a transcript encoding the multiple gRNAs. When a cys4 recognition cite is used, a vector can further comprise sequences for expression of Csy4 RNAse to process the gRNA transcript. Additional information about vectors and use thereof can be found in “Current Protocols in Molecular Biology”, Ausubel et al., John Wiley & Sons, New York, 2003, or “Molecular Cloning: A Laboratory Manual”, Sambrook & Russell, Cold Spring Harbor Press, Cold Spring Harbor, NY, 3rd edition, 2001.

The plasmid or viral vector can also comprise a transit peptide for targeting of a protein product, particularly to a chloroplast, leucoplast or other plastid organelle or vacuole or an extracellular location. For descriptions of the use of chloroplast transit peptides, see U.S. Pat. Nos. 5,188,642 and 5,728,925, herein incorporated by reference in their entirety. Many chloroplast-localized proteins are expressed from nuclear genes as precursors and are targeted to the chloroplast by a chloroplast transit peptide (CTP). Examples of other such isolated chloroplast proteins include, but are not limited to those associated with the small subunit (SSU) of ribulose-1,5,-bisphosphate carboxylase, ferredoxin, ferredoxin oxidoreductase, the light-harvesting complex protein I and protein II, thioredoxin F, enolpyruvyl shikimate phosphate synthase (EPSPS) and transit peptides described in U.S. Pat. No. 7,193,133, herein incorporated by reference. It has been demonstrated in vivo and in vitro that non-chloroplast proteins can be targeted to the chloroplast by use of protein fusions with a heterologous CTP and that the CTP is sufficient to target a protein to the chloroplast. Incorporation of a suitable chloroplast transit peptide, such as, the Arabidopsis thaliana EPSPS CTP (CTP2, Klee et al., Mol. Gen. Genet. 210:437-442), and the Petunia hybrida EPSPS CTP (CTP4, della-Cioppa et al., Proc. Natl. Acad. Sci. USA 83:6873-6877) has been show to target heterologous EPSPS protein sequences to chloroplasts in transgenic plants. The production of glyphosate tolerant plants by expression of a fusion protein comprising an amino-terminal CTP with a glyphosate resistant EPSPS enzyme is well known by those skilled in the art, (U.S. Pat. Nos. 5,627,061, 5,633,435, 5,312,910, EP 0218571, EP 189707, EP 508909, and EP 924299).

In some aspects, when the plant is H. vulgare, the one or more nucleic acid constructs encoding the components of the engineered nucleic acid modification system comprise about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 10108 to base 18139 of SEQ ID NO: 26 (HvuDCL-Binary-vector-pcoCAS9-HvDCL5). In some aspects, the one or more nucleic acid constructs encoding the components of the engineered nucleic acid modification system comprise comprising about 75% or more, at least about 85% or more, at least about 95% or more, or 100% sequence identity with the nucleic acid sequence starting at base 10108 to base 18139 of SEQ ID NO: 26 (HvuDCL-Binary-vector-pcoCAS9-HvDCL5). In some aspects, the one or more nucleic acid constructs comprise a maize polyubiquitin gene promoter operably linked to a nucleic acid sequence encoding a Cas9 nuclease and a wheat TaU6 promoter operably linked to a nucleic acid sequence encoding one or more gRNAs.

In some aspects, when the plant is H. vulgare, the one or more nucleic acid constructs encoding the components of the engineered nucleic acid modification system comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 52 (HvuDCL-Binary-vector-pcoCAS9-HvDCL5). In some aspects, when the plant is H. vulgare, the one or more nucleic acid constructs encoding the components of the engineered nucleic acid modification system comprises a nucleic acid sequence comprising about 75% or more, at least about 85% or more, at least about 95% or more, or 100% sequence identity with the nucleic acid sequence of SEQ ID NO: 52 (HvuDCL-Binary-vector-pcoCAS9-HvDCL5). In some aspects, the one or more nucleic acid constructs comprise a maize polyubiquitin gene promoter operably linked to a nucleic acid sequence encoding a Cas9 nuclease and a wheat TaU6 promoter operably linked to a nucleic acid sequence encoding one or more gRNAs.

In some aspects, when the plant is T. aestivum, the one or more nucleic acid constructs encoding the components of the engineered nucleic acid modification system comprise a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 5722 to base 13656 of SEQ ID NO: 27 (pggg-tadcl-guides135). In some aspects, when the plant is T. aestivum, the one or more nucleic acid constructs encoding the components of the engineered nucleic acid modification system comprise a nucleic acid sequence comprising about 75% or more, at least about 85% or more, at least about 95% or more, or 100% sequence identity with the nucleic acid sequence starting at base 5722 to base 13656 of SEQ ID NO: 27 (pggg-tadcl-guides135). In some aspects, the one or more nucleic acid constructs comprise a maize polyubiquitin gene promoter operably linked to a nucleic acid sequence encoding a Cas9 nuclease and a wheat TaU6 promoter operably linked to a nucleic acid sequence encoding one or more gRNAs.

In some aspects, when the plant is T. aestivum, the one or more nucleic acid constructs encoding the components of the engineered nucleic acid modification system comprise a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 53 (pggg-tadcl-guides135). In some aspects, when the plant is T. aestivum, the one or more nucleic acid constructs encoding the components of the engineered nucleic acid modification system comprise a nucleic acid sequence comprising about 75% or more, at least about 85% or more, at least about 95% or more, or 100% sequence identity with the nucleic acid sequence of SEQ ID NO: 53 (pggg-tadcl-guides135). In some aspects, the one or more nucleic acid constructs comprise a maize polyubiquitin gene promoter operably linked to a nucleic acid sequence encoding a Cas9 nuclease and a wheat TaU6 promoter operably linked to a nucleic acid sequence encoding one or more gRNAs.

In some aspects, when the plant is T. aestivum, the one or more nucleic acid constructs encoding the components of the engineered nucleic acid modification system comprise a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 5722 to base 13656 of SEQ ID NO: 28 (pggg-tadcl-guides246). In some aspects, when the plant is T. aestivum, the one or more nucleic acid constructs encoding the components of the engineered nucleic acid modification system comprise a nucleic acid sequence comprising about 75% or more, at least about 85% or more, at least about 95% or more, or 100% sequence identity with the nucleic acid sequence starting at base 5722 to base 13656 of SEQ ID NO: 28 (pggg-tadcl-guides246). In some aspects, the one or more nucleic acid constructs comprise a maize polyubiquitin gene promoter operably linked to a nucleic acid sequence encoding a Cas9 nuclease and a wheat TaU6 promoter operably linked to a nucleic acid sequence encoding one or more gRNAs.

In some aspects, when the plant is T. aestivum, the one or more nucleic acid constructs encoding the components of the engineered nucleic acid modification system comprise a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 54 (pggg-tadcl-guides246). In some aspects, when the plant is T. aestivum, the one or more nucleic acid constructs encoding the components of the engineered nucleic acid modification system comprise a nucleic acid sequence comprising about 75% or more, at least about 85% or more, at least about 95% or more, or 100% sequence identity with the nucleic acid sequence of SEQ ID NO: 54 (pggg-tadcl-guides246). In some aspects, the one or more nucleic acid constructs comprise a maize polyubiquitin gene promoter operably linked to a nucleic acid sequence encoding a Cas9 nuclease and a wheat TaU6 promoter operably linked to a nucleic acid sequence encoding one or more gRNAs.

In some aspects, when the plant is T. aestivum, the one or more nucleic acid constructs encoding the components of the engineered nucleic acid modification system comprise a nucleic acid construct comprising a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 5722 to base 13656 of SEQ ID NO: 27 (pggg-tadcl-guides135) and a nucleic acid construct comprising a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 5722 to base 13655 of SEQ ID NO: 28 (pggg-tadcl-guides246). In some aspects, the one or more nucleic acid constructs encoding the components of the engineered nucleic acid modification system comprise a nucleic acid construct comprising a nucleic acid sequence comprising about 75% or more, at least about 85% or more, at least about 95% or more, or 100% sequence identity with the nucleic acid sequence starting at base 5722 to base 13656 of SEQ ID NO: 27 (pggg-tadcl-guides135) and a nucleic acid construct comprising a nucleic acid sequence comprising about 75% or more, at least about 85% or more, at least about 95% or more, or 100% sequence identity with the nucleic acid sequence starting at base 5722 to base 13655 of SEQ ID NO: 28 (pggg-tadcl-guides246). In some aspects, the one or more nucleic acid constructs comprise a maize polyubiquitin gene promoter operably linked to a nucleic acid sequence encoding a Cas9 nuclease and a wheat TaU6 promoter operably linked to a nucleic acid sequence encoding one or more gRNAs.

In some aspects, when the plant is T. aestivum, the one or more nucleic acid constructs encoding the components of the engineered nucleic acid modification system comprise a nucleic acid construct comprising a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 53 (pggg-tadcl-guides135) and a nucleic acid construct comprising a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 54 (pggg-tadcl-guides246). In some aspects, the one or more nucleic acid constructs encoding the components of the engineered nucleic acid modification system comprise a nucleic acid construct comprising a nucleic acid sequence comprising about 75% or more, at least about 85% or more, at least about 95% or more, or 100% sequence identity with the nucleic acid sequence of SEQ ID NO: 53 (pggg-tadcl-guides 135) and a nucleic acid construct comprising a nucleic acid sequence comprising about 75% or more, at least about 85% or more, at least about 95% or more, or 100% sequence identity with the nucleic acid sequence of SEQ ID NO: 54 (pggg-tadcl-guides246). In some aspects, the one or more nucleic acid constructs comprise a maize polyubiquitin gene promoter operably linked to a nucleic acid sequence encoding a Cas9 nuclease and a wheat TaU6 promoter operably linked to a nucleic acid sequence encoding one or more gRNAs.

### IV. Methods

A further aspect of the present disclosure encompasses a method of generating a conditionally male-sterile genetically modified plant selected from the Pooideae subfamily or the Bambusoideae subfamily of plants. The method comprises generating a plant comprising a nucleic acid modification in the nucleic acid sequence encoding a reproductive 24-nt phasiRNA or in a polynucleotide in the phasiRNA biogenesis pathway, thereby modifying the expression of the reproductive 24-nt phasiRNA, modifying the expression of the reproductive 24-nt phasiRNA, modifying the expression of a polynucleotide in a biogenesis pathway of the reproductive 24-nt phasiRNA, or any combination thereof. Genetically modified plants generated using methods of the instant disclosure can be as described in Section I herein above.

The method comprises introducing one or more nucleic acid expression constructs for expressing an engineered nucleic acid modification system into a Pooideae or Bambusoideae plant or plant cell. The plant or plant cell is then grown under conditions whereby the nucleic acid expression construct expresses the programmable nucleic acid modification system. Expressing the programmable nucleic acid modification system introduces a nucleic acid modification in the nucleic acid sequence encoding a reproductive 24-nt phasiRNA or in a polynucleotide in the phasiRNA biogenesis pathway, thereby modifying the expression of the reproductive 24-nt phasiRNA, modifying the expression of the reproductive 24-nt phasiRNA, modifying the expression of a polynucleotide in a biogenesis pathway of the reproductive 24-nt phasiRNA, or any combination thereof, thereby generating a genetically modified plant comprising a conditional male-sterile phenotype. The genetically modified plant can be as described in Section I. The engineered nucleic acid modification system for introducing the nucleic acid modification can be as described in Section II, and nucleic acid constructs expressing the engineered nucleic acid modification system can be as described in Section III.

The method comprises introducing a nucleic acid modification into the plant. The genetic modification can comprise an exogenous nucleic acid molecule such as a chimeric nucleic acid of the disclosure. The term “exogenous” as used herein refers to a nucleic acid molecule originating from outside the plant cell. An exogenous nucleic acid molecule can be, for example, the coding sequence of a nucleic acid molecule encoding a factor in the biogenesis pathway of pre-meiotic phasiRNAs, or an element which reduces expression of a factor in the biogenesis pathway of pre-meiotic phasiRNAs. An exogenous nucleic acid molecule can have a naturally occurring or non-naturally occurring nucleotide sequence and can be a heterologous nucleic acid molecule derived from a different organism or a different plant species than the plant cell into which the nucleic acid molecule is introduced or can be a nucleic acid molecule derived from the same plant species as the plant cell into which it is introduced. The exogenous nucleic acid can or can not be integrated in the plant cell's genome. When said exogenous nucleic acid/gene is not integrated, transient expression of the nucleic acid/gene occurs in the plant cell.

Non-limiting examples of methods of introducing genetic modifications in a plant cell can be transposon insertion mutagenesis, T-DNA insertion mutagenesis, T-DNA activation tagging, chemically or radio-induced mutagenesis, TILLING (Targeted Induced Local Lesions In Genomes), site-directed mutagenesis, directed evolution, homologous recombination, introducing and expressing in a plant a nucleic acid encoding a factor in the biogenesis pathway of pre-meiotic phasiRNAs, or an element which reduces expression of a factor in the biogenesis pathway of pre-meiotic phasiRNAs, introducing an engineered nucleic acid modification system such as a CRISPR/Cas system, or any combination thereof.

In some aspects, methods of introducing a nucleic acid modification of the instant disclosure comprise using TILLING. Methods for TILLING are well known in the art and include McCallum et al. (2000) Nat. Biotechnol. 18:455-457; reviewed by Stemple (2004) Nat. Rev. Genet. 5 (2): 145-50, the disclosures of all of which are incorporated herein in their entirety. In short, TILLING is a mutagenesis technology useful to generate and/or identify, and to eventually isolate, mutagenized plants. TILLING also allows selection of plants carrying such mutant plants. TILLING combines high-density mutagenesis with high-throughput screening methods. The steps typically followed in TILLING are: (a) EMS mutagenesis; (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product.

Populations or libraries of plants comprising genetic modifications can also be used in a method of the instant disclosure. When populations of plants comprising genetic modifications are used, the method can comprise the identification of a plant in the population comprising a genetic modification of a polynucleotide in a phasiRNA biogenesis pathway responsible for biogenesis of phasiRNAs. Non-limiting examples of populations of plants comprising genetic modifications include TILLING populations, SNP populations, populations of plants comprising naturally-occurring variations, or any combination thereof. Methods of screening populations of populations of plants comprising genetic modifications to identify are known in the art.

In some aspects, a method of instant disclosure comprises screening TILLING populations of Pooideae and Bambusoideae plants. Non-limiting examples of TILLING populations of Pooideae and Bambusoideae plants include TILLING populations developed in tetraploid durum wheat and hexaploid bread wheat at the University of California Davis, Rothamsted Research, the Earlham Institute, and the John Innes Centre and TILLING populations of barley (Hordeum vulgare) developed as described in Schreiber et al., Plant Methods volume 15, Article number: 99 (2019).

In some aspects, methods of introducing a nucleic acid modification of the instant disclosure comprise using an engineered nucleic acid modification system to generate the genetically modified plant. The methods can comprise introducing an engineered nucleic acid modification system or introducing nucleic acid constructs encoding the components of the engineered nucleic acid modification system. Engineered nucleic acid modification systems can be as described in Section II herein above, and nucleic acid constructs encoding components of the engineered nucleic acid modification systems can be as described in Section III herein above.

The engineered nucleic acid modification system modifies the expression of a nucleic acid sequence encoding a polypeptide or a polynucleotide in a phasiRNA biogenesis pathway responsible for biogenesis of pre-meiotic 24-nt phasiRNAs, mid-meiotic 24-nt phasiRNAs, or both, in male reproductive tissues in a plant in the Pooideae or Bambusoideae subfamilies of plants. The plant or plant cell is then grown under conditions whereby the nucleic acid expression construct expresses the programmable nucleic acid modification system in the plant or plant cell. Expressing the programmable nucleic acid modification system or expressing the polypeptide or polynucleotide introduces a nucleic acid modification of the nucleic acid sequence encoding the polypeptide or polynucleotide, thereby modifying the expression of the polypeptide or polynucleotide in the plant. In some aspects, the engineered nucleic acid modification system is expressed in male reproductive tissues, modifies expression of various factors described herein above in male reproductive tissues, or both.

### (a) Producing Hybrid Seed

Yet another aspect of the present disclosure encompasses a method of producing hybrid seed of a Pooideae or Bambusoideae plant. The method comprises planting seeds of a first Pooideae or Bambusoideae parent plant genetically modified to comprise a conditional male-sterile phenotype and a second parent plant. The method further comprises allowing the seeds to germinate and grow into plants followed by submitting the first parent plants before flowering, during flowering, or both for a time and under conditions sufficient for the plants to develop the conditional male sterile phenotype. The second parent plant is allowed to pollinate the first parent plant to thereby produce the hybrid seed on the first parent plant. Methods of planting, submitting plants to appropriate conditions, pollinating a first and second parent plant to produce hybrid seed are known to individuals of skill in the art.

(b) Introduction into the Cell

The method comprises introducing a nucleic acid construct expressing an engineered protein into a cell of interest. As explained above, an engineered protein can be encoded on more than one nucleic acid sequence. Accordingly, a method of the instant disclosure comprises introducing more than one nucleic acid construct into the cell.

The one or more nucleic acid constructs described above can be introduced into the cell by a variety of means. Suitable delivery means include microinjection, electroporation, sonoporation, biolistics, calcium phosphate-mediated transfection, cationic transfection, liposomes and other lipids, dendrimer transfection, heat shock transfection, nucleofection transfection, gene gun delivery, dip transformation, supercharged proteins, cell-penetrating peptides, viral vectors, magnetofection, lipofection, impalefection, optical transfection, Agrobacterium tumefaciens mediated foreign gene transformation, proprietary agent-enhanced uptake of nucleic acids, and delivery via liposomes, immunoliposomes, virosomes, or artificial virions. The choice of means of introducing the system into a cell can and will vary depending on the cell, or the system or nucleic acid nucleic acid constructs encoding the system, among other variables.

### (c) Culturing a Cell

The method further comprises culturing a cell under conditions suitable for expressing the engineered protein. Methods of culturing cells are known in the art. In some aspects, the cell is from an animal, fungi, oomycete or prokaryote. In some aspects, the cell is a plant cell, plant, or plant part. When the cell is in tissue ex vivo, or in vivo within a plant or within a plant part, the plant part and/or plant can also be maintained under appropriate conditions for insertion of the donor polynucleotide. In general, the plant, plant part, or plant cell is maintained under conditions appropriate for cell growth and/or maintenance. Those of skill in the art appreciate that methods for culturing plant cells are known in the art and can and will vary depending on the cell type. Routine optimization can be used, in all cases, to determine the best techniques for a particular cell type. See for example, in Santiago et al. (2008) PNAS 105:5809-5814; Moehle et al. (2007) PNAS 104:3055-3060; Urnov et al. (2005) Nature 435:646-651; Lombardo et al. (2007) Nat. Biotechnology 25:1298-1306; and Taylor et al. (2012) Tropical Plant Biology 5:127-139.

### V. Kits

A further aspect of the present disclosure provides kits for generating a genetically modified plant or plant cell of a Pooideae or Bambusoideae plant comprising a conditional male-sterile phenotype or for producing hybrid seed of the Pooideae or Bambusoideae plant. The kits comprise one or more genetically modified plants or plant cells in the Pooideae or Bambusoideae subfamily of plants comprising a conditional male-sterile phenotype; one or more expression constructs for introducing a genetic modification of a reproductive 24-nt phasiRNA, modifying the expression of the reproductive 24-nt phasiRNA, modifying the expression of a polynucleotide in a biogenesis pathway of the reproductive 24-nt phasiRNA, or any combination thereof, in a plant or plant cell selected from the Pooideae subfamily or the Bambusoideae subfamily of plants; one or more plants or plant cells comprising one or more expression constructs for expressing a nucleic acid modification system for introducing a genetic modification of a reproductive 24-nt phasiRNA, modifying the expression of the reproductive 24-nt phasiRNA, modifying the expression of a polynucleotide in a biogenesis pathway of the reproductive 24-nt phasiRNA, or any combination thereof; or any combination thereof. The genetically modified plant can be as described in Section I herein above, the engineered nucleic acid modification system can be as described in Section II herein above, the one or more nucleic acid constructs encoding the components of the engineered nucleic acid modification system can be as described in Section III herein above.

The kits can further comprise transfection reagents, cell growth media, selection media, in vitro transcription reagents, nucleic acid purification reagents, protein purification reagents, buffers, and the like. The kits provided herein generally include instructions for carrying out the methods detailed below. Instructions included in the kits can be affixed to packaging material or can be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there can be additional elements other than the listed elements.

A “genetically modified” plant refers to a plant in which the nuclear, organellar or extrachromosomal nucleic acid sequences of a cell has been modified, i.e., the cell contains at least one nucleic acid sequence that has been engineered to contain an insertion of at least one nucleotide, a deletion of at least one nucleotide, and/or a substitution of at least one nucleotide.

As used herein, the term “target nucleic acid sequence of a miRNA trigger of 24-nt phasiRNAs synthesis” refers to a nucleic acid sequence

As used herein, the term “gene” refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

As used herein, the term “engineered” when applied to a targeting protein refers to targeting proteins modified to specifically recognize and bind to a nucleic acid sequence at or near a target nucleic acid locus. A “genetically modified” plant refers to a cell in which the nuclear, organellar or extrachromosomal nucleic acid sequences of a cell have been modified, i.e., the cell contains at least one nucleic acid sequence that has been engineered to contain an insertion of at least one nucleotide, a deletion of at least one nucleotide, and/or a substitution of at least one nucleotide.

The term “nucleic acid modification” refers to processes by which a specific nucleic acid sequence in a polynucleotide is changed such that the nucleic acid sequence is modified. The nucleic acid sequence can be modified to comprise an insertion of at least one nucleotide, a deletion of at least one nucleotide, and/or a substitution of at least one nucleotide. The modified nucleic acid sequence is inactivated such that no product is made. Alternatively, the nucleic acid sequence can be modified such that an altered product is made.

As used herein, “protein expression” includes but is not limited to one or more of the following: transcription of a gene into precursor mRNA; splicing and other processing of the precursor mRNA to produce mature mRNA; mRNA stability; translation of the mature mRNA into protein (including codon usage and tRNA availability); production of a mutant protein comprising a mutation that modifies the activity of the protein, including the calcium channel activity; and glycosylation and/or other modifications of the translation product, if required for proper expression and function. The term “heterologous” refers to an entity that is not native to the cell or species of interest.

The terms “nucleic acid” and “polynucleotide” refer to a deoxyribonucleotide or ribonucleotide polymer, in linear or circular conformation. For the purposes of the present disclosure, these terms are not to be construed as limiting with respect to the length of a polymer. The terms can encompass known analogs of natural nucleotides, as well as nucleotides that are modified in the base, sugar and/or phosphate moieties. In general, an analog of a particular nucleotide has the same base-pairing specificity, i.e., an analog of A will base-pair with T. The nucleotides of a nucleic acid or polynucleotide can be linked by phosphodiester, phosphothioate, phosphoramidite, phosphorodiamidate bonds, or combinations thereof.

The term “nucleotide” refers to deoxyribonucleotides or ribonucleotides. The nucleotides can be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog can be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “polypeptide” and “protein” are used interchangeably to refer to a polymer of amino acid residues.

As used herein, the terms “target site”, “target sequence”, or “nucleic acid locus” refer to a nucleic acid sequence that defines a portion of a nucleic acid sequence to be modified or edited and to which a homologous recombination composition is engineered to target.

The terms “upstream” and “downstream” refer to locations in a nucleic acid sequence relative to a fixed position. Upstream refers to the region that is 5′ (i.e., near the 5′ end of the strand) to the position, and downstream refers to the region that is 3′ (i.e., near the 3′ end of the strand) to the position.

The term “allele” as used herein refers to one of two or more different nucleotide sequences that occur at a specific locus.

“Backcrossing” refers to the process whereby hybrid progeny are repeatedly crossed back to one of the parents. In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. (1995) Marker-assisted backcrossing: a practical example, in Techniques et Utilisations des Marqueurs Moleculaires Les Colloques, Vol. 72, pp. 45-56, and Openshaw et al., (1994) Marker-assisted Selection in Backcross Breeding, Analysis of Molecular marker Data, pp. 41-43. The initial cross gives rise to the F1 generation: the term “BC1” then refers to the second use of the recurrent parent; “BC2” refers to the third use of the recurrent parent, and so on.

The term “crossed” or “cross” means the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

As used herein, an “elite line” is any line that has resulted from breeding and selection for superior agronomic performance.

A “favorable allele” is the allele at a particular locus that confers, or contributes to, a desirable phenotype, e.g., increased GS tolerance, or alternatively, is an allele that allows the identification of plants with decreased GS tolerance that can be removed from a breeding program or planting (“counterselection”). A favorable allele of a marker is a marker allele that segregates with the favorable phenotype, or alternatively, segregates with the unfavorable plant phenotype, therefore providing the benefit of identifying plants.

“Genome” refers to the total DNA, or the entire set of genes, carried by a chromosome or chromosome set.

The terms “phenotype”, or “phenotypic trait” or “trait” refer to one or more traits of an organism. The phenotype can be observable to the naked eye, or by any other means of evaluation known in the art, e.g., microscopy, biochemical analysis, or an electromechanical assay. In some cases, a phenotype is directly controlled by a single gene or genetic locus, i.e., a “single gene trait”. In other cases, a phenotype is the result of several genes.

The term “genotype” is the genetic constitution of an individual (or group of individuals) at one or more genetic loci, as contrasted with the observable trait (the phenotype). Genotype is defined by the allele(s) of one or more known loci that the individual has inherited from its parents. The term genotype can be used to refer to an individual's genetic constitution at a single locus, at multiple led, or, more generally, the term genotype can be used to refer to an individual's genetic make-up for all the genes in its genome.

“Germplasm” refers to genetic material of or from an individual (e.g., a plant), a group of individuals (e.g., a plant line, variety or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants can be grown, or plant parts, such as leaves, stems, pollen, or cells, that can be cultured into a whole plant.

A “haplotype” is the genotype of an individual at a plurality of genetic loci, i.e. a combination of alleles. Typically, the genetic loci described by a haplotype are physically and genetically linked, i.e., on the same chromosome segment. The term “haplotype” can refer to sequence, polymorphisms at a particular locus, such as a single marker locus, or sequence polymorphisms at multiple loci along a chromosomal segment in a given genome. The former can also be referred to as “marker haplotypes” or “marker alleles”, while the latter can be referred to as “long-range haplotypes”.

A “heterotic group” comprises a set of genotypes that perform well when crossed with genotypes from a different heterotic group (Hallauer at al. (1998) Corn breeding, p. 463-564. In G. F. Sprague and J. W. Dudley (ed) Corn and corn improvement). Inbred lines are classified into heterotic groups, and are further subdivided into families within a heterotic group, based on several criteria such as pedigree, molecular marker-based associations, and performance in hybrid combinations (Smith at al. (1990) Theor. Appl. Gen. 80:833-840). The two most widely used heterotic groups in the United States are referred to as “Iowa Stiff Stalk Synthetic” (BSSS) and “Lancaster” or “Lancaster Sure Crop” (sometimes referred to as NSS, or Iron-Stiff Stalk).

The term “heterozygous” means a genetic condition wherein different alleles reside at corresponding loci on homologous chromosomes.

The term “homozygous” means a genetic condition wherein identical alleles reside at corresponding loci on homologous chromosomes.

The term “hybrid” means a progeny of mating between at least two genetically dissimilar parents. Without limitation, examples of mating schemes include single crosses, modified single cross, double modified single cross, three-way cross, modified three-way cross, and double cross wherein at least one parent in a modified cross is the progeny of a cross between sister lines.

“Hybridization” or “nucleic acid hybridization” refers to the pairing of complementary RNA and DNA strands as well as the pairing of complementary DNA single strands.

The term “hybridize” means the formation of base pairs between complementary regions of nucleic acid strands.

The term “inbred” means a line that has been bred for genetic homogeneity.

The term “indel” refers to an insertion or deletion, wherein one line can be referred to as having an insertion relative to a second line, or the second line can be referred to as having a deletion relative to the first line.

The term “introgression” or “introgressing” refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., a selected allele of a marker, a QTL, a transgene, or the like. In any case, offspring comprising the desired allele can be repeatedly backcrossed to a line having a desired genetic background and selected for the desired allele, to result in the allele becoming fixed in a selected genetic background. For example, the GS locus described herein can be introgressed into a recurrent parent that has increased GS tolerance. The recurrent parent line with the introgressed gene or locus then has increased GS tolerance.

A “physical map” of the genome is a map showing the linear order of identifiable landmarks (including genes, markers, etc.) on chromosome DNA. However, in contrast to genetic maps, the distances between landmarks are absolute (for example, measured in base pairs or isolated and overlapping contiguous genetic fragments) and not based on genetic recombination.

A “plant” can be a whole plant, any part thereof, or a cell or tissue culture derived from a plant. Thus, the term “plant” can refer to any of: whole plants, plant components or organs (e.g., leaves, stems, roots, etc.), plant tissues, seeds, plant cells, and/or progeny of the same. A plant cell is a cell of a plant, taken from a plant, or derived through culture from a cell taken from a plant.

A “polymorphism” is a variation in the DNA that is too common to be due merely to new mutation. A polymorphism must have a frequency of at least 1% in a population. A polymorphism can be a single nucleotide polymorphism, or SNP, or an insertion/deletion polymorphism, also referred to herein as an “indel”.

The term “progeny” refers to the offspring generated from a cross.

A “progeny plant” is generated from a cross between two plants.

A “reference sequence” is a defined sequence used as a basis for sequence comparison. The reference sequence is obtained by genotyping a number of lines at the locus, aligning the nucleotide sequences in a sequence alignment program (e.g. Sequencher), and then obtaining the consensus sequence of the alignment.

A “single nucleotide polymorphism (SNP)” is an allelic single nucleotide-A, T, C or G-variation within a DNA sequence representing one locus of at least two individuals of the same species. For example, two sequenced DNA fragments representing the same locus from at least two individuals of the same species, contain a difference in a single nucleotide.

The term “quantitative trait locus (QTL)” means a locus that controls to some degree numerically representable traits that are usually continuously distributed.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14 (6): 6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

As various changes could be made in the above-described cells and methods without departing from the scope of the invention, it is intended that all matter contained in the above description and in the examples given below, shall be interpreted as illustrative and not in a limiting sense.

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Loss-of-Function of Dcl5 Protein Gene Induces Conditional Male Sterility

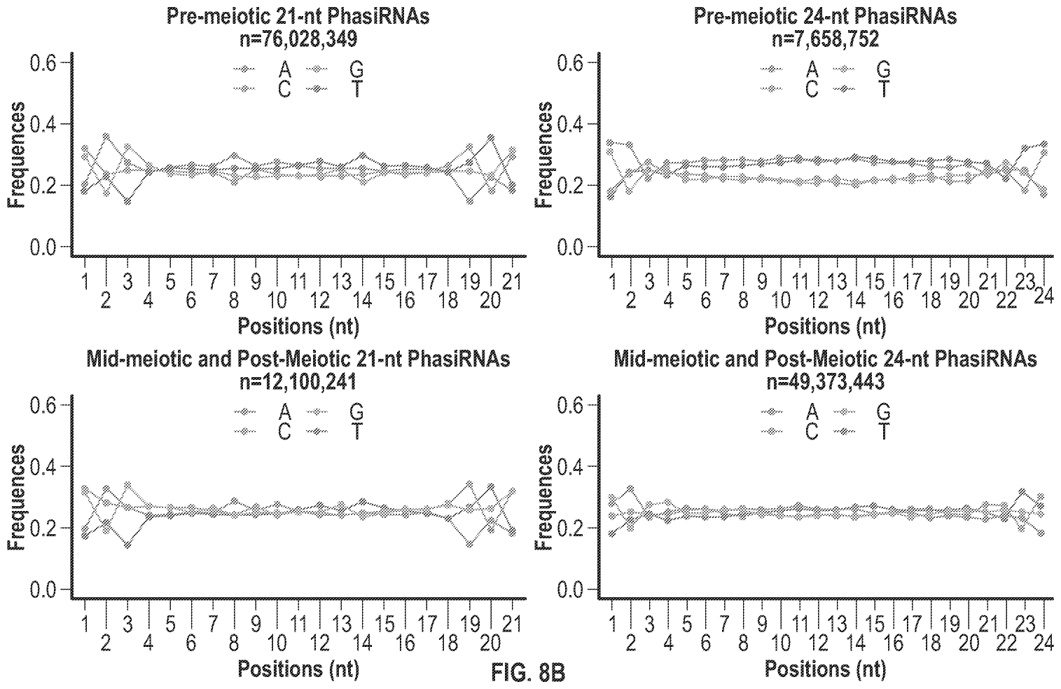

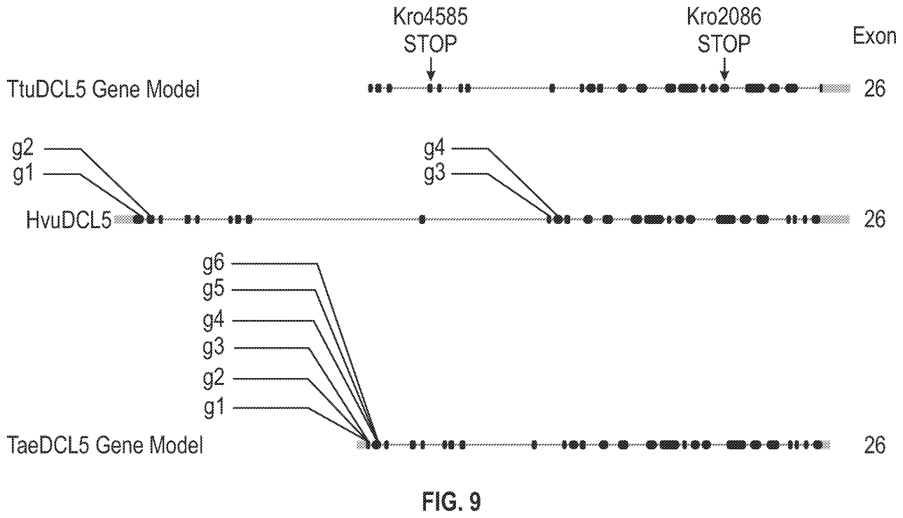

Loss-of-function mutations in the DLC5 gene were generated or obtained (FIG. 9). Anther development and phenotype were assessed in mutant tetraploid wheat lines, to determine the male fertility/sterility status under non-permissive and permissive growth conditions. The genotypes used were aabb, aAbb, aabB, and AABB. No pleiotropic effects were observed in any of the plants comprising mutant dcl5 gene, including aabb plants, when the plants are grown under normal temperature conditions (FIG. 10).

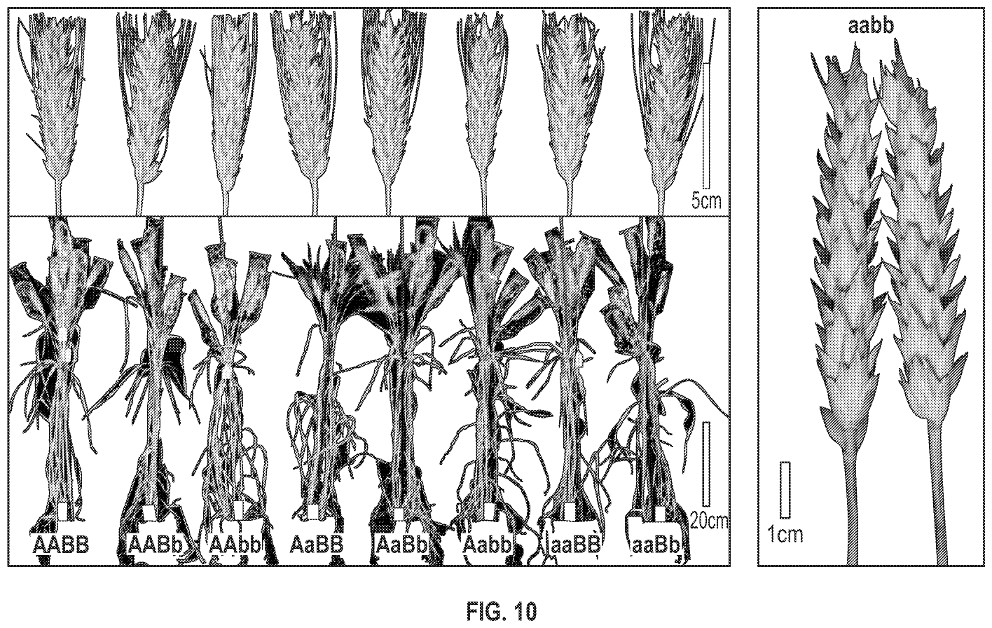

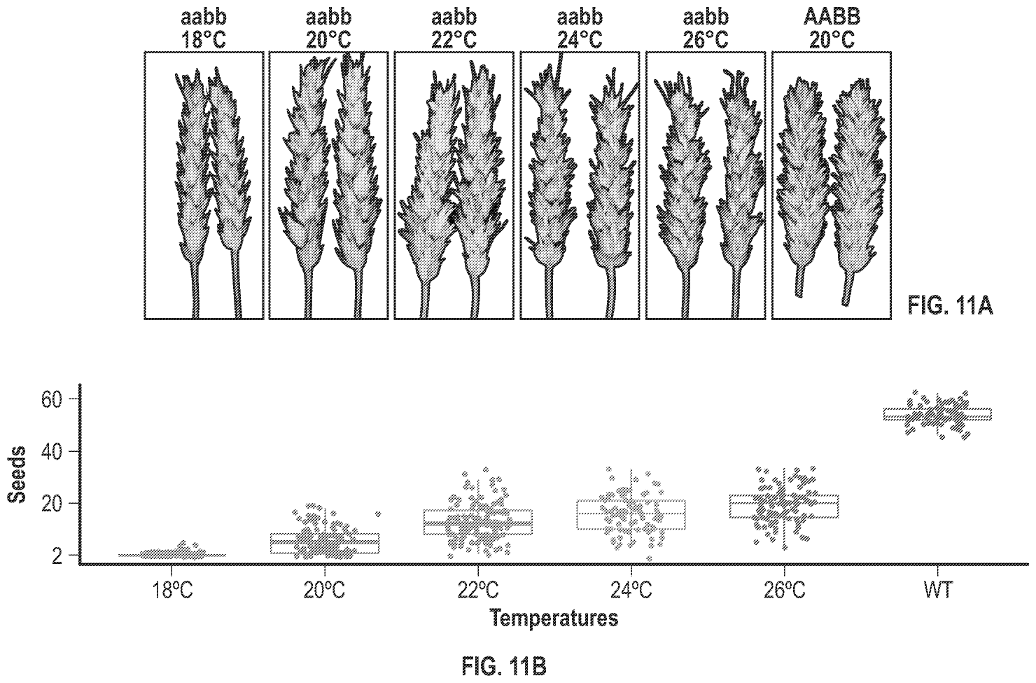

To determine if the male-sterile phenotype observed in the mutant plants is conditional, tetraploid mutant wheat cell lines were grown under various environmental conditions. It was discovered that male-sterility is temperature-sensitive. To further characterize temperature conditions controlling fertile/sterile development of flowers, dcl5 homozygous mutant in tetraploid wheat were grown under temperatures ranging from 18° C. to 26° C. (FIGS. 11A and 11B). As shown in FIG. 11B the homozygous mutant plants exhibit temperature-dependent male sterility, where plants grown under 18° C. produced no seeds, whereas plants grown under higher temperatures were fully fertile. A single allele from the “A” or “B” sub-genome was sufficient to maintain the fertility.

### Example 2. Anther Staging Identifies Developmental Defect Starting after the Meiosis

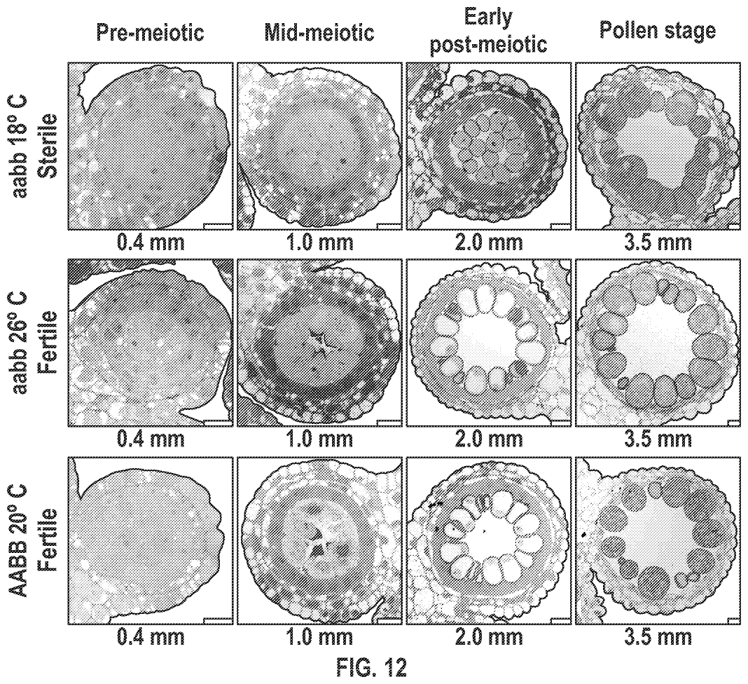

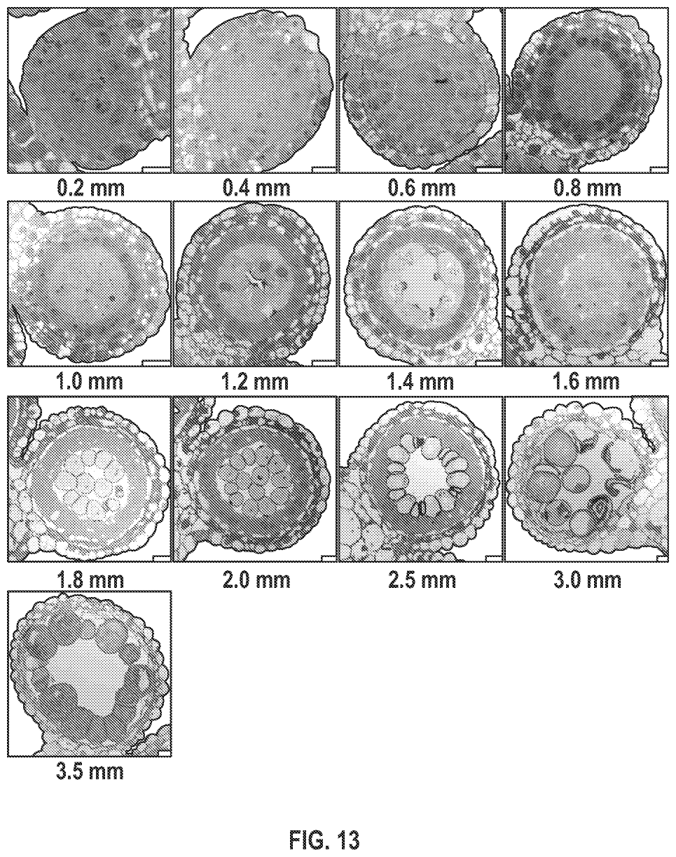

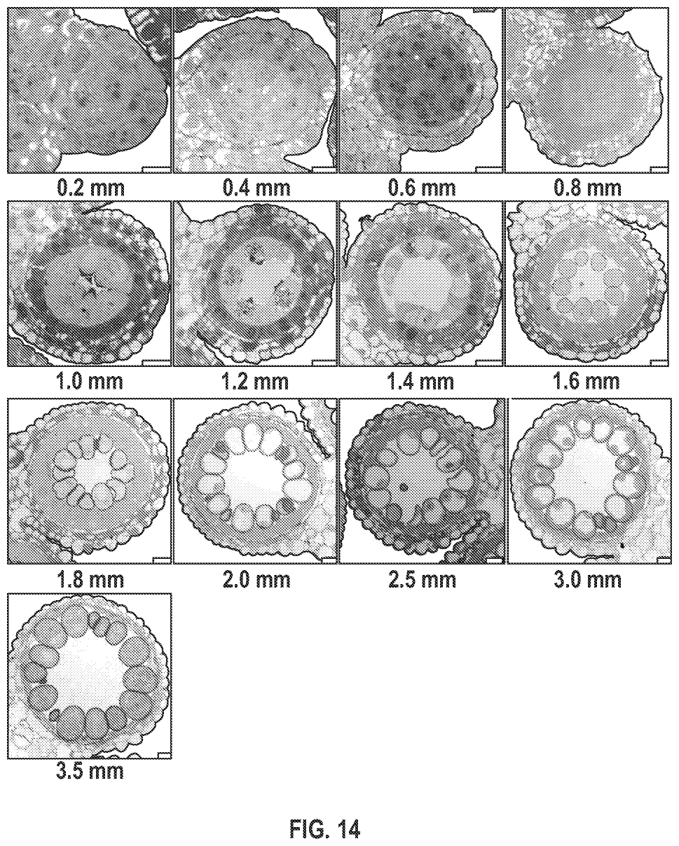

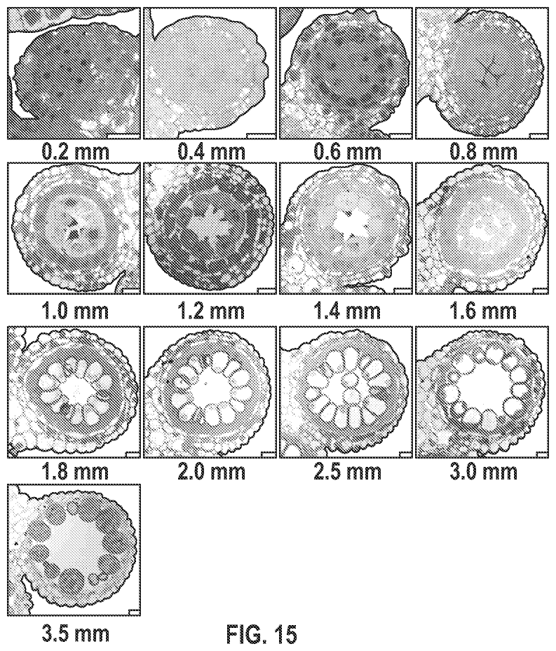

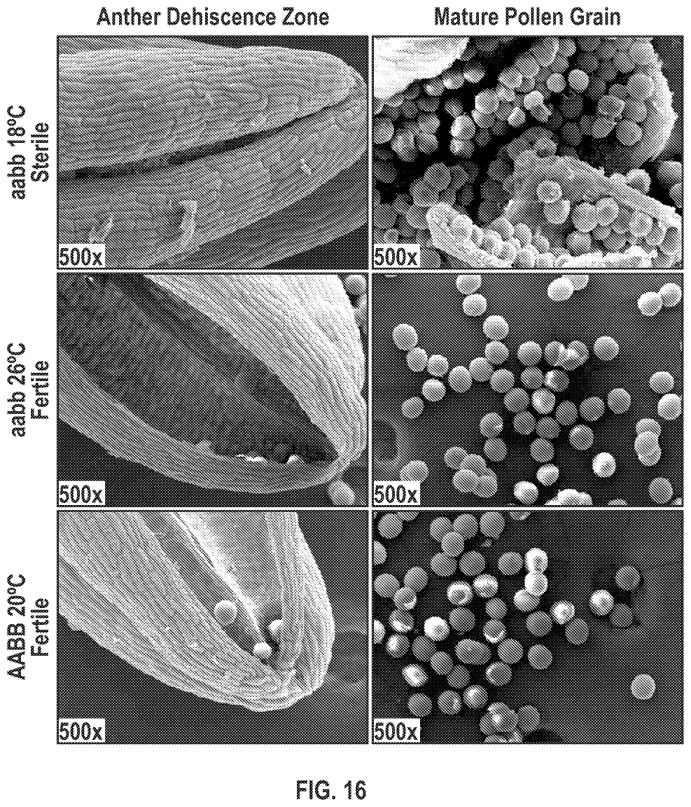

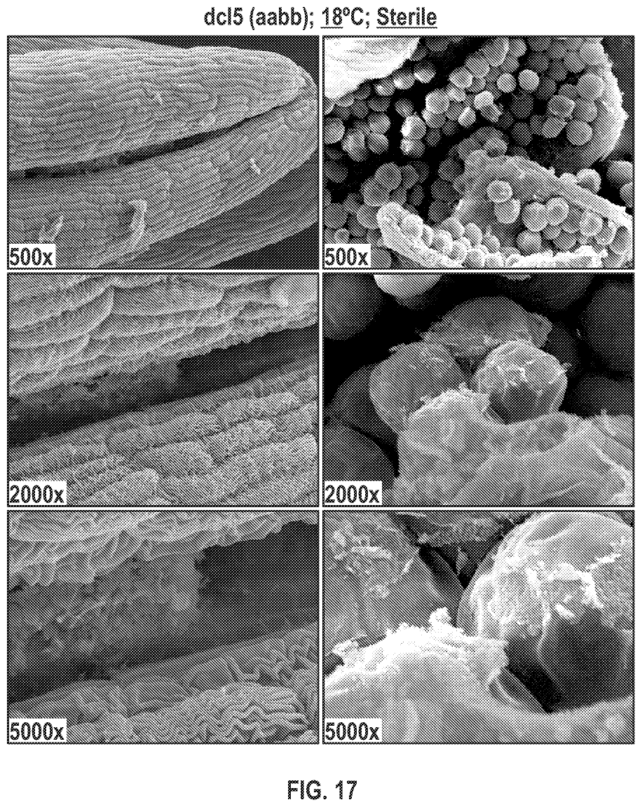

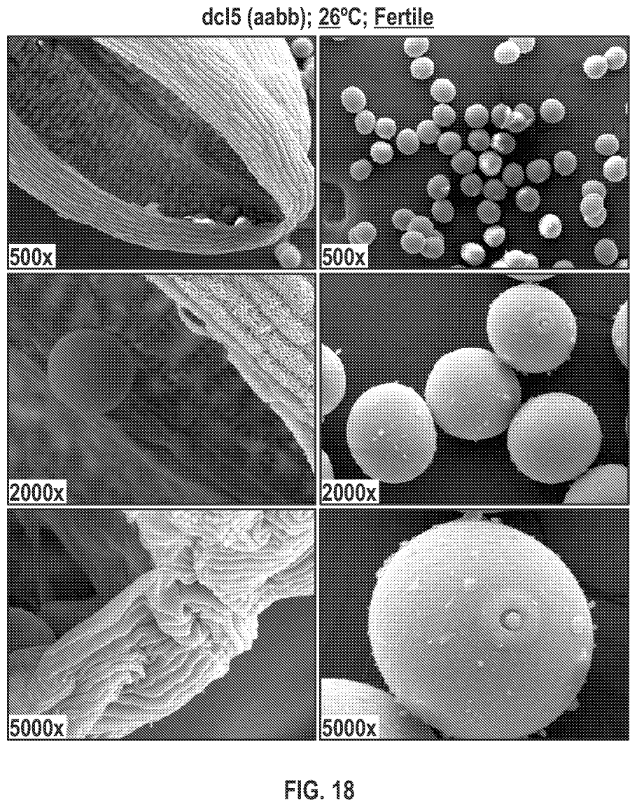

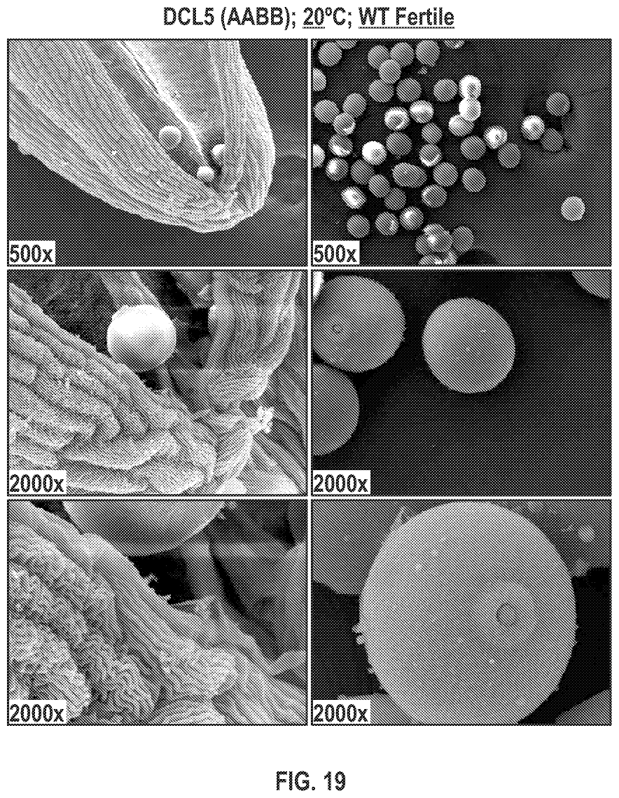

Developmental defects in developing anthers of the following DCL5 tetraploid wheat genotypes were determined: aabb, AABB using light microscopy (FIGS. 12-15) and scanning electron microscopy (SEM) (FIGS. 16-19).

Anthers develop from undifferentiated meristematic cells into an organized set of tissues with a plethora of functions. Anthers were dissected, fixed, and processed for resin embedding, and cross-sectioned to identify pre-meiotic, meiotic, and early post-meiotic stages of anther development in wheat comprising wild type DCL5 gene or mutant dcl5 gene. The developmental progression of meiosis was examined at 13 time points corresponding to 0.2- to 3.5-mm-long anthers (FIGS. 12-15). Histological analyses show developmental defects in the maturation of pollen, while no developmental failure was observed during meiotic development.

Scanning electron microscopy (SEM) shows inviable pollen (lack of pollen production) and defective anther dehiscence (lack of release of pollen) in plants grown at 18° C. Both phenotypes are partially restored when anthers develop at higher temperatures (26° C.)-[viable pollen is produced and released].

Together, these observations reveal that loss-of-function of the dcl5 gene have a major developmental defect during maturation of the pollen and deficient anther dehiscence resulting in male sterility, contrasting with the phenotype previously reported in maize. In maize, developmental defects caused by the loss-of-function of the dcl5 gene include improper tapetum development affecting pollen development at the meiosis stage.

### Example 3. Molecular Characterization of Accumulation of phasiRNAs in Developing Anthers of Dcl5 Mutants

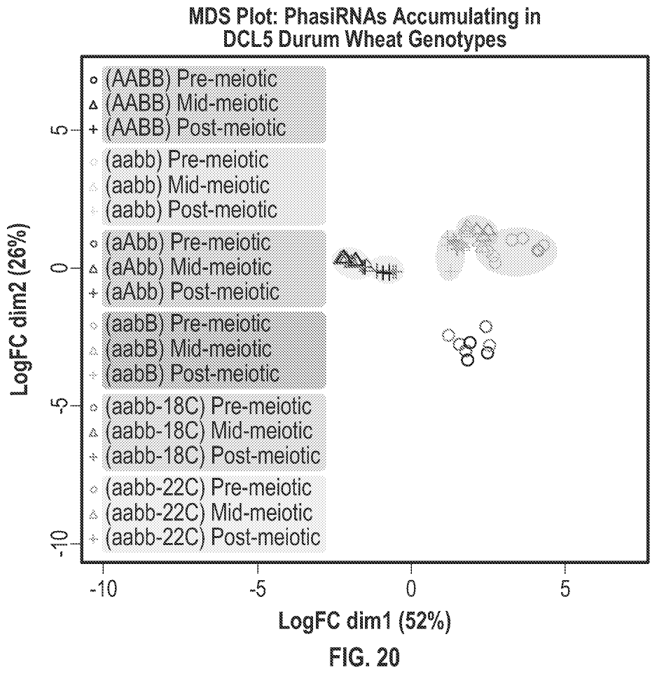

Molecular characterization of 24-nt biosynthesis by DCL5 gene was performed. The accumulation was measured in 54 sRNA libraries at 3 anther developmental stages using 3 replicates in 4 genotypes (one genotype (aabb) at three temperatures). An MDS plot of phasiRNAs accumulation in DCL5 genotypes shows a clear difference in accumulation of reproductive phasiRNAs in that dcl5 doubled mutant (aabb) when compared to wild type plants or plants comprising a single wild type allele (FIG. 20 and Table 2).

TABLE 2
Number of PHAS loci annotated in durum wheat.

| | Pre-meiotic | Mid-meiotic | Post-meiotic | Total |
|---|---|---|---|---|
| 21PHAS | 5,756 | 249 | 69 | 6,074 |
| 24PHAS | 1,449 | 1,039 | 0 | 2,448 |
| Total | 7,205 | 1288 | 69 | 8,562 |

The number of and abundance peak of 24 phasiRNA is different to previously reported in maize and rice comprised numerous 24 PHAS loci-more than ×10 the number of loci found in maize (˜250 loci) and two groups of the loci having distinct temporal accumulation peak in pre-meiotic and mid-meiotic anthers. The two features contrast with maize and rice. It was observed that pre-meiotic 24-nt phasiRNAs accumulate in pre-meiotic anther present in all Pooideae species studied, including Avena sativa (oats), Hordeum vulgare (barley), Secale cereale (rye), Triticum turgidum, Triticum aestivum (bread wheat), and Brachypodium distachyon.

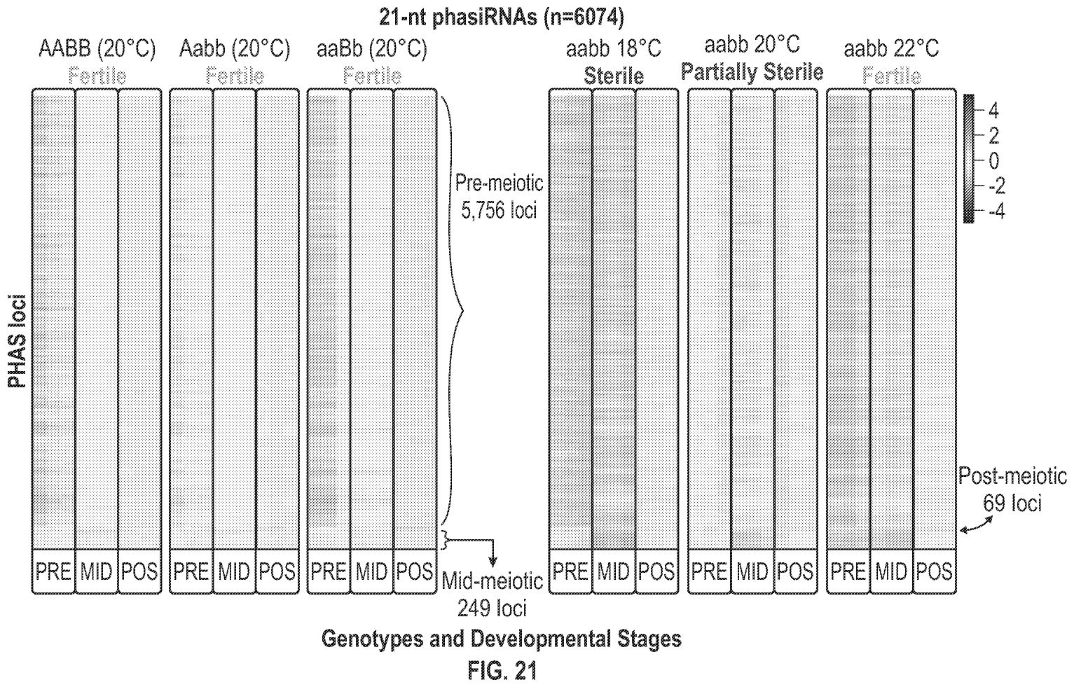

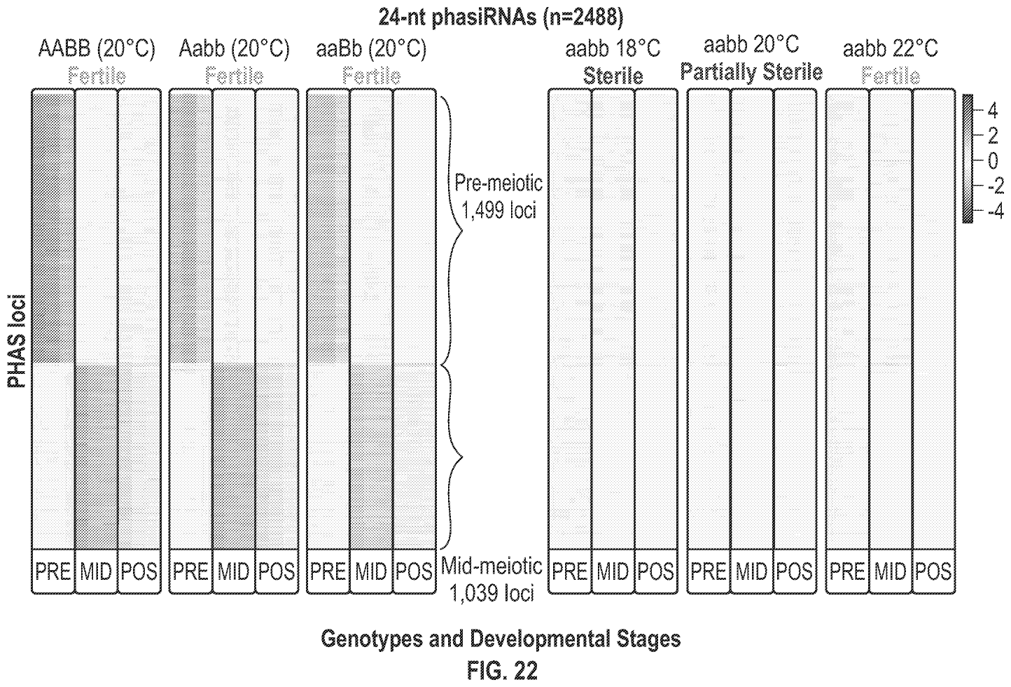

Further analysis showed that there was no change in the abundance of 21-nt phasiRNAs accumulating in wheat dc/5 doubled mutant (aabb) (FIG. 21). Therefore, loss-of-function of DCL5 gene does not affect production of 21-nt phasiRNAs, thus confirming the specificity of DCL5 to 24-nt phasiRNA biogenesis in studied species. Conversely, loss-of-function of DCL5 genes stopped the biogenesis of all 24-nt reproductive phasiRNAs when the plants are grown under permissive (high temperature) or restrictive (low temperature) conditions (FIG. 22). The effect of the loss of function mutation is only seen in homozygous mutant plants (aabb).

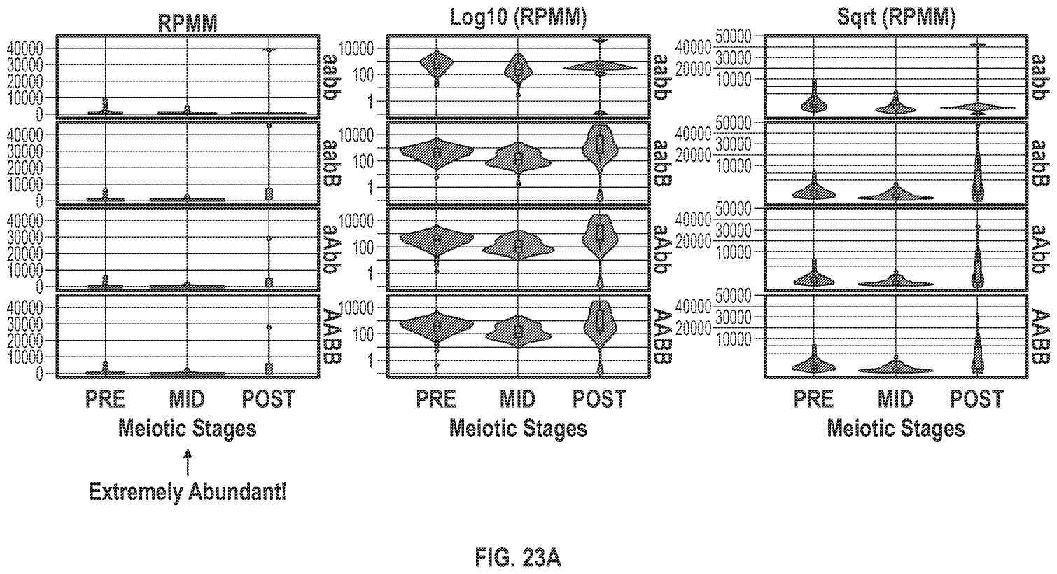

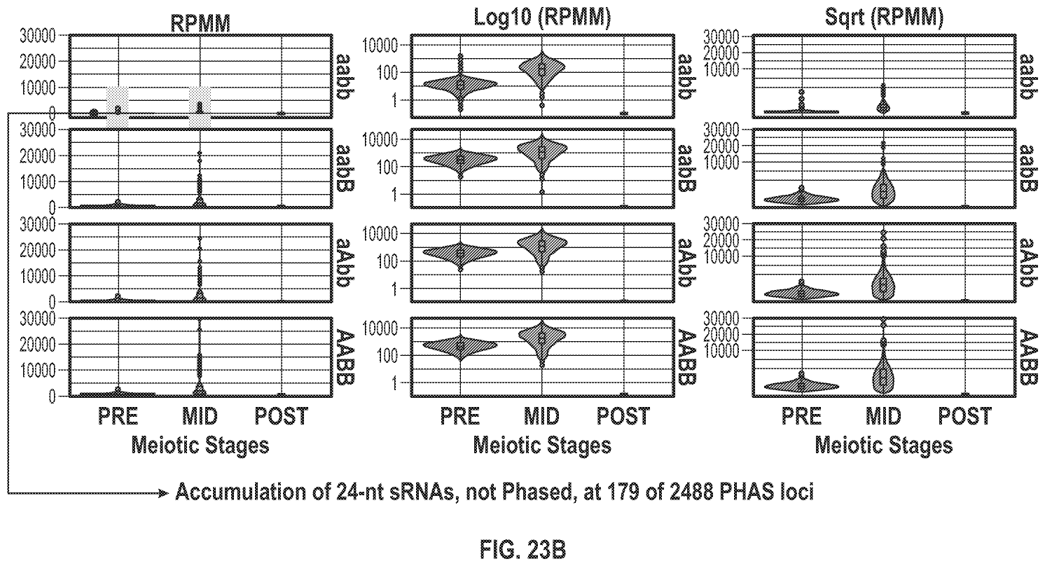

Absolute and distribution of phasiRNA abundance show that only 24-nt reproductive phasiRNAs are impacted and only in the wheat dcl5 doubled mutant (aabb) (FIGS. 23A-23C).

1. A conditionally male sterile plant or plant cell selected from the Pooideae subfamily, the plant comprising a genetic modification in a polynucleotide encoding a DCL5 protein that confers a conditional male-sterile phenotype to the plant, wherein the genetic modification reduces the expression of the DCL5 protein and reduces the expression of a reproductive 24-nt phased, secondary small interfering RNA in male reproductive tissues (reproductive 24-nt phasiRNA) in male reproductive tissues, thereby resulting in conditional male sterility, wherein the conditional male-sterile phenotype is conditional on temperature, and wherein the plant or plant cell exhibits a male-sterile phenotype when grown at a temperature of about 18° C. to about 20° C. or below before flowering, during flowering, or both, and a male-fertile phenotype when grown at a temperature of about 22° C. to about 26° C. or above before flowering, during flowering, or both.

2-28. (canceled)

29. The plant of claim 1, wherein the plant is barley (Hordeum vulgare).

30-31. (canceled)

32. The plant of claim 29, wherein the genetic modification in the polynucleotide encoding the DCL5 protein comprises a deletion of a nucleic acid sequence comprising about 75% or more, at least about 85% or more, at least about 95% or more, or 100% sequence identity with the nucleic acid sequence of SEQ ID NO: 3 or SEQ ID NO: 51, a deletion of a nucleic acid sequence comprising about 75% or more, at least about 85% or more, at least about 95% or more, or 100% sequence identity with the nucleic acid sequence of SEQ ID NO: 19, or both.

33-35. (canceled)

36. The plant of claim 1, wherein the plant is durum wheat (T. turgidum).

37-38. (canceled)

39. The plant of claim 36, wherein the plant comprises a polynucleotide encoding the DCL5 protein comprising a genetic modification encodes a transcript comprising a nucleic acid sequence comprising about 75% or more, at least about 85% or more, at least about 95% or more, or 100% sequence identity with nucleic acid sequence of SEQ ID NO: 44, a polynucleotide encoding the DCL5 protein comprising a genetic modification encodes a transcript comprising a nucleic acid sequence comprising about 75% or more, at least about 85% or more, at least about 95% or more, or 100% sequence identity with nucleic acid sequence of SEQ ID NO: 46, or both.

40. (canceled)

41. One or more expression constructs for introducing a genetic modification in a polynucleotide encoding a DCL5 protein of at least one target site that confers a conditional male-sterile phenotype to a plant or plant cell selected from the Pooideae subfamily of plants, the one or more expression constructs comprising:
a promoter operably linked to a nucleic acid sequence encoding a programmable nucleic acid modification system targeted to a polynucleotide in the polynucleotide encoding a DCL5 protein;
wherein expression of the nucleic acid modification system in the plant or plant cell introduces a genetic modification in the polynucleotide encoding the DCL5 protein, wherein the genetic modification reduces the expression of the DCL5 protein and reduces the expression of a reproductive 24-nt phasiRNA in male reproductive tissues, thereby resulting in conditional male sterility, wherein the conditional male-sterile phenotype is conditional on temperature, and wherein the plant or plant cell exhibits a male-sterile phenotype when grown at a temperature of about 18° C. to about 20° C. or below before flowering, during flowering, or both, and a male-fertile phenotype when grown at a temperature of about 22° C. to about 26° C. or above before flowering, during flowering, or both.

42. The one or more expression constructs of claim 41 wherein the programmable nucleic acid modification system comprises a Cas9 nuclease and a guide RNA (gRNA) comprising a sequence complementary to a target nucleic acid sequence within the polynucleotide encoding the polypeptide.

43-45. (canceled)

46. The one or more expression constructs of claim 42, wherein the plant is H. vulgare.

47. (canceled)

48. The one or more expression constructs of claim 46, wherein the gRNA comprises a nucleic acid sequence selected from SEQ ID NO: 15 (gRNA1), SEQ ID NO: 16 (gRNA2), SEQ ID NO: 17 (gRNA3), SEQ ID NO: 18 (gRNA4), and any combination thereof.

49. The one or more expression constructs of claim 46, wherein the one or more expression constructs comprise an expression construct comprising a nucleic acid sequence comprising about 75% or more, at least about 85% or more, at least about 95% or more, or 100% sequence identity with the nucleic acid sequence of SEQ ID NO: 52 (HvuDCL-Binary-vector-pcoCAS9-HvDCL5).

50. The one or more expression constructs of claim 1, wherein the plant is T. aestivum.

51. (canceled)

52. The one or more expression constructs of claim 50, wherein the gRNA comprises a nucleic acid sequence selected from SEQ ID NO: 20 (gRNA1), SEQ ID NO: 21 (gRNA2), SEQ ID NO: 22 (gRNA3), SEQ ID NO: 23 (gRNA4), SEQ ID NO: 24 (gRNA5), SEQ ID NO: 25 (gRNA6), and any combination thereof.

53. (canceled)

54. The one or more expression constructs of claim 50, wherein the one or more expression constructs comprise an expression construct comprising a nucleic acid sequence comprising about 75% or more, at least about 85% or more, at least about 95% or more, or 100% sequence identity with the nucleic acid sequence of SEQ ID NO: 53 (pggg-tadcl-guides135).

55. The one or more expression constructs of claim 50, wherein the one or more expression constructs comprise an expression construct comprising a nucleic acid sequence comprising about 75% or more, at least about 85% or more, at least about 95% or more, or 100% sequence identity with the nucleic acid sequence of SEQ ID NO: 54 (pggg-tadcl-guides246).

56. The one or more expression constructs of claim 50, wherein the one or more expression constructs comprise an expression construct comprising a nucleic acid sequence comprising about 75% or more, at least about 85% or more, at least about 95% or more, or 100% sequence identity with the nucleic acid sequence of SEQ ID NO: 53 (pggg-tadcl-guides135) and an expression construct comprising a nucleic acid sequence comprising about 75% or more, at least about 85% or more, at least about 95% or more, or 100% sequence identity with the nucleic acid sequence of SEQ ID NO: 54 (pggg-tadcl-guides246).

57-58. (canceled)

59. A method of producing hybrid seed of a Pooideae plant, the method comprising:
a. planting seeds of a first parent genetically modified Pooideae plant of claim 1 comprising a conditional male-sterile phenotype and a second parent plant;
b. allowing the seeds to germinate and grow into plants;
c. submitting the first parent plants before flowering, during flowering, or both for a time and under conditions sufficient for the plants to develop the conditional male-sterile phenotype; and
d. allowing the second parent plants to pollinate the first parent plants to thereby produce the hybrid seed on the first parent plant.

60. A hybrid seed of a plant of a Pooideae plant produced using a method of claim 59.

61. e.;
f.


SEQUENCE LISTING (54 sequences)

SEQ ID NO: 1 - protein, 3246 aa, Hordeum vulgare
MVMAMADDDGPIPPPPPPPPPRRPHMQLQPRRYQVEVFEAALQGNTIAVLDTGSGKTMVAVLLARYHVSRARAGEAPRRIVVFLAPTVHLVHQQFEVIREYTDLDVAECYGASGVGDWSTDRWNKEVGSKEIAVMTPQILLDALRHAFITMSAVSLLIFDECHRACGNHPYTRIMKEFYVGSEWRPAVFGMTASPVATKGTSTVQDCEAHIGQLELTLDAKVYIIKDRSELESFSPPATIVNKYYDAYSIDFEDLKSKLQILYEEYDALLANLQESSPNKFEDTNNILETSRKTLSRYHEKIFYGLNYLGPIITAEVVKIYNESIKTLDDSEDCLFSKASFSLHVAYFKEALDLIEAVLPHGYGELMKSESGTAELSKRGYISSKVDALINIFKSFGSSNEVLCLIFVERIMTAKAVERFMRGIFNFSSFSISYLTGGSTSKDSLSPAVQRYTLDLFRSGKVNLLFSTDVTEEGIDVPNCSCVIRFDLPRTVCSYVQSRGRARRRSSNYVLMIERGNMDQQEHIFRIIQTEYYIKHFALYRQETPNVSSSELPMQDKYAYHVDSTGATITADCCVDLIRKYCEKLPKDRYYMPKPSFVVAIEDGSYQCTLTLPPNAAFQRIVGPLCSTSNLAKQLVSLDACKKLHQLGELNDHLLPLTEEPIDIDVALRDGKFLSGPGTTKRKELHGTRNVLALSGSWIHESESVTLNTYRFDFLCDQEGENYAGFVLLMESALDDDVACSKMDLFLIPNKMVYTTITPCGKVQLDKKQLRDGKLFQELFFNGIFGRLFHGSRTSGVQREFLFRKGHEIQWSSECMYLLLPLRHSPHIQQDLNIYWEAVESCTQVVEQLRNLYLEDENICANSIQHRSIKEKDIIHLANKSLHFSSIKDSVVLSLHTGRIYSVLDLIYDTTAEDSFEEMYNGKASPFSSFVDYYREKYGIIIQHPKQPLLLLKQSHNAHNLLFSKSKYIDGSTGDPLLMEKEQIHARVPPELLIHIDVTIEVLKSFYLLPSVMHRLQSLMLASQLRRDIGYTQHIPSYLILEAITTLRCCETFSLERLELLGDSVLKYLIGCDLFLRYPMKHEGHLSDMRSNAVCNATLHKHGIWRSLQGYVRDSAFDPRRWVAPGQISLRPFPCNCGIETAFVPTNGRYISDDPSFVVGKPCDRAHRWMCSKTISDCVEALVGAYYVGGGIVAALWVMKWFGIEIKCDRKLVQEVKLNASYICYLPKVSVIEELEAKLKYNFSVKGLLLEAITHPSLQELGVDYCYQRLEFLGDSVLDLLITRYLYVTHTDVDPGELTDLRSALVSNENFAEVVLRNNIHSHLQHGSGILLEQITEYVKSNLECQGKVNKFLQHATCKVPKVLGDIMESIAGAIFVDTDFDVDVVWKIVEPLLSPMITPDNLALPPYRELLELCSHLGYFINSKCSSRGEEVTIDMSVQLRDELLIAQGHDRNKKSAKAKAAARILADLKKRGLSMKQCSSKSKQLDIISPDLQYPLLESPLDYCDVNVNPSLGGLPSLKEAVVLQLKMDKGGPRSALFKLCKRLQWPMPEFEFVEQRFRTPIVLDGVTTTNFNSFVSTITLHIPDVTAITIQGKRQTDKKSSQDSASLIMLHKLQELKMVMAMADDDGPIPPPPPPPPPRRPHMQLQPRRYQVEVFEAALQGNTIAVLDTGSGKTMVAVLLARYHVSRARAGEAPRRIVVFLAPTVHLVHQQFEVIREYTDLDVAECYGASGVGDWSTDRWNKEVGSKEIAVMTPQILLDALRHAFITMSAVSLLIFDECHRACGNHPYTRIMKEFYVGSEWRPAVFGMTASPVATKGTSTVQDCEAHIGQLELTLDAKVYIIKDRSELESFSPPATIVNKYYDAYSIDFEDLKSKLQILYEEYDALLANLQESSPNKFEDTNNILETSRKTLSRYHEKIFYGLNYLGPIITAEVVKIYNESIKTLDDSEDCLFSKASFSLHVAYFKEALDLIEAVLPHGYGELMKSESGTAELSKRGYISSKVDALINIFKSFGSSNEVLCLIFVERIMTAKAVERFMRGIFNFSSFSISYLTGGSTSKDSLSPAVQRYTLDLFRSGKVNLLFSTDVTEEGIDVPNCSCVIRFDLPRTVCSYVQSRGRARRRSSNYVLMIERGNMDQQEHIFRIIQTEYYIKHFALYRQETPNVSSSELPMQDKYAYHVDSTGATITADCCVDLIRKYCEKLPKDRYYMPKPSFVVAIEDGSYQCTLTLPPNAAFQRIVGPLCSTSNLAKQLVSLDACKKLHQLGELNDHLLPLTEEPIDIDVALRDGKFLSGPGTTKRKELHGTRNVLALSGSWIHESESVTLNTYRFDFLCDQEGENYAGFVLLMESALDDDVACSKMDLFLIPNKMVYTTITPCGKVQLDKKQLRDGKLFQELFFNGIFGRLFHGSRTSGVQREFLFRKGHEIQWSSECMYLLLPLRHSPHIQQDLNIYWEAVESCTQVVEQLRNLYLEDENICANSIQHRSIKEKDIIHLANKSLHFSSIKDSVVLSLHTGRIYSVLDLIYDTTAEDSFEEMYNGKASPFSSFVDYYREKYGIIIQHPKQPLLLLKQSHNAHNLLFSKSKYIDGSTGDPLLMEKEQIHARVPPELLIHIDVTIEVLKSFYLLPSVMHRLQSLMLASQLRRDIGYTQHIPSYLILEAITTLRCCETFSLERLELLGDSVLKYLIGCDLFLRYPMKHEGHLSDMRSNAVCNATLHKHGIWRSLQGYVRDSAFDPRRWVAPGQISLRPFPCNCGIETAFVPTNGRYISDDPSFVVGKPCDRAHRWMCSKTISDCVEALVGAYYVGGGIVAALWVMKWFGIEIKCDRKLVQEVKLNASYICYLPKVSVIEELEAKLKYNFSVKGLLLEAITHPSLQELGVDYCYQRLEFLGDSVLDLLITRYLYVTHTDVDPGELTDLRSALVSNENFAEVVLRNNIHSHLQHGSGILLEQITEYVKSNLECQGKVNKFLQHATCKVPKVLGDIMESIAGAIFVDTDFDVDVVWKIVEPLLSPMITPDNLALPPYRELLELCSHLGYFINSKCSSRGEEVTIDMSVQLRDELLIAQGHDRNKKSAKAKAAARILADLKKRGLSMKQCSSKSKQLDIISPDLQYPLLESPLDYCDVNVNPSLGGLPSLKEAVVLQLKMDKGGPRSALFKLCKRLQWPMPEFEFVEQRFRTPIVLDGVTTTNFNSFVSTITLHIPDVTAITIQGKRQTDKKSSQDSASLIMLHKLQELKVCICKT

SEQ ID NO: 2 - DNA, 17767 bp, Hordeum vulgare
  source 1..17767: mol_type = genomic DNA
atggtcatggccatggccgacgacgacggccccatccccccgcctcctcctccgccgccgcctcgtcgcccccacatgcagctccagccgaggaggtctcgatcggctcccattaccctccccacagctctctaccccctgcattcctagaactgacgggggtctctcgttccgtgtctgcgtcggcgcaggtatcaggtggaggtgttcgaggcggcgctgcaggggaacaccatcgcagtgctcgacaccggctccgggaagaccatggtcgccgtcctgctcgcgcgctaccacgtcagccgcgcgcgcgccggggaggcgccccgccggatcgtcgtcttcctcgcgcctaccgtgcacctcgtccaccaggtgcgcttctgaagtctgcagcgaccgtttagcgacggcaacgtctgcaccatttttccgcctcgcgttcgcagctgccgtggccctgatccctggttcttgtctggctcgcgccgtagcaattcgaggtgattcgggagtacaccgacctcgatgtcgcggagtgttacggagcttcgggtgtcggcgactggagtaccgaccgctggaacaaggaagttgggagcaaggaggtaagtgtcagctgtcagggtttactcttatccagcgacgcggtcattggttgaatttgaatgcacattgaatttgtacacgtaccgtgctcaccaagtgcttgatgaatgacttgttgacagggaaccaacagtgttagtttctgggtggtagcagcttcggatcgcatgtagattgaggccattgttattagagtagtattctttagcatgatgagagggctgtcgactaaagcaatttcctgaagggttgtgcctatgaaatagccgaggtttggacacaagtcctagaaaaaggaaacccccctttcattgcagtcactgctaaatttggacactgcttcaaatccaaaagatctcacaactcctgtaaatatgttgcttaaaaataatcttgtaaatatgccacttggtaaagtaatctggattatgtgtctttctaaattctaatacagttttgcggatgttcctatgtttttcttgtacctaccatgctgcgcttattatgcatactattgatatatttgactggaaatataaattctccttgaacaatgcatcttattatgccttccattatttgtttcagattgctgttatgacgccgcagatattgttggatgccctgaggcatgccttcataacaatgagtgcagtgagcctgctaatatttgatgaatgccatcgtgcctgtggaaaccacccgtatacacgaataatgaaggtaatggttttcttttcttgttttaacatcatcggtagataggatttggcctgcctcatgcagtttcttgacagttaagaagttccttaggatagcagtatttgtaatggtttggcatgtcaaatttcctaacggtttatgtttctcttgagtctgtaggaattttatgttggctctgagtggaggccagctgtatttggaatgacagcatctcctgttgctactaaaggcaagaattcttctccctctgtaaactaatataagagtgtttagatcaatattttagtgatctaaacgttcttatattagtttacagagggagtacacatgactatcatgtgcttcctttctttctaccctctgccatatatcttcgtcaatatttccagcaaacgaccactattttggttcttgaaatcagcatgttgtatgactgttatgtaccatgtgtcgtgctctcatcttctccctcacaagatcatacacttcctgaagtgtgatactctctgttgtgccaacagtgccccactgctctgcactataagttgcagttagctacctatacatcattgcttccttcttcatctggatgactgggaacacaacctgtacgtttgacatgatttttattggcttatttcattttgacaaaaaggttaactgtgcctgctattcatcctacttgaaggcactcgcgacttgggaccaatttatcaaatgaaaattattttcctgcactaggatgatttgttagacgctaaattttgtaatgctgcagccccccctccctccctccccgacatgcgcttattatttattacaagcttctatatttacttttgataaaagaacagtggaggagaggcaagtttcatttttaaatcaccgtccttttcaggcacttccacggtacaagattgtgaagcacacattggtcaacttgaacttacattggatgcaaaggttatctcagcatcctcagcttctttcacttcggattttattgagcctttggaaacctatattgcagtaagaaatagcctccatacctcacatgccattccaggtatatatcataaaggatcgtagtgaacttgagagcttttcccctcctgcaacaattgtgaacaaatactatgatgcttactcgattgatttcgaagatctgaaatcgaagctacagatattgtatgaagaggtgatttgattcaactatgttttgactctttcataagatacatctgtagtttggtgtagccacacctatagtgtgttgtatttctctctttgcatctatattactgtacaaatgcttatttgttaaaacttgtcattgttgggcagtatgatgctttgttggctaatctgcaagaatcatcaccaaataaatttgaagacacgaataacatattagagacatcaagaaagaccttgtctagataccatgagaagatattttatggcctaaattatcttggtccaatcatcaccgctgaggtatgtattatctaatcattgagtagctttaccaaaattttcgtaagacttaaaatggtcattgctatatctgatctagttcgttgacttcacttaagtccctgattccaactgaagttactcaaactttttggccatgtgtagaatatttgtactgccatattcctagctgatatactaccacaacatcccttatgttcagcagttgctgataagatctctgaattagcgatgctgggcagaaattttacctaacattaagaagattaactgctttgcaaggtcatttaatatatttttattgtggggaaaatcatgcgcatcaagtctcttgtacattttgagggaagaaatgctgcatcacgagcaagtagaccagcgatttagctacctatcatttccctctcaacgaaaaagtatatttcttagcattatgctgttgttgagttcatccctaatccttaatggagtaaatggacagttggattgccaggattgttggttaacaaaggatcactttatgttgaatatggaacatatccttcttatgaagtataagttaacatattcaacataaagtgatcctttgttaaccaacaatcctggcaatccaactgggccactttatgttaacccgggcgcctcgctcgtgcgccttgaccgggtgctctgcaccactgactgggaggatagtcatggcgagtgtcacctccgctgcctggcatccgttgtctccgaccactgccctctgcttttggattgctcgcccatgccatcagctcacaaacgcttccacttcgaggagttttggatgcgcctcgacggcttccacgatacggtagccgcggcctagggctcggtgtccgacccagaccccttcaggcggctggtccggcgcctgcaggctacgggccgcgccctcacgagctggagcgccaagtccacgggcaacatcagggacaagcttgccatctcccgcgagctcattctgcgcttcgaccaagccatggagacccgcacgctctcccccctggaggattggcttgcgaccgcacgctctcccccctggaggattggctgcgcaagcagaccaagctttcgtacctgggcttggcttcgcttgagcgcatgattgcccgacagcgtgctcgcatctcctccctctcggacggcgacgcgaacacgagcttcttccacaggcaatgcacttatcgtcggcagaaaaacagagtgtacaacttgcttgttgatgggcagacgatcagcgaccacgacgagatggcccaggcggcctttgctcacttcggcgggctgctgggcacagcggccgaccgcgcccacacgctggacttcgagcacctcatccagccagttgacctgtcaggcctagaggcgccgttcagccccgacgagatatgggaggctgtgcggcgcatgccggcgcacaaggcactggggccggacggctttaccgcggagttcctgcgagcgtgctggagcaccattaagcaggcgtgcttgaggtctttgagcagctctatgcgctgagggggtgtggattccacaggctcaaccaagcactgctcaccttgctgccaaagcgctccgatgctcacaagctcggcgactaccgaccgatttgcctgatacatattgtggctaagctcttcgcgaaggtcctttccctgcgcttggcaccgatgctcgacggcctagtgagccgctatcagaatgctttcatcgccggacgtaccctccacgacaacttcatccttgtccggcaatccctcaagttcctgcaccaactgggcgcgccgaggatcatgctcaagctcgacctcacccgggccttcgactccatatcatggcccttcctcttcgaggccctgcgccaatatggcttcggagcgaggttccgggagtggctggccatcctcctctcctcggccagcacccgcgtcttgctcaatggcgtgccgggacccccgatctggcaccgccgcgggttgagacagggtgactcgacatccccgcagctcttcgtcctggccgtcgacacgctcagccgcctcatgcaacgggcgctccaggccggcattctccggagactccacccccgacgcaacatcccggctatctcgctttacgccgatgatgtgatgctgttctgtcacgcctcgaccgacgaggtcctcgctgtcaagggcatcttagggatctttggctctgcctccggcctgcacgtgaactacggcaagagctctgccacaacacttcatggagatgagtcgaccgccgaatcactggtgattctgggctgcgagacggcggctctgcccatcacgtatctgggtatccccctctccacgcgccgcccctccgcggctcagatgcagcctctagtcgacaaggtggccgcccgcctcccatcgtggaaggcatggctcatgaacaaggccgggcggctggcgctcgtcaagtctgtgctcagcgccatccccgtccaccaaatgctcgccctagcgcccccgaagaaaactctgaagcagcttgagaagattcagcgtgggttcctctgggccgggcgcgaggctgcccacggtgggcactgccacgtgaactggcgccgggtatgccgaccgctggagtacggcggcctgggtgtccgcgacctagagcgcacggggcttgcccttcggctctgatggctatggctctcacggacggacactagccacgcatggtatggcctggacctgcagttcacccccgaggagtatggcctcttctatgcctccaccaccatgcagattggagacggatcgacagcgctcttctgggaggaccgatggctcctcggcctctccatccgcgagcacgcgccctcgctatacatgtgcatccccaaacatcgtagaaagtcccggacggtggcagagggcatcgccggcaatgcccgggcgagggacattcacgacactattggccttcaggagattgggcagtatctacagatctggctgctggtgtcccgcatcaccctctcctcagagccgtacacgctctcctggaagtggacagccagcgacaactactcagccaggtcctgctatcttgccaccttccccggatctatcacttgccggtcctggaagttgatctggaagggttgggcccctccgaaggtcaagttcttccactggctcgctaatcaggaccgctgttggactgcggagcgccttgctcgccatggcctaacgcatcacccttggtgccttctttgcgaccaagaaccggagacgatacaacacctgctgctttctcgcccattcgcgaagcagacttggcacgggattatgtcttggctgcgactaccggcacctgcgcccgaccaggacgcctcgctgcaggactggtggctacgcgctcgcgatgccatgcctcacccgctccgcaaagcgctggcctcgattgcgcttctcgtgccttggatgatctggaagcataggaacgcgtgtgtatttgaccatgtaaccccatcactagttgagctgccgatgcgatcaaggacgagtccaggaattgggcgaaggcaggggccaaggggcttagggtcaccctgccaccctcatgggacgtgcattgatctggttgtaacacctttgaactagcctcctaggaggttgtctctccctcttttcaatacaatgaaacgcaaagtcttttgcgttttctcgaaaaaatgaagtataagttgcatgtgaagagaaacttgagtctactaaaaggtcaactagtattgtcattcaaatgattgtgaagcgtattaattggttacaataccacttttttaatttgctgttaattgaccattacacttagagctatttatctttgtaaatacattaaaactctaaattaagaggttcacaaaggggggaaatatggacgccccggtaatattgctttagtaattcggtttttttttttttgctttagtaacatgacggactgaatgtgttagatgatgcaaaacaggtagttagaagtcggacagaataatttagttgaggtagttgctgaaccatttgagctgcaactggagttaacctatattgagtgttagagtttgctcctgggacattgtaggaaacatcatgaagaaaccttacaacgagaataatggtagcatttttccattttcttgtgcctccctcaccatatgaattgtaaattgggtcactttaatgtatactatttggtaaaaaaaactcgattcttccctaccacttccacagttccagtcttaagaagaaggtatggtccaaccatttcccttgcctgtgggagttagcataccttttcattattcttccatatcattttttattatatgaaagactaggtttttttcctggagaataaactgtgtattgttttcttaacaggtagtgaagatatataatgaaagcattaagacattggatgattctgaagattgcctcttctctaaagctagcttcagtctgcatgtagcttattttaaagaagctttagatttaatcgaggcagtattgccacatggtaagctacaaacaaatatccatcagttagatgtgttatatctgtctgtattcacttgctattgctcataaatagtactccctccgtttctaagtataagtctttttggatatttcaataaggaccacatacagatgtatatagacgtattttagagtgtagattcattcattttgctccgtatgtagtccatattgaaatctctaaaaagacttatatttaggtacggagggagtaatatagtatacaactagaatcttcagcatctttctgtatataaagggataaaatcagttgcaagcggcattaagatccttagtaagttgtatttatttcagtactttgcctgctatatctcggaataagaacgaaaataaaatagcattgcatttgaagaagtgtaatacaacaacaacaacaaccaagcctttcagtcccaaacaagttggggtaggctatagttgaaacccataagatctcgaagccaagtcatggctctggaacgtggatagctaacttccacgcacccctgtccatggctaagtctgtgtcgatattccaaaccttcaggtctctcttaacagactcctcccatgtcaagtttggtctaccacgacccctcttaacattcttagcacgctttatccgtctgctatgcaccgacgcttccggtggcctccgttcaatatgtccaaaccatttgagacgatgctggaccagcttctcttcaatcggtgctatcccaagtctctctcgtatatcgtcatttcgtacccgatccttccttgtgtgtccacatatccatctcaacatgcgcatctctgctacacctaactgttggatatgtcgtctcttggttggccaacactccgtgccatacaacatcgcacgtcggatagctgtcctataaaatctgccttttagcttttgtggcactctcttgtcacagagtacgccagaagcttggcgccacttcatccacctagccttgattcggtggcccacgtcttcatcaatatcgccatccttctgcaacatggaccccaaatatcgaaacgtgtctctctccggtaccacctgcccaccaaggctaacctctccatcctcgtgcctagtagcactaaaactgcacctcatgtattcagttttagttctactaagcctaaaacctttcgattctagagtccacctccataactctaactttctattaacccccgttcggctatcatcgactagcaccacatcatccgcaaagagcatacaccatgggatatctccttgtatatcccttgtgacctcatccatcaccaaatcaaaaggataagggctcaaagctgacccttggtgcagccctattctaatcggaaagtcatcggtgtcgccatcacttgttcgaacacttgtcacaacattatcatacatatccttgatgagggtaatgtactttattgggactttgtgtttctccaaggcccaccacatgacattccgaggtatcttttcataggccttctccaaatcaatgaacaccatatgaaggtccttcttttgctccctgtatctctccatcagctgtcgtaccaagaagatggcttccatggtagacctcccaggcatgaaaccaaattggttttttgtcacgcttgtcaaccttcttaagcggtgttcaatgactctctcccatagcttcatagtatggctcatcagcttgattccgcggtaattagtacaactttgaacatctcccttgttcttaaagattggtactaaaatactccgcctccattcttcgggcatcttgtttgaccgaaaaatgaggttgaaaagcttagttagccatactatcgctatgtctccaaggcctctccacacctcgatggggataccacacctcgataaagaagtgtaatactaaaaaaatgataatccaaatccgcgtcttgacagtgtcgtcgtagcatgaagagtcttcaaaacttgtcgagtcgccgaaggagataacaaagagatggcacattagaggtagacaccgcatcacttgaagatacaagataagtatcaagagaagatgggttgtcaaaagaagatggcacatcagaggaagacaccgaagagatggcacatcagaggaagacaccacatcacttgaagatacaagataagtatcaagagaagatgggttgtctaaagaagatggcacatcaagagaataaaacattgtgcagaaaatcatagtccacgacaaccgtcggcgaaagaagctcggctgataagacacgatgaacgtagatcgacaatgcaaaagaagtccataaaatcctatagaaaacaacaagggcaaataggccacaaaagttgtatagaaaggatcaggaccggagaaaggctgacggactaaggtatggtggactcgcatggcatgagaaaacacaaaatatcaacggatttggtggacgtagcggaaaacaagaaagctaaaagctaggaagcatacacgacacaaagatgcaaaatccatcgtgtatgcagaagacattgtagcccgagttcctgagcgccctccgcggcgtgagcgccgccttcatctccgcggccgacggtgcgttgaaggccacgtcgaggaggtcgaggaacgggtgcgacgcgtcggtgtcgtcaccggcggccgcacccgccgcgggggacgggcgcgcggcgcggatgaggggagggccgaggaggaggcacggcgccgcggcgggggcttctgtctccgtcgccatggctgctgctgcgcgggggacgagcgctggcacgaggaggagccgctgcctgacgggaacaggaaaaaaattggatcgtgaggacgagttgcggcgtccggagacctaaacctaggctctaataccatgttaggaaagcaacttatgtttattgaaggcccaaggggcatatatatattacacatgacttgaggtgcaaggaaagtaaacatagactaataaggactcctagactaatactaatagactaataaggactcctagactaatactaatacttcctaacatactggtcaattattttcggttgaatgtaggacactcctcgttgtgtgaatctatagccttaaagactgaaatcgcctgtttcttgatgcacctatgttttttgatttctgaagcattgtattattgatatctcactagactgtaagctacactgtagtgcattgatagtttcttcttctaattctgctgatgtaattgcatcataggttatggtgaactaatgaaatcagaatctggtaccgcggaattaagtaaaagggggtatatttcttcaaaagtggatgctctaatcaacatttttaaatcgtttgggtaaagccatattctgctcaagatgcttagttttgcctacttgtgctcttgtgtcagcaaaaatgggtaaatccacatgtattttctccacaggtcatcaaacgaagtgctttgcctaatttttgtagaaagaattatgacagctaaagctgttgagaggttcatgagaggaatttttaatttctctagtttttcaatttcttacttgactggagggagtacatcaaaagattctctgagcccagcagtgcagagatatactttggacttgttccgatctggaaaggtatgccatttaaattgcaaagattgatttaaacaggattcaaaatgatacatcaactccatgtcaatttctaggtgaacttgcttttcagcacagatgtaactgaagagggtattgatgtacctaactgttcttgtgtcatacgctttgacctgcccagaactgtttgtagctatgttcaatctcgtggtcgtgccagaaggagaagctcaaattatgttcttatgattgagaggtacaataatcgtccactgcataagctacatgctggtatagcatattaaattttagagatatgtttggccaccatttattcttattctaagcaaatttgccgcgcatattatattcatagattggatacaatttacgtctcataatcattattttttctggagaagtatatgctcttttacataatttggtttggcatgaacggtatgttttgtcagtcagcagtctgagtttctagcccaggaattcctgaatccctaccctctcatctaaaaaaatgttacaaccatgtttaaaaaatgttttaataatttttgtgagtacctattcaccttttgatccttcacatgattttttcaggggaaacatggatcagcaggaacacatattccgcataatacagactgagtactatattaaacacttcgctctgtatagacaggaaacccccaatgtttcatcctctgagttgcctatgcaagataagtatgcataccatgttgattcaacaggagcaactataactgcagactgctgtgtcgatctgattcgtaaatattgtgagaaacttcctaaagataggtacacctatgcttaccatttttgtagcttttgttgttctgtcagtctgcttggtaatgcacctcatgcataatcactaagaatggtggtgtgcttaatattcctgaacttgtaaccaattacatggatagcatgttggtgaaaaagttgcttaatttgaatgtcacacacagcacaaagaaaaactatttctcatactccccatggcacgatcttgctgtatttcattagatagaagtgattgatcattataacattcttctgaaggtattacatgccaaagccttcctttgtggtggctatagaagatggatcataccaatgtaccttgactttgcctccaaatgcggcgtttcaaaggatagttggccctttatgcagtacaagtaatttagccaagcagcttgtatccctagatgcgtgcaagaaactgcatcagctaggagaactcaatgatcatcttctaccgttgaccgaagaacctattgatattgatgttgctttgagagatggaaaattcctttctggaccaggtatgtgtctaacccctttttagtcttgcttacataaaagttctttaaataagaacatttatttgcctgttttgctatttactttcactcatctattttatacacttcatcaatataacattgatcagagtaagactgagcttgccaatttgtcgtacatcgttaaacttgtgcaactaatgctggcgggctttgtttttatttttttgcacagtaatggcaactctgtgtgtatgaggttgtctttcgaacatgtttgtgcaaggcttgggacaaatatgaaatatctccctactgttgaaatctgctggccttacatagttattctccattcttttactttaccatttcctctgagtggtgctgttacattgcatactgctgtctgtagtgaactgtgcaagttccaacaggaaccccttcttccaaaatggtatgcgtgcttctctcatcgcagatgataatgatgtttccactgttcttttttcattttggcaggaacgactaaaagaaaggagctccatggaacaagaaatgttcttgccctgtcaggatcgtggattcatgaaagtgaaagtgtcacattgaatacttatagattcgattttctttgtgaccaagagggtgaaaactatgctgggtttgttctcttaatggagtcagcacttgatgatgacgtagcttgttcaaaaatggatctattcctgatccctaataaaatggtctacaccaccataactccgtgtggaaaagttcaattggacaaaaaacaggtgttcaagaaataatcgcatactgtaattctcttctataaagtttgtttggccatcgcttatttttatcatttcttcagctacgtgacgggaagttgttccaagaattatttttcaatggaatatttggtagattatttcatgggtctcgaacaagtggagtgcaaagggaatttcttttcagaaaaggtcatgaaatacaatggagctcagagtgcatgtacttacttttacctttgaggcattcaccacatatccagcaagatttaaacatatactgggaggcagttgaatcctgcacccaggtggtggagcagttgagaaatttgtatctggaagatgaaaatatctgtgcaaattctattcaacatagaagtatcaaggagaaagacattattcatctagccaacaaatctcttcatttttctagcatcaaagattcagttgtgctgtcgcttcatactgggaggatatactctgttcttgacttgatctatgacacaactgctgaggactcgtttgaagagatgtataatggaaaagcttcaccgttttcttcatttgtggactactaccgtgaaaagtaatacctccattacatgtcttaaactcctaattacaattaaaatttattctgatatgcttcctgacaaacttccttttttaccactcttttgtaggtacggtattattattcaacatccaaaacaaccgttgttgctgttaaagcaaagccacaatgcacacaatcttcttttttcaaaatcaaaatacataggtactagctaaatcctagtccggtccagttccccttttcctgttatgatgaactgccagatagctgttgctttactatttggcctgctttttacatgaaactctacataacctatggcagatggttctacgggtgatcctttgctcatggaaaaagaacaaatacatgcccgggtcccacctgaattgttaatccacattgatgtgacaattgaagttctcaaatcattttatttattgccttctgtaatgcatcgacttcagtctctcatgctagccagccagcttcgcagagatattggttacactcaacatataccaagttacctggtctgttctctttcatgagaatataattttattgctatggctggttatgcatacctgaataatttgatccgtttcaatgtcagattttggaagctatcacaaccttaaggtgctgcgagacattttccctggagcgtttagaactattgggagactcagtactaaaatatttgataggatgtgacctgtttttaaggtatcctatgaaacatgaaggtcatctttctgatatgagatcaaatgctgtctgcaatgctacacttcataagcacggaatctggcgatccttgcaggtagatggcgtaaatccaagcctaatataatggtttcaacttcctgaagtttatgatgagttctgttagacaaataattttaattttgttagttcatgtgcacttggccagtcaaccagttatatttgagctttccccgcaataaataaataaatatttgagcctgagggccaacaatcacatatgcacttcatatagcaagtggttttcttctattctgctccatcttgagttttaattttatttgagcccatgccagtcagatgttttctagaggtgcttcatcaatgttcttttcattcaaaacgcatgtttcatgatactgtagaaaggcagaagttatatacgagcagagtcaagttgcgtataggagatactccctccgtcccaaatttcttgtcttagatttatttagatatgaatgtatctagtcaagttttagtatttagatacattcatttctagacaaactaagacaagaattatgggacggagggagtataatataacctagataccattgagatttgcaaggttgagaatacaattctgaggtgtatatgtttactggctatttgttatcttgctttccagggttacgtgcgggatagtgcattcgacccacggcgttgggttgctcctgggcagatatccttgcgcccttttccttgtaactgcggaattgagactgcatttgttccaaccaatggaaggtatatcagcgacgacccatcttttgtcgttggaaaaccatgtgatagagctcacagatggatgtgctcgaaaacaatatctgattgtgttgaagccctagttggagcatattatgttggtggtggcatagttgctgcactttgggttatgaagtggttcggtattgaaataaaatgtgataggaagttggtacaggaagtaaagctcaatgcatcttatatatgctacttacctaaagtaagtgttattgaggagttggaagcaaaattgaagtacaacttctcggtcaagggccttctactggaagccataactcatccatctctgcaggaattaggggttgactactgttaccaggtcagtgtatagcagcatatttctatattggttcatggggttatcattagcttattgtgcttcactcagaaattgcttcaaaacaagatatatccatgtcttctgtccttaacatttttgtgtctaacttgtgcctttacttcagcgtctggaatttctgggtgattctgtgctggacctactaattacacgctatctctatgttacccatactgatgttgatcctggagaattgacagacttacgttccgctttggttagtaacgagaattttgcagaagtagttttaagaaacaacattcacagtcatctacagcatgggtctggaatactcttggagcaaatcacagaatatgttaaatccaatttggagtgccaagggaaggttaataaattccttcaacatgctacatgtaaagtacctaaggttagaaatacagtttcacttatgttcattatcttagcagcctaccatttcaattttgtgcttttattgtttctgcgatggccttttctctgttaaacctttcccagccaacattacacgttgaagttaacaatattattttgtttgctctacaggtacttggagacattatggaaagcattgccggtgcaatatttgtagacacagattttgatgttgatgtggtttggaagattgttgaaccattgctttccccgatgataacccctgataatcttgcattgccaccctatcgggagttgttagagctgtgtagtcaccttggttatttcataaattcaaaatgcagtagtagaggagaagaagtaactatagatatgtcagtgcaattacgagatgaactgctgatagcccaaggacatgacagaaacaagaagagtgcaaaggcaaaagcagcagctcgtatattggcagatctgaaggtaggtgctttaaacataaaaatgaatgctatattagtgtgtatttcatggcaggtgcttcttggttttaattgttgttcagttgaatgggtagcagaacaatacccttagcatactttcctttactattccgaaggaaacatacattatctcgtatacagtatactcttccctggggtatttaactagctgatgtttttctgcttagaaattactccctctgtacctaaatacttgttctccccaactacaagtatttaggtacagagggagtagatgatttgtttatgccatttgtctattatggaaactgacgtttaagtttgaagcagaaaagaggtctttcgatgaaacaatgttcctctaaatctaagcagttagatattatatctccggacctgcagtatcctttggcaagtgcaagcttctacatagttttccagcaaatgctttaacaaagtgttgcattttattataatataatgactattattacttttctgtagttggaatcaccacttgattattgtgacgtgaatgtcaatcctagcctaggaggccttccctcactgaaggaagcaggtatgtcacatttcctatttttgtcgtcctttggtttaattgtgttgcttgttggttcacagcctgtcaaacatgacatactaaaatgtacagtagtatatacctttttctctttgcatggataagatgcatgtttgcttggtaaccaggcaattttgtttgttttcagttgttcttcagctcaaaatggacaaaggtggaccacgaagtgctctttttaagctatgcaagaggttgcagtggcccatgccagaattcgaatttgtggaacaaaggttcaggtatgtccaggaatacaagatatatatgacctctagcaaccaaggcagaaactccattccacttgaatgttggcaatgccgacccttctggtcttttaacaaagctgtgctaaattattgtactgggttgatccaacaggactcctattgttctggacggggtaaccacaacgaacttcaatagcttcgtgtcaaccatcaccttgcacatacctgatgtaactgccattacaattcaaggcaaacggcaaactgacaaaaagagctcccaggattcagcgtcgctgataatgctccacaagcttcaagagctcaaggtctgtatatgcaagacttag

SEQ ID NO: 3 - DNA, 560 bp, Hordeum vulgare
  source 1..560: mol_type = genomic DNA
cccccacccccactcgcctgcaccgaacccaaccccatggtcatggccatggccgacgacgacggccccatccccccgcctcctcctccgccgccgcctcgtcgcccccacatgcagctccagccgaggaggtctcgatcggctcccattaccctccccacagctctctaccccctgcattcctagaactgacgggggtctctcgttccgtgtctgcgtcggcgcaggtatcaggtggaggtgttcgaggcggcgctgcaggggaacaccatcgcagtgctcgacaccggctccgggaagaccatggtcgccgtcctgctcgcgcgctaccacgtcagccgcgcgcgcgccggggaggcgccccgccggatcgtcgtcttcctcgcgcctaccgtgcacctcgtccaccaggtgcgcttctgaagtctgcagcgaccgtttagcgacggcaacgtctgcaccatttttccgcctcgcgttcgcagctgccgtggccctgatccctggttcttgtctggctcgcgccgtagcaattcgaggtgattcgggagtacaccgac

SEQ ID NO: 4 - protein, 1627 aa, Triticum aestivum
MAMADDDDGPLPPPPPPPPPRRPHRQLQPRRYQVEVFEAALQGNTIAVLDTGSGKTMVAVLLARHHVGRVRAGEVPRRIVFFLAPTVHLVHQQFEVIREYTDLDAAECYGASGVGDWSTDRWNKEVGSKEIAVMTPQILLDALRHAFVTMSAVSLLIFDECHRACGNHPYTRIMKEFYLGSQWRPAVFGMTASPVATKGTCTVQDCEAHIGQLELTLDAKVYIIKDRSELESFSPPATIVHEYYDAYSIDFEDLKSKLQILYEEYDALLASLQESSPNKFEDTNNILETSRKTLFRYHEKIFYGLNYLGPIITAEVVKIYNESIKTLGDSEDCLFSKASFSLHVAYFKEALDLIEEVLPHGYGELMKSESGSAELIKRGYISSKVDALINIFKSFGSSNEVLCLIFVDRIMTAKAVERFMRGIVSFSSFSISYLTGGSTSKDSLSPAVQRYTLDLFRSGKVNLLFTTDVTEEGIDVPNCSCVIRFDLPRTVCSYVQSRGRARRSSSNYVLMIERGNMDQQEHIFRIIRTEYYIKHFALYRHKNPNVSSSEWPMQDKYAYYVDSTGATITADCCVDLIRKYCEKLPKDRYYMPKPSFAVAIEDGSYQCTLTLPPNAAFQRIVGPLCSTSNLAKQLVSLDACKKLHQLGELNDHLLPFTEEPIEIDVALRDGKFLSGPGTTKRKELHGTRNVLALSGSWIHESESVTLNAYRFDFLCDQEGENYAGFVLLMESALDDDVACSKMDLFLIPNKMVYTTVTPCGKVQLDKKQLRDGKLFQEFFFNGIFGRLFHGSRTSGVQREFLFRKGHEIQWSSESMYLLLPLRHSPHIQQDLNIYWEAVESCTQAVEQLRNLYLEDENIRANFIQHRSIKEKDIIHLANKSLHFSSIKDSVVLSLHTGRIYSVLDLIYDTTAEDSFEEMYNGKASPFSSFMDYYREKYGIVIQHPKQPLLLLKQSHNAHNLLFSKSKYIDGSTGDPLLMEKEQIHARVPPELLIHIDVTIEVLKSFYLLPSVMHRLQSLMLASQLRRDIGYTQHIPSYLILEAITTLRCCETFSLERLELLGDSVLKYVIGCDLFLRYPMKHEGHLSDMRSNAVCNATLHKHGIWRSLQGYVRDSAFDPRRWVAPGQISLRPFPCNCGIETAFVPTNGRYISDDPSFVVGKPCDRAHRWMCSKAISDCVEALVGAYYVGGGIVAALWVMKWFGIEIKCDRKLVQEVKLNASYICYLPKISVIEELEAKLKYNFSVKGLLLEAITHPSLQELGVDYCYQRLEFLGDSVLDLLITRYLYVTHTDVDPGELTDLRSALVSNENFAEVVLRNNIHSHLQHGSGILLEQITEYVRSNLECQGKVNKFLQHATCKVPKVLGDIMESIAGAIFVDTDFNVDVVWKIVEPLLSPMITPDNLALPPYRELLELCSHLGYFINSKCSSKGEEVSIEMSVQLRDELLIAQGQDRNKKSAKAKAAARILADLKKRGLSMKQCPSKAKQSGIISSDQQYPLASLESPLDYCQVNVNPSLGGLPSLKEAVVLRLKMDKGGPRSALFKLCKRLQWPMPEFEFVEQRFRTPIVLDGVTTTNFNSFVSTITLHIPDVAAITLQGERRTDKKSSQDSASLTMLHKLQELKVCICKT

SEQ ID NO: 5 - DNA, 12302 bp, Triticum aestivum
  source 1..12302: mol_type = genomic DNA
accccacccccgcctcgcccactcgcccgcaccgaacccaaccccatggccatggccgacgacgacgacggccccctgcccccgcctccgccgccgccgccgcctcgccgtccccacaggcagctccagccgaggaggtcgcgcattactctctccacagctctctggcgcccgcattcctagagctgacgggggttacccattctgtgtctgtgtcggcgcaggtaccaggtggaggtgttcgaggcggcgctgcaggggaacaccatcgcggtgctcgacaccggctccgggaagaccatggtcgccgtcctgctcgcgcgccaccacgtgggccgcgtgcgcgccggggaggtgccccgccggatcgtcttcttcctcgcgcccaccgtgcacctcgtccaccaggtgcgcttctgcactctgcagcgaccgattcgcgacggcaacgtctgcgccattttttcgcctcgcgttcgcagcttccatggccctgatcgctggttgctgtctgggctcgcgctgtagcaattcgaggtgattcgggagtacacagacctcgacgccgcggagtgctacggagcttcgggggtcggcgactggagtacagaccgctggaacaaggaagttgggagcaaggaggtaagtgtcaggttttactctatcccgcggcacggtcattggttgatccgaatgcacactgaattggtacacgtaccgtgctcaccaagtgcttgatgaatgacttgttgacaaggaaccaacattgttagtttctgggtcgtagcagcttcggatcgcatgtagattgaggacatttgttatcggagtagtattctttagcgtgagggcagtcgactaaagcaatttgctgaagggccgtgactatgaaatagctggggtctggagtctggacacaagtcctaataaaagaaaatttcctttcaatgcagtcactgctaaatttggacactgcttcgaatccacatgttctcacaactcttgtaaatatgttgcttaaaaaaactcttgtaaatatgccacttggtaaagttatctggattatgtgtctttctaaattctaatccagttttgtggatattcctatgttttttacctgccgtattgcgcttattatgcgttctattgacaactttgattgaaaatatgaattttcctgggacaatgatcttattatgcgttctattatgtgtttcagattgctgttatgacgccccagatattgttggatgccctgaggcatgcctttgtaacaatgagtgcagtgagcctgctaatatttgatgaatgccatcgtgcctgtggaaaccacccatatacacgaataatgaaggtaatgggatttttcttgttttaacaacatctgtagatatgctttggcttgcctcatgcagttttttgaaagtaaagaagtttcgtaggatagcagtatttttcatagtttggcatgtcgaatttcctaacggtttatttctcttttgagtctgtaggaattttaccttggttctcagtggaggccagctgtatttggaatgacagcatctcctgttgctactaaaggcaagaattctacacatgactcgtgtgcttcctttctttctaccctctgcgattattcttcagcaaatatttccagcaaacgaccgctattttggttcttgaaatcagcatgttgtatgactgttatgtaccatgtgtcgtgctcttatcttctcccccacaagatcatacacttgctgaagtgcgatactctgttgtgccaacagtgccctactgctgtacactatatataaattgcagttagctacctgtacattattgcttccttcttcatctggatgactgggaacacaacttgtacgtttgacatgatttttattggtttatttcattttgacaaaaaggaaaattgtgcctgctattcatcctacctgaaggcacttgcgactcgggaccaatttatcaaatcaaaattattttcctgcactaggatgatttgttagacgctaacttcagtggtggagaggcaagtttcacttctaaaaattaccgtccttttcaggcacttgcacggtacaagattgtgaagcacacattggtcaacttgaacttacattggatgcaaaggttacctcagcatcctcaacttctttcactttgaatttcattgagcctttggaaatggtttccataccttacatgccattccaggtatatatcataaaagatcgtagtgaacttgagagcttttcccctcctgcaacaattgtgcacgaatactatgatgcttactcgattgatttcgaagatctgaaatcgaagctccagatattgtatgaagaggtgatttaatttaactatgttttgactcattcataagatacatctgtagtttggactttggattagccacacctataatgcgttgtatttctcgctttgcatctatattactgtacaaatgtttatttgttaaaagttggcattgttgggcagtatgatgctttgttggctagtctgcaagaatcgtcaccaaataaatttgaagacacgaataacatattagagacatcaagaaagaccttgttcagataccatgagaagatattttatggcctaaattatcttggtccaatcatcaccgctgaggtattgtataatctaatcattgagtagctttaccaaatttttcgtaagagttacaatggttattgttctatctgatctaattcgttgacttcacataagtccctgattctaactgaagttattcaaactttttggccatgtgtagaatattcgcattggcaaattcctagccgatatactatcacaatatcccttatgttcagcagttgctgataagatctttgaattagcgatgatgggcagaaattttacctaacattaagaagattaactgctttgcaaggtcatttaatatgtttttagtgtggagaaaatcgtgcgcatcaagtctcttgtacactttgagggaagaaatgctgcatcacgagcaagtagactagcgatttagctacctatatgaacctatcattttcctctcaacggaaaagtatatttcttagcattatgctgctgttgagttcgtccctaatccttgaaggccaaaataatggagtaaatggactgttaaattgccaggattgttttttatttgagggaatgccatcatgacttagctttattaacttcaaaaaacgtttacatcgtctatgagcttactgaccacaaagtcaggtggctcgtccaaccaactaaaggaagacctattacaataactaaagtttgctaggacatgagtcacttgattacatgaactataacaatgcttataaatgaccgtcccaatcaaggaactaatatcaacacattccgcgaaaattgccgccgccgaatcccaccaactagtttgcccggtgtaatagttaatgacctgcagggagtctgattctgcttctactcgattaaatctgagagaattagcaagagcaagtccatctctcatcgccattgcctccattgtgatcacatctgctgcaagaggaatgaatttaccatttccaggattgttggttaacaaaggatcgctttatgttgatatggaacatatcttctcatgaagtataagctgcatgtgaggagatgcttgagtctactaaaaggtctactagtattatcattcaaatgattgtgaagcgtattaattggtcacaatactactttttaatttgctgtgaactgaccattacacttacacctattatcttggtaaatacattaaaattctaaattaagatgctcacaaggggggggggggatatggacgccccggtaatgttgctttggtaacatgacggaccgaatgtgttagatgaggcaaaatagcgtagttagaagtcggacagaataatttagttgaggtagttactgaaccatctgagctgcaacttgagttaacctatattgagttgagtgtagagtttgctcctgggacattgtacgaaacatcacgaagaaaccatacactgagatttcttgagaataatggtaccatttttccattttcttgtgcctcaccatataaattgtaaattgggtctctttaatgtatactatttggaaaaagatactagattcttccctaccatttccacagttccagtcttattaagaaggttgtggtccaaccatttcccttgcctgtcgcagttagcatacctttttcatcattcttccataccattttttattataggaaatactagattttttcccctggagaacaaactgtgtattgttttcttaacaggtagtgaagatatataatgaaagcattaagacactgggtgattctgaagattgtctcttctcgaaagctagcttcagcctgcatgtagcttattttaaagaagctttagatttaattgaggaagtattgccacatggtaagccacaaacaaatatccatcagttagatgtgttatatctgtctgttttcacttgttattgctcataaatagtaatatactccctccgtcccataatgtaacgtcttacattatgggacggagggagtagtatacaactagaatctgcagtatctttctgtatataataaaggggtaggttataaaatcagttgcaaactgcattaagatcctccgaatccttagataagttgtatttatttcagtactttgcctgctatatctcgaaataaaataatggttctacacaaacctcttaatttgcacactctatggaggtgagctatgaggaagtacctgcaacgtgtatttctatagcaaatagccgaatagggagttaagtgggtgatcatcagccattcagtagccatatgtgctgcacatatactgtccattattttgggttgaatgtagggcactcctcgttgtgtgaatctatatccttaaagactgaaatcgcctgtttcttaatgcacctatgtgttttgatttctgaagcattgtattattgatatctcactagactgaagcagcttggaaactacaatgtagtgcattgatagtttcttcttctaattctgctgatgtaattgcatcataggttatggtgaactaatgaaatcagaatctggttctgcggaattaattaaaagggggtatatttcttcaaaagtggatgctctaatcaacatttttaaatcatttgggtaaagccatattctgctcaagatgcttagttttgcctacttgtgctctcgtgtcagcaaaaaagggtaaatccacatgtattttctccacaggtcatcaaacgaagtgctttgcctaatttttgtagatagaattatgacagctaaagctgttgagaggttcatgagaggaattgttagtttctctagtttttcaatttcttacttgactggagggagtacatcaaaagattctctgagcccagcagtgcagagatatactttagacttgttccgatctggaaaggtatgccatttaaattgcaaagattgatttaaacaggattcaaaatgatacattaactccatgtcaatttctaggtgaacttgcttttcaccacagatgtaactgaagagggtattgatgtacctaactgttcttgtgtcatacgcttcgacctgcccagaactgtttgtagctacgttcaatctcgtggtcgtgccagaaggagcagctcaaattatgttcttatgattgagaggtacaataatcgtcgcagttcataagttgcatgctggtatagcatattaaattatagagaaatgttttggccaccatttattcttattctaagcacatttatcatgcatattatattcatagattgggtacaatttatgcctcataatcattaatttttctggaaaaggatatgctcttctacataatttggtttggcatgaacggtattttttgtcagtcagcactcagcagtctgagtttctagcccaggaattcctgaatctcgatcctctcatctaaaaaatattacaaccatgtttaaacattgttttaataatttttatgactacctattcaccttttgatacttcacatgaaattttcaggggaaacatggatcagcaggaacacatattccgcataatacggactgagtactatattaaacacttcgctctgtataggcacaaaaaccccaatgtttcatcctctgagtggcctatgcaagataagtatgcatactatgttgattcaacaggagcaactataactgcagactgctgtgttgatctgatccgtaaatattgtgagaaacttcctaaagataggtacacatatgcttaccatttttgtagcttttgttgctctgtcagcctgcttggtaatgcatctcatgcataatcactaagaatgctggtgtgcttaagattcctgaactgtgatgcaaccaattacatggatagcatgttggcgaaaatgttggttaatctgattgtcacacacagcacaacgaaaaactatttctcatacttcccatggcacgatcttactgtctttcattagatagaagtgattgatcattgtaacattcttctgcaggtattacatgccaaagccttcctttgcggtggctatcgaagatggatcataccaatgcaccttgactttgcctccaaatgcggcgtttcaaaggatagttggccctttatgcagtacaagtaatttagccaagcagcttgtatccctagatgcgtgcaagaaattgcatcagctaggagaactcaatgatcatcttctacccttcaccgaagaacctattgaaattgatgttgctttaagagatgggaaattcctttctggaccaggtatgtgtataccctttttcagtcttgctctcattaaaattctttagataagaacatttatttgcctgttttgctattttctttcactcatctattttatacacttcatcaatataacagtgatcagagtaagactgagcttgcccatttgtcgtacatggttaaacttgtgcaactaatgctggtgggctttgtttttattgttttgcacagtaatgcaactctgtgtgtatgaggttgtcttttggtcatgtttgtgcaatgtttggggcaaatatgaagtatctcccgactgttgagatctgctgaccttacgtagttattctccattcttttactttaccattccatctgagtggtgctgttacattgcatactgctgtctgtagtgaactgtgcaagtttcaacagcaatcccttcttccaaaatggtatgcatgcttctctcgtcgtagatgataacgatgttcccattgtttttttctcattttggcaggaacgactaaaagaaaggagctccatggaacaagaaatgttcttgccctgtcaggatcgtggattcatgaaagtgaaagtgtcacattgaatgcttatagattcgattttctttgtgaccaagagggtgaaaactatgctgggtttgttctcttaatggaatcagcacttgatgatgatgtagcttgttctaaaatggatctattcctgatccctaataaaatggtgtacaccaccgtaactccgtgtggaaaagttcaactggacaaaaaacaggttgttcaagaaataattgcatactgtaattctcttctataaagtttgtttggccatcgcttatttttgtcatttcttcagctacgtgacgggaagttgttccaagaattctttttcaatggaatatttggtagattatttcatggatctcgaacaagtggagtgcaaagggaatttcttttcagaaaaggtcatgaaatacaatggagctcagagagcatgtacttacttttacctttgaggcattcaccacacatccagcaagatttaaacatatactgggaggcagttgaatcctgcacccaggcggtggagcagttgagaaatttgtatctggaagatgaaaatatccgtgcaaattttattcaacatagaagtatcaaggagaaagacattattcatctggccaacaaatctcttcatttttctagcatcaaagattcagttgtgctatcgcttcatactggaaggatatactctgttcttgacttgatctatgacacaactgctgaagactcgtttgaagagatgtataatggaaaagcttcaccgttttcttcattcatggactactaccgtgaaaagtaatacctctattacatgtcttaaactcttaattacagtaaaatttattctgacatgcttcctgacaaacttctctttttactactcttttgtaggtatggtattgttattcaacatccaaagcaaccgttgttgctgttaaagcaaagccacaatgcacacaatcttcttttttcaaaatcgaagtacataggtactagctataaatcctagtccggtccagttccccttttcccgttatgatgaattgcaagatagctgttgctttactatttggtctgcttttacatgaaactctacataacctatggcagatggttctacgggtgatcctttgctcatggaaaaagagcaaatacacgcccgggtcccacctgaattgttaatccacattgatgtgacaattgaagttctcaaatctttttatttattgccttctgtaatgcatcgacttcagtctctcatgctagccagccagctccgcagagatattggttacactcaacatataccaagttacctggtctgttctctttcgtgagaatataattttattgctatggctggttatgcatacctgaataatttgatccgtttcaatgtcagattttggaagctatcacaactttaaggtgctgcgaaacattttccctggagcgtttagaattattgggagactcagtactaaaatatgtgataggatgtgacctgtttttaaggtatcctatgaaacatgaaggtcatctttctgatatgagatcaaatgctgtctgcaatgctacgcttcataagcacggaatctggcgatccttgcaggtggatggcctaaatccaagcctattataatggtttccacttcctgaagtttatgatgagttctagtattagacaaataattttaatttagttagttcatgtgcacttggccagtcaaccagttatatttgagccttccctgcattaaataaataaatatttgagcctgagggccaacagtcacatatgaacttcatatagcaagtggttttcttctgttctgctcttgagttttaattttgtttgagcccatgccagtcagatgttttctagaggtgcttcgtcaatgttcattcaaaaagcatgtttgatgatattgtaaaaaggcagaatttatatacaagcagagtcaagttgcgtataggagatagaatataacctagataccgttgagatttgcaaggttgagaatacaattccgaggtgtgtatgtttactggctatttgttatcttgctttccagggttacgtgcgggatagtgcatttgacccacggcgttgggttgctcctgggcagatatctttgcgcccttttccttgtaactgcggaattgagactgcatttgttccaactaatggaaggtatatcagcgacgacccatcttttgtcgttggaaaaccatgtgatagagctcacagatggatgtgctcgaaagcaatatctgattgtgttgaagccctagttggagcatattatgtcggtggtggcatagttgctgcactttgggttatgaagtggttcggtattgaaataaaatgcgataggaagttggtacaggaagtaaagctcaacgcatcttatatatgctacttacctaaaataagtgttattgaggagttggaagcaaaactgaagtacaacttctcagtcaagggccttctactggaagccataactcatccatctctgcaggaattaggggttgactactgttaccaggtcagtgtatagcagcatatttctatattggttcgtgggcaatcagaaattgcttcaaaacaagatatatccatgtcttctgtccttaacatttttgcgtctaacttgtgcctttatttcagcgtctggaatttctgggtgattctgtgctggatctactaattacacgctatctctatgttacccatactgatgttgatcctggagaattgacagacttacgttccgctttggttagtaatgagaattttgcagaagtagttttaagaaataacattcacagtcatctacagcatgggtctggaatacttttggagcaaatcacagaatatgttagatccaatttggagtgccaagggaaggttaataaattccttcaacatgctacatgtaaagtacctaaggttagaaatacagtttcacttatgttcattatcttaacagcctaccttttcaattttgtgcttttattgtttctgcgatggccttttctctgtgaagcctttcccagccaacattacacgtagaagttaataagattattttctttgttttacaggtgcttggagacattatggaaagcattgccggtgcaatatttgtagacacagattttaatgttgatgtggtttggaagattgttgaaccgttgctttccccaatgataacccctgataatcttgcattgccaccttatcgggagttgttagagctgtgtagtcaccttggttatttcataaattcaaaatgcagtagtaaaggagaagaagtaagtatagagatgtcagtgcaattacgagatgaactgctgatagcgcaaggacaagacagaaataagaagagtgcaaaggcaaaagcagcagctcgtatattggcagatctgaaggtaggttctttaaacataaagaggaatgctgtattagtgtgtagttcatgacagtgcctcgtggtttaaattgttgttcagttgaatcggtagcagaactatatcctgagcatactagctgatgttttctgcttagaaattagatgatttgtctatgccatttatctatgacagaaactgatgtttaagtttgaagcagaaaagaggcctttcaatgaaacaatgtccttctaaagctaagcagtcgggtattatatcttcggaccagcagtatcctttggcaagtgtaagcttccacatagttttccagcaaatgctttaacaaagtggtgtattttattatagataatgactattattacttttctgtagttggaatcaccacttgattattgtcaggtgaatgtcaatcctagcctaggaggccttccctcactgaaagaagcaggtatctgacgattcccattttgatcgtcctttggtttaattgtgttgcttgatagttcacagcctgtcaaacatgacatactaaaatgtatatatacctttttctctttgcacggataagatgcatgtctactcagtaaccatgcaattttgtttgttttcagttgttcttcggctcaaaatggacaaaggtggaccacgaagtgctctttttaagctatgcaagaggttgcagtggccaatgccagaattcgaatttgtggaacaaaggttcaggtatgtccaggaatacaagatatatatgacctctagcagccaaaacaaaaactccattccacttgaatgttggcaatgccgatccttctggtcttctcacgaagatgtgctaaattcttgtactggtttgatgcaacaggactcctattgttctggacggggtaaccacaacgaacttcaatagcttcgtgtcaaccatcaccttgcacatacctgatgtagcagccattacacttcaaggtgagcggcgaactgacaaaaagagttcccaggattcagcatcgctgacaatgctccacaagcttcaagagctcaaggtctgtatatgcaagacttagcaaagcatcatggatccacttgccagctaactgctaaggctcgttaacatggggatctgatcctccatcccacagtgaattatgttgaaattttttatgagaggcccagtggtggtgctagattatcctgcaggatatctcagaaacaattagtgcttgtcttccgaaaaaaacaatagacaatttcacttgctggacaaagaaaacattttgcaagaattgatatcggtttggtttgaatccacatttgggtcaactatcgaccttaggttcttagtggaattgagtgcaatttttttagctccttagcagagttcacatgtagttacatttctctacttcactccacaaatgtcc

SEQ ID NO: 6 - protein, 1036 aa, Triticum aestivum
MPKPSFAVAIEDGSYQCTLTLPPNAAFQRIVGPLCSTSNLAKQLVSLDACKKLHHLGELNDHLLPFTEEPIDIDVALRDGKFLSGPGTTKRKELHGTRNVLALSGSWIHESGSVTLNAYRFGFLCDQEGENYAGFVLLMESALDDDVAYSKMDLFLIPNKMVYTTVTPCGKVQLDKKQLRDGKLFQEFFFNGIFGRLFHGSRTSGVQREFLFRKGHEIQWSSESMYLLLPLRHSPHIQQDLNIYWEAVESCTQAVERLRNLYLEEENIRANFIQHRSIKEKDIIHLANKSLHFSSIKDSVVLSLHTGRIYSVLDLIYDITAEDAFEEMYNGKASPFSSFVDYYREKYGIVIQHPKQPLLLLKQSHNAHNLLFSKSKYIDGSTGDPLLMEKEQIHARVPPELLIHIDVTIEVLKSFYLLPSVMHRLQSLMLASQLRRDIGYTQHIPSYLILEAITTLRCCETFSLERLELLGDSVLKYVIGCDLFLRYPMKHEGHLSDMRSNAVCNATLHKHGIWRSLQGYVRDSAFDPRRWVAPGQISLRPFPCNCGIETAFVPTNGRYISDDPSFVVGKPCDRAHRWMCSKAISDCVEALVGAYYVGGGIVAALWVMKWFGIEIKCDRTLVQEVKLNASYICYLPKISVIEELEAKLKYNFSVKGLLLEAITHPSLQELGVDYCYQRLEFLGDSVLDLLITRYLYVTHTDVDPGELTDLRSALVSNENFAEVVLRNNIHSHLQHGSGILLEQITEYVRSNLECQGKVNKFLQHATCKAPKVLGDIMESIAGAIFVDADFNVDVVWKIVEPLLSPMITPDNLALPPYRELLELCSHLGYFINSKCSSKGEELSIEMSVQLRDELLIAQGQDRNKKSAKAKAAARILADLKKRGLSMKQCSSKAKQLDIISSEQYPLASLESPLDYCHVNANPSLGGLPSLKEAVVLRLKMDKGGPRSALFKLCKRLPWPMPEFEFVEQRFRTPIVLDGVTTTNFNSFVSTITLHIPDVTAITLQGERRTDKRSSQDSASLAMLHKLQELKVCICKT

SEQ ID NO: 7 - DNA, 5723 bp, Triticum aestivum
  source 1..5723: mol_type = genomic DNA
atgccaaagccttcctttgcggtggctatcgaagatggatcataccaatgcaccttgactttgcctccaaatgcggcgtttcaaaggatagttggccctttatgcagtacaagtaatttagccaagcagcttgtatccctagatgcgtgcaagaaattgcatcatctaggagaactcaatgatcatcttctacccttcaccgaagaacctattgatattgatgttgctttaagagatgggaaattcctttctggaccaggtatgtgtataccctttttcagtcttgctctcattaaaattctttagataagaacatttatttgcctgttttgctattttctttcactcatctattttatacacttcatcaatataacagtgatcagagtaaggctgagcttgcccatttgtcgtacatggttaaacttgtgcaactaatgctggtgggctttgtttttattgttttgcacagtaatgcaactctgtgtgtatgaggttgtcttttggtcatgtttgtgcaaggtttggggcaaatatgaagtatctcccgactgttgagatctgctgaccttacatagttattctccattcttttactttgccatttcatctgagtggtcctgttacattgcatactgctgtctgtagtgaactgtgcaagtttcaacagcaatcccttcttccaaaatggtatgcgtgcttctctcgccgtagatgatgacgattttcccattgttcttttctcattttggcaggaacgactaaaagaaaggagctccatggaacaagaaatgttcttgccctgtcaggatcgtggattcatgaaagtggaagtgtcacattgaatgcttatagattcggttttctttgtgaccaagagggtgaaaactatgctgggtttgttctcttaatggaatcagcacttgatgatgatgtagcttattcaaaaatggatctattcctgatccctaataaaatggtctacaccaccgtaactccgtgtggaaaagttcaactggacaaaaaacaggttgttcaagaaataatcgcatactgtaattctcttgtataaagtttgtttggccatcgcttatttttgtcatttcttcagctacgtgacgggaagttgtttcaagaattctttttcaatggaatatttggtagattatttcatggatctcgaacaagtggagtgcaaagggaatttcttttcagaaaaggtcatgaaatacaatggagctcagagagcatgtacttacttttacctttgaggcattcaccacacatccagcaagatttaaacatatactgggaggcagttgaatcctgcacccaggcggtggagcggttgagaaatttgtatctggaagaggaaaatatccgtgcaaattttattcaacatagaagtatcaaggagaaagacattattcatctggccaacaaatctcttcatttttctagcatcaaagattcagttgtgctatcgcttcatactggaaggatatactctgttcttgacttgatctatgacataactgctgaagacgcgtttgaagagatgtataatggaaaagcttcaccgttttcttcattcgtggactactatcgtgaaaagtaatacctctattacatgtcttaaactcttaattactagtaaaatttattctgacatgcttcctgacaaacttctctttttaccactcttttgtaggtatggtattgttattcaacatccaaaacaaccgttgttgctgttaaagcaaagccacaatgcacacaatcttcttttttcaaaatcgaagtacataggtactagctataaatcctagtccggtccagttccccttttcccgttatgatgaattgcaagatagctgttgctttactatttggtctgcttttacatgaaactctacataacctatggcagatggttctacgggcgatcctttgcttatggaaaaagagcaaatacacgcccgggtcccacctgaattgttaatccacattgatgtgacaattgaagttctcaaatctttttacttattgccttctgtaatgcatcgacttcagtctctcatgctagccagccagctccgcagagatattggttacactcaacatataccaagttacctggtctgttctctttcgtgagaatataattttattgctatggctggttatgcatacctgaataatttgatccgtttcaatgtcagattttggaagctatcacaactttaaggtgctgtgaaacattttccctggagcgtttagaactattgggagactcagtactaaaatatgtgataggatgtgacctgtttttaaggtatcctatgaaacatgaaggtcatctttctgatatgagatcaaatgctgtctgcaatgctacacttcataagcacggaatctggcgatccttgcaggtagatggcctaaatccaagcctattataatggtttcaacttcctgaagtttatgatgagttctagtattggacaaataattttaatttagttagttcatgtgcacttggccagtcaaccagttatatttgagccttcccgcagtaaataaataaatatttgagcctgagggccaacaatcacatatgaacttcatatagcaagtggttttcttctgttctgctcttgagttttaattttgtttgagcccatgccagtcagatgttttctagaggtgcttcgtcaatgttcattcaaaaagcatgtttgatgatattgtaaaaaggcagaagttatatacgagcagagtcaagttgcgtataggagatagaatataacctagatactgttgagatttgcaaggttgagaatacaattccgaggtgtgtatgtttactggctatttgttatcttgctttccagggttacgtgcgggatagtgcatttgacccacggcgttgggttgctcctgggcagatatctttgcgcccttttccttgtaactgcggaattgagactgcatttgttccaactaatggaaggtatatcagcgacgacccatcttttgtagttggaaaaccatgtgatagagctcacagatggatgtgctcgaaagcaatatctgattgtgttgaagccctagttggagcatattatgttggtggtggcatagttgctgcactttgggttatgaagtggttcggtattgaaataaaatgcgataggacgttggtacaggaagtaaagctcaatgcatcttatatatgctacttacctaaaataagtgttattgaggagttggaagcaaaactgaagtacaacttctcggtcaagggccttctactggaagccataactcatccatctctgcaggaattaggggttgactactgttaccaggtcagtgtatagcagcatatttctacattggttcgtggggttatcattagcttattgtgcttcaatcagaaacaagatatatccatgtcttctgtccttaacatttttgcgtctaacttgtgcctttatttcagcgtctggaatttctgggtgattctgtgctggatctactaattacacgctatctctatgttacccatactgatgttgatcctggagaattgacagacttacgttccgctttggttagtaacgagaattttgcagaagtagttttaagaaacaacattcacagtcatctacagcatgggtctggaatacttttggagcaaatcacagaatatgttagatccaatttggagtgccaagggaaggttaataaattccttcaacatgctacatgtaaagcacctaaggttagaaatacagtttcacttatgttcattatcttaacagcctaccttttcaattttgtgcttttattgtttctgcgatggccttttctctgtgaagcctttcccagccaacattacacatagaagttaacaagattattttgtttgttttacaggtacttggtgacattatggaaagcattgccggtgcaatatttgttgacgcagattttaatgttgatgtggtttggaagattgttgaaccgttgctttccccaatgataacccctgataatcttgcattgccaccttatcgggagttgttagagctgtgtagtcaccttggttatttcataaattcaaaatgcagtagtaaaggagaagaattaagtatagagatgtcagtgcaattacgagatgaactgttgatagcgcaaggacaagacagaaacaagaagagtgcaaaggcaaaagcagcagctcgtatattggcagatctgaaggtaggttctttaaacttaaagaggaatgctgtattagtgtgtagttcatgacagtgcctcgtggtttaaattgttgttcagttgaatcggtagcagaactatatcctgagcatactttcctgtatttaactagctgatgttttctgcttagaaattactccctccgttcctaaatataagtctttttaggcatttcaaatggactataacatatggatgtatgtagacatattttagagtgtagattcactcattttgctccgtatgtagtcacttgttggaatctctagaaagacttatatttgggaacggagggagtagatgatttgtctatgccatttatttatgacagaaactgatgtttaagtttgaagcagaaaagaggcctttcaatgaaacaatgttcttctaaagctaagcagttggatattatatcatcggagcagtatcctttggcaagtgtaagcttctacatagttttccagcaaatgctttaacaaagtggtgtattttattatagataatgactattattacatttctgtagttggaatcaccacttgattattgtcacgtgaatgccaatcctagcctaggaggccttccctcactgaaagaagcaggtatctgacgattcccattttgatcgtcctttggtttaattgtgttgcttgatcgctgacagcctgtcaaacatgacatactaaaatgtatatatacctctttctttttacatggataagatgcatgtctagtcagtaaccatgcatttttgtttgttttcagttgttcttcggcttaaaatggacaaaggtggaccacgaagtgctctttttaagctatgcaagaggttgccgtggccaatgccagaattcgaatttgttgaacaaaggttcaggtatgtccaggaatacaaaatatatatgacctctagcaacaaaggcaaaaacttcattccacttgaatgttggcaatgccgatccttctgctcttttaacgaacatgtgctaaattcttttactggtttgatgcaacaggactcctattgttctggacggggtaaccacaacgaacttcaatagcttcgtgtcaaccatcaccttgcacatacctgatgtaacagccattacacttcaaggcgagcggcggactgacaaaaggagttcccaggattcagcatcgctggcaatgctccacaagcttcaagagctcaaggtctgtatatgcaagacttagcaaagcatcatggatccacttgcgagctaactgctaaggctcgttaacctggggatctgatcctccaccccaaagtgaattatgttgaaaatgttttatgaaaggctcggtgtggtgctagattatcctgcaggatatcttagaaatagttaatgcctgtcttctgaaaataacagtctacaatttcacttgctggacaatgaaaacattttgcaaga

SEQ ID NO: 8 - protein, 1628 aa, Triticum aestivum
MAMAMADDDGPIPPPPPPPPPRRPHRQLQPRRYQVEVFEAALQGNTIAVLDTGSGKTMVAVLLARHHVGRVRAGEVPRRIVVFLAPTVHLVHQQFEVIREYTDLDAAECYGASGVGDWSTDRWNKEVGSKEIAVMTPQILLDALRHAFVTMSAVSLLIFDECHRACGNHPYTRIMKEFYLGSQWRPAVFGMTASPVATKGTCTVQDCEAHIGQLELTLDAKVYIIKDRSELESFSPPATIVNKYYDAYSIDFEDLKSKLQILYEEYDALLASLQESSPNKFEDTNNILETSRKTLFRYHEKIFYGLNYLGPIITAEVVKIYNESIKTLGDSEDCLFSKASFSLHVAYFKEALDLVEEVLPHGYGELMKSESGSAELIKSGYISSKVDALINIFKSFGSSNEVLCLIFVERIMTAKAVERFMRGIVNFSSFSISYLTGGSTSKDSLSPAVQRYTLDLFRSGKVNLLFTTDVTEEGIDVPNCSCVIRFDLPRTVCSYVQSRGRARRSSSNYVLMIERGNMDQQEHIFRIIQTEYYIKHFALYRHRNPNVSSSEWPMQDKYAYHVDSTGATITADCCVDLIRKYCEKLPKDRYYIPKPSFAVAIEDGSYQCTLTLPPNAAFQRIVGPLCSTSNLAKQLVSLDACKKLHQLGELNDHLLPFTEEPIDIDVALRDGKFLSGPGTTKRKELHGTRNVLALSGSWIHESESVTLNAYRFDFLCDQEGENYAGFVLLMESALDDDVACSKMDLFLIPNKMVYTTVTPCGKVQLDKKQLRDGKLFQEFFFNGIFGRLFHGSRTSGVQREFLFRKGHEIQWSSQSMYLLLPLRHSPHIQQDLNIYWEAVESCTQAVEQLRNLYLEDENIRANFIQHRSIKEKDIIHLANKSLHFSSIKDSVVLSLHTGRIYSVLDLIYGTTAEDSFEEMYNGKASPFSSFVDYYREKYGIVIQHPKQPLLLLKQSHNAHNLLFSKSKYIDGSTGDPLLMEKEQIHARVPPELLIHIDVTIEVLKSFYLLPSVMHRLQSLMLASQLRRDIGYTQHIPSYLILEAITTLRCCETFSLERLELLGDSVLKYVIGCDLFLRYPMKHEGHLSDMRSNAVCNATLHKHGIWRSLQGYVRDSAFDPRRWVAPGQISLRPFPCNCRIETAFVPSNGRYISDDPSFVVGKPCDRAHRWMCSKAISDCVEALVGAYYVGGGIVGALWVMKWFGIEIKCDRKLVQEVKLNASYICYLPKISVIEELEAKLKYNFSVKGLLLEAITHPSLQELGADYCYQRLEFLGDSVLDLLITRYLYVTHTDVDPGELTDLRSALVSNENFAEVVLRNNIHSHLQHGSGILLEQITEYVRSNLECQGKVNKFLQHATCKVPKVLGDIMESIAGAIFVDTDFNVDVVWKIVEPLLSPMITPDNLVLPPYRELLELCSHLGYFINSKCSSKGEEVIIEMSVQLRDELLIAQGQDRNKKSAKSKAAARILADLKKRGLSMKQCSSKAKQLDIISSDQQYPLASLESPLDYCHVDANPSLGGLPSLKEAVVLRLKLDKGGPRSALFKLCKRLQWPMPEFEFVEQRFRTPIVLDGVTTTNFNSFVSTITLHIPDVTAITLQGERRTDKKSSQDSASLIMLHKLQELKVCICKT

SEQ ID NO: 9 - DNA, 12540 bp, Triticum aestivum
  source 1..12540: mol_type = genomic DNA
actcttgagtcattttcctccccccaaacctcctctccgcgccaacgaacgcagcgcaacccacacctccacttcacgcactttgccttctcccctcctccgcccagtctcccacctcccgtcccatccccggcgccgatttatatacacggctggcctacagacatgcgcttcccccacccccgcctcgcccactcgcccgcaccgaacccaaccccatggccatggccatggccgacgacgacggccccatccccccgcctccgccgccgccgccgcctcgccgcccccacaggcagctccagccgaggaggtcgcgcattactctctccgcagttctctggcgcccgcattcctagagctgatgggggttacccattccgtgtctgtgtcggcgcaggtaccaggtggaggtgttcgaggcggcgctgcaggggaacaccatcgcggtgctcgacaccggctccggaaagaccatggtcgccgtcctgctcgcgcgccaccacgtgggccgcgtgcgcgccggggaggtgccccgccggatcgtcgtcttcctcgcgcccaccgtgcacctcgtccaccaggtgcgcttctggactctgcagcgaccgattcgcgacggcaacgtctgcgccgttttctcgcttcgcattcgcagctgccatggccctgatcgccgaatgctgcttgggctcgcgctgtagcaattcgaggtgattcgggagtacacggacctcgacgccgctgagtgctacggagcttcgggggtcggcgactggagtaccgaccgctggaacaaggaagttgggagcaaggaggtaagtgtcggggtttactctatcccgcggcacggtcattggttgatccgaatgcacactgaattggtacacgtaccgtgctcaccaagtgcttgatgaatgacttgttgacaaggaaccaacattgttggtttctgggtgatagcagcttcggatcgcatgtagatcgatgccattgttatttgagtagtattctttagcgtgagggcagtcgactaaagcaatttgctgaagggccgtgactttgaaatagctggggtctcgagtctggacacaagtcctaataaaagaaaatttcctttcaatgcagtcactgctaaatttggacactgctttgaatccgcaagatctcacaactcctgtaaatatgttgcttaaaaaaactcttgcaaatttgccacttggtaaagttatctggattatgtgtctttctaaattctaatccagttttgtggatattcctatgttttttacctgccgtattgcgcttattatgcgttctattgacaactttgattgaaaatatgaattttcctggaacaatgatcttattatgcgttctattatgtgtttcagattgctgttatgacgccccagatattgttggatgccctgaggcatgcctttgtaacaatgagtgcagtgagcctgctaatatttgatgaatgccatcgtgcctgtggaaaccacccatatacacgaataatgaaggtaatgggatttttcttgttttaacaacatctgtagatatgctttggcttgcctctcatgcagttttttgaaagtaaagaagtttcgtagaatagcagtatttttcatagtttggcatgtcgaatttcctaacggtttatttctcttttgagtctgtaggaattttaccttggctctcagtggaggccagctgtatttggaatgacagcatctcctgttgctactaaaggtaagcattctacacatgactcatgtgcttcctttctttataccctctgcgattattcttcagcaatatttccggcaaacgaccactattttggttcttgaaatcagcatgttgtatgactgttatgtaccatgtgtcgtgctcagtgctcttatcttctcccccacaagatcatacacttcctgaagtgcgatactctgttgtgccaacagtgccctactgctgtacactatatataaattgcagttagctacctgtacatgattgcttccttcttcatctggatgactgggaacacaacttgtacctttgacatgatttttattggtttatttcattttgacaaaaaggaaaattgtgcctgctattcatcctacctgaaggcacttgtgacttgggaccaatttatcaaatcaaaattattttcctgcactaggatgatttgttagacgctaacttcagtagaggagaggcaagtttcatttctaaaaattaccgtccttttcaggcacttgcacggtacaagattgtgaagcacacattggtcaacttgaacttacattggatgcgaaggttacctcagcatcctcaacttctttcactttgaatttcattgagcctttggaaatagcctccataccttacatgccattccaggtatatatcataaaagatcgtagtgaacttgagagcttttcccctcctgcaacaattgtgaacaaatactatgatgcttactcgattgatttcgaagatctgaaatcgaagctccagatattgtatgaagaggtgatttaatttaactatgttttgactcattcataagatacatctgtagtttggactttggagtagccacacctataatgcgttgtatttcttgctttgcatctatattactgtacaaatgtttatttgttaaaagttggcattgttgggcagtatgatgctttgttggctagtctgcaagaatcgtcaccaaataaatttgaagacacgaataacatattagagacatcaagaaagaccttgttcagataccatgagaagatattttatggcctaaattatcttggtccaatcatcaccgctgaggtattgtataatctaataattgagtagctttaccaaatttttcgtaagagttacaatggttattgttctatctgatctagttcgttgacttcacataagtccctgattccaactgaagttattcaaactttttggccatgtgtagaatatttgcattggcaaattcctagccgatatactatcacaatatcccttatgttcagcagttgctgataagatctctgaattagcgatgatgggcagaaaatttacctaacattaagaagattaactgctttgcaaggtcatttaatatgtttttagtgtggagaaaatcatgcgcatgaagtctcttgtacactttgagggaagaaatgctgcatcacgagtagactagcgatttagctacctatgaacctatcattttcctctcaacggaaaagtatatttcttagcattatgctgctgttgagttcgtccctaatccttgaaggacaaaataatggagtaaatggacagttaaattaccaggattgttttttatttgagggaaggccatcacgacttagctttattaacttcaaaaaacgtttacatcgtctatgagcttactgaccacaaagtcaggtggctcgtccaaccaactaaaggaagacctattacaataactaaagtttgctaggacatgagtcacttgattacatgaactataacaatgcttataaatgaccatcccaatcaaggaactaatatcgacgcattccgcgaaaattgccaccgccgaatcccaccaactagtttgcccggtgtaatagttaatgacctgtagggagtctgattctgcttctactcgattaaatctgagagaattagcaagagcaagtccatctctcattgccattgcctccattgtgatcacatccgctgcaagaggaatgaatttaccattgccaggattgttggttaacaaaggatcgctttatgttgaatatggaacatatccttctcatgaagtataagttgcatgtgaggagaagcttgagtctactaaaaggtcaactggtattgtcattcaaatgattgtgaagcgtattaattggtcacaatactacttttgctgtgaactgaccattacacttagagctatttatcttggtaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngacggatcgaatgtgttagatgaggcaaaatagcgtagttagaagtcggacagaataatttagttgaggtagttgctgaaccatctgagctgcaactggagtttacctatattgagttgagtggtagagtttgctcctgggacattgtacgaaacatcacgaagaaaccatacactgagatttcatgagaataatggtagcatttttccattttcttgtgcctcaccatataaattgtaaattgggtctctttaatgtatactattcggaaaaagatactagattcttccctaccatttccacagttccagtcttattaagaaggttctggtccaaccatttcccttgcctgtcgcagttagcatacctttttcattattcttccataccatttattataatatgaaatactagattttttcccctggagaacaaactgtgtattgttttcttaacaggtagtgaagatatataatgaaagcattaagacactgggtgattctgaagattgcctcttctcgaaagctagcttcagcctgcatgtagcttattttaaagaagctttagatttagttgaggaagtattgccacatggtaagccacaaacaaatatccatcagttagatgtgttatatctgtctgttttcacttgttattgctcataaatagtaatatactccctccgttccataatgtaagacgttttttgacactacactaaagtgaaaaaacgtcttacattatggggacagagggagtagtatacaactagaatctgcagtatctttctgtatataataaaggggtaggttataaaatcagttgcaaactgcattaagatcctccgaatccttagataagttgtatttatttcagtactttgcctgctatatctcgaaataaaataatggttctacacaaacctcttaatttgcacactctatagaggtgagctatgaggaagtacctgcaacgtgtacagtttctatagcaaatagccgaatagggagttaagtgggtgacatcaaccattcagtagccatatgtgctgcacatatactgtccattattttgggttgaatgtacggcactcctcgttgtgtgaatctatagccttaaagactgaaatcgcctgtttcttaatgcacctatgtgttttgatttctgaagcattgtattattgatatctcactagactgtaagcagcttggaaactacaatgtagtgcattgatagtttctttttctaattctgctgatataattgcatcataggttatggtgaactaatgaaatcagaatctggttctgcggaattaattaaaagcgggtatatttcttcaaaagtggatgctctaatcaacatttttaaatcatttgggtaaagccatattctgctcaagatgcttagttttgcctacttgtgctctcatgtcagcaaaaaagggtaaatccacatgtattttctccacaggtcatcaaacgaagtgctttgcctaatttttgtagaaagaattatgacagctaaagctgttgagaggttcatgagaggaattgttaatttctctagtttttcaatttcttacttgactggagggagtacatcaaaagattctctgagcccagcagtgcagagatatactttggacttgttccgatctggaaaggtatgccatttaaattgcaaagattgatttaaacaggattcaaaatgatacattaactccatgtcaatttctaggtgaacttgcttttcaccacagatgtaactgaagagggtattgatgtacctaactgttcttgtgtcatacgcttcgacctgcccagaactgtttgtagctacgttcaatctcgtggtcgtgccagaaggagcagctcaaattatgttcttatgattgagaggtacaataatcgtcgcacttcataagttgcgtgctggtatagcatattaaattatagagaaatgttttggccaccatttattcttattctaagcacatttatcatgcatattatattcatagattgggtacaatttatgcctcataatcattaatttttctggaaaaggatatgctcttctacataatttggtttggcatgaacggtatgttttgtcagtcagcagtctgagtttctagcccaggaattcctgaatcccgatcctctcatctaaaaaacgttacaaccatgtttaaacattgttttaataatttttatgactacctattcaccttttgatacttcacatgaaattttcaggggaaacatggatcagcaggaacacatattccgcataatacagactgagtactatattaaacacttcgctctgtataggcacagaaaccccaatgtttcatcctctgagtggcctatgcaagataagtatgcataccatgttgattcaacaggagcaactataactgcagactgctgtgttgatctgatccgtaaatattgtgagaaacttcctaaagataggtacacatatgcttaccattcatgtagcttttgttgttctgtcagccagcttggtaatgcatctcatgcataatcactaagaatgctggtgtgcttaagattcctgaactgtgatgcaaccaattacatggatagcatgttggtgaaaaagtcggttaatttgattgtcacacacagcacaacgaaaaactatttctcgtacttcccatggcacgatcttactgtctttcattagatagaagtgattgatcattgtaacattcttctgcaggtattacattccaaagccttcctttgcggtggctatcgaagatggatcataccaatgcaccttgactttgcctccaaatgcggcgtttcaaaggatagttggccctttatgcagtacaagtaatttagccaagcagcttgtatccctagatgcgtgcaagaaattgcatcagctaggagaactcaatgatcatcttctacccttcaccgaagaacctattgatattgatgttgctttaagagatgggaaattcctttctggaccaggtatgtgtataccctttttcagtcttgctctcattaaaattctttagataagaacatttatttgcctgtttttctattttctttcactcatctattttatacacttcatcaatataacagtgatcagagtaagactgagcttgcccatttgtcgttcatggttaaacttgtgcaactaatgctggtgggctttgtttttattgttttgcacagtaatgcaactctgtgtgtatgaggttgtcttttggtcatgtttgtgcaaggtttggggcaaatatgaagtatctcccgactgttgagatctgctgaccttacatagttattctccattcttttactttaccatttcatctgagtggtgctgttacattgcatactgctgtctgtagtgaactgtgcaagtttcaacagcaatcccttcttccaaaatggtatgcgtgcttctctcgtcgtagatgataacgatgttcccattgttcttttctcattttggcaggaacgactaaaagaaaggagctccatggaacaagaaatgttcttgccctgtcaggatcgtggattcatgaaagtgaaagtgtcacattgaatgcttatagattcgattttctttgtgaccaagagggtgaaaactatgctgggtttgttctcttaatggaatcagcacttgatgatgatgtagcttgttcaaaaatggatctattcctgatccctaataaaatggtctacaccaccgtaactccgtgtggaaaagttcaactggacaaaaaacaggttgttcaaggaataatcgcatactgtaattctcttctataaagtttgtttggccatctcttatttttgtcatttcttcagctacgtgacgggaagttgttccaagaattctttttcaatggaatatttggtagattatttcatggatctcgaacaagtggagtgcaaagggaatttcttttcagaaaaggtcatgaaatacaatggagctcacagagcatgtacttacttttacctttgaggcattcaccacacatccagcaagatttaaacatatactgggaggcagttgaatcctgcacccaggcggtggagcagttgagaaatttgtatctggaagatgaaaatatccgtgcaaattttattcaacatagaagtatcaaggagaaagacattattcatctggccaacaaatctcttcatttttctagcatcaaagattcagttgtgctatcgcttcatactggaaggatatactctgttcttgacttgatctatggcacaactgctgaagactcgtttgaagagatgtataatggaaaagcttcaccgttttcttcattcgtggactactaccgtgaaaagtaatacctctattacatgtcttaaactcttaattactagtaaaatttattctgacatgcttcctgacaaacttatctttttaccactcttttgtaggtatggtattgttattcaacatccaaaacaaccgttgttgctgttaaagcaaagccacaatgcacacaatcttcttttttcaaaatcgaagtacataggtactagctataaatcctagtctggtccagttccccttttcccattatgatgaattgcaagatagctgttgctttactatttggtctgcttttacatgaaactctacataacctatggcagatggttctacgggcgatcctttgctcatggaaaaagagcaaatacacgcccgggtcccacctgaattgttaatccacattgatgtgacaattgaagttctcaaatctttttatttattgccttctgtaatgcatcgacttcagtctctcatgctagccagccagctccgcagagatattggttacactcaacatataccaagttacctggtctgttctctttcgtgagaatataattttactgctatggctggttatgcatacctgaataatttgatccgtttcaatgtcagattttggaagctatcacaactttaaggtgctgcgaaacattttccctggagcgtttagaactattgggagactcagtactaaaatatgtgataggatgtgacctgtttttaaggtatcctatgaaacatgaaggtcatctttctgatatgagatcaaatgctgtctgcaatgctacacttcataagcacggaatctggcgatccttgcaggtagatggcctaaatccaagcctattataatggtttccacttcctgaagtttatgatgagttctagtattagacaaataattttgatttagttagttcatgtgcacttggccagtcaagcagttatatttgagtcttccccgcagtaaataaataaaaaatttagcctgagggccaacaatcacatatgaacttcgtatagcaagtggttttcttctgttctgttcttgagttttaattttgtttgagcccatgccagtcagatgttttctagaggtgcttcgtcaatgttcattcaaaaagcatgtttgatgatattgtaaaaaggcagaagttatatacgagcagagtcaagttgcgtataggagatagaatataacctagataccgttgagatttgcaaggttgagaatacaattccaaggtgtgtatatttactggctatttgttattttgctttccagggttacgtgcgggatagtgcatttgacccacggcgttgggttgctcctgggcagatatctttgcgcccttttccttgtaactgcagaattgagactgcatttgttccaagtaatggaaggtatatcagcgacgacccatcttttgtcgttggaaaaccatgtgatagagctcacagatggatgtgctcgaaagcaatatctgattgtgttgaagccctagttggagcatattatgtcggtggtggcatagttggtgcactttgggttatgaagtggttcggtattgaaataaaatgtgataggaagttggtacaggaagtaaagctcaatgcgtcttatatatgctacttacctaaaataagtgttattgaggagttggaagcaaaactgaagtacaacttctcagtcaagggccttctactggaagccataactcatccatctctgcaggaattaggggctgactactgttaccaggtcagtgtatagcagcatatttctatattggtttgtggggttatcattagcttattgtgcttcaatcagaaattgcttcaaaacaagatatatccatgtcttctgtccttaacatttttgcgtctaacttgtgcctttatttcagcgtttggaatttctgggtgattctgtgctggatctactaattacacgctatctgtatgttacccatactgatgttgatcctggagaattgacagacctacgttccgctttggttagcaatgagaattttgcagaagtagttttaagaaataacattcacagtcatctacagcatgggtctggaatacttttggagcaaatcacagaatatgttagatccaatttggagtgccaagggaaggttaataaattccttcaacatgctacatgtaaagtacctaaggttagaaatacagtttcacttatgttcattatcttaacagcctaccttttcaattttgtgcttttattgtttctgcgatggccttttctctgtgaagcctttcccagccaacattacacgtagaagttaataagattattttgtttgttttacaggtgcttggagacattatggaaagcattgccggtgcaatatttgtagacacagattttaatgttgatgtggtttggaagattgttgaaccgttgctttccccaatgataacccctgataatcttgtattgccaccttatcgggagttgttagagctgtgtagtcaccttggttatttcataaattcaaaatgcagtagtaaaggagaagaagtaattatagagatgtcagtgcaattacgagatgaactgctgatagcgcaaggacaagacagaaataagaagagtgcaaagtcaaaagcagcagctcgtatattggcagatctgaaggtaggttctttaaacataagaggaatgctgtattagtgtgtagttcatgacagtgcctcgtggtttaaattgttgttcagttgaatcggtagcagaactatatcctgagcatactagctgatgttttctgcttagaaattactccctccgttccaaaatagatgactcaactattttggaacagagggagtagatgatttgtctatgccatttatctatgacagaaactgatgtttaagtttgaagcagaaaagaggcctttcaatgaaacaatgttcttctaaagctaagcagttggatattatatcttcggaccagcagtatcctttggcaagtgtaagcttctacatagttttccagcaaatgctttaacaaagtggtgtattttattatagataatgactattattacttttctgtagttggaatcaccacttgattattgtcacgtggatgccaatcctagcctaggaggccttccctcactgaaagaagcaggtatctgacgattcccattttgatcgtcctttggtttaattgtgttgcttgatagttacagcctgtcaaacatgacatactcaaatgtatatatacctttttctctttgcacggataagatgcatgtctactcagtaaccatgcaattttgtttgttttcagttgttcttcggcttaaactggacaaaggtggaccacggagtgctctttttaagctatgcaagaggttgcagtggccaatgccagaattcgaatttgtggaacaaaggttcaggtatgtccagtaattcaagatatatatgacctctagcaaccaaaggcgaaaacttcattccacttgaatgttggcgatgccgatccttctggtcttttcacgaagatgtgctaaattcttgtactggtttgatgcaacaggactcctattgttctggacggggtaaccacaacgaacttcaatagcttcgtgtcaaccatcaccttgcacatacctgatgtaacagccattacacttcaaggcgagcggcgaactgacaaaaagagttcccaggattcagcatcgctgataatgctccacaagcttcaagagctcaaggtctgtatatgcaagacttagcaaagcatcatggatccacgtgccagctaactgctaaggctcgttaacctggggatatgatcctccatcccaaagtgaattatgttgaaaatgtttatgagaggctcggcggtggtgctagattatcttgcaggatatcttagaaacaattagtgcttgtcttccgaaaaaaacaatagacaatttcacttgctggacaaagaaaacattttgcaagaattgatattggt

SEQ ID NO: 10 - protein, 1627 aa, Triticum turgidum
MAMADDDDGPLPPPPPPPPPRRPHRQLQPRRYQVEVFEAALQGNTIAVLDTGSGKTMVAVLLARHHVGRVRAGEVPRRIVFFLAPTVHLVHQQVEVIREYTDLDAAECYGASGVGDWSTDRWNKEVGSKEIAVMTPQILLDALRHAFVTMSAVSLLIFDECHRACGNHPYTRIMKEFYLGSQWRPAVFGMTASPVATKGTCTVQDCEAHIGQLELTLDAKVYIIKDRSELESFSPPATIVHEYYDAYSIDFEDLKSKLQILYEEYDALLASLQESSPNKFEDTNNILETSRKTLFRYHEKIFYGLNYLGPIITAEVVKIYNESIKTLGDSEDCLFSKASFSLHVAYFKEALDLIEEVLPHGYGELMKSESGSAELIKRGYISSKVDALINIFKSFGSSNEVLCLIFVDRIMTAKAVERFMRGIVSFSSFSISYLTGGSTSKDSLSPAVQRYTLDLFRSGKVNLLFTTDVTEEGIDVPNCSCVIRFDLPRTVCSYVQSRGRARRSSSNYVLMIERGNMDQQEHIFRIIRTEYYIKHFALYRHKNPNVSSSEWPMQDKYAYYVDSTGATITADCCVDLIRKYCEKLPKDRYYMPKPSFAVAIEDGSYQCTLTLPPNAAFQRIVGPLCSTSNLAKQLVSLDACKKLHQLGELNDHLLPFTEEPIEIDVALRDGKFLSGPGTTKRKELHGTRNVLALSGSWIHESESVTLNAYRFDFLCDQEGENYAGFVLLMESALDDDVACSKMDLFLIPNKMVYTTVTPCGKVQLDKKQLRDGKLFQEFFFNGIFGRLFHGSRTSGVQREFLFRKGHEIQWSSESMYLLLPLRHSPHIQQDLNIYWEAVESCTQAVEQLRNLYLEDENIRANFIQHRSIKEKDIIHLANKSLHFSSIKDSVVLSLHTGRIYSVLDLIYDTTAEDSFEEMYNGKASPFSSFMDYYREKYGIVIQHPKQPLLLLKQSHNAHNLLFSKSKYIDGSTGDPLLMEKEQIHARVPPELLIHIDVTIEVLKSFYLLPSVMHRLQSLMLASQLRRDIGYTQHIPSYLILEAITTLRCCETFSLERLELLGDSVLKYVIGCDLFLRYPMKHEGHLSDMRSNAVCNATLHKHGIWRSLQGYVRDSAFDPRRWVAPGQISLRPFPCNCGIETAFVPTNGRYISDDPSFVVGKPCDRAHRWMCSKAISDCVEALVGAYYVGGGIVAALWVMKWFGIEIKCDRKLVQEVKLNASYICYLPKISVIEELEAKLKYNFSVKGLLLEAITHPSLQELGVDYCYQRLEFLGDSVLDLLITRYLYVTHTDVDPGELTDLRSALVSNENFAEVVLRNNIHSHLQHGSGILLEQITEYVRSNLECQGKVNKFLQHATCKVPKVLGDIMESIAGAIFVDTDFNVDVVWKIVEPLLSPMITPDNLALPPYRELLELCSHLGYFINSKCSSKGEEVSIEMSVQLRDELLIAQGQDRNKKSAKAKAAARILADLKKRGLSMKQCPSKAKQSGIISSDQQYPLASLESPLDYCQVNVNPSLGGLPSLKEAVVLRLKMDKGGPRSALFKLCKRLQWPMPEFEFVEQRFRTPIVLDGVTTTNFNSFVSTITLHIPDVAAITLQGERRTDKKSSQDSASLTMLHKLQELKVCICKT

SEQ ID NO: 11 - DNA, 4884 bp, synthetic construct
  source 1..4884: mol_type = other DNA
atggccatggccgacgacgacgacggccccctgcccccgcctccgccgccgccgccgcctcgccgtccccacaggcagctccagccgaggaggtaccaggtggaggtgttcgaggcggcgctgcaggggaacaccatcgcggtgctcgacaccggctccgggaagaccatggtcgccgtcctgctcgcgcgccaccacgtgggccgcgtgcgcgccggggaggtgccccgccggatcgtcttcttcctcgcgcccaccgtgcacctcgtccaccagcaagtcgaggtgattcgggagtacacagacctcgacgccgcggagtgctacggagcttcgggggtcggcgactggagtacagaccgctggaacaaggaagttgggagcaaggagattgctgttatgacgccccagatattgttggatgccctgaggcatgcctttgtaacaatgagtgcagtgagcctgctaatatttgatgaatgccatcgtgcctgtggaaaccacccatatacacgaataatgaaggaattttaccttggttctcagtggaggccagctgtatttggaatgacagcatctcctgttgctactaaaggcacttgcacggtacaagattgtgaagcacacattggtcaacttgaacttacattggatgcaaaggtatatatcataaaagatcgtagtgaacttgagagcttttcccctcctgcaacaattgtgcacgaatactatgatgcttactcgattgatttcgaagatctgaaatcgaagctccagatattgtatgaagagtatgatgctttgttggctagtctgcaagaatcgtcaccaaataaatttgaagacacaaataacatattagagacatcaagaaagaccttgttcagataccatgagaagatattttatggcctaaattatcttggtccaatcatcaccgctgaggtagtgaagatatataatgaaagcattaagacactgggtgattctgaagattgtctcttctcgaaagctagcttcagcctgcatgtagcttattttaaagaagctttagatttaattgaggaagtattgccacatggttatggtgaactaatgaaatcagaatctggttctgcggaattaattaaaagggggtatatttcttcaaaagtggatgctctaatcaacatttttaaatcatttgggtcatcaaacgaagtgctttgcctaatttttgtggatagaattatgacagctaaagctgttgagaggttcatgagaggaattgttagtttctctagtttttcaatttcttacttgactggagggagtacatcaaaagattctctgagcccagcagtgcagagatatactttagacttgttccgatctggaaaggtgaacttgcttttcaccacagatgtaactgaagagggtattgatgtacctaactgttcttgtgtcatacgcttcgacctgcccagaactgtttgtagctacgttcaatctcgtggtcgtgccagaaggagcagctcaaattatgttcttatgattgagaggggaaacatggatcagcaggaacacatattccgcataatacggactgagtactatattaaacacttcgctctgtataggcacaaaaaccccaatgtttcatcctctgagtggcctatgcaagataagtatgcatactatgttgattcaacaggagcaactataactgcagattgctgtgttgatctgatccgtaaatattgtgagaaacttcctaaagataggtattacatgccaaagccttcctttgcggtggctatcgaagatggatcataccaatgcaccttgactttgcctccaaatgcggcgtttcaaaggatagttggccctttatgcagtacaagtaatttagccaagcagcttgtatccctagatgcgtgcaagaaattgcatcagctaggagaactcaatgatcatcttctacccttcaccgaagaacctattgaaattgatgttgctttaagagatgggaaattcctttctggaccaggaacgactaaaagaaaggagctccatggaacaagaaatgttcttgccctgtcaggatcgtggattcatgaaagtgaaagtgtcacattgaatgcttatagattcgattttctttgtgaccaagagggtgaaaactatgctgggtttgttctcttaatggaatcagcacttgatgatgatgtagcttgttctaaaatggatctattcctgatccctaataaaatggtgtacaccaccgtaactccgtgtggaaaagttcaactggacaaaaaacagctacgtgacgggaagttgttccaagaattctttttcaatggaatatttggtagattatttcatggatctcgaacaagtggagtgcaaagggaatttcttttcagaaaaggtcatgaaatacaatggagctcagagagcatgtacttacttttacctttgaggcattcaccacacatccagcaagatttaaacatatactgggaggcagttgaatcctgcacccaggcggtggagcagttgagaaatttgtatctggaagatgaaaatatccgtgcaaattttattcaacatagaagtatcaaggagaaagacattattcatctggccaacaaatctcttcatttttctagcatcaaagattcagttgtgctatcgcttcatactggaaggatatactctgttcttgacttgatctatgacacaactgctgaagactcgtttgaagagatgtataatggaaaagcttcaccgttttcttcattcatggactactaccgtgaaaagtatggtattgttattcaacatccaaaacaaccgttgttgctgttaaagcaaagccacaatgcacacaatcttcttttttcaaaatcgaagtacatagatggttctacgggtgatcctttgctcatggaaaaagagcaaatacacgcccgggtcccacctgaattgttaatccacattgatgtgacaattgaagttctcaaatctttttatttattgccttctgtaatgcatcgacttcagtctctcatgctagccagccagctccgcagagatattggttacactcaacatataccaagttacctgattttggaagctatcacaactttaaggtgctgcgaaacattttccctggagcgtttagaattattgggagactcagtactaaaatatgtgataggatgtgacctgtttttaaggtatcctatgaaacatgaaggtcatctttctgatatgagatcaaatgctgtctgcaatgctacgcttcataagcacggaatctggcgatccttgcagggttacgtgcgggatagtgcatttgacccacggcgttgggttgctcctgggcagatatcattgcgcccttttccttgtaactgcggaattgagactgcatttgttccaactaatggaaggtatatcagcgacgacccatcttttgtcgttggaaaaccatgtgatagagctcacagatggatgtgctcgaaagcaatatctgattgtgttgaagccctagttggagcatattatgtcggtggtggcatagttgctgcactttgggttatgaagtggttcggtattgaaataaaatgcgataggaagttggtacaggaagtaaagctcaacgcatcttatatatgctacttacctaaaataagtgttattgaggagttggaagcaaaactgaagtacaacttctcagtcaagggccttctactggaagccataactcatccatctctgcaggaattaggggttgactactgttaccagcgtctggaatttctgggtgattctgtgctggatctactaattacacgctatctctatgttacccatactgatgttgatcctggagaattgacagacttacgttccgctttggttagtaatgagaattttgcagaagtagttttaagaaataacattcacagtcatctacagcatgggtctggaatacttttggagcaaatcacagaatatgttagatccaatttggagtgccaagggaaggttaataaattccttcaacatgctacatgtaaagtacctaaggtgcttggagacattatggaaagcattgccggtgcaatatttgtagacacagattttaatgttgatgtggtttggaagattgttgaaccgttgctttccccaatgataacccctgataatcttgcattgccaccttatcgggagttgttagagctgtgtagtcaccttggttatttcataaattcaaaatgcagtagtaaaggagaagaagtaagtatagagatgtcagtgcaattacgagatgaactgctgatagcgcaaggacaagacagaaataagaagagtgcaaaggcaaaagcagcagctcgtatattggcagatctgaagaaaagaggcctttcaatgaaacaatgtccttctaaagctaagcagtcgggtattatatcttcggaccagcagtatcctttggcaagtttggaatcaccacttgattattgtcaggtgaatgtcaatcctagcctaggaggccttccctcactgaaagaagcagttgttcttcggctcaaaatggacaaaggtggaccacgaagtgctctttttaagctatgcaagaggttgcagtggccaatgccagaattcgaatttgtggaacaaaggttcaggactcctattgttctggacggggtaaccacaacgaacttcaatagcttcgtgtcaaccatcaccttgcacatacctgatgtagcagccattacacttcaaggtgagcggcgaactgacaaaaagagttcccaggattcagcatcgctgacaatgctccacaagcttcaagagctcaaggtctgtatatgcaagacttag

SEQ ID NO: 12 - protein, 1701 aa, Triticum turgidum
MKPPSLSLHFLPPNLLCAPTNAAQLTPPLHALRLLPSAQSPTSRPSPGADLYTRLVYRHALPPPPPRPLARTEPNPMAMADDDGPLPPPPPPPPPRRPHRQLQPRRYQVEVFEAALQGNTIAVLDTGSGKTMVAVLLARHHVGRVRAGEVPRRIVVFLAPTVHLVHQQFEVIREYTDLDAAECYGALGVGDWSTDRWNKEVGSKEIAVMTPQILLDALRHAFVTMSAVSLLIFDECHRACGNHPYTRIMKEFYLGSQWRPAVFGMTAFPVATKGTCTVQDCEAHIGQLELILDAKVYIIKDRSELESFSPPATIVHKYYDAYSIDFKDLKSKLQILYEEYDALLASLQESSPNKFEDTNNILETSRKTLFRYHEKIFYGLNFLGPIITAEVVKIYNESIKTLGDSEDCLFSKASFSLHVAYFKEALDLIEKVLPHGYGELMKSESGSAELIKSGYISSKVDALINIFKSFGSSNEVLCLIFVERIMTAKAVERFMRGIVNFSSFSISYLTGGSTSKDSLSPAVQRYTLDLFRSGKVNLLFTTDVTEEGIDVPNCSCVIRFDLPRTVCSYVQSRGRARRSSSNYVLMIERGNKDQQEHIFRIIQTEYYIKHFALYRHRNPNVSSSEWPMQDKYAYHVDSTGATITADCCVDLIRKYCEKLPKDRYYMPKPSFAVAIEDGSYQCTLTLPPNAAFQRIVGPLCSTSNLAKQLVSLDACKKLHQLGELNDHLLPFTEEPIDIDVALRDGKFLSGPGTTKRKELHGTRNVLALSGSWIHESGSVTLNAYRFGFLCDQEGENYAGFVLLMESALDDDVAYSKMDLFLIPNKMVYTTVTPCGKVQLDKKQLRDGKLFQEFFFNGIFGRLFHGSRTSGVQREFLFRKGHEIQWSSESMYLLLPLRHSPHIQQDLNIYWEAVESCTQAVERLRNLYLEEENIRANFIQHRSIKEKDIIHLANKSVHFSSIKDSVVLSLHTGRIYSVLDLIYDITAEDSFEEMYNGKASPFSSFVDYYREKYGIVIQHPKQPLLLLKQSHNAHNLLFSKSKYIDGSTGDPLLMEKEQIHARVPPELLIHIDVTIEVLKSFYLLPSVMHRLQSLMLASQLRRDIGYTQHIPSYLILEAITTLRCCETFSLERLELLGDSVLKYVIGCDLFLRYPMKHEGHLSDMRSNAVCNATLHKHGIWRSLQGYVRDSAFDPRRWVAPGQISLRPFPCNCGIETAFVPTNGRYISDDPSFVVGKPCDRAHRWMCSKAISDCVEALVGAYYVGGGIVAALWVMKWFGIEIKCDRTLVQEVKLNASYICYLPKISVIEELEAKLKYNFSVKGLLLEAITHPSLQELGVDYCYQRLEFLGDSVLDLLITRYLYVTHTDVDPGELTDLRSALVSNENFAEVVLRNNIHSHLQHGSGILLEQITEYVRSNLECQGKVNKFLQHATCKAPKVLGDIMESIAGAIFVDADFNVDVVWKIVEPLLSPMITPDNLALPPYRELLELCSHLGYFINSKCSSKGEELSIEMSVQLRDELLIAQGQDRNKKSAKAKAAARILADLKKRGLSMKQCSSKAKQLDIISSEQYPLASLESPLDYCHVNANPSLGGLPSLKEAVVLRLKMDKGGPRSALFKLCKRLQWPMPEFEFVEQRFRTPIVLDGVTTTNFNSFVSTITLHIPDVTAITLQGERRTDKRSSQDSASLAMLHKLQELKVCICKT

SEQ ID NO: 13 - DNA, 5106 bp, synthetic construct
  source 1..5106: mol_type = other DNA
atgaagcctccgtcactctcattgcatttcctacccccaaacctcctctgcgcgccaacgaacgcagcgcaactcacacctccgcttcacgcacttcgccttctcccctccgcccagtctcccacctcccgtcccagccccggcgccgatttatatacacggctggtctacagacatgcgcttcccccacccccgcctcgcccactcgcccgcaccgaacccaaccccatggccatggccgacgacgacggccccctccccccgcctccgccgccgccgccgcctcgccgcccccacaggcagctccagccgaggaggtaccaggtggaggtgttcgaggcggcgctgcaggggaacaccatcgcggtgctcgacaccggctccgggaagaccatggtcgccgtcctgctcgcgcgccatcacgtgggccgcgtgcgcgccggggaggtgccccgccggatcgtcgtcttcctcgcgcccaccgtgcacctcgtccaccagcaattcgaggtgattcgggagtacacggacctcgacgccgcggagtgctacggagctttgggggtcggcgactggagtaccgaccgctggaacaaggaagttgggagcaaggagattgctgttatgacgcctcagatattgttggatgccctgaggcatgcctttgtaacaatgagtgcagtgagcctgctaatatttgatgaatgccatcgtgcctgtggaaaccacccatatacacgaataatgaaggaattttaccttggctctcagtggaggccagctgtatttggaatgacagcatttcctgttgctactaaaggcacttgcacggtacaagattgtgaagcacacattggtcaacttgaacttatattggacgcaaaggtatatatcataaaagatcgtagtgaacttgagagcttttcccctcctgcaacaattgtgcacaaatactatgatgcttactcgattgatttcaaagatctgaaatcgaagctccagatattgtatgaagagtatgatgctttgttggctagtctgcaagaatcgtcaccaaataaatttgaagacacgaataacatattagagacatcaagaaagaccttgttcagataccatgagaagatattttatggcctaaattttcttggtccaatcatcaccgctgaggtagtgaagatatataatgaaagcattaagacactgggtgattctgaagattgcctcttctcgaaagctagcttcagcctgcatgtagcttattttaaagaagctttagatttaattgagaaagtattgccacatggttatggtgaactaatgaaatcagaatctggttctgcggaattaattaaaagtgggtatatttcttcaaaagtggatgctctaatcaacatttttaaatcatttgggtcatccaacgaagtgctttgcctaatttttgtagaaagaattatgacagctaaagctgttgagaggttcatgagaggaattgttaatttctctagtttttcaatttcttacttgactggagggagcacatcaaaagattctctgagcccagcagtgcagagatatactttggacttgttccgatctggaaaggtgaacttgcttttcaccacagacgtaactgaagagggtattgatgtacctaactgttcttgtgtcatacgcttcgacctgcccagaactgtttgtagctatgttcaatctcgtggtcgtgccagaaggagcagctcaaattatgttcttatgattgagaggggaaacaaggatcagcaggaacacatattccgcataatacagactgagtactatattaaacacttcgctctgtataggcacagaaaccccaatgtttcatcctctgagtggcctatgcaagataagtatgcataccatgttgattcaacaggagcaactataactgcagactgctgtgttgatctgatccgtaaatattgtgagaaacttcctaaagataggtattacatgccaaagccttcctttgcggtggctatcgaagatggatcataccaatgcaccttgactttgcctccaaatgcggcgtttcaaaggatagttggccctttatgcagtacaagtaatttagccaagcagcttgtatccctagatgcgtgcaagaaattgcatcagctaggagaactcaatgatcatcttctacccttcaccgaagaacctattgatattgatgttgctttaagagatgggaaattcctttctggaccaggaacgactaaaagaaaggagctccatggaacaagaaatgttcttgccctgtcaggatcgtggattcatgaaagtggaagtgtcacattgaatgcttatagattcggttttctttgtgaccaagagggtgaaaactatgctgggtttgttctcttaatggaatcagcacttgatgatgatgtagcttattcaaaaatggatctattcctgatccctaataaaatggtctacaccaccgtaactccgtgtggaaaagttcaactggacaaaaaacagctacgtgacgggaagttgtttcaagaattctttttcaatggaatatttggtagattatttcatggatctcgaacaagtggagtgcaaagggaatttcttttcagaaaaggtcatgaaatacaatggagctcagagagcatgtacttacttttacctttgaggcattcaccacacatccagcaagatttaaacatatactgggaggcagttgaatcctgcacccaggcggtggagcggttgagaaatttgtatctggaagaggaaaatatccgtgcaaattttattcaacatagaagtatcaaggagaaagacattattcatctggccaacaaatctgttcatttttctagcatcaaagattcagttgtgctatcgcttcatactggaaggatatactctgttcttgacttgatctatgacataactgctgaagactcgtttgaagagatgtataatggaaaagcttcaccgttttcttcattcgtggactactatcgtgaaaagtatggtattgttattcaacatccaaaacaaccgttgttgctgttaaagcaaagccacaatgcacacaatcttcttttttcaaaatcgaagtacatagatggttctacgggcgatcctttgcttatggaaaaagagcaaatacacgcccgggtcccacctgaattgttaatccacattgatgtgacaattgaagttctcaaatctttttacttattgccttctgtaatgcatcgacttcagtctctcatgctagccagccagctccgcagagatattggttacactcaacatataccaagttacctgattttggaagctatcacaactttaaggtgctgtgaaacattttccctggagcgtttagaactattgggagactcagtactaaaatatgtgataggatgtgacctgtttttaaggtatcctatgaaacatgaaggtcatctttctgatatgagatcaaatgctgtctgcaatgctacacttcataagcacggaatctggcgatccttgcagggttacgtgcgggatagtgcatttgacccacggcgttgggttgctcctgggcagatatctttgcgcccttttccttgtaactgcggaattgagactgcatttgttccaactaatggaaggtatatcagcgacgacccatcttttgtagttggaaaaccatgtgatagagctcacagatggatgtgctcgaaagcaatatctgattgtgttgaagccctagttggagcatattatgttggtggtggcatagttgctgcactttgggttatgaagtggttcggtattgaaataaaatgcgataggacgttggtacaggaagtaaagctcaatgcatcttatatatgctacttacctaaaataagtgttattgaggagttggaagcaaaactgaagtacaacttctcggtcaagggccttctactggaagccataactcatccatctctgcaggaattaggggttgactactgttaccagcgtctggaatttctgggtgattctgtgctggatctactaattacacgctatctctatgttacccatactgatgttgatcctggagaattgacagacttacgttccgctttggttagtaatgagaattttgcagaagtagttttaagaaacaacattcacagtcatctacagcatgggtctggaatacttttggagcaaatcacagaatatgttagatccaatttggagtgccaagggaaggttaataaattccttcaacatgctacatgtaaagcacctaaggtacttggtgacattatggaaagcattgccggtgcaatatttgttgacgcagattttaatgttgatgtggtttggaagattgttgaaccgttgctttccccaatgataacccctgataatcttgcattgccaccttatcgggagttgttagagctgtgtagtcaccttggttatttcataaattcaaaatgcagtagtaaaggagaagaattaagtatagagatgtcagtgcaattacgagatgaactgttgatagcgcaaggacaagacagaaacaagaagagtgcaaaggcaaaagcagcagctcgtatattggcagatctgaagaaaagaggcctttcaatgaaacaatgttcttctaaagctaagcagttggatattatatcatcggagcagtatcctttggcaagtttggaatcaccacttgattattgtcacgtgaatgccaatcctagcctaggaggccttccctcactgaaagaagcagttgttcttcggcttaaaatggacaaaggtggaccacgaagtgctctttttaagctatgcaagaggttgcagtggccaatgccagaattcgaatttgttgaacaaaggttcaggactcctattgttctggacggggtaaccacaacgaacttcaatagcttcgtgtcaaccatcaccttgcacatacctgatgtaacagccattacacttcaaggcgagcggcggactgacaaaaggagttcccaggattcagcatcgctggcaatgctccacaagcttcaagagctcaaggtctgtatatgcaagacttag

SEQ ID NO: 14 - protein, 1367 aa, Escherichia coli
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 15 - DNA, 23 bp, synthetic construct
  source 1..23: mol_type = other DNA
gtgcaggcgagtgggggtggggg

SEQ ID NO: 16 - DNA, 23 bp, synthetic construct
  source 1..23: mol_type = other DNA
tgttcgaggcggcgctgcagggg

SEQ ID NO: 17 - DNA, 23 bp, synthetic construct
  source 1..23: mol_type = other DNA
gaaatcagaatctggtaccgcgg

SEQ ID NO: 18 - DNA, 23 bp, synthetic construct
  source 1..23: mol_type = other DNA
gctgttgagaggttcatgagagg

SEQ ID NO: 19 - DNA, 239 bp, synthetic construct
  source 1..239: mol_type = other DNA
gaaatcagaatctggtaccgcggaattaagtaaaagggggtatatttcttcaaaagtggatgctctaatcaacatttttaaatcgtttgggtaaagccatattctgctcaagatgcttagttttgcctacttgtgctcttgtgtcagcaaaaatgggtaaatccacatgtattttctccacaggtcatcaaacgaagtgctttgcctaatttttgtagaaagaattatgacagctaaag

SEQ ID NO: 20 - DNA, 20 bp, synthetic construct
  source 1..20: mol_type = other DNA
ctcggctggagctgcctgtg

SEQ ID NO: 21 - DNA, 20 bp, synthetic construct
  source 1..20: mol_type = other DNA
ctcggctggagctgcctgtg

SEQ ID NO: 22 - DNA, 20 bp, synthetic construct
  source 1..20: mol_type = other DNA
taatgcgcgacctcctcggc

SEQ ID NO: 23 - DNA, 20 bp, synthetic construct
  source 1..20: mol_type = other DNA
acaggcagctccagccgagg

SEQ ID NO: 24 - DNA, 20 bp, synthetic construct
  source 1..20: mol_type = other DNA
ccaggtggaggtgttcgagg

SEQ ID NO: 25 - DNA, 20 bp, synthetic construct
  source 1..20: mol_type = other DNA
gctgcaggggaacaccatcg

SEQ ID NO: 26 - DNA, 18493 bp, synthetic construct
  source 1..18493: mol_type = other DNA
cgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagctttcgcagatccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgttcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccgggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagggaaggtgcgaacaagtccctgatatgagatcatgtttgtcatctggagccatagaacagggttcatcatgagtcatcaacttaccttcgccgacagtgaattcagcagtaagcgccgtcagaccagaaaagagattttcttgtcccgcatggagcagattctgccatggcaaaacatggtggaagtcatcgagccgttttaccccaaggctggtaatggccggcgaccttatccgctggaaaccatgctacgcattcactgcatgcagcattggtacaacctgagcgatggcgcgatggaagatgctctgtacgaaatcgcctccatgcgtctgtttgcccggttatccctggatagcgccttgccggaccgcaccaccatcatgaatttccgccacctgctggagcagcatcaactggcccgccaattgttcaagaccatcaatcgctggctggccgaagcaggcgtcatgatgactcaaggcaccttggtcgatgccaccatcattgaggcacccagctcgaccaagaacaaagagcagcaacgcgatccggagatgcatcagaccaagaaaggcaatcagtggcactttggcatgaaggcccacattggtgtcgatgccaagagtggcctgacccacagcctggtcaccaccgcggccaacgagcatgacctcaatcagctgggtaatctgctgcatggagaggagcaatttgtctcagccgatgccggctaccaaggggcgccacagcgcgaggagctggccgaggtggatgtggactggctgatcgccgagcgccccggcaaggtaagaaccttgaaacagcatccacgcaagaacaaaacggccatcaacatcgaatacatgaaagccagcatccgggccagggtggagcacccatttcgcatcatcaagcgacagttcggcttcgtgaaagccagatacaaggggttgctgaaaaacgataaccaactggcgatgttattcacgctggccaacctgtttcgggcggaccaaatgatacgtcagtgggagagatctcactaaaaactggggataacgccttaaatggcgaagaaacggtctaaataggctgattcaaggcatttacgggagaaaaaatcggctcaaacatgaagaaatgaaatgactgagtcagccgagaagaatttccccgcttattcgcaccttccttagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatcccctcgaggcgcgccaagctatcaaacaagtttgtacaaaaaagcaggctccgaattcgcccttcaccatggattacaaggatgatgatgataaggattacaaggatgatgatgataagatggctccaaagaagaagagaaaggttggaatccacggagttccagctgctgataagaagtactctatcggacttgacatcggaaccaactctgttggatgggctgttatcaccgatgagtacaaggttccatctaagaagttcaaggttcttggaaacaccgatagacactctatcaagaagaaccttatcggtgctcttcttttcgattctggagagaccgctgaggctaccagattgaagagaaccgctagaagaagatacaccagaagaaagaacagaatctgctaccttcaggaaatcttctctaacgagatggctaaggttgatgattctttcttccacagacttgaggagtctttccttgttgaggaggataagaagcacgagagacacccaatcttcggaaacatcgttgatgaggttgcttaccacgagaagtacccaaccatctaccaccttagaaagaagttggttgattctaccgataaggctgatcttagacttatctaccttgctcttgctcacatgatcaagttcagaggacacttccttatcgagggagaccttaacccagataactctgatgttgataagttgttcatccagcttgttcagacctacaaccagcttttcgaggagaacccaatcaacgcttctggagttgatgctaaggctatcctttctgctagactttctaagtctcgtagacttgagaaccttatcgctcagcttccaggagagaagaagaacggacttttcggaaaccttatcgctctttctcttggacttaccccaaacttcaagtctaacttcgatcttgctgaggatgctaagttgcagctttctaaggatacctacgatgatgatcttgataaccttcttgctcagatcggagatcagtacgctgatcttttccttgctgctaagaacctttctgatgctatccttctttctgacatccttagagttaacaccgagatcaccaaggctccactttctgcttctatgatcaagagatacgatgagcaccaccaggatcttacccttttgaaggctcttgttagacagcagcttccagagaagtacaaggaaatcttcttcgatcagtctaagaacggatacgctggatacatcgatggaggagcttctcaggaggagttctacaagttcatcaagccaatccttgagaagatggatggaaccgaggagcttcttgttaagttgaacagagaggatcttcttagaaagcagagaaccttcgataacggatctatcccacaccagatccaccttggagagcttcacgctatccttcgtagacaggaggatttctacccattcttgaaggataacagagagaagatcgagaagatccttaccttcagaatcccatactacgttggaccacttgctagaggaaactctcgtttcgcttggatgaccagaaagtctgaggagaccatcaccccttggaacttcgaggaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggttgttgataagggagcttctgctcagtctttcatcgagagaatgaccaacttcgataagaaccttccaaacgagaaggttcttccaaagcactctcttctttacgagtacttcaccgtttacaacgagcttaccaaggttaagtacgttaccgagggaatgagaaagccagctttcctttctggagagcagaagaaggctatcgttgatcttcttttcaagaccaacagaaaggttaccgttaagcagttgaaggaggattacttcaagaagatcgagtgcttcgattctgttgaaatctctggagttgaggatagattcaacgcttctcttggaacctaccacgatcttttgaagatcatcaaggataaggatttccttgataacgaggagaacgaggacatccttgaggacatcgttcttacccttacccttttcgaggatagagagatgatcgaggagagactcaagacctacgctcaccttttcgatgataaggttatgaagcagttgaagagaagaagatacaccggatggggtagactttctcgtaagttgatcaacggaatcagagataagcagtctggaaagaccatccttgatttcttgaagtctgatggattcgctaacagaaacttcatgcagcttatccacgatgattctcttaccttcaaggaggacatccagaaggctcaggtttctggacagggagattctcttcacgagcacatcgctaaccttgctggatctccagctatcaagaagggaatccttcagaccgttaaggttgttgatgagcttgttaaggttatgggtagacacaagccagagaacatcgttatcgagatggctagagagaaccagaccacccagaagggacagaagaactctcgtgagagaatgaagagaatcgaggagggaatcaaggagcttggatctcaaatcttgaaggagcacccagttgagaacacccagcttcagaacgagaagttgtacctttactaccttcagaacggaagagatatgtacgttgatcaggagcttgacatcaacagactttctgattacgatgttgatcacatcgttccacagtctttcttgaaggatgattctatcgataacaaggttcttacccgttctgataagaacagaggaaagtctgataacgttccatctgaggaggttgttaagaagatgaagaactactggagacagcttcttaacgctaagttgatcacccagagaaagttcgataaccttaccaaggctgagagaggaggactttctgagcttgataaggctggattcatcaagagacagcttgttgagaccagacagatcaccaagcacgttgctcagatccttgattctcgtatgaacaccaagtacgatgagaacgataagttgatcagagaggttaaggttatcaccttgaagtctaagttggtttctgatttcagaaaggatttccagttctacaaggttagagagatcaacaactaccaccacgctcacgatgcttaccttaacgctgttgttggaaccgctcttatcaagaagtacccaaagttggagtctgagttcgtttacggagattacaaggtttacgatgttagaaagatgatcgctaagtctgagcaggagatcggaaaggctaccgctaagtacttcttctactctaacatcatgaacttcttcaagaccgagatcacccttgctaacggagagatcagaaagagaccacttatcgagaccaacggagagaccggagagatcgtttgggataagggaagagatttcgctaccgttagaaaggttctttctatgccacaggttaacatcgttaagaaaaccgaggttcagaccggaggattctctaaggagtctatccttccaaagagaaactctgataagttgatcgctagaaagaaggattgggacccaaagaagtacggaggattcgattctccaaccgttgcttactctgttcttgttgttgctaaggttgagaagggaaagtctaagaagttgaagtctgttaaggagcttcttggaatcaccatcatggagcgttcttctttcgagaagaacccaatcgatttccttgaggctaagggatacaaggaggttaagaaggatcttatcatcaagttgccaaagtactctcttttcgagcttgagaacggaagaaagagaatgcttgcttctgctggagagcttcagaagggaaacgagcttgctcttccatctaagtacgttaacttcctttaccttgcttctcactacgagaagttgaagggatctccagaggataacgagcagaagcagcttttcgttgagcagcacaagcactaccttgatgagatcatcgagcaaatctctgagttctctaagagagttatccttgctgatgctaaccttgataaggttctttctgcttacaacaagcacagagataagccaatcagagagcaggctgagaacatcatccaccttttcacccttaccaaccttggtgctccagctgctttcaagtacttcgataccaccatcgatagaaaaagatacacctctaccaaggaggttcttgatgctacccttatccaccagtctatcaccggactttacgagaccagaatcgatctttctcagcttggaggagataagagaccagctgctaccaagaaggctggacaggctaagaagaagaagtgagacgtccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgatcccccctcgacagcttccggaaagggcgaattcgcaactttgtatacaaaagttgccccatggcgttccctctagataacgcaggatccccaagtggtggctatgaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttggtgcaggcgagtgggggtgggtttaagagctatgctggaaacagcatagcaagtttaaataaggctagtccgttatcaacttgaaaaagtggcaccgagtcggtgcaacaaagcaccagtggtctagtggtagaatagtaccctgccacggtacagacccgggttcgattcccggctggtgcatgttcgaggcggcgctgcaggtttaagagctatgctggaaacagcatagcaagtttaaataaggctagtccgttatcaacttgaaaaagtggcaccgagtcggtgcaacaaagcaccagtggtctagtggtagaatagtaccctgccacggtacagacccgggttcgattcccggctggtgcagaaatcagaatctggtaccggtttaagagctatgctggaaacagcatagcaagtttaaataaggctagtccgttatcaacttgaaaaagtggcaccgagtcggtgcaacaaagcaccagtggtctagtggtagaatagtaccctgccacggtacagacccgggttcgattcccggctggtgcagctgttgagaggttcatgaggtttaagagctatgctggaaacagcatagcaagtttaaataaggctagtccgttatcaacttgaaaaagtggcaccgagtcggtgcaacaaagcaccagtggtctagtggtagaatagtaccctgccacggtacagacccgggttcgattcccggctggtgcatttttttgttttttatgtctccagactagtaagggcaaattcgacccagctttcttgtacaaagtggttcgataattcttaattaactagttctagagcggccgccaccgcggtggagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaatt

SEQ ID NO: 27 - DNA, 13655 bp, synthetic construct
  source 1..13655: mol_type = other DNA
gggacacgaagtgatccgtttccttgacaggatatattggcgggtaaactaagtcgctgtatgtgtttgtttgagatctcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaagaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgtgtaacattggtctagtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacaacattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcgatccccatccaacagcccgccgtcgagcgggcttttttatccccggaagcctgtggatagagggtagttatccacgtgaaaccgctaatgccccgcaaagccttgattcacggggctttccggcccgctccaaaaactatccacgtgaaatcgctaatcagggtacgtgaaatcgctaatcggagtacgtgaaatcgctaataaggtcacgtgaaatcgctaatcaaaaaggcacgtgagaacgctaatagccctttcagatcaacagcttgcaaacacccctcgctccggcaagtagttacagcaagtagtatgttcaattagcttttcaattatgaatatatatatcaattattggtcgcccttggcttgtggacaatgcgctacgcgcaccggctccgcccgtggacaaccgcaagcggttgcccaccgtcgagcgccagcgcctttgcccacaacccggcggccggccgcaacagatcgttttataaatttttttttttgaaaaagaaaaagcccgaaaggcggcaacctctcgggcttctggatttccgatccccggaattagatcttggcaggatatattgtggtgtaacgtttagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactctgtgggtctcatgccgaattcggatccagcgtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttgacaagcctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagttatcatcatcatcatagacacacgaaataaagtaatcagattatcagttaaagctatgtaatatttacaccataaccaatcaattaaaaaatagatcagtttaaagaaagatcaaagctcaaaaaaataaaaagagaaaagggtcctaaccaagaaaatgaaggagaaaaactagaaatttacctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcattggcttctacctacaaaaaagctccgcacgaggctgcatttgtcacaaatcatgaaaagaaaaactaccgatgaacaatgctgagggattcaaattctacccacaaaaagaagaaagaaagatctagcacatctaagcctgacgaagcagcagaaatatataaaaatataaaccatagtgcccttttcccctcttcctgatcttgtttagcatggcggaaattttaaaccccccatcatctcccccaacaacggcggatcgcagatctacatccgagagccccattccccgcgagatccgggccggatccacgccggcgagagccccagccgcgagatcccgcccctcccgcgcaccgatctgggcgcgcacgaagccgcctctcgcccacccaaactaccaaggccaaagatcgtgtccgagacggaaaaaaaaaacggagaaagaaagaggagaggggcggggtggttaccggcgcggcggcggcggagggggaggggggaggagctcgtcgtccggcagcgaggggggaggaggtggaggtggtggtggtggtggtggtagggttggggggatgggaggagaggggggggtatgtatatagtggcgatggggggcgtttctttggaagcggagggagggccggcctcgtcgctggctcgcgatcctcctcgcgtttccggcccccacgacccggacccacctgctgttttttctttttcttttttttctttctttttttttttttggctgcgagacgtgcggtgcgtgcggacaactcacggtgatagtgggggggtgtggagactattgtccagttggctggactggggtgggttgggttgggttgggttgggctgggcttgctatggatcgtggatagcactttgggctttaggaactttaggggttgtttttgtaaatgttttgagtctaagtttatcttttatttttactagaaaaaatacccatgcgctgcaacgggggaaagctattttaatcttattattgttcattgtgagaattcgcctgaatatatatttttctcaaaaattatgtcaaattagcatatgggtttttttaaagatatttcttatacaaatccctctgtatttacaaaagcaaacgaacttaaaacccgactcaaatacagatatgcatttccaaaagcgaataaacttaaaaaccaattcatacaaaaatgacgtatcaaagtaccgacaaaaacatcctcaatttttataatagtagaaaagagtaaatttcactttgggccaccttttattaccgatattttactttataccaccttttaactgatgttttcacttttgaccaggtaatcttacctttgttttattttggactatcccgactctcttctcaagcatatgaatgacctcgagtatgctagctccgcaagaattcaagcttggaggtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggaatggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagactatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagattatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtctccacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtttcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaaggatctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtgagcttgtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgctactagaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgcctcggctggagctgcctgtgttttagagctagaaatagcaagttaaaataaggctagtccgttatcaacttgaaaaagtggcaccgagtcggtgctttttttctagacccagctttcttgtacaaagttggcattacgctttacttacgaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgacaggcagctccagccgagggttttagagctagaaatagcaagttaaaataaggctagtccgttatcaacttgaaaaagtggcaccgagtcggtgctttttttctagacccagctttcttgtacaaagttggcattacgctttaccagaaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgctgcaggggaacaccatcggttttagagctagaaatagcaagttaaaataaggctagtccgttatcaacttgaaaaagtggcaccgagtcggtgctttttttctagacccagctttcttgtacaaagttggcattacgctttac

SEQ ID NO: 28 - DNA, 13656 bp, synthetic construct
  source 1..13656: mol_type = other DNA
gggacacgaagtgatccgtttccttgacaggatatattggcgggtaaactaagtcgctgtatgtgtttgtttgagatctcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaagaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgtgtaacattggtctagtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacaacattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcgatccccatccaacagcccgccgtcgagcgggcttttttatccccggaagcctgtggatagagggtagttatccacgtgaaaccgctaatgccccgcaaagccttgattcacggggctttccggcccgctccaaaaactatccacgtgaaatcgctaatcagggtacgtgaaatcgctaatcggagtacgtgaaatcgctaataaggtcacgtgaaatcgctaatcaaaaaggcacgtgagaacgctaatagccctttcagatcaacagcttgcaaacacccctcgctccggcaagtagttacagcaagtagtatgttcaattagcttttcaattatgaatatatatatcaattattggtcgcccttggcttgtggacaatgcgctacgcgcaccggctccgcccgtggacaaccgcaagcggttgcccaccgtcgagcgccagcgcctttgcccacaacccggcggccggccgcaacagatcgttttataaatttttttttttgaaaaagaaaaagcccgaaaggcggcaacctctcgggcttctggatttccgatccccggaattagatcttggcaggatatattgtggtgtaacgtttagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactctgtgggtctcatgccgaattcggatccagcgtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttgacaagcctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagttatcatcatcatcatagacacacgaaataaagtaatcagattatcagttaaagctatgtaatatttacaccataaccaatcaattaaaaaatagatcagtttaaagaaagatcaaagctcaaaaaaataaaaagagaaaagggtcctaaccaagaaaatgaaggagaaaaactagaaatttacctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcattggcttctacctacaaaaaagctccgcacgaggctgcatttgtcacaaatcatgaaaagaaaaactaccgatgaacaatgctgagggattcaaattctacccacaaaaagaagaaagaaagatctagcacatctaagcctgacgaagcagcagaaatatataaaaatataaaccatagtgcccttttcccctcttcctgatcttgtttagcatggcggaaattttaaaccccccatcatctcccccaacaacggcggatcgcagatctacatccgagagccccattccccgcgagatccgggccggatccacgccggcgagagccccagccgcgagatcccgcccctcccgcgcaccgatctgggcgcgcacgaagccgcctctcgcccacccaaactaccaaggccaaagatcgtgtccgagacggaaaaaaaaaacggagaaagaaagaggagaggggcggggtggttaccggcgcggcggcggcggagggggaggggggaggagctcgtcgtccggcagcgaggggggaggaggtggaggtggtggtggtggtggtggtagggttggggggatgggaggagaggggggggtatgtatatagtggcgatggggggcgtttctttggaagcggagggagggccggcctcgtcgctggctcgcgatcctcctcgcgtttccggcccccacgacccggacccacctgctgttttttctttttcttttttttctttctttttttttttttggctgcgagacgtgcggtgcgtgcggacaactcacggtgatagtgggggggtgtggagactattgtccagttggctggactggggtgggttgggttgggttgggttgggctgggcttgctatggatcgtggatagcactttgggctttaggaactttaggggttgtttttgtaaatgttttgagtctaagtttatcttttatttttactagaaaaaatacccatgcgctgcaacgggggaaagctattttaatcttattattgttcattgtgagaattcgcctgaatatatatttttctcaaaaattatgtcaaattagcatatgggtttttttaaagatatttcttatacaaatccctctgtatttacaaaagcaaacgaacttaaaacccgactcaaatacagatatgcatttccaaaagcgaataaacttaaaaaccaattcatacaaaaatgacgtatcaaagtaccgacaaaaacatcctcaatttttataatagtagaaaagagtaaatttcactttgggccaccttttattaccgatattttactttataccaccttttaactgatgttttcacttttgaccaggtaatcttacctttgttttattttggactatcccgactctcttctcaagcatatgaatgacctcgagtatgctagctccgcaagaattcaagcttggaggtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggaatggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagactatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagattatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtctccacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtttcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaaggatctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtgagcttgtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgctactagaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgctcggctggagctgcctgtggttttagagctagaaatagcaagttaaaataaggctagtccgttatcaacttgaaaaagtggcaccgagtcggtgctttttttctagacccagctttcttgtacaaagttggcattacgctttacttacgaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgtaatgcgcgacctcctcggcgttttagagctagaaatagcaagttaaaataaggctagtccgttatcaacttgaaaaagtggcaccgagtcggtgctttttttctagacccagctttcttgtacaaagttggcattacgctttaccagaaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgccaggtggaggtgttcgagggttttagagctagaaatagcaagttaaaataaggctagtccgttatcaacttgaaaaagtggcaccgagtcggtgctttttttctagacccagctttcttgtacaaagttggcattacgctttac

SEQ ID NO: 29 - DNA, 182 bp, Triticum aestivum
  source 1..182: mol_type = genomic DNA
ccccacaggcagctccagccgaggaggtcgcgcattactctctccacagctctctggcgcccgcattcctagagctgacgggggttacccattctgtgtctgtgtcggcgcaggtaccaggtggaggtgttcgaggcggcgctgcaggggaacaccatcgcggtgctcgacaccggctccgg

SEQ ID NO: 30 - DNA, 22 bp, synthetic construct
  source 1..22: mol_type = other DNA
tgagatattrgaggaaamcaaa

SEQ ID NO: 31 - DNA, 22 bp, synthetic construct
  source 1..22: mol_type = other DNA
tttataytaaacttttwtaytt

SEQ ID NO: 32 - DNA, 4881 bp, synthetic construct
  source 1..4881: mol_type = other DNA
atggtcatggccatggccgacgacgacggccccatccccccgcctcctcctccgccgccgcctcgtcgcccccacatgcagctccagccgaggaggtatcaggtggaggtgttcgaggcggcgctgcaggggaacaccatcgcagtgctcgacaccggctccgggaagaccatggtcgccgtcctgctcgcgcgctaccacgtcagccgcgcgcgcgccggggaggcgccccgccggatcgtcgtcttcctcgcgcctaccgtgcacctcgtccaccagcaattcgaggtgattcgggagtacaccgacctcgatgtcgcggagtgttacggagcttcgggtgtcggcgactggagtaccgaccgctggaacaaggaagttgggagcaaggagattgctgttatgacgccgcagatattgttggatgccctgaggcatgccttcataacaatgagtgcagtgagcctgctaatatttgatgaatgccatcgtgcctgtggaaaccacccgtatacacgaataatgaaggaattttatgttggctctgagtggaggccagctgtatttggaatgacagcatctcctgttgctactaaaggcacttccacggtacaagattgtgaagcacacattggtcaacttgaacttacattggatgcaaaggtatatatcataaaggatcgtagtgaacttgagagcttttcccctcctgcaacaattgtgaacaaatactatgatgcttactcgattgatttcgaagatctgaaatcgaagctacagatattgtatgaagagtatgatgctttgttggctaatctgcaagaatcatcaccaaataaatttgaagacacgaataacatattagagacatcaagaaagaccttgtctagataccatgagaagatattttatggcctaaattatcttggtccaatcatcaccgctgaggtagtgaagatatataatgaaagcattaagacattggatgattctgaagattgcctcttctctaaagctagcttcagtctgcatgtagcttattttaaagaagctttagatttaatcgaggcagtattgccacatggttatggtgaactaatgaaatcagaatctggtaccgcggaattaagtaaaagggggtatatttcttcaaaagtggatgctctaatcaacatttttaaatcgtttgggtcatcaaacgaagtgctttgcctaatttttgtagaaagaattatgacagctaaagctgttgagaggttcatgagaggaatttttaatttctctagtttttcaatttcttacttgactggagggagtacatcaaaagattctctgagcccagcagtgcagagatatactttggacttgttccgatctggaaaggtgaacttgcttttcagcacagatgtaactgaagagggtattgatgtacctaactgttcttgtgtcatacgctttgacctgcccagaactgtttgtagctatgttcaatctcgtggtcgtgccagaaggagaagctcaaattatgttcttatgattgagaggggaaacatggatcagcaggaacacatattccgcataatacagactgagtactatattaaacacttcgctctgtatagacaggaaacccccaatgtttcatcctctgagttgcctatgcaagataagtatgcataccatgttgattcaacaggagcaactataactgcagactgctgtgtcgatctgattcgtaaatattgtgagaaacttcctaaagataggtattacatgccaaagccttcctttgtggtggctatagaagatggatcataccaatgtaccttgactttgcctccaaatgcggcgtttcaaaggatagttggccctttatgcagtacaagtaatttagccaagcagcttgtatccctagatgcgtgcaagaaactgcatcagctaggagaactcaatgatcatcttctaccgttgaccgaagaacctattgatattgatgttgctttgagagatggaaaattcctttctggaccaggaacgactaaaagaaaggagctccatggaacaagaaatgttcttgccctgtcaggatcgtggattcatgaaagtgaaagtgtcacattgaatacttatagattcgattttctttgtgaccaagagggtgaaaactatgctgggtttgttctcttaatggagtcagcacttgatgatgacgtagcttgttcaaaaatggatctattcctgatccctaataaaatggtctacaccaccataactccgtgtggaaaagttcaattggacaaaaaacagctacgtgacgggaagttgttccaagaattatttttcaatggaatatttggtagattatttcatgggtctcgaacaagtggagtgcaaagggaatttcttttcagaaaaggtcatgaaatacaatggagctcagagtgcatgtacttacttttacctttgaggcattcaccacatatccagcaagatttaaacatatactgggaggcagttgaatcctgcacccaggtggtggagcagttgagaaatttgtatctggaagatgaaaatatctgtgcaaattctattcaacatagaagtatcaaggagaaagacattattcatctagccaacaaatctcttcatttttctagcatcaaagattcagttgtgctgtcgcttcatactgggaggatatactctgttcttgacttgatctatgacacaactgctgaggactcgtttgaagagatgtataatggaaaagcttcaccgttttcttcatttgtggactactaccgtgaaaagtacggtattattattcaacatccaaaacaaccgttgttgctgttaaagcaaagccacaatgcacacaatcttcttttttcaaaatcaaaatacatagatggttctacgggtgatcctttgctcatggaaaaagaacaaatacatgcccgggtcccacctgaattgttaatccacattgatgtgacaattgaagttctcaaatcattttatttattgccttctgtaatgcatcgacttcagtctctcatgctagccagccagcttcgcagagatattggttacactcaacatataccaagttacctgattttggaagctatcacaaccttaaggtgctgcgagacattttccctggagcgtttagaactattgggagactcagtactaaaatatttgataggatgtgacctgtttttaaggtatcctatgaaacatgaaggtcatctttctgatatgagatcaaatgctgtctgcaatgctacacttcataagcacggaatctggcgatccttgcagggttacgtgcgggatagtgcattcgacccacggcgttgggttgctcctgggcagatatccttgcgcccttttccttgtaactgcggaattgagactgcatttgttccaaccaatggaaggtatatcagcgacgacccatcttttgtcgttggaaaaccatgtgatagagctcacagatggatgtgctcgaaaacaatatctgattgtgttgaagccctagttggagcatattatgttggtggtggcatagttgctgcactttgggttatgaagtggttcggtattgaaataaaatgtgataggaagttggtacaggaagtaaagctcaatgcatcttatatatgctacttacctaaagtaagtgttattgaggagttggaagcaaaattgaagtacaacttctcggtcaagggccttctactggaagccataactcatccatctctgcaggaattaggggttgactactgttaccagcgtctggaatttctgggtgattctgtgctggacctactaattacacgctatctctatgttacccatactgatgttgatcctggagaattgacagacttacgttccgctttggttagtaacgagaattttgcagaagtagttttaagaaacaacattcacagtcatctacagcatgggtctggaatactcttggagcaaatcacagaatatgttaaatccaatttggagtgccaagggaaggttaataaattccttcaacatgctacatgtaaagtacctaaggtacttggagacattatggaaagcattgccggtgcaatatttgtagacacagattttgatgttgatgtggtttggaagattgttgaaccattgctttccccgatgataacccctgataatcttgcattgccaccctatcgggagttgttagagctgtgtagtcaccttggttatttcataaattcaaaatgcagtagtagaggagaagaagtaactatagatatgtcagtgcaattacgagatgaactgctgatagcccaaggacatgacagaaacaagaagagtgcaaaggcaaaagcagcagctcgtatattggcagatctgaagaaaagaggtctttcgatgaaacaatgttcctctaaatctaagcagttagatattatatctccggacctgcagtatcctttgttggaatcaccacttgattattgtgacgtgaatgtcaatcctagcctaggaggccttccctcactgaaggaagcagttgttcttcagctcaaaatggacaaaggtggaccacgaagtgctctttttaagctatgcaagaggttgcagtggcccatgccagaattcgaatttgtggaacaaaggttcaggactcctattgttctggacggggtaaccacaacgaacttcaatagcttcgtgtcaaccatcaccttgcacatacctgatgtaactgccattacaattcaaggcaaacggcaaactgacaaaaagagctcccaggattcagcgtcgctgataatgctccacaagcttcaagagctcaaggtctgtatatgcaagacttag

SEQ ID NO: 33 - DNA, 4881 bp, synthetic construct; identical to SEQ ID NO: 32
  source 1..4881: mol_type = other DNA

SEQ ID NO: 34 - DNA, 4884 bp, synthetic construct
  source 1..4884: mol_type = other DNA
atggccatggccgacgacgacgacggccccctgcccccgcctccgccgccgccgccgcctcgccgtccccacaggcagctccagccgaggaggtaccaggtggaggtgttcgaggcggcgctgcaggggaacaccatcgcggtgctcgacaccggctccgggaagaccatggtcgccgtcctgctcgcgcgccaccacgtgggccgcgtgcgcgccggggaggtgccccgccggatcgtcttcttcctcgcgcccaccgtgcacctcgtccaccagcaattcgaggtgattcgggagtacacagacctcgacgccgcggagtgctacggagcttcgggggtcggcgactggagtacagaccgctggaacaaggaagttgggagcaaggagattgctgttatgacgccccagatattgttggatgccctgaggcatgcctttgtaacaatgagtgcagtgagcctgctaatatttgatgaatgccatcgtgcctgtggaaaccacccatatacacgaataatgaaggaattttaccttggttctcagtggaggccagctgtatttggaatgacagcatctcctgttgctactaaaggcacttgcacggtacaagattgtgaagcacacattggtcaacttgaacttacattggatgcaaaggtatatatcataaaagatcgtagtgaacttgagagcttttcccctcctgcaacaattgtgcacgaatactatgatgcttactcgattgatttcgaagatctgaaatcgaagctccagatattgtatgaagagtatgatgctttgttggctagtctgcaagaatcgtcaccaaataaatttgaagacacgaataacatattagagacatcaagaaagaccttgttcagataccatgagaagatattttatggcctaaattatcttggtccaatcatcaccgctgaggtagtgaagatatataatgaaagcattaagacactgggtgattctgaagattgtctcttctcgaaagctagcttcagcctgcatgtagcttattttaaagaagctttagatttaattgaggaagtattgccacatggttatggtgaactaatgaaatcagaatctggttctgcggaattaattaaaagggggtatatttcttcaaaagtggatgctctaatcaacatttttaaatcatttgggtcatcaaacgaagtgctttgcctaatttttgtagatagaattatgacagctaaagctgttgagaggttcatgagaggaattgttagtttctctagtttttcaatttcttacttgactggagggagtacatcaaaagattctctgagcccagcagtgcagagatatactttagacttgttccgatctggaaaggtgaacttgcttttcaccacagatgtaactgaagagggtattgatgtacctaactgttcttgtgtcatacgcttcgacctgcccagaactgtttgtagctacgttcaatctcgtggtcgtgccagaaggagcagctcaaattatgttcttatgattgagaggggaaacatggatcagcaggaacacatattccgcataatacggactgagtactatattaaacacttcgctctgtataggcacaaaaaccccaatgtttcatcctctgagtggcctatgcaagataagtatgcatactatgttgattcaacaggagcaactataactgcagactgctgtgttgatctgatccgtaaatattgtgagaaacttcctaaagataggtattacatgccaaagccttcctttgcggtggctatcgaagatggatcataccaatgcaccttgactttgcctccaaatgcggcgtttcaaaggatagttggccctttatgcagtacaagtaatttagccaagcagcttgtatccctagatgcgtgcaagaaattgcatcagctaggagaactcaatgatcatcttctacccttcaccgaagaacctattgaaattgatgttgctttaagagatgggaaattcctttctggaccaggaacgactaaaagaaaggagctccatggaacaagaaatgttcttgccctgtcaggatcgtggattcatgaaagtgaaagtgtcacattgaatgcttatagattcgattttctttgtgaccaagagggtgaaaactatgctgggtttgttctcttaatggaatcagcacttgatgatgatgtagcttgttctaaaatggatctattcctgatccctaataaaatggtgtacaccaccgtaactccgtgtggaaaagttcaactggacaaaaaacagctacgtgacgggaagttgttccaagaattctttttcaatggaatatttggtagattatttcatggatctcgaacaagtggagtgcaaagggaatttcttttcagaaaaggtcatgaaatacaatggagctcagagagcatgtacttacttttacctttgaggcattcaccacacatccagcaagatttaaacatatactgggaggcagttgaatcctgcacccaggcggtggagcagttgagaaatttgtatctggaagatgaaaatatccgtgcaaattttattcaacatagaagtatcaaggagaaagacattattcatctggccaacaaatctcttcatttttctagcatcaaagattcagttgtgctatcgcttcatactggaaggatatactctgttcttgacttgatctatgacacaactgctgaagactcgtttgaagagatgtataatggaaaagcttcaccgttttcttcattcatggactactaccgtgaaaagtatggtattgttattcaacatccaaagcaaccgttgttgctgttaaagcaaagccacaatgcacacaatcttcttttttcaaaatcgaagtacatagatggttctacgggtgatcctttgctcatggaaaaagagcaaatacacgcccgggtcccacctgaattgttaatccacattgatgtgacaattgaagttctcaaatctttttatttattgccttctgtaatgcatcgacttcagtctctcatgctagccagccagctccgcagagatattggttacactcaacatataccaagttacctgattttggaagctatcacaactttaaggtgctgcgaaacattttccctggagcgtttagaattattgggagactcagtactaaaatatgtgataggatgtgacctgtttttaaggtatcctatgaaacatgaaggtcatctttctgatatgagatcaaatgctgtctgcaatgctacgcttcataagcacggaatctggcgatccttgcagggttacgtgcgggatagtgcatttgacccacggcgttgggttgctcctgggcagatatctttgcgcccttttccttgtaactgcggaattgagactgcatttgttccaactaatggaaggtatatcagcgacgacccatcttttgtcgttggaaaaccatgtgatagagctcacagatggatgtgctcgaaagcaatatctgattgtgttgaagccctagttggagcatattatgtcggtggtggcatagttgctgcactttgggttatgaagtggttcggtattgaaataaaatgcgataggaagttggtacaggaagtaaagctcaacgcatcttatatatgctacttacctaaaataagtgttattgaggagttggaagcaaaactgaagtacaacttctcagtcaagggccttctactggaagccataactcatccatctctgcaggaattaggggttgactactgttaccagcgtctggaatttctgggtgattctgtgctggatctactaattacacgctatctctatgttacccatactgatgttgatcctggagaattgacagacttacgttccgctttggttagtaatgagaattttgcagaagtagttttaagaaataacattcacagtcatctacagcatgggtctggaatacttttggagcaaatcacagaatatgttagatccaatttggagtgccaagggaaggttaataaattccttcaacatgctacatgtaaagtacctaaggtgcttggagacattatggaaagcattgccggtgcaatatttgtagacacagattttaatgttgatgtggtttggaagattgttgaaccgttgctttccccaatgataacccctgataatcttgcattgccaccttatcgggagttgttagagctgtgtagtcaccttggttatttcataaattcaaaatgcagtagtaaaggagaagaagtaagtatagagatgtcagtgcaattacgagatgaactgctgatagcgcaaggacaagacagaaataagaagagtgcaaaggcaaaagcagcagctcgtatattggcagatctgaagaaaagaggcctttcaatgaaacaatgtccttctaaagctaagcagtcgggtattatatcttcggaccagcagtatcctttggcaagtttggaatcaccacttgattattgtcaggtgaatgtcaatcctagcctaggaggccttccctcactgaaagaagcagttgttcttcggctcaaaatggacaaaggtggaccacgaagtgctctttttaagctatgcaagaggttgcagtggccaatgccagaattcgaatttgtggaacaaaggttcaggactcctattgttctggacggggtaaccacaacgaacttcaatagcttcgtgtcaaccatcaccttgcacatacctgatgtagcagccattacacttcaaggtgagcggcgaactgacaaaaagagttcccaggattcagcatcgctgacaatgctccacaagcttcaagagctcaaggtctgtatatgcaagacttag

SEQ ID NO: 35 - DNA, 5286 bp, synthetic construct
  source 1..5286: mol_type = other DNA
accccacccccgcctcgcccactcgcccgcaccgaacccaaccccatggccatggccgacgacgacgacggccccctgcccccgcctccgccgccgccgccgcctcgccgtccccacaggcagctccagccgaggaggtaccaggtggaggtgttcgaggcggcgctgcaggggaacaccatcgcggtgctcgacaccggctccgggaagaccatggtcgccgtcctgctcgcgcgccaccacgtgggccgcgtgcgcgccggggaggtgccccgccggatcgtcttcttcctcgcgcccaccgtgcacctcgtccaccagcaattcgaggtgattcgggagtacacagacctcgacgccgcggagtgctacggagcttcgggggtcggcgactggagtacagaccgctggaacaaggaagttgggagcaaggagattgctgttatgacgccccagatattgttggatgccctgaggcatgcctttgtaacaatgagtgcagtgagcctgctaatatttgatgaatgccatcgtgcctgtggaaaccacccatatacacgaataatgaaggaattttaccttggttctcagtggaggccagctgtatttggaatgacagcatctcctgttgctactaaaggcacttgcacggtacaagattgtgaagcacacattggtcaacttgaacttacattggatgcaaaggtatatatcataaaagatcgtagtgaacttgagagcttttcccctcctgcaacaattgtgcacgaatactatgatgcttactcgattgatttcgaagatctgaaatcgaagctccagatattgtatgaagagtatgatgctttgttggctagtctgcaagaatcgtcaccaaataaatttgaagacacgaataacatattagagacatcaagaaagaccttgttcagataccatgagaagatattttatggcctaaattatcttggtccaatcatcaccgctgaggtagtgaagatatataatgaaagcattaagacactgggtgattctgaagattgtctcttctcgaaagctagcttcagcctgcatgtagcttattttaaagaagctttagatttaattgaggaagtattgccacatggttatggtgaactaatgaaatcagaatctggttctgcggaattaattaaaagggggtatatttcttcaaaagtggatgctctaatcaacatttttaaatcatttgggtcatcaaacgaagtgctttgcctaatttttgtagatagaattatgacagctaaagctgttgagaggttcatgagaggaattgttagtttctctagtttttcaatttcttacttgactggagggagtacatcaaaagattctctgagcccagcagtgcagagatatactttagacttgttccgatctggaaaggtgaacttgcttttcaccacagatgtaactgaagagggtattgatgtacctaactgttcttgtgtcatacgcttcgacctgcccagaactgtttgtagctacgttcaatctcgtggtcgtgccagaaggagcagctcaaattatgttcttatgattgagaggggaaacatggatcagcaggaacacatattccgcataatacggactgagtactatattaaacacttcgctctgtataggcacaaaaaccccaatgtttcatcctctgagtggcctatgcaagataagtatgcatactatgttgattcaacaggagcaactataactgcagactgctgtgttgatctgatccgtaaatattgtgagaaacttcctaaagataggtattacatgccaaagccttcctttgcggtggctatcgaagatggatcataccaatgcaccttgactttgcctccaaatgcggcgtttcaaaggatagttggccctttatgcagtacaagtaatttagccaagcagcttgtatccctagatgcgtgcaagaaattgcatcagctaggagaactcaatgatcatcttctacccttcaccgaagaacctattgaaattgatgttgctttaagagatgggaaattcctttctggaccaggaacgactaaaagaaaggagctccatggaacaagaaatgttcttgccctgtcaggatcgtggattcatgaaagtgaaagtgtcacattgaatgcttatagattcgattttctttgtgaccaagagggtgaaaactatgctgggtttgttctcttaatggaatcagcacttgatgatgatgtagcttgttctaaaatggatctattcctgatccctaataaaatggtgtacaccaccgtaactccgtgtggaaaagttcaactggacaaaaaacagctacgtgacgggaagttgttccaagaattctttttcaatggaatatttggtagattatttcatggatctcgaacaagtggagtgcaaagggaatttcttttcagaaaaggtcatgaaatacaatggagctcagagagcatgtacttacttttacctttgaggcattcaccacacatccagcaagatttaaacatatactgggaggcagttgaatcctgcacccaggcggtggagcagttgagaaatttgtatctggaagatgaaaatatccgtgcaaattttattcaacatagaagtatcaaggagaaagacattattcatctggccaacaaatctcttcatttttctagcatcaaagattcagttgtgctatcgcttcatactggaaggatatactctgttcttgacttgatctatgacacaactgctgaagactcgtttgaagagatgtataatggaaaagcttcaccgttttcttcattcatggactactaccgtgaaaagtatggtattgttattcaacatccaaagcaaccgttgttgctgttaaagcaaagccacaatgcacacaatcttcttttttcaaaatcgaagtacatagatggttctacgggtgatcctttgctcatggaaaaagagcaaatacacgcccgggtcccacctgaattgttaatccacattgatgtgacaattgaagttctcaaatctttttatttattgccttctgtaatgcatcgacttcagtctctcatgctagccagccagctccgcagagatattggttacactcaacatataccaagttacctgattttggaagctatcacaactttaaggtgctgcgaaacattttccctggagcgtttagaattattgggagactcagtactaaaatatgtgataggatgtgacctgtttttaaggtatcctatgaaacatgaaggtcatctttctgatatgagatcaaatgctgtctgcaatgctacgcttcataagcacggaatctggcgatccttgcagggttacgtgcgggatagtgcatttgacccacggcgttgggttgctcctgggcagatatctttgcgcccttttccttgtaactgcggaattgagactgcatttgttccaactaatggaaggtatatcagcgacgacccatcttttgtcgttggaaaaccatgtgatagagctcacagatggatgtgctcgaaagcaatatctgattgtgttgaagccctagttggagcatattatgtcggtggtggcatagttgctgcactttgggttatgaagtggttcggtattgaaataaaatgcgataggaagttggtacaggaagtaaagctcaacgcatcttatatatgctacttacctaaaataagtgttattgaggagttggaagcaaaactgaagtacaacttctcagtcaagggccttctactggaagccataactcatccatctctgcaggaattaggggttgactactgttaccagcgtctggaatttctgggtgattctgtgctggatctactaattacacgctatctctatgttacccatactgatgttgatcctggagaattgacagacttacgttccgctttggttagtaatgagaattttgcagaagtagttttaagaaataacattcacagtcatctacagcatgggtctggaatacttttggagcaaatcacagaatatgttagatccaatttggagtgccaagggaaggttaataaattccttcaacatgctacatgtaaagtacctaaggtgcttggagacattatggaaagcattgccggtgcaatatttgtagacacagattttaatgttgatgtggtttggaagattgttgaaccgttgctttccccaatgataacccctgataatcttgcattgccaccttatcgggagttgttagagctgtgtagtcaccttggttatttcataaattcaaaatgcagtagtaaaggagaagaagtaagtatagagatgtcagtgcaattacgagatgaactgctgatagcgcaaggacaagacagaaataagaagagtgcaaaggcaaaagcagcagctcgtatattggcagatctgaagaaaagaggcctttcaatgaaacaatgtccttctaaagctaagcagtcgggtattatatcttcggaccagcagtatcctttggcaagtttggaatcaccacttgattattgtcaggtgaatgtcaatcctagcctaggaggccttccctcactgaaagaagcagttgttcttcggctcaaaatggacaaaggtggaccacgaagtgctctttttaagctatgcaagaggttgcagtggccaatgccagaattcgaatttgtggaacaaaggttcaggactcctattgttctggacggggtaaccacaacgaacttcaatagcttcgtgtcaaccatcaccttgcacatacctgatgtagcagccattacacttcaaggtgagcggcgaactgacaaaaagagttcccaggattcagcatcgctgacaatgctccacaagcttcaagagctcaaggtctgtatatgcaagacttagcaaagcatcatggatccacttgccagctaactgctaaggctcgttaacatggggatctgatcctccatcccacagtgaattatgttgaaattttttatgagaggcccagtggtggtgctagattatcctgcaggatatctcagaaacaattagtgcttgtcttccgaaaaaaacaatagacaatttcacttgctggacaaagaaaacattttgcaagaattgatatcggtttggtttgaatccacatttgggtcaactatcgaccttaggttcttagtggaattgagtgcaatttttttagctccttagcagagttcacatgtagttacatttctctacttcactccacaaatgtcc

SEQ ID NO: 36 - DNA, 3111 bp, synthetic construct
  source 1..3111: mol_type = other DNA
atgccaaagccttcctttgcggtggctatcgaagatggatcataccaatgcaccttgactttgcctccaaatgcggcgtttcaaaggatagttggccctttatgcagtacaagtaatttagccaagcagcttgtatccctagatgcgtgcaagaaattgcatcatctaggagaactcaatgatcatcttctacccttcaccgaagaacctattgatattgatgttgctttaagagatgggaaattcctttctggaccaggaacgactaaaagaaaggagctccatggaacaagaaatgttcttgccctgtcaggatcgtggattcatgaaagtggaagtgtcacattgaatgcttatagattcggttttctttgtgaccaagagggtgaaaactatgctgggtttgttctcttaatggaatcagcacttgatgatgatgtagcttattcaaaaatggatctattcctgatccctaataaaatggtctacaccaccgtaactccgtgtggaaaagttcaactggacaaaaaacagctacgtgacgggaagttgtttcaagaattctttttcaatggaatatttggtagattatttcatggatctcgaacaagtggagtgcaaagggaatttcttttcagaaaaggtcatgaaatacaatggagctcagagagcatgtacttacttttacctttgaggcattcaccacacatccagcaagatttaaacatatactgggaggcagttgaatcctgcacccaggcggtggagcggttgagaaatttgtatctggaagaggaaaatatccgtgcaaattttattcaacatagaagtatcaaggagaaagacattattcatctggccaacaaatctcttcatttttctagcatcaaagattcagttgtgctatcgcttcatactggaaggatatactctgttcttgacttgatctatgacataactgctgaagacgcgtttgaagagatgtataatggaaaagcttcaccgttttcttcattcgtggactactatcgtgaaaagtatggtattgttattcaacatccaaaacaaccgttgttgctgttaaagcaaagccacaatgcacacaatcttcttttttcaaaatcgaagtacatagatggttctacgggcgatcctttgcttatggaaaaagagcaaatacacgcccgggtcccacctgaattgttaatccacattgatgtgacaattgaagttctcaaatctttttacttattgccttctgtaatgcatcgacttcagtctctcatgctagccagccagctccgcagagatattggttacactcaacatataccaagttacctgattttggaagctatcacaactttaaggtgctgtgaaacattttccctggagcgtttagaactattgggagactcagtactaaaatatgtgataggatgtgacctgtttttaaggtatcctatgaaacatgaaggtcatctttctgatatgagatcaaatgctgtctgcaatgctacacttcataagcacggaatctggcgatccttgcagggttacgtgcgggatagtgcatttgacccacggcgttgggttgctcctgggcagatatctttgcgcccttttccttgtaactgcggaattgagactgcatttgttccaactaatggaaggtatatcagcgacgacccatcttttgtagttggaaaaccatgtgatagagctcacagatggatgtgctcgaaagcaatatctgattgtgttgaagccctagttggagcatattatgttggtggtggcatagttgctgcactttgggttatgaagtggttcggtattgaaataaaatgcgataggacgttggtacaggaagtaaagctcaatgcatcttatatatgctacttacctaaaataagtgttattgaggagttggaagcaaaactgaagtacaacttctcggtcaagggccttctactggaagccataactcatccatctctgcaggaattaggggttgactactgttaccagcgtctggaatttctgggtgattctgtgctggatctactaattacacgctatctctatgttacccatactgatgttgatcctggagaattgacagacttacgttccgctttggttagtaacgagaattttgcagaagtagttttaagaaacaacattcacagtcatctacagcatgggtctggaatacttttggagcaaatcacagaatatgttagatccaatttggagtgccaagggaaggttaataaattccttcaacatgctacatgtaaagcacctaaggtacttggtgacattatggaaagcattgccggtgcaatatttgttgacgcagattttaatgttgatgtggtttggaagattgttgaaccgttgctttccccaatgataacccctgataatcttgcattgccaccttatcgggagttgttagagctgtgtagtcaccttggttatttcataaattcaaaatgcagtagtaaaggagaagaattaagtatagagatgtcagtgcaattacgagatgaactgttgatagcgcaaggacaagacagaaacaagaagagtgcaaaggcaaaagcagcagctcgtatattggcagatctgaagaaaagaggcctttcaatgaaacaatgttcttctaaagctaagcagttggatattatatcatcggagcagtatcctttggcaagtttggaatcaccacttgattattgtcacgtgaatgccaatcctagcctaggaggccttccctcactgaaagaagcagttgttcttcggcttaaaatggacaaaggtggaccacgaagtgctctttttaagctatgcaagaggttgccgtggccaatgccagaattcgaatttgttgaacaaaggttcaggactcctattgttctggacggggtaaccacaacgaacttcaatagcttcgtgtcaaccatcaccttgcacatacctgatgtaacagccattacacttcaaggcgagcggcggactgacaaaaggagttcccaggattcagcatcgctggcaatgctccacaagcttcaagagctcaaggtctgtatatgcaagacttag

SEQ ID NO: 37 - DNA, 3329 bp, synthetic construct
  source 1..3329: mol_type = other DNA
atgccaaagccttcctttgcggtggctatcgaagatggatcataccaatgcaccttgactttgcctccaaatgcggcgtttcaaaggatagttggccctttatgcagtacaagtaatttagccaagcagcttgtatccctagatgcgtgcaagaaattgcatcatctaggagaactcaatgatcatcttctacccttcaccgaagaacctattgatattgatgttgctttaagagatgggaaattcctttctggaccaggaacgactaaaagaaaggagctccatggaacaagaaatgttcttgccctgtcaggatcgtggattcatgaaagtggaagtgtcacattgaatgcttatagattcggttttctttgtgaccaagagggtgaaaactatgctgggtttgttctcttaatggaatcagcacttgatgatgatgtagcttattcaaaaatggatctattcctgatccctaataaaatggtctacaccaccgtaactccgtgtggaaaagttcaactggacaaaaaacagctacgtgacgggaagttgtttcaagaattctttttcaatggaatatttggtagattatttcatggatctcgaacaagtggagtgcaaagggaatttcttttcagaaaaggtcatgaaatacaatggagctcagagagcatgtacttacttttacctttgaggcattcaccacacatccagcaagatttaaacatatactgggaggcagttgaatcctgcacccaggcggtggagcggttgagaaatttgtatctggaagaggaaaatatccgtgcaaattttattcaacatagaagtatcaaggagaaagacattattcatctggccaacaaatctcttcatttttctagcatcaaagattcagttgtgctatcgcttcatactggaaggatatactctgttcttgacttgatctatgacataactgctgaagacgcgtttgaagagatgtataatggaaaagcttcaccgttttcttcattcgtggactactatcgtgaaaagtatggtattgttattcaacatccaaaacaaccgttgttgctgttaaagcaaagccacaatgcacacaatcttcttttttcaaaatcgaagtacatagatggttctacgggcgatcctttgcttatggaaaaagagcaaatacacgcccgggtcccacctgaattgttaatccacattgatgtgacaattgaagttctcaaatctttttacttattgccttctgtaatgcatcgacttcagtctctcatgctagccagccagctccgcagagatattggttacactcaacatataccaagttacctgattttggaagctatcacaactttaaggtgctgtgaaacattttccctggagcgtttagaactattgggagactcagtactaaaatatgtgataggatgtgacctgtttttaaggtatcctatgaaacatgaaggtcatctttctgatatgagatcaaatgctgtctgcaatgctacacttcataagcacggaatctggcgatccttgcagggttacgtgcgggatagtgcatttgacccacggcgttgggttgctcctgggcagatatctttgcgcccttttccttgtaactgcggaattgagactgcatttgttccaactaatggaaggtatatcagcgacgacccatcttttgtagttggaaaaccatgtgatagagctcacagatggatgtgctcgaaagcaatatctgattgtgttgaagccctagttggagcatattatgttggtggtggcatagttgctgcactttgggttatgaagtggttcggtattgaaataaaatgcgataggacgttggtacaggaagtaaagctcaatgcatcttatatatgctacttacctaaaataagtgttattgaggagttggaagcaaaactgaagtacaacttctcggtcaagggccttctactggaagccataactcatccatctctgcaggaattaggggttgactactgttaccagcgtctggaatttctgggtgattctgtgctggatctactaattacacgctatctctatgttacccatactgatgttgatcctggagaattgacagacttacgttccgctttggttagtaacgagaattttgcagaagtagttttaagaaacaacattcacagtcatctacagcatgggtctggaatacttttggagcaaatcacagaatatgttagatccaatttggagtgccaagggaaggttaataaattccttcaacatgctacatgtaaagcacctaaggtacttggtgacattatggaaagcattgccggtgcaatatttgttgacgcagattttaatgttgatgtggtttggaagattgttgaaccgttgctttccccaatgataacccctgataatcttgcattgccaccttatcgggagttgttagagctgtgtagtcaccttggttatttcataaattcaaaatgcagtagtaaaggagaagaattaagtatagagatgtcagtgcaattacgagatgaactgttgatagcgcaaggacaagacagaaacaagaagagtgcaaaggcaaaagcagcagctcgtatattggcagatctgaagaaaagaggcctttcaatgaaacaatgttcttctaaagctaagcagttggatattatatcatcggagcagtatcctttggcaagtttggaatcaccacttgattattgtcacgtgaatgccaatcctagcctaggaggccttccctcactgaaagaagcagttgttcttcggcttaaaatggacaaaggtggaccacgaagtgctctttttaagctatgcaagaggttgccgtggccaatgccagaattcgaatttgttgaacaaaggttcaggactcctattgttctggacggggtaaccacaacgaacttcaatagcttcgtgtcaaccatcaccttgcacatacctgatgtaacagccattacacttcaaggcgagcggcggactgacaaaaggagttcccaggattcagcatcgctggcaatgctccacaagcttcaagagctcaaggtctgtatatgcaagacttagcaaagcatcatggatccacttgcgagctaactgctaaggctcgttaacctggggatctgatcctccaccccaaagtgaattatgttgaaaatgttttatgaaaggctcggtgtggtgctagattatcctgcaggatatcttagaaatagttaatgcctgtcttctgaaaataacagtctacaatttcacttgctggacaatgaaaacattttgcaaga

SEQ ID NO: 38 - DNA, 4887 bp, synthetic construct
  source 1..4887: mol_type = other DNA
atggccatggccatggccgacgacgacggccccatccccccgcctccgccgccgccgccgcctcgccgcccccacaggcagctccagccgaggaggtaccaggtggaggtgttcgaggcggcgctgcaggggaacaccatcgcggtgctcgacaccggctccggaaagaccatggtcgccgtcctgctcgcgcgccaccacgtgggccgcgtgcgcgccggggaggtgccccgccggatcgtcgtcttcctcgcgcccaccgtgcacctcgtccaccagcaattcgaggtgattcgggagtacacggacctcgacgccgctgagtgctacggagcttcgggggtcggcgactggagtaccgaccgctggaacaaggaagttgggagcaaggagattgctgttatgacgccccagatattgttggatgccctgaggcatgcctttgtaacaatgagtgcagtgagcctgctaatatttgatgaatgccatcgtgcctgtggaaaccacccatatacacgaataatgaaggaattttaccttggctctcagtggaggccagctgtatttggaatgacagcatctcctgttgctactaaaggcacttgcacggtacaagattgtgaagcacacattggtcaacttgaacttacattggatgcgaaggtatatatcataaaagatcgtagtgaacttgagagcttttcccctcctgcaacaattgtgaacaaatactatgatgcttactcgattgatttcgaagatctgaaatcgaagctccagatattgtatgaagagtatgatgctttgttggctagtctgcaagaatcgtcaccaaataaatttgaagacacgaataacatattagagacatcaagaaagaccttgttcagataccatgagaagatattttatggcctaaattatcttggtccaatcatcaccgctgaggtagtgaagatatataatgaaagcattaagacactgggtgattctgaagattgcctcttctcgaaagctagcttcagcctgcatgtagcttattttaaagaagctttagatttagttgaggaagtattgccacatggttatggtgaactaatgaaatcagaatctggttctgcggaattaattaaaagcgggtatatttcttcaaaagtggatgctctaatcaacatttttaaatcatttgggtcatcaaacgaagtgctttgcctaatttttgtagaaagaattatgacagctaaagctgttgagaggttcatgagaggaattgttaatttctctagtttttcaatttcttacttgactggagggagtacatcaaaagattctctgagcccagcagtgcagagatatactttggacttgttccgatctggaaaggtgaacttgcttttcaccacagatgtaactgaagagggtattgatgtacctaactgttcttgtgtcatacgcttcgacctgcccagaactgtttgtagctacgttcaatctcgtggtcgtgccagaaggagcagctcaaattatgttcttatgattgagaggggaaacatggatcagcaggaacacatattccgcataatacagactgagtactatattaaacacttcgctctgtataggcacagaaaccccaatgtttcatcctctgagtggcctatgcaagataagtatgcataccatgttgattcaacaggagcaactataactgcagactgctgtgttgatctgatccgtaaatattgtgagaaacttcctaaagataggtattacattccaaagccttcctttgcggtggctatcgaagatggatcataccaatgcaccttgactttgcctccaaatgcggcgtttcaaaggatagttggccctttatgcagtacaagtaatttagccaagcagcttgtatccctagatgcgtgcaagaaattgcatcagctaggagaactcaatgatcatcttctacccttcaccgaagaacctattgatattgatgttgctttaagagatgggaaattcctttctggaccaggaacgactaaaagaaaggagctccatggaacaagaaatgttcttgccctgtcaggatcgtggattcatgaaagtgaaagtgtcacattgaatgcttatagattcgattttctttgtgaccaagagggtgaaaactatgctgggtttgttctcttaatggaatcagcacttgatgatgatgtagcttgttcaaaaatggatctattcctgatccctaataaaatggtctacaccaccgtaactccgtgtggaaaagttcaactggacaaaaaacagctacgtgacgggaagttgttccaagaattctttttcaatggaatatttggtagattatttcatggatctcgaacaagtggagtgcaaagggaatttcttttcagaaaaggtcatgaaatacaatggagctcacagagcatgtacttacttttacctttgaggcattcaccacacatccagcaagatttaaacatatactgggaggcagttgaatcctgcacccaggcggtggagcagttgagaaatttgtatctggaagatgaaaatatccgtgcaaattttattcaacatagaagtatcaaggagaaagacattattcatctggccaacaaatctcttcatttttctagcatcaaagattcagttgtgctatcgcttcatactggaaggatatactctgttcttgacttgatctatggcacaactgctgaagactcgtttgaagagatgtataatggaaaagcttcaccgttttcttcattcgtggactactaccgtgaaaagtatggtattgttattcaacatccaaaacaaccgttgttgctgttaaagcaaagccacaatgcacacaatcttcttttttcaaaatcgaagtacatagatggttctacgggcgatcctttgctcatggaaaaagagcaaatacacgcccgggtcccacctgaattgttaatccacattgatgtgacaattgaagttctcaaatctttttatttattgccttctgtaatgcatcgacttcagtctctcatgctagccagccagctccgcagagatattggttacactcaacatataccaagttacctgattttggaagctatcacaactttaaggtgctgcgaaacattttccctggagcgtttagaactattgggagactcagtactaaaatatgtgataggatgtgacctgtttttaaggtatcctatgaaacatgaaggtcatctttctgatatgagatcaaatgctgtctgcaatgctacacttcataagcacggaatctggcgatccttgcagggttacgtgcgggatagtgcatttgacccacggcgttgggttgctcctgggcagatatctttgcgcccttttccttgtaactgcagaattgagactgcatttgttccaagtaatggaaggtatatcagcgacgacccatcttttgtcgttggaaaaccatgtgatagagctcacagatggatgtgctcgaaagcaatatctgattgtgttgaagccctagttggagcatattatgtcggtggtggcatagttggtgcactttgggttatgaagtggttcggtattgaaataaaatgtgataggaagttggtacaggaagtaaagctcaatgcgtcttatatatgctacttacctaaaataagtgttattgaggagttggaagcaaaactgaagtacaacttctcagtcaagggccttctactggaagccataactcatccatctctgcaggaattaggggctgactactgttaccagcgtttggaatttctgggtgattctgtgctggatctactaattacacgctatctgtatgttacccatactgatgttgatcctggagaattgacagacctacgttccgctttggttagcaatgagaattttgcagaagtagttttaagaaataacattcacagtcatctacagcatgggtctggaatacttttggagcaaatcacagaatatgttagatccaatttggagtgccaagggaaggttaataaattccttcaacatgctacatgtaaagtacctaaggtgcttggagacattatggaaagcattgccggtgcaatatttgtagacacagattttaatgttgatgtggtttggaagattgttgaaccgttgctttccccaatgataacccctgataatcttgtattgccaccttatcgggagttgttagagctgtgtagtcaccttggttatttcataaattcaaaatgcagtagtaaaggagaagaagtaattatagagatgtcagtgcaattacgagatgaactgctgatagcgcaaggacaagacagaaataagaagagtgcaaagtcaaaagcagcagctcgtatattggcagatctgaagaaaagaggcctttcaatgaaacaatgttcttctaaagctaagcagttggatattatatcttcggaccagcagtatcctttggcaagtttggaatcaccacttgattattgtcacgtggatgccaatcctagcctaggaggccttccctcactgaaagaagcagttgttcttcggcttaaactggacaaaggtggaccacggagtgctctttttaagctatgcaagaggttgcagtggccaatgccagaattcgaatttgtggaacaaaggttcaggactcctattgttctggacggggtaaccacaacgaacttcaatagcttcgtgtcaaccatcaccttgcacatacctgatgtaacagccattacacttcaaggcgagcggcgaactgacaaaaagagttcccaggattcagcatcgctgataatgctccacaagcttcaagagctcaaggtctgtatatgcaagacttag

SEQ ID NO: 39 - DNA, 5335 bp, synthetic construct
  source 1..5335: mol_type = other DNA
actcttgagtcattttcctccccccaaacctcctctccgcgccaacgaacgcagcgcaacccacacctccacttcacgcactttgccttctcccctcctccgcccagtctcccacctcccgtcccatccccggcgccgatttatatacacggctggcctacagacatgcgcttcccccacccccgcctcgcccactcgcccgcaccgaacccaaccccatggccatggccatggccgacgacgacggccccatccccccgcctccgccgccgccgccgcctcgccgcccccacaggcagctccagccgaggaggtaccaggtggaggtgttcgaggcggcgctgcaggggaacaccatcgcggtgctcgacaccggctccggaaagaccatggtcgccgtcctgctcgcgcgccaccacgtgggccgcgtgcgcgccggggaggtgccccgccggatcgtcgtcttcctcgcgcccaccgtgcacctcgtccaccagcaattcgaggtgattcgggagtacacggacctcgacgccgctgagtgctacggagcttcgggggtcggcgactggagtaccgaccgctggaacaaggaagttgggagcaaggagattgctgttatgacgccccagatattgttggatgccctgaggcatgcctttgtaacaatgagtgcagtgagcctgctaatatttgatgaatgccatcgtgcctgtggaaaccacccatatacacgaataatgaaggaattttaccttggctctcagtggaggccagctgtatttggaatgacagcatctcctgttgctactaaaggcacttgcacggtacaagattgtgaagcacacattggtcaacttgaacttacattggatgcgaaggtatatatcataaaagatcgtagtgaacttgagagcttttcccctcctgcaacaattgtgaacaaatactatgatgcttactcgattgatttcgaagatctgaaatcgaagctccagatattgtatgaagagtatgatgctttgttggctagtctgcaagaatcgtcaccaaataaatttgaagacacgaataacatattagagacatcaagaaagaccttgttcagataccatgagaagatattttatggcctaaattatcttggtccaatcatcaccgctgaggtagtgaagatatataatgaaagcattaagacactgggtgattctgaagattgcctcttctcgaaagctagcttcagcctgcatgtagcttattttaaagaagctttagatttagttgaggaagtattgccacatggttatggtgaactaatgaaatcagaatctggttctgcggaattaattaaaagcgggtatatttcttcaaaagtggatgctctaatcaacatttttaaatcatttgggtcatcaaacgaagtgctttgcctaatttttgtagaaagaattatgacagctaaagctgttgagaggttcatgagaggaattgttaatttctctagtttttcaatttcttacttgactggagggagtacatcaaaagattctctgagcccagcagtgcagagatatactttggacttgttccgatctggaaaggtgaacttgcttttcaccacagatgtaactgaagagggtattgatgtacctaactgttcttgtgtcatacgcttcgacctgcccagaactgtttgtagctacgttcaatctcgtggtcgtgccagaaggagcagctcaaattatgttcttatgattgagaggggaaacatggatcagcaggaacacatattccgcataatacagactgagtactatattaaacacttcgctctgtataggcacagaaaccccaatgtttcatcctctgagtggcctatgcaagataagtatgcataccatgttgattcaacaggagcaactataactgcagactgctgtgttgatctgatccgtaaatattgtgagaaacttcctaaagataggtattacattccaaagccttcctttgcggtggctatcgaagatggatcataccaatgcaccttgactttgcctccaaatgcggcgtttcaaaggatagttggccctttatgcagtacaagtaatttagccaagcagcttgtatccctagatgcgtgcaagaaattgcatcagctaggagaactcaatgatcatcttctacccttcaccgaagaacctattgatattgatgttgctttaagagatgggaaattcctttctggaccaggaacgactaaaagaaaggagctccatggaacaagaaatgttcttgccctgtcaggatcgtggattcatgaaagtgaaagtgtcacattgaatgcttatagattcgattttctttgtgaccaagagggtgaaaactatgctgggtttgttctcttaatggaatcagcacttgatgatgatgtagcttgttcaaaaatggatctattcctgatccctaataaaatggtctacaccaccgtaactccgtgtggaaaagttcaactggacaaaaaacagctacgtgacgggaagttgttccaagaattctttttcaatggaatatttggtagattatttcatggatctcgaacaagtggagtgcaaagggaatttcttttcagaaaaggtcatgaaatacaatggagctcacagagcatgtacttacttttacctttgaggcattcaccacacatccagcaagatttaaacatatactgggaggcagttgaatcctgcacccaggcggtggagcagttgagaaatttgtatctggaagatgaaaatatccgtgcaaattttattcaacatagaagtatcaaggagaaagacattattcatctggccaacaaatctcttcatttttctagcatcaaagattcagttgtgctatcgcttcatactggaaggatatactctgttcttgacttgatctatggcacaactgctgaagactcgtttgaagagatgtataatggaaaagcttcaccgttttcttcattcgtggactactaccgtgaaaagtatggtattgttattcaacatccaaaacaaccgttgttgctgttaaagcaaagccacaatgcacacaatcttcttttttcaaaatcgaagtacatagatggttctacgggcgatcctttgctcatggaaaaagagcaaatacacgcccgggtcccacctgaattgttaatccacattgatgtgacaattgaagttctcaaatctttttatttattgccttctgtaatgcatcgacttcagtctctcatgctagccagccagctccgcagagatattggttacactcaacatataccaagttacctgattttggaagctatcacaactttaaggtgctgcgaaacattttccctggagcgtttagaactattgggagactcagtactaaaatatgtgataggatgtgacctgtttttaaggtatcctatgaaacatgaaggtcatctttctgatatgagatcaaatgctgtctgcaatgctacacttcataagcacggaatctggcgatccttgcagggttacgtgcgggatagtgcatttgacccacggcgttgggttgctcctgggcagatatctttgcgcccttttccttgtaactgcagaattgagactgcatttgttccaagtaatggaaggtatatcagcgacgacccatcttttgtcgttggaaaaccatgtgatagagctcacagatggatgtgctcgaaagcaatatctgattgtgttgaagccctagttggagcatattatgtcggtggtggcatagttggtgcactttgggttatgaagtggttcggtattgaaataaaatgtgataggaagttggtacaggaagtaaagctcaatgcgtcttatatatgctacttacctaaaataagtgttattgaggagttggaagcaaaactgaagtacaacttctcagtcaagggccttctactggaagccataactcatccatctctgcaggaattaggggctgactactgttaccagcgtttggaatttctgggtgattctgtgctggatctactaattacacgctatctgtatgttacccatactgatgttgatcctggagaattgacagacctacgttccgctttggttagcaatgagaattttgcagaagtagttttaagaaataacattcacagtcatctacagcatgggtctggaatacttttggagcaaatcacagaatatgttagatccaatttggagtgccaagggaaggttaataaattccttcaacatgctacatgtaaagtacctaaggtgcttggagacattatggaaagcattgccggtgcaatatttgtagacacagattttaatgttgatgtggtttggaagattgttgaaccgttgctttccccaatgataacccctgataatcttgtattgccaccttatcgggagttgttagagctgtgtagtcaccttggttatttcataaattcaaaatgcagtagtaaaggagaagaagtaattatagagatgtcagtgcaattacgagatgaactgctgatagcgcaaggacaagacagaaataagaagagtgcaaagtcaaaagcagcagctcgtatattggcagatctgaagaaaagaggcctttcaatgaaacaatgttcttctaaagctaagcagttggatattatatcttcggaccagcagtatcctttggcaagtttggaatcaccacttgattattgtcacgtggatgccaatcctagcctaggaggccttccctcactgaaagaagcagttgttcttcggcttaaactggacaaaggtggaccacggagtgctctttttaagctatgcaagaggttgcagtggccaatgccagaattcgaatttgtggaacaaaggttcaggactcctattgttctggacggggtaaccacaacgaacttcaatagcttcgtgtcaaccatcaccttgcacatacctgatgtaacagccattacacttcaaggcgagcggcgaactgacaaaaagagttcccaggattcagcatcgctgataatgctccacaagcttcaagagctcaaggtctgtatatgcaagacttagcaaagcatcatggatccacgtgccagctaactgctaaggctcgttaacctggggatatgatcctccatcccaaagtgaattatgttgaaaatgtttatgagaggctcggcggtggtgctagattatcttgcaggatatcttagaaacaattagtgcttgtcttccgaaaaaaacaatagacaatttcacttgctggacaaagaaaacattttgcaagaattgatattggt

SEQ ID NO: 40 - DNA, 4884 bp, synthetic construct; identical to SEQ ID NO: 11
  source 1..4884: mol_type = other DNA

SEQ ID NO: 41 - DNA, 11900 bp, Triticum turgidum
  source 1..11900: mol_type = genomic DNA
atggccatggccgacgacgacgacggccccctgcccccgcctccgccgccgccgccgcctcgccgtccccacaggcagctccagccgaggaggtcgcgcattactctctccacagctctctggcgcccgcattcctagagctgacgggggttacccattctgtgtctgtgtcggcgcaggtaccaggtggaggtgttcgaggcggcgctgcaggggaacaccatcgcggtgctcgacaccggctccgggaagaccatggtcgccgtcctgctcgcgcgccaccacgtgggccgcgtgcgcgccggggaggtgccccgccggatcgtcttcttcctcgcgcccaccgtgcacctcgtccaccaggtgcgcttctgcactctgcagcgaccgattcgcgacggcaacgtctgcgccattttttcgcctcgcgttcgcagcttccatggccctgatcgctggttgctgtctgggctcgcgctgtagcaagtcgaggtgattcgggagtacacagacctcgacgccgcggagtgctacggagcttcgggggtcggcgactggagtacagaccgctggaacaaggaagttgggagcaaggaggtaagtgtcaggttttactctatcccgcggcacggtcattggttgatccgaatgcacactgaattggtacacgtaccgtgctcaccaagtgcttgatgaatgacttgttgacaaggaaccaacattgttagtttctgggtcgtagcagcttcggatcgcatgtagattgaggacatttgttatcggagtagtattctttagcgtgagggcagtcgactaaagcaatttgctgaagggccgtgactatgaaatagctggggtctggagtctggacacaagtcctaataaaagaaaatttcctttcaatgcagtcactgctaaatttggacactgcttcgaatccacatgttctcacaactcttgtaaatatgttgcttaaaaaaactcttgtaaatatgccacttggtaaagttatctggattatgtgtctttctaaattctaatccagttttgtggatattcctatgttttttacctgccgtattgcgcttattatgcgttctattgacaactttgattgaaaatatgaattttcctgggacaatgatcttattatgcgttctattatgtgtttcagattgctgttatgacgccccagatattgttggatgccctgaggcatgcctttgtaacaatgagtgcagtgagcctgctaatatttgatgaatgccatcgtgcctgtggaaaccacccatatacacgaataatgaaggtaatgggatttttcttgttttaacaacatctgtagatatgctttggcttgcctcatgcagttttttgaaagtaaagaagtttcgtaggatagcagtatttttcatagtttggcatgtcgaatttcctaacggtttatttctcttttgagtctgtaggaattttaccttggttctcagtggaggccagctgtatttggaatgacagcatctcctgttgctactaaaggcaagaattctacacatgactcgtgtgcttcctttctttctaccctctgcgattattcttcagcaaatatttccagcaaacgaccgctattttggttcttgaaatcagcatgttgtatgactgttatgtaccatgtgtcgtgctcttatcttctcccccacaagatcatacacttgctgaagtgcgatactctgttgtgccaacagtgccctactgctgtacactatatataaattgcagttagctacctgtacattattgcttccttcttcatctggatgactgggaacacaacttgtacgtttgacatgatttttattggtttatttcattttgacaaaaaggaaaattgtgcctgctattcatcctacctgaaggcacttgcgactcgggaccaatttatcaaatcaaaattattttcctgcactaggatgatttgttagacgctaacttcagtggtggagaggcaagtttcacttctaaaaattaccgtccttttcaggcacttgcacggtacaagattgtgaagcacacattggtcaacttgaacttacattggatgcaaaggttacctcagcatcctcaacttctttcactttgaatttcattgagcctttggaaatggtttccataccttacatgccattccaggtatatatcataaaagatcgtagtgaacttgagagcttttcccctcctgcaacaattgtgcacgaatactatgatgcttactcgattgatttcgaagatctgaaatcgaagctccagatattgtatgaagaggtgatttaatttaactatgttttgactcattcataagatacatctgtagtttggactttggattagccacacctataatgcgttgtatttctcgctttgcatctatattactgtacaaatgtttatttgttaaaagttggcattgttgggcagtatgatgctttgttggctagtctgcaagaatcgtcaccaaataaatttgaagacacaaataacatattagagacatcaagaaagaccttgttcagataccatgagaagatattttatggcctaaattatcttggtccaatcatcaccgctgaggtattgtataatctaatcattgagtagctttaccaaatttttcgtaagagttacaatggttattgttctatctgatctaattcgttgacttcacataagtccctgattctaactgaagttattcaaactttttggccatgtgtagaatattcgcattggcaaattcctagccgatatactatcacaatatcccttatgttcagcagttgctgataagatctttgaattagcgatgatgggcagaaattttacctaacattaagaagattaactgctttgcaaggtcatttaatatgtttttagtgtggagaaaatcgtgcgcatcaagtctcttgtacactttgagggaagaaatgctgcatcacgagcaagtagactagcgatttagctacctatatgaacctatcattttcctctcaacggaaaagtatatttcttagcattatgctgctgttgagttcgtccctaatccttgaaggccaaaataatggagtaaatggactgttaaattgccaggattgttttttatttgagggaatgccatcatgacttagctttattaacttcaaaaaacgtttacatcgtctatgagcttactgaccacaaagtcaggtggctcgtccaaccaactaaaggaagacctattacaataactaaagtttgctaggacatgagtcacttgattacatgaactataacaatgcttataaatgaccgtcccaatcaaggaactaatatcaacacattccgcgaaaattgccgccgccgaatcccaccaactagtttgcccggtgtaatagttaatgacctgcagggagtctgattctgcttctactcgattaaatctgagagaattagcaagagcaagtccatctctcatcgccattgcctccattgtgatcacatctgctgcaagaggaatgaatttaccatttccaggattgttggttaacaaaggatcgctttatgttgatatggaacatatcttctcatgaagtataagctgcatgtgaggagatgcttgagtctactaaaaggtctactagtattgtcattcaaatgattgtgaagcgtattaattggtcacaatactactttttaatttgctgtgaactgaccattacacttacacctattatcttggtaaatacattaaaattctaaattaagatgctcacaaggggggggggggatatggacgccccggtaatgttgctttggtaacatgacggaccgaatgtgttagatgaggcaaaatagcgtagttagaagtcggacagaataatttagttgaggtagttactgaaccatctgagctgcaacttgagttaacctatattgagttgagtgtagagtttgctcctgggacattgtacgaaacatcacgaagaaaccatacactgagatttcttgagaataatggtaccatttttccattttcttgtgcctcaccatataaattgtaaattgggtctctttaatgtatactatttggaaaaagatactagattcttccctaccatttccacagttccagtcttattaagaaggttgtggtccaaccatttcccttgcctgtcgcagttagcatacctttttcatcattcttccataccattttttattatatgaaatactagattttttcccctggagaacaaactgtgtattgttttcttaacaggtagtgaagatatataatgaaagcattaagacactgggtgattctgaagattgtctcttctcgaaagctagcttcagcctgcatgtagcttattttaaagaagctttagatttaattgaggaagtattgccacatggtaagccacaaacaaatatccatcagttagatgtgttatatctgtctgttttcacttgttattgctcataaatagtaatatactccctccgtcccataatgtaacgtcttacattatgggacggagggagtagtatacaactagaatctgcagtatctttctgtatataataaaggggtaggttataaaatcagttgcaaactgcattaagatcctccgaatccttagataagttgtatttatttcagtactttgcctgctatatctcgaaataaaataatggttctacacaaacctcttaatttgcacactctatggaggtgagctatgaggaagtacctgcaacgtgtatttctatagcaaatagccgaatagggagttaagtgggtgatcatcaaccattcagtagccatatgtgctgcacatatactgtccattattttgggttgaatgtagggcactcctcgttgtgtgaatctatatccttaaagactgaaatcgcctgtttcttaatgcacctatgtgttttgatttctgaagcattgtattattgatatctcactagactgaagcagcttggaaactacaatgtagtgcattgatagtttcttcttctaattctgctgatgtaattgcatcataggttatggtgaactaatgaaatcagaatctggttctgcggaattaattaaaagggggtatatttcttcaaaagtggatgctctaatcaacatttttaaatcatttgggtaaagccatattctgctcaagatgcttagttttgcctacttgtgctctcgtgtcagcaaaaaagggtaaatccacatgtattttctccacaggtcatcaaacgaagtgctttgcctaatttttgtggatagaattatgacagctaaagctgttgagaggttcatgagaggaattgttagtttctctagtttttcaatttcttacttgactggagggagtacatcaaaagattctctgagcccagcagtgcagagatatactttagacttgttccgatctggaaaggtatgccatttaaattgcaaagattgatttaaacaggattcaaaatgatacattaactccatgtcaatttctaggtgaacttgcttttcaccacagatgtaactgaagagggtattgatgtacctaactgttcttgtgtcatacgcttcgacctgcccagaactgtttgtagctacgttcaatctcgtggtcgtgccagaaggagcagctcaaattatgttcttatgattgagaggtacaataatcgtcgcagttcataagttgcatgctggtatagcatattaaattatagagaaatgttttggccaccatttattcttattctaagcacatttatcatgcatattatattcatagattgggtacaatttatgcctcataatcattaatttttctggaaaaggatatgctcttctacataatttggtttggcatgaacggtattttttgtcagtcagcactcagcagtctgagtttctagcccaggaattcctgaatctcgatcctctcatctaaaaaatattacaaccatgtttaaacattgttttaataatttttatgactacctattcaccttttgatacttcacatgaaattttcaggggaaacatggatcagcaggaacacatattccgcataatacggactgagtactatattaaacacttcgctctgtataggcacaaaaaccccaatgtttcatcctctgagtggcctatgcaagataagtatgcatactatgttgattcaacaggagcaactataactgcagattgctgtgttgatctgatccgtaaatattgtgagaaacttcctaaagataggtacacatatgcttaccatttttgtagcttttgttgctctgtcagcctgcttggtaatgcatctcatgcataatcactaagaatgctggtgtgcttaagattcctgaactgtgatgcaaccaattacatggatagcatgttggcgaaaatgttggttaatctgattgtcacacacagcacaacgaaaaactatttctcatacttcccatggcacgatcttactgtctttcattagatagaagtgattgatcattgtaacattcttctgcaggtattacatgccaaagccttcctttgcggtggctatcgaagatggatcataccaatgcaccttgactttgcctccaaatgcggcgtttcaaaggatagttggccctttatgcagtacaagtaatttagccaagcagcttgtatccctagatgcgtgcaagaaattgcatcagctaggagaactcaatgatcatcttctacccttcaccgaagaacctattgaaattgatgttgctttaagagatgggaaattcctttctggaccaggtatgtgtataccctttttcagtcttgctctcattaaaattctttagataagaacatttatttgcctgttttgctattttctttcactcatctattttatacacttcatcaatataacagtgatcagagtaagactgagcttgcccatttgtcgtacatggttaaacttgtgcaactaatgctggtgggctttgtttttattgttttgcacagtaatgcaactctgtgtgtatgaggttgtcttttggtcatgtttgtgcaatgtttggggcaaatatgaagtatctcccgactgttgagatctgctgaccttacgtagttattctccattcttttactttaccattccatctgagtggtgctgttacattgcatactgctgtctgtagtgaactgtgcaagtttcaacagcaatcccttcttccaaaatggtatgcatgcttctctcgtcgtagatgataacgatgttcccattgtttttttctcattttggcaggaacgactaaaagaaaggagctccatggaacaagaaatgttcttgccctgtcaggatcgtggattcatgaaagtgaaagtgtcacattgaatgcttatagattcgattttctttgtgaccaagagggtgaaaactatgctgggtttgttctcttaatggaatcagcacttgatgatgatgtagcttgttctaaaatggatctattcctgatccctaataaaatggtgtacaccaccgtaactccgtgtggaaaagttcaactggacaaaaaacaggttgttcaagaaataattgcatactgtaattctcttctataaagtttgtttggccatcgcttatttttgtcatttcttcagctacgtgacgggaagttgttccaagaattctttttcaatggaatatttggtagattatttcatggatctcgaacaagtggagtgcaaagggaatttcttttcagaaaaggtcatgaaatacaatggagctcagagagcatgtacttacttttacctttgaggcattcaccacacatccagcaagatttaaacatatactgggaggcagttgaatcctgcacccaggcggtggagcagttgagaaatttgtatctggaagatgaaaatatccgtgcaaattttattcaacatagaagtatcaaggagaaagacattattcatctggccaacaaatctcttcatttttctagcatcaaagattcagttgtgctatcgcttcatactggaaggatatactctgttcttgacttgatctatgacacaactgctgaagactcgtttgaagagatgtataatggaaaagcttcaccgttttcttcattcatggactactaccgtgaaaagtaatacctctattacatgtcttaaactcttaattacagtaaaatttattctgacatgcttcctgacaaacttctctttttactactcttttgtaggtatggtattgttattcaacatccaaaacaaccgttgttgctgttaaagcaaagccacaatgcacacaatcttcttttttcaaaatcgaagtacataggtactagctataaatcctagtccggtccagttccccttttcccgttatgatgaattgcaagatagctgttgctttactatttggtctgcttttacatgaaactctacataacctatggcagatggttctacgggtgatcctttgctcatggaaaaagagcaaatacacgcccgggtcccacctgaattgttaatccacattgatgtgacaattgaagttctcaaatctttttatttattgccttctgtaatgcatcgacttcagtctctcatgctagccagccagctccgcagagatattggttacactcaacatataccaagttacctggtctgttctctttcgtgagaatataattttattgctatggctggttatgcatacctgaataatttgatccgtttcaatgtcagattttggaagctatcacaactttaaggtgctgcgaaacattttccctggagcgtttagaattattgggagactcagtactaaaatatgtgataggatgtgacctgtttttaaggtatcctatgaaacatgaaggtcatctttctgatatgagatcaaatgctgtctgcaatgctacgcttcataagcacggaatctggcgatccttgcaggtggatggcctaaatccaagcctattataatggtttccacttcctgaagtttatgatgagttctagtattagacaaataattttaatttagttagttcatgtgcacttggccagtcaaccagttatatttgagccttccctgcattaaataaataaatatttgagcctgagggccaacagtcacatatgaacttcatatagcaagtggttttcttctgttctgctcttgagttttaattttgtttgagcccatgccagtcagatgttttctagaggtgcttcgtcaatgttcattcaaaaagcatgtttgatgatattgtaaaaaggcagaatttatatacaagcagagtcaagttgcgtataggagatagaatataacctagataccgttgagatttgcaaggttgagaatacaattccgaggtgtgtatgtttactggctatttgttatcttgctttccagggttacgtgcgggatagtgcatttgacccacggcgttgggttgctcctgggcagatatcattgcgcccttttccttgtaactgcggaattgagactgcatttgttccaactaatggaaggtatatcagcgacgacccatcttttgtcgttggaaaaccatgtgatagagctcacagatggatgtgctcgaaagcaatatctgattgtgttgaagccctagttggagcatattatgtcggtggtggcatagttgctgcactttgggttatgaagtggttcggtattgaaataaaatgcgataggaagttggtacaggaagtaaagctcaacgcatcttatatatgctacttacctaaaataagtgttattgaggagttggaagcaaaactgaagtacaacttctcagtcaagggccttctactggaagccataactcatccatctctgcaggaattaggggttgactactgttaccaggtcagtgtatagcagcatatttctatattggttcgtgggcaatcagaaattgcttcaaaacaagatatatccatgtcttctgtccttaacatttttgcgtctaacttgtgcctttatttcagcgtctggaatttctgggtgattctgtgctggatctactaattacacgctatctctatgttacccatactgatgttgatcctggagaattgacagacttacgttccgctttggttagtaatgagaattttgcagaagtagttttaagaaataacattcacagtcatctacagcatgggtctggaatacttttggagcaaatcacagaatatgttagatccaatttggagtgccaagggaaggttaataaattccttcaacatgctacatgtaaagtacctaaggttagaaatacagtttcacttatgttcattatcttaacagcctaccttttcaattttgtgcttttattgtttctgcgatggccttttctctgtgaagcctttcccagccaacattacacgtagaagttaataagattattttctttgttttacaggtgcttggagacattatggaaagcattgccggtgcaatatttgtagacacagattttaatgttgatgtggtttggaagattgttgaaccgttgctttccccaatgataacccctgataatcttgcattgccaccttatcgggagttgttagagctgtgtagtcaccttggttatttcataaattcaaaatgcagtagtaaaggagaagaagtaagtatagagatgtcagtgcaattacgagatgaactgctgatagcgcaaggacaagacagaaataagaagagtgcaaaggcaaaagcagcagctcgtatattggcagatctgaaggtaggttctttaaacataaagaggaatgctgtattagtgtgtagttcatgacagtgcctcgtggtttaaattgttgttcagttgaatcggtagcagaactatatcctgagcatactagctgatgttttctgcttagaaattagatgatttgtctatgccatttatctatgacagaaactgatgtttaagtttgaagcagaaaagaggcctttcaatgaaacaatgtccttctaaagctaagcagtcgggtattatatcttcggaccagcagtatcctttggcaagtgtaagcttccacatagttttccagcaaatgctttaacaaagtggtgtattttattatagataatgactattattacttttctgtagttggaatcaccacttgattattgtcaggtgaatgtcaatcctagcctaggaggccttccctcactgaaagaagcaggtatctgacgattcccattttgatcgtcctttggtttaattgtgttgcttgatagttcacagcctgtcaaacatgacatactaaaatgtatatatacctttttctctttgcacggataagatgcatgtctactcagtaaccatgcaattttgtttgttttcagttgttcttcggctcaaaatggacaaaggtggaccacgaagtgctctttttaagctatgcaagaggttgcagtggccaatgccagaattcgaatttgtggaacaaaggttcaggtatgtccaggaatacaagatatatatgacctctagcagccaaaacaaaaactccattccacttgaatgttggcaatgccgatccttctggtcttctcacgaagatgtgctaaattcttgtactggtttgatgcaacaggactcctattgttctggacggggtaaccacaacgaacttcaatagcttcgtgtcaaccatcaccttgcacatacctgatgtagcagccattacacttcaaggtgagcggcgaactgacaaaaagagttcccaggattcagcatcgctgacaatgctccacaagcttcaagagctcaaggtctgtatatgcaagacttag

SEQ ID NO: 42 - DNA, 5106 bp, synthetic construct; identical to SEQ ID NO: 13
  source 1..5106: mol_type = other DNA

SEQ ID NO: 43 - DNA, 13708 bp, Triticum turgidum
  source 1..13708: mol_type = genomic DNA
atgaagcctccgtcactctcattgcatttcctacccccaaacctcctctgcgcgccaacgaacgcagcgcaactcacacctccgcttcacgcacttcgccttctcccctccgcccagtctcccacctcccgtcccagccccggcgccgatttatatacacggctggtctacagacatgcgcttcccccacccccgcctcgcccactcgcccgcaccgaacccaaccccatggccatggccgacgacgacggccccctccccccgcctccgccgccgccgccgcctcgccgcccccacaggcagctccagccgaggaggtcgcgcattacttctccacatctctctggcgccagcattcctagagctgacgggggttaccaattccgtgtccgcgtcggcgcaggtaccaggtggaggtgttcgaggcggcgctgcaggggaacaccatcgcggtgctcgacaccggctccgggaagaccatggtcgccgtcctgctcgcgcgccatcacgtgggccgcgtgcgcgccggggaggtgccccgccggatcgtcgtcttcctcgcgcccaccgtgcacctcgtccaccaggtgcgcttctggactctgcactctgcaacgaccgattcgcgacggcaacgtctgcgccattttttcgcttcgcattcgcagctgctgtggccctgatcgctggttgctgcttgggctcgcgctgtagcaattcgaggtgattcgggagtacacggacctcgacgccgcggagtgctacggagctttgggggtcggcgactggagtaccgaccgctggaacaaggaagttgggagcaaggaggtaagtgtcggggtttactctatcccgcggcacggtcattggttgatccgaatgcacactgaattggtacacgtatcgtgctcaccaagtgcttgatgaatgacttgttgacaaggaaccagcattgttggtttctgggtgatagcagcttcggatcgcatgtagatcgatgccattgttatttgagtagtattctttagcgtgagggcagtcgactaaagcaatttgctgaagggccgtgactatgaaatagctggggtctctactctggacacaagtcctaataaaagaaaattgcctttcaatgcagtcactgctaaatttggacactgcttcgaaatccgcaagatcccacaactcttgtaaatatgttgcttaaaaaaactcttgcaaatatgccacttggtaaagttatctggattatgtgtctttctaaattctaatccagttttgtggatattcctatgctttttaccgaaaaaaaattatatgagaccaggtctcatataaaccaggtgagaccnnnnnnnnnnnnnnnnntgatttgtgaatgccacgtgtcatccatcaggatgggtctcacctgctaacccgtgagacctggtctcatagaatttttttccctttttacctgccgtattgtgcttattatgcgttctttgacaactttgattgaaaatatgaattttcctggaacaatgatcttattatgcgttctattgtgtgtttcagattgctgttatgacgcctcagatattgttggatgccctgaggcatgcctttgtaacaatgagtgcagtgagcctgctaatatttgatgaatgccatcgtgcctgtggaaaccacccatatacacgaataatgaaggtaatgggatttttcttgttttaacaacatctgtagatatgttttggcttgcctctcatgcagctttttgagtcatgagaagaatatgtttttcatagtttggcatgttgaatttcctaacggtttatttctcttttgagtctgtaggaattttaccttggctctcagtggaggccagctgtatttggaatgacagcatttcctgttgctactaaaggcaagaattctacacatgactcgtgtgcttcctttctttctaccctctgcgattattcttcagcaaatatttccagcaaacgaccgctattttggttcttgaaatcagcatgttgtatgactgttatgtaccatgtgtcgtgctcttatgatcttatcttctcccccacaagatcatacacttcctgaagtgcgatactctgttgtgccaacagtgccctactgctgtacactatatataaattgcagttagctacctgtacatgactgcttccttcttcatctggatgactgggaacacaacttgtacatttgacatgatttttattggtttatttcattttgacaaaaaggaaaattgtgcctgctattcatcctatctgaaggcacttgcgacttgggaccaatttatcaaatcaaaattattttcctgcactaggatgatttgttagacgctaacttcagtggaggagaggcaagtttcatttctaaaaattaccttccttttcaggcacttgcacggtacaagattgtgaagcacacattggtcaacttgaacttatattggacgcaaaggttacctcagcatcctcaacttctttcactttgaatttcattgagcctttggaaatagcctccataccttgcatgccattccaggtatatatcataaaagatcgtagtgaacttgagagcttttcccctcctgcaacaattgtgcacaaatactatgatgcttactcgattgatttcaaagatctgaaatcgaagctccagatattgtatgaagaggtgatttaatttaactatgttttgactcattcataagatacatctgtagtttggactttagagtagccacacctataatgcgctgtatttctcactttgcatctatattactgtacaaatatttatttgttaaaagttggcattgttgggcagtatgatgctttgttggctagtctgcaagaatcgtcaccaaataaatttgaagacacgaataacatattagagacatcaagaaagaccttgttcagataccatgagaagatattttatggcctaaattttcttggtccaatcatcaccgctgaggtattgtataatctaatcattgagtagctttaccaaatttttcgtaagagttacaatggttattgttctatctgatctagtttgttgacttcacataagtccctgattccaactgaagttattcaaactttttggccatgtgtagaatattcgcattggcaaattcctaaccggtatactatcacaatatcccttatgttcagcagttgctgataagatctctgaattagcgatgatgggcagaaattttacctaacattaagaagattaactgctttgcaaggtcatttaatatgtttttagtgtggagaaaatcatgcgcatcaagtcccttgtacactttgagggaagaaatgctgcatcacgagcaagtagactagcgatttagctacctatgaacctatcattttcctctcaacggaaaagtatatttcttagcattatgctgctgttgagttcgtccctaatccttgaaggccaaaataatggagtaaatggacagttaaattgccagggttgttttttatttgagggaaggccatcatgacttagctttattaacttcaaaaaacgtttacatcgtctatgagcttactgaccacaaagtcaggtgtctcgtccaaccaactaaaggaagacctattacaataactaaagtttgctaggacatgagtcacttgattacatgaactgtaacaatgcttataaatgaccatcccaatcaaggaactaatatcggcacattccgtgaaaattgccgccgccgaatcccaccaactagtttggccggtgtaatagttaatgacctgtagggagtctgattctgcttttactcgattaaatctgagagaattagcgagagcaagtccatctctcatcgccattgcctccattgtgatcacatccgctgcaagaggaatgaatttaccattgccaggattgttggttaacaaaggatcgctttatgttgaatatggaacatatccttctcatgaagtataagttgcatgtgaggagaagcttgagtctactaaaaggtcaactggtattgtcattcaaatgattgtgaagcatattaattggtcacaatactactttttaatttgctgtgaactgaccattacacttagagctatttatcttggtaaatacattaaaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggacgccccagtaatattgctttggtaacatgacggaccgaatgtgttagatgaggcaaaatagcgtagttagaagtcgaacagaataatttagttgaggtagttgctgaaccatctgagctgcaactggagttaacctatattgagttgagtggtagagtttgctcctgggacattgtacgaaacatcacgaagaaaccatacactgagatttcttgagaataatggtagcatttttccattttcttgtgcctcaccatataaattgtaaattgggtctctttaatgtatactattcggaaaaagatactagattcttccctaccatttccacagttccagtcttattaagaaggttgtggtccaaccatttcccttgcctgtcgcagttagcatacctttttcattattcttctataccattttttattatttgaaatactagattttttcccctggagaacaaactgtgtattgttttcttaacaggtagtgaagatatataatgaaagcattaagacactgggtgattctgaagattgcctcttctcgaaagctagcttcagcctgcatgtagcttattttaaagaagctttagatttaattgagaaagtattgccacatggtaagccacaaacaaatatccatcagttagatgtgttatatctgtctgttttcacttgttattgctcataaatagtaatatactccctccgtcccataatagtaatatatagtgtagtgtcaaaaaacgtcttacattatgggacgtagggagtagtatacaactagaatctgcagtatctttctgtatataataaaggggtaggttataaaatcagttgcaaactgcattaagatcctccgaatccttagataagttgtatttatttcagtactttgcctgctacatcttgaaataaaataatggttctacacaaacctcttaatttgcacactctacagaggtgagctatgaggaagtacctgcaacgtgtacagtttctatagcaaatagccaaatagggagttaagtgggtgatcatcaaccattcagtagccatatgtgctgcacatatactgtccattattttgggttgaatgtagggcactcctcgttgtgtgaatctatagccttaaagactgaaatcgcctgtttcttaatgcacctatgtgttttgatttctgaagcattgtattattgatatctcactagactgtaagcagcttggaaactacaatgtagtgcattgataatttcttcttctaattctgctgatgtaattgcatcataggttatggtgaactaatgaaatcagaatctggttctgcggaattaattaaaagtgggtatatttcttcaaaagtggatgctctaatcaacatttttaaatcatttgggtaaagccatattctgctcaagatgcttagttttgcctacgtgtgctctcgtgtcagtaaaaaagggtaaatccacatgtattttctccacaggtcatccaacgaagtgctttgcctaatttttgtagaaagaattatgacagctaaagctgttgagaggttcatgagaggaattgttaatttctctagtttttcaatttcttacttgactggagggagcacatcaaaagattctctgagcccagcagtgcagagatatactttggacttgttccgatctggaaaggtatgcaatttaaattgcaaagattgatttaaacaggattcaaaatgatacattaactccatgtcaatttctaggtgaacttgcttttcaccacagacgtaactgaagagggtattgatgtacctaactgttcttgtgtcatacgcttcgacctgcccagaactgtttgtagctatgttcaatctcgtggtcgtgccagaaggagcagctcaaattatgttcttatgattgagaggtacaataatcgtcgcacttcataagttgcatgctggtatagcatattaaattatagagaaatgttttggccaccatttattcttattctaagcacatttatcatgcatattttatcaaaaaaaattatcatgcatattataatcatagattgggtacaatttatgcctcataatcattaatttttctggaaaaggatatgctcttctacataatttggtttggcatgaatggtatgttttgtaagtcagcaatctgagtttctagcccaggaattcctgaatcccgaccctctcatctaaaaaatgttacaaccacgtttaaacattgttttaataatttttatgactacctattcacctttttatacttcacatgaaattttcaggggaaacaaggatcagcaggaacacatattccgcataatacagactgagtactatattaaacacttcgctctgtataggcacagaaaccccaatgtttcatcctctgagtggcctatgcaagataagtatgcataccatgttgattcaacaggagcaactataactgcagactgctgtgttgatctgatccgtaaatattgtgagaaacttcctaaagataggtacacatatgcttaccatttttgtaacttttgttgttctgtcagcctgcttggtaatgcacctcatgcataatcactaagaatgccggtgtgcttaagattcctgaactgtgaggcaaccaattacatggatagcatgttggtaaaaaagttggttaatttgattgtcacacacagcacaacgaaaaactatttctcgtacttcccatggcacgatcttactgtctttcattagatagaagtgattgatcattgtaacattcttctgcaggtattacatgccaaagccttcctttgcggtggctatcgaagatggatcataccaatgcaccttgactttgcctccaaatgcggcgtttcaaaggatagttggccctttatgcagtacaagtaatttagccaagcagcttgtatccctagatgcgtgcaagaaattgcatcagctaggagaactcaatgatcatcttctacccttcaccgaagaacctattgatattgatgttgctttaagagatgggaaattcctttctggaccaggtatgtgtataccctttttcagtcttgctctcattaaaattctttagataagaacatttatttgcctgttttgctattttctttcactcatctattttatacacttcatcaatataacagtgatcagagtaaggctgagcttgcccatttgtcgtacatggttaaacttgtgcaactaatgctggtgggctttgtttttattgttttgcacagtaatgcaactctgtgtgtatgaggttgtcttttggtcatgtttgtgcaaggtttggggcaaatatgaagtatctcccgactgttgagatctgctgaccttacatagttattctccattcttttactttgccatttcatctgagtggtcctgttacattgcatactgctgtctgtagtgaactgtgcaagtttcaacagcaatcccttcttccaaaatggtatgcgtgcttctctcgccgtagatgatgacgattttcccattgttcttttctcattttggcaggaacgactaaaagaaaggagctccatggaacaagaaatgttcttgccctgtcaggatcgtggattcatgaaagtggaagtgtcacattgaatgcttatagattcggttttctttgtgaccaagagggtgaaaactatgctgggtttgttctcttaatggaatcagcacttgatgatgatgtagcttattcaaaaatggatctattcctgatccctaataaaatggtctacaccaccgtaactccgtgtggaaaagttcaactggacaaaaaacaggttgttcaagaaataatcgcatactgtaattctcttgtataaagtttgtttggccatcgcttatttttgtcatttcttcagctacgtgacgggaagttgtttcaagaattctttttcaatggaatatttggtagattatttcatggatctcgaacaagtggagtgcaaagggaatttcttttcagaaaaggtcatgaaatacaatggagctcagagagcatgtacttacttttacctttgaggcattcaccacacatccagcaagatttaaacatatactgggaggcagttgaatcctgcacccaggcggtggagcggttgagaaatttgtatctggaagaggaaaatatccgtgcaaattttattcaacatagaagtatcaaggagaaagacattattcatctggccaacaaatctgttcatttttctagcatcaaagattcagttgtgctatcgcttcatactggaaggatatactctgttcttgacttgatctatgacataactgctgaagactcgtttgaagagatgtataatggaaaagcttcaccgttttcttcattcgtggactactatcgtgaaaagtaatacctctattacatgtcttaaactcttaattactagtaaaatttattctgacatgcttcctgacaaacttctctttttaccactcttttgtaggtatggtattgttattcaacatccaaaacaaccgttgttgctgttaaagcaaagccacaatgcacacaatcttcttttttcaaaatcgaagtacataggtactagctataaatcctagtccggtccagttccccttttcccgttatgatgaattgcaagatagctgttgctttactatttggtctgcttttacatgaaactctacataacctatggcagatggttctacgggcgatcctttgcttatggaaaaagagcaaatacacgcccgggtcccacctgaattgttaatccacattgatgtgacaattgaagttctcaaatctttttacttattgccttctgtaatgcatcgacttcagtctctcatgctagccagccagctccgcagagatattggttacactcaacatataccaagttacctggtctgttctctttcgtgagaatataattttattgctatggctggttatgcatacctgaataatttgatccgtttcaatgtcagattttggaagctatcacaactttaaggtgctgtgaaacattttccctggagcgtttagaactattgggagactcagtactaaaatatgtgataggatgtgacctgtttttaaggtatcctatgaaacatgaaggtcatctttctgatatgagatcaaatgctgtctgcaatgctacacttcataagcacggaatctggcgatccttgcaggtagatggcctaaatccaagcctattataatggtttcaacttcctgaagtttatgatgagttctagtattggacaaataattttaatttagttagttcatgtgcacttggccagtcaaccagttatatttgagccttcccgcagtaaataaataaatatttgagcctgagggccaacaatcacatatgaacttcatatagcaagtggttttcttctgttctgctcttgagttttaattttgtttgagcccatgccagtcagatgttttctagaggtgcttcgtcaatgttcattcaaaaagcatgtttgatgatattgtaaaaaggcagaagttatatacgagcagagtcaagttgcgtataggagatagaatataacctagatactgttgagatttgcaaggttgagaatacaattccgaggtgtgtatgtttactggctatttgttatcttgctttccagggttacgtgcgggatagtgcatttgacccacggcgttgggttgctcctgggcagatatctttgcgcccttttccttgtaactgcggaattgagactgcatttgttccaactaatggaaggtatatcagcgacgacccatcttttgtagttggaaaaccatgtgatagagctcacagatggatgtgctcgaaagcaatatctgattgtgttgaagccctagttggagcatattatgttggtggtggcatagttgctgcactttgggttatgaagtggttcggtattgaaataaaatgcgataggacgttggtacaggaagtaaagctcaatgcatcttatatatgctacttacctaaaataagtgttattgaggagttggaagcaaaactgaagtacaacttctcggtcaagggccttctactggaagccataactcatccatctctgcaggaattaggggttgactactgttaccaggtcagtgtatagcagcatatttctacattggttcgtggggttatcattagcttattgtgcttcaatcagaaacaagatatatccatgtcttctgtccttaacattttgagtgaagtgcattgtaggtccttaaactattttaggggtgtcacataggtactcgaactatgaaaatcgtcatttaggtccttgaaatacagtaagtgtgtcatccaggtccaaaatctcactaaccccctctaaatggccagctggcatggtgaacagtgccaaaaataaaaaaatatgaacaaaaaaatctgaaaaactttggcatcaaagatacccctggtgcgtgaatagtaaaaatgttcattaattcaaaaaaatgttcgcgaatatgggaaaaataatcgcgactttaaaaaagttaacaaacaataaatttgaaaatatgttcgcgaaccacaaatattgttcgtgggtatggaaagaatgttcgcgaaccacaaaaattcgtgactttaaaaaatgttagtgaactttttattaaagtcgcaaatatgttttcctaattcatagttcgcgaacattttattaaagtcacgaatattttttccaaattcatcgttcgcgaacatttttttaaagtcatgaatattttttccaaattcatcgttcgcgaactttttttaaactcgcgaatatttttcccaaattcatcgttcgcgaacatttttttaaagtcgtgaatattttttccaaattcatcgttcgcaaactttttttaaactcgcgaatatttttttaaagttgcgaatattttttttccaaattcatcgttcgcgaacattttattaaagtcacaaatttttttccaaattcatcgttcgcgaactttttttgaattaatgacatttttactgttcacgcgccagggtctctttgatgacagaggatctcagttttttgatatttttttatatttattctttgttgtgtactgttcatcacgcacaggaatttaggattgtttgccgcgtactgttacggttactgtttatgtgccagctggccagttagagcctgtcagggagattttggacctgaatgacacacttactgcactttgaggatctagatgacgattttcatagttcgaggacctacgtgacatccctggaatagttcaaggacctacagtgcacttcactctaacatttttgcgtctaacttgtgcctttatttcagcgtctggaatttctgggtgattctgtgctggatctactaattacacgctatctctatgttacccatactgatgttgatcctggagaattgacagacttacgttccgctttggttagtaatgagaattttgcagaagtagttttaagaaacaacattcacagtcatctacagcatgggtctggaatacttttggagcaaatcacagaatatgttagatccaatttggagtgccaagggaaggttaataaattccttcaacatgctacatgtaaagcacctaaggttagaaatacagtttcacttatgttcattatcttaacagcctaccttttcaattttgtgcttttattgtttctgcgatggccttttctctgtgaagcctttcccagccaacattacacatagaagttaacaagattattttgtttgttttacaggtacttggtgacattatggaaagcattgccggtgcaatatttgttgacgcagattttaatgttgatgtggtttggaagattgttgaaccgttgctttccccaatgataacccctgataatcttgcattgccaccttatcgggagttgttagagctgtgtagtcaccttggttatttcataaattcaaaatgcagtagtaaaggagaagaattaagtatagagatgtcagtgcaattacgagatgaactgttgatagcgcaaggacaagacagaaacaagaagagtgcaaaggcaaaagcagcagctcgtatattggcagatctgaaggtaggttctttaaacttaaagaggaatgctgtattagtgtgtagttcatgacagtgcctcgtggtttaaattgttgttcagttgaatcggtagcagaactatatcctgagcatactttcctgtatttaactagctgatgttttctgcttagaaattactccctccgttcctaaatataagtctttttaggcatttcaaatggactataacatatggatgtatgtagacatattttagaatgtagattcactcattttgctccgtatgtagtcacttgttggaatctctagaaagacttatatttgggaacggagggagtagatgatttgtctatgccatttatttatgacagaaactgatgtttaagtttgaagcagaaaagaggcctttcaatgaaacaatgttcttctaaagctaagcagttggatattatatcatcggagcagtatcctttggcaagtgtaagcttctacatagttttccagcaaatgctttaacaaagtggtgtattttattatagataatgactattattacatttctgtagttggaatcaccacttgattattgtcacgtgaatgccaatcctagcctaggaggccttccctcactgaaagaagcaggtatctgacgattcccattttgatcgtcctttggtttaattgtgttgcttgatcgctgacagcctgtcaaacatgacatactaaaatgtatatatacctctttctttttacatggataagatgcatgtctagtcagtaaccatgcatttttgtttgttttcagttgttcttcggcttaaaatggacaaaggtggaccacgaagtgctctttttaagctatgcaagaggttgcagtggccaatgccagaattcgaatttgttgaacaaaggttcaggtatgtccaggaatacaaaatatatatgatctctagcaacaaaggcaaaaacttcattccacttgaatgttggcaatgccgatccttctgctcttttaacgaacatgtgctaaattcttttactggtttgatgcaacaggactcctattgttctggacggggtaaccacaacgaacttcaatagcttcgtgtcaaccatcaccttgcacatacctgatgtaacagccattacacttcaaggcgagcggcggactgacaaaaggagttcccaggattcagcatcgctggcaatgctccacaagcttcaagagctcaaggtctgtatatgcaagacttag

SEQ ID NO: 44 - DNA, 4884 bp, synthetic construct
  source 1..4884: mol_type = other DNA
atggccatggccgacgacgacgacggccccctgcccccgcctccgccgccgccgccgcctcgccgtccccacaggcagctccagccgaggaggtaccaggtggaggtgttcgaggcggcgctgcaggggaacaccatcgcggtgctcgacaccggctccgggaagaccatggtcgccgtcctgctcgcgcgccaccacgtgggccgcgtgcgcgccggggaggtgccccgccggatcgtcttcttcctcgcgcccaccgtgcacctcgtccaccagcaagtcgaggtgattcgggagtacacagacctcgacgccgcggagtgctacggagcttcgggggtcggcgactggagtacagaccgctggaacaaggaagttgggagcaaggagattgctgttatgacgccccagatattgttggatgccctgaggcatgcctttgtaacaatgagtgcagtgagcctgctaatatttgatgaatgccatcgtgcctgtggaaaccacccatatacacgaataatgaaggaattttaccttggttctcagtagaggccagctgtatttggaatgacagcatctcctgttgctactaaaggcacttgcacggtacaagattgtgaagcacacattggtcaacttgaacttacattggatgcaaaggtatatatcataaaagatcgtagtgaacttgagagcttttcccctcctgcaacaattgtgcacgaatactatgatgcttactcgattgatttcgaagatctgaaatcgaagctccagatattgtatgaagagtatgatgctttgttggctagtctgcaagaatcgtcaccaaataaatttgaagacacaaataacatattagagacatcaagaaagaccttgttcagataccatgagaagatattttatggcctaaattatcttggtccaatcatcaccgctgaggtagtgaagatatataatgaaagcattaagacactgggtgattctgaagattgtctcttctcgaaagctagcttcagcctgcatgtagcttattttaaagaagctttagatttaattgaggaagtattgccacatggttatggtgaactaatgaaatcagaatctggttctgcggaattaattaaaagggggtatatttcttcaaaagtggatgctctaatcaacatttttaaatcatttgggtcatcaaacgaagtgctttgcctaatttttgtggatagaattatgacagctaaagctgttgagaggttcatgagaggaattgttagtttctctagtttttcaatttcttacttgactggagggagtacatcaaaagattctctgagcccagcagtgcagagatatactttagacttgttccgatctggaaaggtgaacttgcttttcaccacagatgtaactgaagagggtattgatgtacctaactgttcttgtgtcatacgcttcgacctgcccagaactgtttgtagctacgttcaatctcgtggtcgtgccagaaggagcagctcaaattatgttcttatgattgagaggggaaacatggatcagcaggaacacatattccgcataatacggactgagtactatattaaacacttcgctctgtataggcacaaaaaccccaatgtttcatcctctgagtggcctatgcaagataagtatgcatactatgttgattcaacaggagcaactataactgcagattgctgtgttgatctgatccgtaaatattgtgagaaacttcctaaagataggtattacatgccaaagccttcctttgcggtggctatcgaagatggatcataccaatgcaccttgactttgcctccaaatgcggcgtttcaaaggatagttggccctttatgcagtacaagtaatttagccaagcagcttgtatccctagatgcgtgcaagaaattgcatcagctaggagaactcaatgatcatcttctacccttcaccgaagaacctattgaaattgatgttgctttaagagatgggaaattcctttctggaccaggaacgactaaaagaaaggagctccatggaacaagaaatgttcttgccctgtcaggatcgtggattcatgaaagtgaaagtgtcacattgaatgcttatagattcgattttctttgtgaccaagagggtgaaaactatgctgggtttgttctcttaatggaatcagcacttgatgatgatgtagcttgttctaaaatggatctattcctgatccctaataaaatggtgtacaccaccgtaactccgtgtggaaaagttcaactggacaaaaaacagctacgtgacgggaagttgttccaagaattctttttcaatggaatatttggtagattatttcatggatctcgaacaagtggagtgcaaagggaatttcttttcagaaaaggtcatgaaatacaatggagctcagagagcatgtacttacttttacctttgaggcattcaccacacatccagcaagatttaaacatatactgggaggcagttgaatcctgcacccaggcggtggagcagttgagaaatttgtatctggaagatgaaaatatccgtgcaaattttattcaacatagaagtatcaaggagaaagacattattcatctggccaacaaatctcttcatttttctagcatcaaagattcagttgtgctatcgcttcatactggaaggatatactctgttcttgacttgatctatgacacaactgctgaagactcgtttgaagagatgtataatggaaaagcttcaccgttttcttcattcatggactactaccgtgaaaagtatggtattgttattcaacatccaaaacaaccgttgttgctgttaaagcaaagccacaatgcacacaatcttcttttttcaaaatcgaagtacatagatggttctacgggtgatcctttgctcatggaaaaagagcaaatacacgcccgggtcccacctgaattgttaatccacattgatgtgacaattgaagttctcaaatctttttatttattgccttctgtaatgcatcgacttcagtctctcatgctagccagccagctccgcagagatattggttacactcaacatataccaagttacctgattttggaagctatcacaactttaaggtgctgcgaaacattttccctggagcgtttagaattattgggagactcagtactaaaatatgtgataggatgtgacctgtttttaaggtatcctatgaaacatgaaggtcatctttctgatatgagatcaaatgctgtctgcaatgctacgcttcataagcacggaatctggcgatccttgcagggttacgtgcgggatagtgcatttgacccacggcgttgggttgctcctgggcagatatcattgcgcccttttccttgtaactgcggaattgagactgcatttgttccaactaatggaaggtatatcagcgacgacccatcttttgtcgttggaaaaccatgtgatagagctcacagatggatgtgctcgaaagcaatatctgattgtgttgaagccctagttggagcatattatgtcggtggtggcatagttgctgcactttgggttatgaagtggttcggtattgaaataaaatgcgataggaagttggtacaggaagtaaagctcaacgcatcttatatatgctacttacctaaaataagtgttattgaggagttggaagcaaaactgaagtacaacttctcagtcaagggccttctactggaagccataactcatccatctctgcaggaattaggggttgactactgttaccagcgtctggaatttctgggtgattctgtgctggatctactaattacacgctatctctatgttacccatactgatgttgatcctggagaattgacagacttacgttccgctttggttagtaatgagaattttgcagaagtagttttaagaaataacattcacagtcatctacagcatgggtctggaatacttttggagcaaatcacagaatatgttagatccaatttggagtgccaagggaaggttaataaattccttcaacatgctacatgtaaagtacctaaggtgcttggagacattatggaaagcattgccggtgcaatatttgtagacacagattttaatgttgatgtggtttggaagattgttgaaccgttgctttccccaatgataacccctgataatcttgcattgccaccttatcgggagttgttagagctgtgtagtcaccttggttatttcataaattcaaaatgcagtagtaaaggagaagaagtaagtatagagatgtcagtgcaattacgagatgaactgctgatagcgcaaggacaagacagaaataagaagagtgcaaaggcaaaagcagcagctcgtatattggcagatctgaagaaaagaggcctttcaatgaaacaatgtccttctaaagctaagcagtcgggtattatatcttcggaccagcagtatcctttggcaagtttggaatcaccacttgattattgtcaggtgaatgtcaatcctagcctaggaggccttccctcactgaaagaagcagttgttcttcggctcaaaatggacaaaggtggaccacgaagtgctctttttaagctatgcaagaggttgcagtggccaatgccagaattcgaatttgtggaacaaaggttcaggactcctattgttctggacggggtaaccacaacgaacttcaatagcttcgtgtcaaccatcaccttgcacatacctgatgtagcagccattacacttcaaggtgagcggcgaactgacaaaaagagttcccaggattcagcatcgctgacaatgctccacaagcttcaagagctcaaggtctgtatatgcaagacttag

SEQ ID NO: 45 - protein, 182 aa, Triticum turgidum
MAMADDDDGPLPPPPPPPPPRRPHRQLQPRRYQVEVFEAALQGNTIAVLDTGSGKTMVAVLLARHHVGRVRAGEVPRRIVFFLAPTVHLVHQQVEVIREYTDLDAAECYGASGVGDWSTDRWNKEVGSKEIAVMTPQILLDALRHAFVTMSAVSLLIFDECHRACGNHPYTRIMKEFYLGSQ

SEQ ID NO: 46 - DNA, 3111 bp, synthetic construct
  source 1..3111: mol_type = other DNA
atgccaaagccttcctttgcggtggctatcgaagatggatcataccaatgcaccttgactttgcctccaaatgcggcgtttcaaaggatagttggccctttatgcagtacaagtaatttagccaagcagcttgtatccctagatgcgtgcaagaaattgcatcatctaggagaactcaatgatcatcttctacccttcaccgaagaacctattgatattgatgttgctttaagagatgggaaattcctttctggaccaggaacgactaaaagaaaggagctccatggaacaagaaatgttcttgccctgtcaggatcgtggattcatgaaagtggaagtgtcacattgaatgcttatagattcggttttctttgtgaccaagagggtgaaaactatgctgggtttgttctcttaatggaatcagcacttgatgatgatgtagcttattcaaaaatggatctattcctgatccctaataaaatggtctacaccaccgtaactccgtgtggaaaagttcaactggacaaaaaacagctacgtgacgggaagttgtttcaagaattctttttcaatggaatatttggtagattatttcatggatctcgaacaagtggagtgcaaagggaatttcttttcagaaaaggtcatgaaatacaatggagctcagagagcatgtacttacttttacctttgaggcattcaccacacatccagcaagatttaaacatatactgggaggcagttgaatcctgcacccaggcggtggagcggttgagaaatttgtatctggaagaggaaaatatccgtgcaaattttattcaacatagaagtatcaaggagaaagacattattcatctggccaacaaatctcttcatttttctagcatcaaagattcagttgtgctatcgcttcatactggaaggatatactctgttcttgacttgatctatgacataactgctgaagacgcgtttgaagagatgtataatggaaaagcttcaccgttttcttcattcgtggactactatcgtgaaaagtatggtattgttattcaacatccaaaacaaccgttgttgctgttaaagcaaagccacaatgcacacaatcttcttttttcaaaatcgaagtacatagatggttctacgggcgatcctttgcttatggaaaaagagcaaatacacgcccgggtcccacctgaattgttaatccacattgatgtgacaattgaagttctcaaatctttttacttattgccttctgtaatgcatcgacttcagtctctcatgctagccagccagctccgcagagatattggttacactcaacatataccaagttacctgattttggaagctatcacaactttaaggtgctgtgaaacattttccctggagcgtttagaactattgggagactcagtactaaaatatgtgataggatgtgacctgtttttaaggtatcctatgaaacatgaaggtcatctttctgatatgagatcaaatgctgtctgcaatgctacacttcataagcacggaatctgacgatccttgcagggttacgtgcgggatagtgcatttgacccacggcgttgggttgctcctgggcagatatctttgcgcccttttccttgtaactgcggaattgagactgcatttgttccaactaatggaaggtatatcagcgacgacccatcttttgtagttggaaaaccatgtgatagagctcacagatggatgtgctcgaaagcaatatctgattgtgttgaagccctagttggagcatattatgttggtggtggcatagttgctgcactttgggttatgaagtggttcggtattgaaataaaatgcgataggacgttggtacaggaagtaaagctcaatgcatcttatatatgctacttacctaaaataagtgttattgaggagttggaagcaaaactgaagtacaacttctcggtcaagggccttctactggaagccataactcatccatctctgcaggaattaggggttgactactgttaccagcgtctggaatttctgggtgattctgtgctggatctactaattacacgctatctctatgttacccatactgatgttgatcctggagaattgacagacttacgttccgctttggttagtaacgagaattttgcagaagtagttttaagaaacaacattcacagtcatctacagcatgggtctggaatacttttggagcaaatcacagaatatgttagatccaatttggagtgccaagggaaggttaataaattccttcaacatgctacatgtaaagcacctaaggtacttggtgacattatggaaagcattgccggtgcaatatttgttgacgcagattttaatgttgatgtggtttggaagattgttgaaccgttgctttccccaatgataacccctgataatcttgcattgccaccttatcgggagttgttagagctgtgtagtcaccttggttatttcataaattcaaaatgcagtagtaaaggagaagaattaagtatagagatgtcagtgcaattacgagatgaactgttgatagcgcaaggacaagacagaaacaagaagagtgcaaaggcaaaagcagcagctcgtatattggcagatctgaagaaaagaggcctttcaatgaaacaatgttcttctaaagctaagcagttggatattatatcatcggagcagtatcctttggcaagtttggaatcaccacttgattattgtcacgtgaatgccaatcctagcctaggaggccttccctcactgaaagaagcagttgttcttcggcttaaaatggacaaaggtggaccacgaagtgctctttttaagctatgcaagaggttgccgtggccaatgccagaattcgaatttgttgaacaaaggttcaggactcctattgttctggacggggtaaccacaacgaacttcaatagcttcgtgtcaaccatcaccttgcacatacctgatgtaacagccattacacttcaaggcgagcggcggactgacaaaaggagttcccaggattcagcatcgctggcaatgctccacaagcttcaagagctcaaggtctgtatatgcaagacttag

SEQ ID NO: 47 - protein, 513 aa, Triticum turgidum
MPKPSFAVAIEDGSYQCTLTLPPNAAFQRIVGPLCSTSNLAKQLVSLDACKKLHHLGELNDHLLPFTEEPIDIDVALRDGKFLSGPGTTKRKELHGTRNVLALSGSWIHESGSVTLNAYRFGFLCDQEGENYAGFVLLMESALDDDVAYSKMDLFLIPNKMVYTTVTPCGKVQLDKKQLRDGKLFQEFFFNGIFGRLFHGSRTSGVQREFLFRKGHEIQWSSESMYLLLPLRHSPHIQQDLNIYWEAVESCTQAVERLRNLYLEEENIRANFIQHRSIKEKDIIHLANKSLHFSSIKDSVVLSLHTGRIYSVLDLIYDITAEDAFEEMYNGKASPFSSFVDYYREKYGIVIQHPKQPLLLLKQSHNAHNLLFSKSKYIDGSTGDPLLMEKEQIHARVPPELLIHIDVTIEVLKSFYLLPSVMHRLQSLMLASQLRRDIGYTQHIPSYLILEAITTLRCCETFSLERLELLGDSVLKYVIGCDLFLRYPMKHEGHLSDMRSNAVCNATLHKHGI

SEQ ID NO: 48 - RNA, 22 nt, synthetic construct
  source 1..22: mol_type = other RNA
ttyrkaytrracttttwtactt

SEQ ID NO: 49 - RNA, 22 nt, synthetic construct
  source 1..22: mol_type = other RNA
aartawaaaagtttagtmyraa

SEQ ID NO: 50 - RNA, 22 nt, synthetic construct
  source 1..22: mol_type = other RNA
tttgtactaaacttttatactt

SEQ ID NO: 51 - DNA, 140 bp, Hordeum vulgare
  source 1..140: mol_type = genomic DNA
tgttcgaggcggcgctgcaggggaacaccatcgcagtgctcgacaccggctccgggaagaccatggtcgccgtcctgctcgcgcgctaccacgtcagccgcgcgcgcgccggggaggcgccccgccggatcgtcgtcttc

SEQ ID NO: 52 - DNA, 8031 bp, synthetic construct
  source 1..8031: mol_type = other DNA
tgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatcccctcgaggcgcgccaagctatcaaacaagtttgtacaaaaaagcaggctccgaattcgcccttcaccatggattacaaggatgatgatgataaggattacaaggatgatgatgataagatggctccaaagaagaagagaaaggttggaatccacggagttccagctgctgataagaagtactctatcggacttgacatcggaaccaactctgttggatgggctgttatcaccgatgagtacaaggttccatctaagaagttcaaggttcttggaaacaccgatagacactctatcaagaagaaccttatcggtgctcttcttttcgattctggagagaccgctgaggctaccagattgaagagaaccgctagaagaagatacaccagaagaaagaacagaatctgctaccttcaggaaatcttctctaacgagatggctaaggttgatgattctttcttccacagacttgaggagtctttccttgttgaggaggataagaagcacgagagacacccaatcttcggaaacatcgttgatgaggttgcttaccacgagaagtacccaaccatctaccaccttagaaagaagttggttgattctaccgataaggctgatcttagacttatctaccttgctcttgctcacatgatcaagttcagaggacacttccttatcgagggagaccttaacccagataactctgatgttgataagttgttcatccagcttgttcagacctacaaccagcttttcgaggagaacccaatcaacgcttctggagttgatgctaaggctatcctttctgctagactttctaagtctcgtagacttgagaaccttatcgctcagcttccaggagagaagaagaacggacttttcggaaaccttatcgctctttctcttggacttaccccaaacttcaagtctaacttcgatcttgctgaggatgctaagttgcagctttctaaggatacctacgatgatgatcttgataaccttcttgctcagatcggagatcagtacgctgatcttttccttgctgctaagaacctttctgatgctatccttctttctgacatccttagagttaacaccgagatcaccaaggctccactttctgcttctatgatcaagagatacgatgagcaccaccaggatcttacccttttgaaggctcttgttagacagcagcttccagagaagtacaaggaaatcttcttcgatcagtctaagaacggatacgctggatacatcgatggaggagcttctcaggaggagttctacaagttcatcaagccaatccttgagaagatggatggaaccgaggagcttcttgttaagttgaacagagaggatcttcttagaaagcagagaaccttcgataacggatctatcccacaccagatccaccttggagagcttcacgctatccttcgtagacaggaggatttctacccattcttgaaggataacagagagaagatcgagaagatccttaccttcagaatcccatactacgttggaccacttgctagaggaaactctcgtttcgcttggatgaccagaaagtctgaggagaccatcaccccttggaacttcgaggaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggttgttgataagggagcttctgctcagtctttcatcgagagaatgaccaacttcgataagaaccttccaaacgagaaggttcttccaaagcactctcttctttacgagtacttcaccgtttacaacgagcttaccaaggttaagtacgttaccgagggaatgagaaagccagctttcctttctggagagcagaagaaggctatcgttgatcttcttttcaagaccaacagaaaggttaccgttaagcagttgaaggaggattacttcaagaagatcgagtgcttcgattctgttgaaatctctggagttgaggatagattcaacgcttctcttggaacctaccacgatcttttgaagatcatcaaggataaggatttccttgataacgaggagaacgaggacatccttgaggacatcgttcttacccttacccttttcgaggatagagagatgatcgaggagagactcaagacctacgctcaccttttcgatgataaggttatgaagcagttgaagagaagaagatacaccggatggggtagactttctcgtaagttgatcaacggaatcagagataagcagtctggaaagaccatccttgatttcttgaagtctgatggattcgctaacagaaacttcatgcagcttatccacgatgattctcttaccttcaaggaggacatccagaaggctcaggtttctggacagggagattctcttcacgagcacatcgctaaccttgctggatctccagctatcaagaagggaatccttcagaccgttaaggttgttgatgagcttgttaaggttatgggtagacacaagccagagaacatcgttatcgagatggctagagagaaccagaccacccagaagggacagaagaactctcgtgagagaatgaagagaatcgaggagggaatcaaggagcttggatctcaaatcttgaaggagcacccagttgagaacacccagcttcagaacgagaagttgtacctttactaccttcagaacggaagagatatgtacgttgatcaggagcttgacatcaacagactttctgattacgatgttgatcacatcgttccacagtctttcttgaaggatgattctatcgataacaaggttcttacccgttctgataagaacagaggaaagtctgataacgttccatctgaggaggttgttaagaagatgaagaactactggagacagcttcttaacgctaagttgatcacccagagaaagttcgataaccttaccaaggctgagagaggaggactttctgagcttgataaggctggattcatcaagagacagcttgttgagaccagacagatcaccaagcacgttgctcagatccttgattctcgtatgaacaccaagtacgatgagaacgataagttgatcagagaggttaaggttatcaccttgaagtctaagttggtttctgatttcagaaaggatttccagttctacaaggttagagagatcaacaactaccaccacgctcacgatgcttaccttaacgctgttgttggaaccgctcttatcaagaagtacccaaagttggagtctgagttcgtttacggagattacaaggtttacgatgttagaaagatgatcgctaagtctgagcaggagatcggaaaggctaccgctaagtacttcttctactctaacatcatgaacttcttcaagaccgagatcacccttgctaacggagagatcagaaagagaccacttatcgagaccaacggagagaccggagagatcgtttgggataagggaagagatttcgctaccgttagaaaggttctttctatgccacaggttaacatcgttaagaaaaccgaggttcagaccggaggattctctaaggagtctatccttccaaagagaaactctgataagttgatcgctagaaagaaggattgggacccaaagaagtacggaggattcgattctccaaccgttgcttactctgttcttgttgttgctaaggttgagaagggaaagtctaagaagttgaagtctgttaaggagcttcttggaatcaccatcatggagcgttcttctttcgagaagaacccaatcgatttccttgaggctaagggatacaaggaggttaagaaggatcttatcatcaagttgccaaagtactctcttttcgagcttgagaacggaagaaagagaatgcttgcttctgctggagagcttcagaagggaaacgagcttgctcttccatctaagtacgttaacttcctttaccttgcttctcactacgagaagttgaagggatctccagaggataacgagcagaagcagcttttcgttgagcagcacaagcactaccttgatgagatcatcgagcaaatctctgagttctctaagagagttatccttgctgatgctaaccttgataaggttctttctgcttacaacaagcacagagataagccaatcagagagcaggctgagaacatcatccaccttttcacccttaccaaccttggtgctccagctgctttcaagtacttcgataccaccatcgatagaaaaagatacacctctaccaaggaggttcttgatgctacccttatccaccagtctatcaccggactttacgagaccagaatcgatctttctcagcttggaggagataagagaccagctgctaccaagaaggctggacaggctaagaagaagaagtgagacgtccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgatcccccctcgacagcttccggaaagggcgaattcgcaactttgtatacaaaagttgccccatggcgttccctctagataacgcaggatccccaagtggtggctatgaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttggtgcaggcgagtgggggtgggtttaagagctatgctggaaacagcatagcaagtttaaataaggctagtccgttatcaacttgaaaaagtggcaccgagtcggtgcaacaaagcaccagtggtctagtggtagaatagtaccctgccacggtacagacccgggttcgattcccggctggtgcatgttcgaggcggcgctgcaggtttaagagctatgctggaaacagcatagcaagtttaaataaggctagtccgttatcaacttgaaaaagtggcaccgagtcggtgcaacaaagcaccagtggtctagtggtagaatagtaccctgccacggtacagacccgggttcgattcccggctggtgcagaaatcagaatctggtaccggtttaagagctatgctggaaacagcatagcaagtttaaataaggctagtccgttatcaacttgaaaaagtggcaccgagtcggtgcaacaaagcaccagtggtctagtggtagaatagtaccctgccacggtacagacccgggttcgattcccggctggtgcagctgttgagaggttcatgaggtttaagagctatgctggaaacagcatagcaagtttaaataaggctagtccgttatcaacttgaaaaagtggcaccgagtcggtgcaacaaagcaccagtggtctagtggtagaatagtaccctgccacggtacagacccgggttcgattcccggctggtgcatttttttgttttttatgtctccagactagtaagggca

SEQ ID NO: 53 - DNA, 7934 bp, synthetic construct
  source 1..7934: mol_type = other DNA
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggaatggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagactatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagattatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtctccacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtttcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaaggatctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtgagcttgtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgctactagaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgcctcggctggagctgcctgtgttttagagctagaaatagcaagttaaaataaggctagtccgttatcaacttgaaaaagtggcaccgagtcggtgctttttttctagacccagctttcttgtacaaagttggcattacgctttacttacgaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgacaggcagctccagccgagggttttagagctagaaatagcaagttaaaataaggctagtccgttatcaacttgaaaaagtggcaccgagtcggtgctttttttctagacccagctttcttgtacaaagttggcattacgctttaccagaaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgctgcaggggaacaccatcggttttagagctagaaatagcaagttaaaataaggctagtccgttatcaacttgaaaaagtggcaccgagtcggtgctttttttctagacccagctttcttgtacaaagttggcattacgctttac

SEQ ID NO: 54 - DNA, 7935 bp, synthetic construct
  source 1..7935: mol_type = other DNA
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggaatggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagactatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagattatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtctccacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtttcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaaggatctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtgagcttgtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgctactagaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgctcggctggagctgcctgtggttttagagctagaaatagcaagttaaaataaggctagtccgttatcaacttgaaaaagtggcaccgagtcggtgctttttttctagacccagctttcttgtacaaagttggcattacgctttacttacgaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgtaatgcgcgacctcctcggcgttttagagctagaaatagcaagttaaaataaggctagtccgttatcaacttgaaaaagtggcaccgagtcggtgctttttttctagacccagctttcttgtacaaagttggcattacgctttaccagaaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgccaggtggaggtgttcgagggttttagagctagaaatagcaagttaaaataaggctagtccgttatcaacttgaaaaagtggcaccgagtcggtgctttttttctagacccagctttcttgtacaaagttggcattacgctttac